(19) United States
(12) Patent Application Publication
(10) Pub. No.: US 2023/0132112 A1
(43) Pub. Date: Apr. 27, 2023

(54) LIGHT EMITTING DEVICE

(71) Applicant: Samsung Display Co., Ltd., Yongin-si (KR)

(72) Inventors: MUN-KI SIM, Seoul (KR); TAEIL KIM, Hwaseong-si (KR); SUN YOUNG PAK, Suwon-si (KR); JUNHA PARK, Gwacheon-si (KR); JANG YEOL BAEK, Yongin-si (KR); Kyoung SUNWOO, Hwaseong-si (KR); CHANSEOK OH, Seoul (KR); MINJUNG JUNG, Hongcheon-gun (KR)

(73) Assignee: Samsung Display Co., Ltd., Yongin-si (KR)

(21) Appl. No.: 17/825,408

(22) Filed: May 26, 2022

(30) Foreign Application Priority Data
Sep. 17, 2021 (KR) 10-2021-0124460

(51) Int. Cl. C07F 5/02; C07D 209/84; C07D 405/14; C07D 409/14; C07D 251/24; C07F 7/08; C07D 403/04; C07D 403/14; C07F 15/00; H01L 51/00

(52) U.S. Cl. CPC C07F 5/027; C07D 209/84; C07D 405/14; C07D 409/14; C07D 251/24; C07F 7/0812; C07D 403/04; C07D 403/14; C07F 15/0086; H01L 51/0094; H01L 51/0087; H01L 51/008; H01L 51/0072; H01L 51/0073; H01L 51/0074; H01L 51/0067; H01L 51/0071; H01L 51/5024

(57) ABSTRACT

A light emitting device of an embodiment includes a first electrode, a second electrode oppositely disposed to the first electrode, and an emission layer disposed between the first electrode and the second electrode. The emission layer includes a first host represented by Formula H-1, a second host represented by Formula H-2, and a first dopant represented by Formula 1, wherein Formulas H-1, H-2, and 1 are explained in the specification. The light emitting device shows improved emission efficiency properties.

### CROSS-REFERENCE TO RELATED APPLICATION(S)

This application claims priority to and benefits of Korean Patent Application No. 10-2021-0124460 under 35 U.S.C. § 119, filed on Sep. 17, 2021 in the Korean Intellectual Property Office, the entire contents of which are incorporated herein by reference.

### BACKGROUND

### 1. Technical Field

The disclosure relates to a light emitting device including multiple emission layer materials in an emission layer.

### 2. Description of the Related Art

Active development continues for an organic electroluminescence display as an image display. The organic electroluminescence display is different from a liquid crystal display and is a so-called self-luminescent display in which holes and electrons respectively injected from a first electrode and a second electrode recombine in an emission layer so that a light emitting material including an organic compound in the emission layer emits light to achieve display.

In the application of an organic electroluminescence device to an image display, there is a demand for decreasing driving voltage and increasing emission efficiency and the life of the organic electroluminescence device, and continuous development is required on materials for an organic electroluminescence device which is capable of stably achieving such characteristics.

Recently, in order to implement an organic electroluminescence device with high efficiency, techniques on phosphorescence emission which uses energy in a triplet state or delayed fluorescence emission which uses the generating phenomenon of singlet excitons by the collision of triplet excitons (triplet-triplet annihilation, TTA) are being developed, and development on a material for thermally activated delayed fluorescence (TADF) using delayed fluorescence phenomenon is being conducted.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

The disclosure provides a light emitting device showing excellent emission efficiency.

An embodiment provides a light emitting device which may include a first electrode, a second electrode facing the first electrode, and an emission layer disposed between the first electrode and the second electrode. The emission layer includes a first host represented by Formula H-1, a second host represented by Formula H-2, and a first dopant represented by Formula 1.

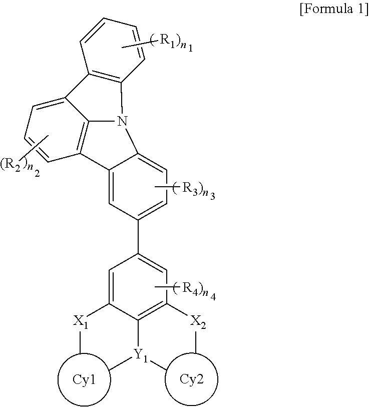

In Formula 1, X₁ and X₂ may each independently be N(R₅), O, or S; Y₁ may be B; Cy1 and Cy2 may each independently be a substituted or unsubstituted aromatic hydrocarbon ring of 6 to 30 ring-forming carbon atoms, or a substituted or unsubstituted aromatic heterocycle of 2 to 30 ring-forming carbon atoms, or may be combined with an adjacent group to form a ring; R₁ to R₄ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring; R₅ may be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring; n₁ may be an integer from 0 to 4; n₂ and n₃ may each independently be an integer from 0 to 3; and n₄ may be an integer from 0 to 2.

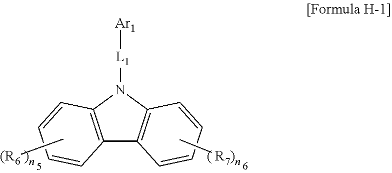

In Formula H-1, L₁ may be a direct linkage, a substituted or unsubstituted arylene group of 6 to 30 ring-forming carbon atoms, or a substituted or unsubstituted heteroarylene group of 2 to 30 ring-forming carbon atoms; An may be a substituted or unsubstituted aryl group of 6 to 30 ring-forming carbon atoms, or a substituted or unsubstituted heteroaryl group of 2 to 30 ring-forming carbon atoms; R₆ and R₇ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; and n₅ and n₆ may each independently be an integer from 0 to 4.

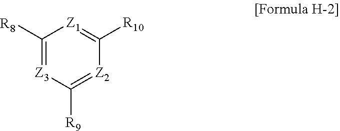

In Formula H-2, Z₁ to Z₃ may each independently be C(R₁₁) or N; at least one of Z₁ to Z₃ may be N; and R₈ to R₁₁ may each independently be a hydrogen atom, a deuterium atom, a cyano group, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms.

In an embodiment, Cy1 and Cy2 may each independently be a group represented by Formula 2.

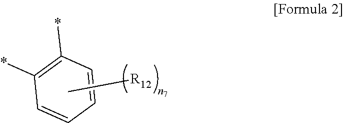

In Formula 2, R₁₂ may be a hydrogen atom, a deuterium atom, a halogen atom, a cyano group, a substituted or unsubstituted amine group, a substituted or unsubstituted oxy group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring; n₇ may be an integer from 0 to 4, and [symbol] are bonding sites to X₁ and Y₁ of Formula 1, or are bonding sites to X₂ and Y₁ of Formula 1.

In an embodiment, if Cy1 and Cy2 are each a group represented by Formula 2, then Cy1 and Cy2 may each independently be a group represented by Formula 2-1 or Formula 2-2.

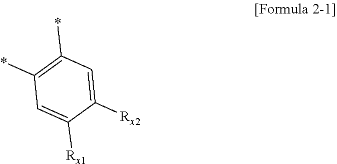

In Formula 2-1, Rₓ₁ and Rₓ₂ may each independently be a hydrogen atom, a deuterium atom, a halogen atom, a substituted or unsubstituted alkyl group of 1 to 20 carbon atoms, a substituted or unsubstituted phenyl group, a substituted or unsubstituted amine group, a substituted or unsubstituted carbazole group, or a substituted or unsubstituted indolocarbazole group, or may be combined with an adjacent group to form a ring; and [symbol] are bonding sites to X₁ and Y₁ of Formula 1, or are bonding sites to X₂ and Y₁ of Formula 1.

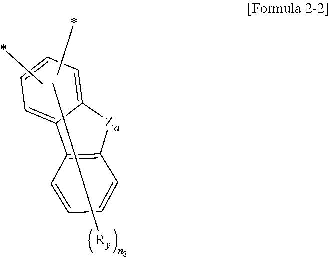

In Formula 2-2, Zₐ may be N(R₁₃) or O; R_{y} may be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring; R₁₃ may be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; n₈ may be an integer from 0 to 6; and [symbol] are bonding sites to X₁ and Y₁ of Formula 1, or are bonding sites to X₂ and Y₁ of Formula 1.

In an embodiment, Rig may be a substituted or unsubstituted alkyl group of 1 to 10 carbon atoms, a substituted or unsubstituted amine group, a substituted or unsubstituted oxy group, or a group represented by one of Formula 3-1 to Formula 3-4.

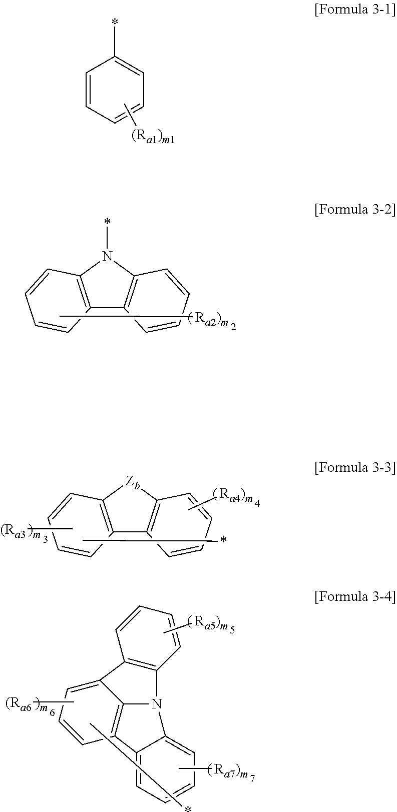

In Formula 3-1 to Formula 3-4, Z_{b} may be N(R₁₄) or O; Rₐ₁ to Rₐ₇ and R₁₄ may each independently be a hydrogen atom, a deuterium atom, a halogen atom, a cyano group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; m₁ may be an integer from 0 to 5; m₂ may be an integer from 0 to 8; m₃ to m₅, and m₇ may each independently be an integer from 0 to 4; m₆ may be an integer from 0 to 3, a sum of m₃ and m₄ may be equal to or less than 7, and a sum of m₆ and m₇ may be equal to or less than 6.

In an embodiment, the first dopant represented by Formula 1 may be represented by any one of Formula 4-1 or Formula 4-2.

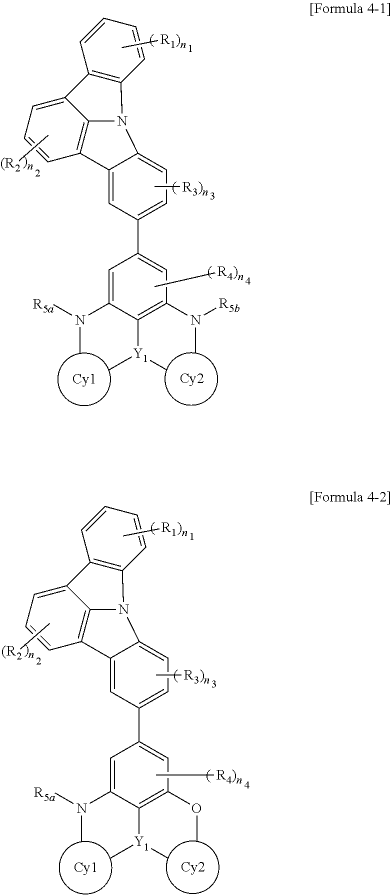

In Formula 4-1 and Formula 4-2, R₅ₐ and R_{5b} may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula 4-1 and Formula 4-2, Cy1, Cy2, Y₁, R₁ to R₄, and n₁ to n₄ are the same as defined in Formula 1.

In an embodiment, the first dopant represented by Formula 1 may be represented by any one of Formula 5-1 to Formula 5-3.

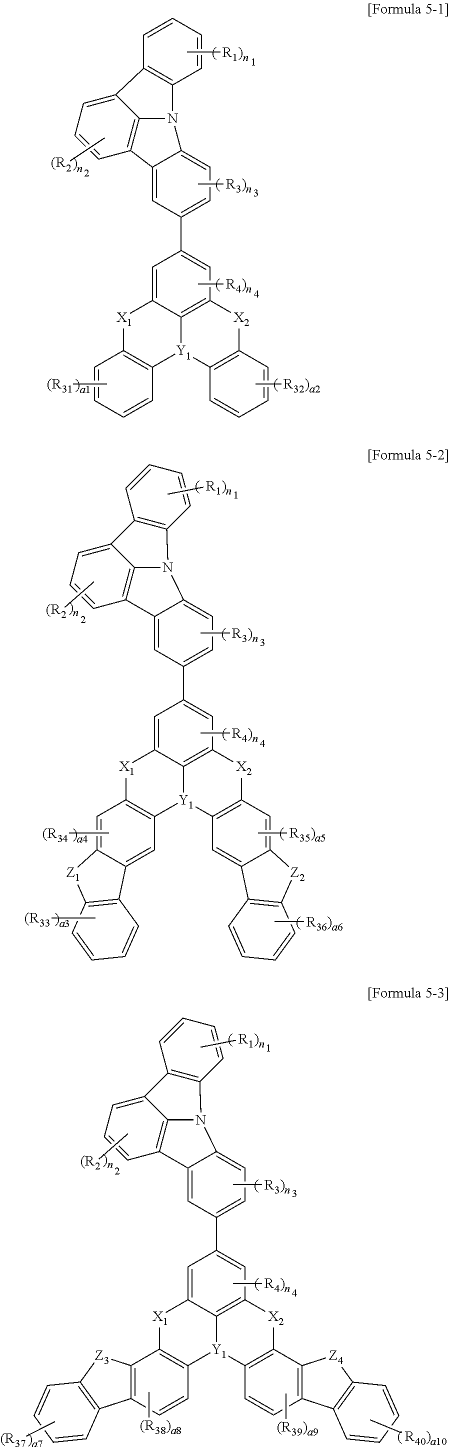

In Formula 5-1 to Formula 5-3, Z₁ to Z₄ may each independently be N(R₄₁) or O; R₃₁ to R₄₀ may each independently be a hydrogen atom, a deuterium atom, a halogen atom, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; R₄₁ may be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; a1 to a3, a6, a7, and a10 may each independently be an integer from 0 to 4; and a4, a5, a8, and a9 may each independently be an integer from 0 to 2.

In Formula 5-1 to Formula 5-3, X₁, X₂, Y₁, R₁ to R₄, and n₁ to n₄ are the same as defined in Formula 1.

In an embodiment, in Formula 1, if each of X₁ and X₂ in Formula 1 is N(R₅), then R₅ may be a group represented by any one of Formula 6-1 to Formula 6-4.

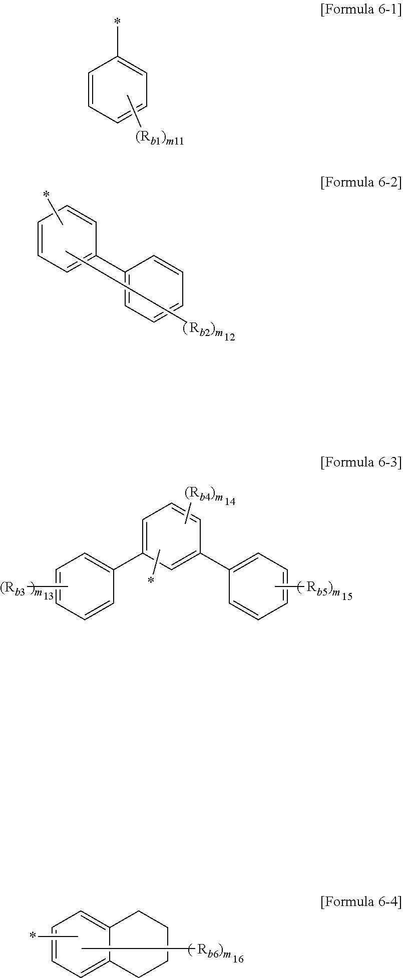

In Formula 6-1 to Formula 6-4, R_{b1} to R_{b6} may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; m₁₁, m₁₃, and m₁₅ may each independently be an integer from 0 to 5; m₁₂ may be an integer from 0 to 9; m₁₄ may be an integer from 0 to 3; and m₁₆ may be an integer from 0 to 11.

In an embodiment, the first dopant may include at least one selected from Compound Group 1, which is explained below.

In an embodiment, the first host may include at least one selected from Compound Group 2, which is explained below.

In an embodiment, the second host may include at least one selected from Compound Group 3, which is explained below.

In an embodiment, the emission layer may emit delayed fluorescence.

In an embodiment, the emission layer may emit light having a central emission wavelength in a range of about 430 nm to about 490 nm.

In an embodiment, the emission layer may further include a second dopant which is different from the first dopant, and the second dopant may be represented by Formula D-2.

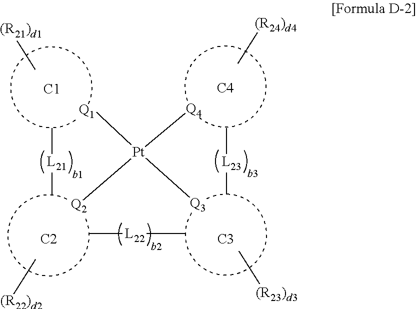

In Formula D-2, Q₁ to Q₄ may each independently be C or N; C1 to C4 may each independently be a substituted or unsubstituted hydrocarbon ring of 5 to 30 ring-forming carbon atoms, or a substituted or unsubstituted heterocycle of 2 to 30 ring-forming carbon atoms; L₂₁ to L₂₃ may each independently be a direct linkage,

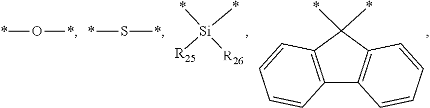

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms; b1 to b3 may each independently be 0 or 1; R₂₁ to R₂₆ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 1 to 30 ring-forming carbon atoms, or may be combined with an adjacent group to form a ring; and d1 to d4 may each independently be an integer from 0 to 4.

In an embodiment, the second dopant may include at least one selected from Compound Group 4, which is explained below.

In an embodiment, the light emitting device may further include a hole transport region disposed between the first electrode and the emission layer, wherein the hole transport region may include a compound represented by Formula H-a.

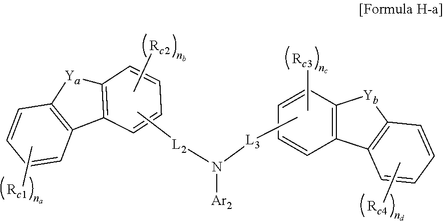

In Formula H-a, Yₐ and Y_{b} may each independently be C(R_{c5})(R_{c6}), N(R_{c7}), O, or S; Ar₂ may be a substituted or unsubstituted aryl group of 6 to 30 ring-forming carbon atoms, or a substituted or unsubstituted heteroaryl group of 2 to 30 ring-forming carbon atoms; L₂ and L₃ may each independently be a direct linkage, a substituted or unsubstituted arylene group of 6 to 30 ring-forming carbon atoms, or a substituted or unsubstituted heteroarylene group of 2 to 30 ring-forming carbon atoms; R_{c1} to R_{c7} may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring; nₐ and n_{d} may each independently be an integer from 0 to 4; and n_{b} and n_{c} may each independently be an integer from 0 to 3.

An embodiment provides a light emitting device which may include a first electrode, a second electrode facing the first electrode, and an emission layer disposed between the first electrode and the second electrode. The emission layer may include a host and a dopant, and the dopant may include a first dopant represented by Formula 1, and a second dopant represented by Formula D-2.

In an embodiment, Cy1 and Cy2 may each independently be a group represented by Formula 2.

In an embodiment, if Cy1 and Cy2 are each a group represented by Formula 2, then Cy1 and Cy2 may each independently be a group represented by Formula 2-1 or Formula 2-2.

In an embodiment, the host may include a first host represented by Formula H-1, and a second host represented by Formula H-2.

In an embodiment, the first dopant may include at least one selected from Compound Group 1, which is explained below.

### BRIEF DESCRIPTION OF THE DRAWINGS

The accompanying drawings are included to provide a further understanding of the embodiments, and are incorporated in and constitute a part of this specification. The drawings illustrate embodiments of the disclosure and principles thereof. The above and other aspects and features of the disclosure will become more apparent by describing in detail embodiments thereof with reference to the attached drawings, in which:

FIG. 1 is a plan view of a display apparatus according to an embodiment;

FIG. 2 is a schematic cross-sectional view of a display apparatus according to an embodiment;

FIG. 3 is a schematic cross-sectional view showing a light emitting device according to an embodiment;

FIG. 4 is a schematic cross-sectional view showing a light emitting device according to an embodiment;

FIG. 5 is a schematic cross-sectional view showing a light emitting device according to an embodiment;

FIG. 6 is a schematic cross-sectional view showing a light emitting device according to an embodiment;

FIG. 7 is a schematic cross-sectional view showing a display apparatus according to an embodiment;

FIG. 8 is a schematic cross-sectional view showing a display apparatus according to an embodiment;

FIG. 9 is a schematic cross-sectional view showing a display apparatus according to an embodiment; and

FIG. 10 is a schematic cross-sectional view showing a display apparatus according to an embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like numbers refer to like elements throughout.

In the specification, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the specification, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

The term “at least one of” is intended to include the meaning of “at least one selected from the group of” for the purpose of its meaning and interpretation. For example, “at least one of A and B” may be understood to mean “A, B, or A and B.” When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

Hereinafter, embodiments will be explained with reference to the drawings.

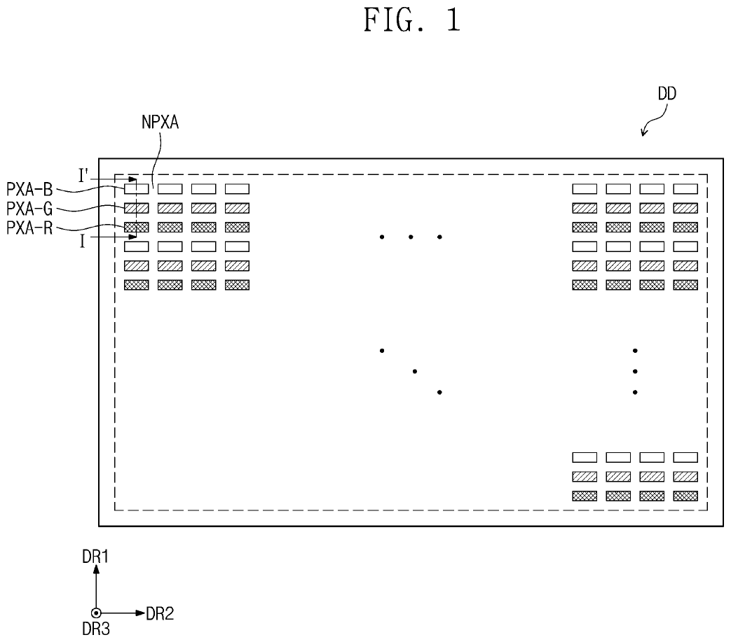

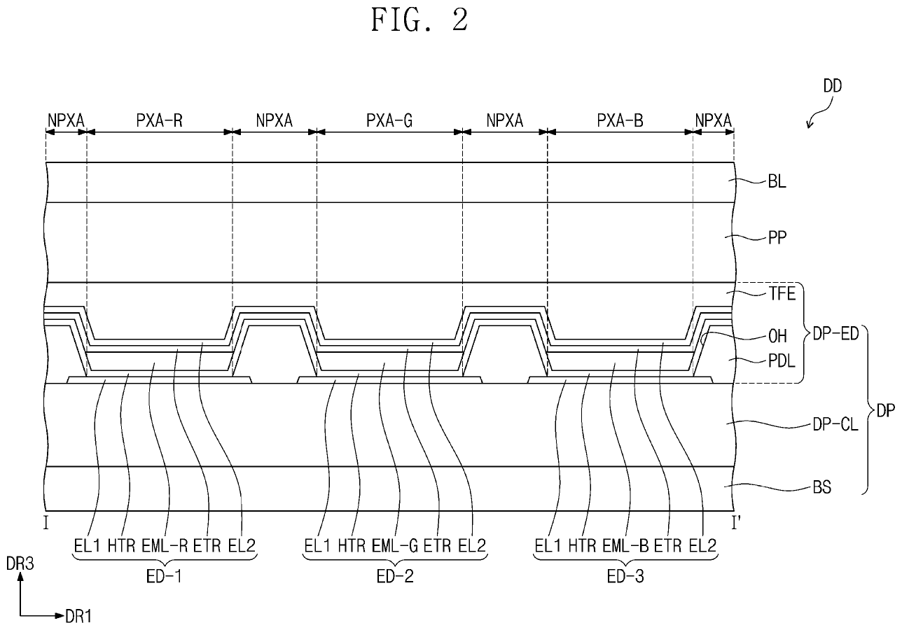

FIG. 1 is a plan view showing an embodiment of a display apparatus DD. FIG. 2 is a schematic cross-sectional view of a display apparatus DD of an embodiment. FIG. 2 is a schematic cross-sectional view showing a part corresponding to line I-I′ of FIG. 1.

The display apparatus DD may include a display panel DP and an optical layer PP disposed on the display panel DP. The display panel DP includes light emitting devices ED-1, ED-2, and ED-3. The display apparatus DD may include multiples of each of the light emitting devices ED-1, ED-2, and ED-3. The optical layer PP may be disposed on the display panel DP and may control light reflected at the display panel DP from an external light. The optical layer PP may include, for example, a polarization layer or a color filter layer. Although not shown in the drawings, in an embodiment, the optical layer PP may be omitted from the display apparatus DD.

A base substrate BL may be disposed on the optical layer PP. The base substrate BL may provide a base surface on which the optical layer PP is disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

The display apparatus DD according to an embodiment may further include a plugging layer (not shown). The plugging layer (not shown) may be disposed between a display device layer DP-ED and a base substrate BL. The plugging layer (not shown) may be an organic layer. The plugging layer (not shown) may include at least one of an acrylic resin, a silicon-based resin, or an epoxy-based resin.

The display panel DP may include a base layer BS, a circuit layer DP-CL provided on the base layer BS, and a display device layer DP-ED. The display device layer DP-ED may include a pixel definition layer PDL, light emitting devices ED-1, ED-2, and ED-3 disposed in the pixel definition layer PDL, and an encapsulating layer TFE disposed on the light emitting devices ED-1, ED-2, and ED-3.

The base layer BS may provide a base surface on which the display device layer DP-ED is disposed. The base layer BS may be a glass substrate, a metal substrate, a plastic substrate, etc. However, embodiments are not limited thereto, and the base layer BS may include an inorganic layer, an organic layer, or a composite material layer.

In an embodiment, the circuit layer DP-CL is disposed on the base layer BS, and the circuit layer DP-CL may include transistors (not shown). Each of the transistors (not shown) may include a control electrode, an input electrode, and an output electrode. For example, the circuit layer DP-CL may include switching transistors and driving transistors for driving the light emitting devices ED-1, ED-2, and ED-3 of the display device layer DP-ED.

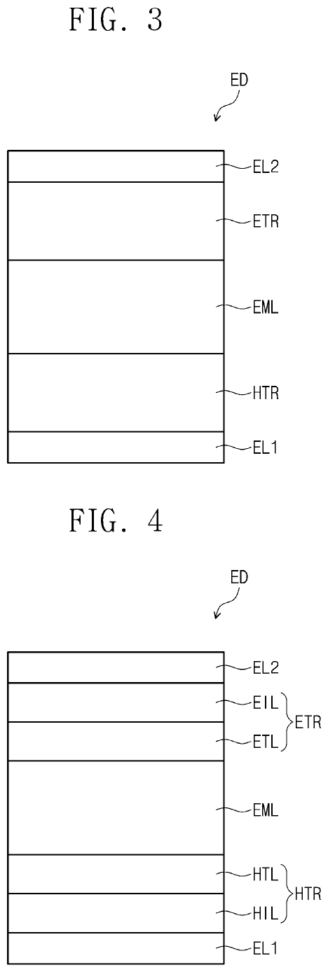

Each of the light emitting devices ED-1, ED-2, and ED-3 may have a structure of a light emitting device ED of embodiments according to FIG. 3 to FIG. 6, which will be explained later. Each of the light emitting devices ED-1, ED-2, and ED-3 may include a first electrode EL1, a hole transport region HTR, emission layers EML-R, EML-G, and EML-B, an electron transport region ETR, and a second electrode EL2.

FIG. 2 shows an embodiment where the emission layers EML-R, EML-G, and EML-B of light emitting devices ED-1, ED-2 and ED-3 are disposed in openings OH defined in a pixel definition layer PDL, and a hole transport region HTR, an electron transport region ETR, and a second electrode EL2 are each provided as common layers for all of the light emitting devices ED-1, ED-2, and ED-3. However, embodiments are not limited thereto. Although not shown in FIG. 2, in an embodiment, the hole transport region HTR and the electron transport region ETR may each be patterned and provided in the openings OH defined in the pixel definition layer PDL. For example, in an embodiment, the hole transport region HTR, the emission layers EML-R, EML-G, and EML-B, and the electron transport region ETR of the light emitting devices ED-1, ED-2, and ED-3 may each be patterned by an ink jet printing method and provided.

An encapsulating layer TFE may cover the light emitting devices ED-1, ED-2, and ED-3. The encapsulating layer TFE may encapsulate the display device layer DP-ED. The encapsulating layer TFE may be a thin film encapsulating layer. The encapsulating layer TFE may be a single layer or a stack of multiple layers. The encapsulating layer TFE may include at least one insulating layer. The encapsulating layer TFE according to an embodiment may include at least one inorganic layer (hereinafter, an encapsulating inorganic layer). The encapsulating layer TFE according to an embodiment may include at least one organic layer (hereinafter, an encapsulating organic layer) and at least one encapsulating inorganic layer.

The encapsulating inorganic layer may protect the display device layer DP-ED from moisture and/or oxygen, and the encapsulating organic layer may protect the display device layer DP-ED from foreign materials such as dust particles. The encapsulating inorganic layer may include silicon nitride, silicon oxy nitride, silicon oxide, titanium oxide, or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be disposed on the second electrode EL2 and may be disposed to fill the openings OH.

Referring to FIG. 1 and FIG. 2, the display apparatus DD may include non-luminous areas NPXA and luminous areas PXA-R, PXA-G, and PXA-B. The luminous areas PXA-R, PXA-G, and PXA-B may each be an area emitting light produced from the light emitting devices ED-1, ED-2, and ED-3, respectively. The luminous areas PXA-R, PXA-G, and PXA-B may be spaced apart from each other in a plan view.

The luminous areas PXA-R, PXA-G, and PXA-B may be areas separated by the pixel definition layer PDL. The non-luminous areas NPXA may be areas between neighboring luminous areas PXA-R, PXA-G, and PXA-B and may be areas corresponding to the pixel definition layer PDL. For example, in an embodiment, each of the luminous areas PXA-R, PXA-G, and PXA-B may correspond to a pixel. The pixel definition layer PDL may separate the light emitting devices ED-1, ED-2, and ED-3. The emission layers EML-R, EML-G, and EML-B of the light emitting devices ED-1, ED-2, and ED-3 may be disposed in the openings OH defined in the pixel definition layer PDL and separated from each other.

The luminous areas PXA-R, PXA-G, and PXA-B may be divided into groups according to the color of light produced from the light emitting devices ED-1, ED-2, and ED-3. In the display apparatus DD of an embodiment shown in FIG. 1 and FIG. 2, three luminous areas PXA-R, PXA-G, and PXA-B emitting red light, green light, and blue light are illustrated as an embodiment. For example, the display apparatus DD of an embodiment may include a red luminous area PXA-R, a green luminous area PXA-G, and a blue luminous area PXA-B, which are separated from each other.

In the display apparatus DD according to an embodiment, light emitting devices ED-1, ED-2, and ED-3 may emit light having different wavelength regions. For example, in an embodiment, the display apparatus DD may include a first light emitting device ED-1 emitting red light, a second light emitting device ED-2 emitting green light, and a third light emitting device ED-3 emitting blue light. For example, the red luminous area PXA-R, the green luminous area PXA-G, and the blue luminous area PXA-B of the display apparatus DD may respectively correspond to the first light emitting device ED-1, the second light emitting device ED-2, and the third light emitting device ED-3.

However, embodiments are not limited thereto, and the first to third light emitting devices ED-1, ED-2, and ED-3 may emit light in a same wavelength region, or at least one thereof may emit light in a different wavelength region. For example, the first to third light emitting devices ED-1, ED-2, and ED-3 may all emit blue light.

The luminous areas PXA-R, PXA-G, and PXA-B in the display apparatus DD according to an embodiment may be arranged in a stripe configuration. Referring to FIG. 1, red luminous areas PXA-R, green luminous areas PXA-G, and blue luminous areas PXA-B may be arranged along a second directional DR2. In another embodiment, the red luminous area PXA-R, the green luminous area PXA-G, and the blue luminous area PXA-B may be arranged by turns along a first directional DR1.

In FIG. 1 and FIG. 2, the areas of the luminous areas PXA-R, PXA-G, and PXA-B are shown as similar in size, but embodiments are not limited thereto. The areas of the luminous areas PXA-R, PXA-G, and PXA-B may be different from each other according to a wavelength region of light emitted. The areas of the luminous areas PXA-R, PXA-G, and PXA-B may be areas in a plan view that are defined by the first directional axis DR1 and the second directional axis DR2.

The arrangement type of the luminous areas PXA-R, PXA-G, and PXA-B is not limited to the configuration shown in FIG. 1, and the arrangement order of the red luminous areas PXA-R, the green luminous areas PXA-G, and the blue luminous areas PXA-B may be provided in various combinations according to the display quality characteristics which are required for the display apparatus DD. For example, the arrangement type of the luminous areas PXA-R, PXA-G, and PXA-B may be a PENTILE® configuration or a diamond configuration.

In an embodiment, the areas of the luminous areas PXA-R, PXA-G, and PXA-B may be different from each other. For example, in an embodiment, an area of the green luminous area PXA-G may be smaller in size than an area of the blue luminous area PXA-B, but embodiments are not limited thereto.

Hereinafter, FIG. 3 to FIG. 6 are each a schematic cross-sectional view showing a light emitting device according to embodiments. The light emitting device ED according to an embodiment may include a first electrode EL1, a hole transport region HTR, an emission layer EML, an electron transport region ETR, and a second electrode EL2, stacked in that order.

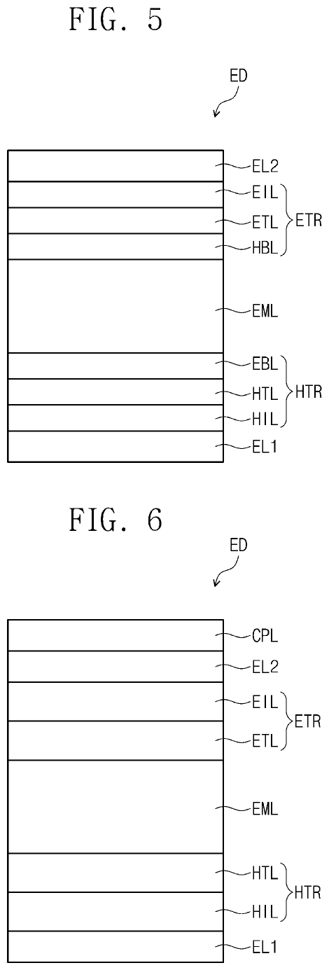

In comparison to FIG. 3, FIG. 4 shows a schematic cross-sectional view of a light emitting device ED of an embodiment, wherein a hole transport region HTR includes a hole injection layer HIL and a hole transport layer HTL, and an electron transport region ETR includes an electron injection layer EIL and an electron transport layer ETL. In comparison to FIG. 3, FIG. 5 shows a schematic cross-sectional view of a light emitting device ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. In comparison to FIG. 4, FIG. 6 shows a schematic cross-sectional view of a light emitting device ED of an embodiment that includes a capping layer CPL disposed on the second electrode EL2.

The first electrode EL1 has conductivity. The first electrode EL1 may be formed of a metal material, a metal alloy, or a conductive compound. The first electrode EL1 may be an anode or a cathode. However, embodiments are not limited thereto. For example, the first electrode EL1 may be a pixel electrode. The first electrode EL1 may be a transmissive electrode, a transflective electrode, or a reflective electrode. If the first electrode EL1 is a transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, W, compounds thereof, or mixtures thereof (for example, a mixture of Ag and Mg). In another embodiment, the first electrode EL1 may have a structure including multiple layers including a reflective layer or a transflective layer formed of the above materials, and a transmissive conductive layer formed of ITO, IZO, ZnO, or ITZO. For example, the first electrode EL1 may include a three-layer structure of ITO/Ag/ITO. However, embodiments are not limited thereto. The first electrode EL1 may include the above-described metal materials, combinations of two or more metal materials selected from the above-described metal materials, or oxides of the above-described metal materials. A thickness of the first electrode EL1 may be in a range of about 700 Å to about 10,000 Å. For example, the thickness of the first electrode EL1 may be in a range of about 1,000 Å to about 3,000 Å.

The hole transport region HTR is provided on the first electrode EL1. The hole transport region HTR may include at least one of a hole injection layer HIL, a hole transport layer HTL, a buffer layer (not shown), an emission auxiliary layer (not shown), or an electron blocking layer EBL. A thickness of the hole transport region HTR may be in a range of about 50 Å to about 15,000 Å.

The hole transport region HTR may be a layer formed of a single material, a layer formed of different materials, or a multilayer structure including layers formed of different materials.

For example, the hole transport region HTR may have the structure of a single layer of a hole injection layer HIL or a hole transport layer HTL, or may have a structure of a single layer formed of a hole injection material and a hole transport material. In other embodiments, the hole transport region HTR may have a structure of a single layer formed of different materials, or a structure in which a hole injection layer HIL/hole transport layer HTL, a hole injection layer HIL/hole transport layer HTL/buffer layer (not shown), a hole injection layer HIL/buffer layer (not shown), a hole transport layer HTL/buffer layer (not shown), or a hole injection layer HIL/hole transport layer HTL/electron blocking layer EBL, are stacked in its respective stater order from the first electrode EL1, but embodiments are not limited thereto.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The hole transport region HTR may include a compound represented by Formula H.

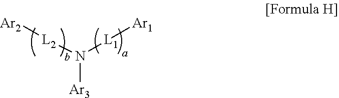

In Formula H, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. In Formula H, a and b may each independently be an integer from 0 to 10. If a or b is 2 or more, multiple L₁ groups and multiple L₂ groups may each independently be a substituted or unsubstituted arylene group of 6 to 30 ring-forming carbon atoms, or a substituted or unsubstituted heteroarylene group of 2 to 30 ring-forming carbon atoms.

In Formula H, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. In Formula H, Ar₃ may be a substituted or unsubstituted aryl group of 6 to 30 ring-forming carbon atoms.

In an embodiment, the compound represented by Formula H may be a monoamine compound. In another embodiment, the compound represented by Formula H may be a diamine compound in which at least one of Ar₁ to Ar₃ includes an amine group as a substituent. In still another embodiment, the compound represented by Formula H may be a carbazole-based compound in which at least one of Ar₁ and Ar₂ includes a substituted or unsubstituted carbazole group, or the compound represented by Formula H may be a fluorene-based compound in which at least one of Ar₁ and Ar₂ includes a substituted or unsubstituted fluorene group.

The compound represented by Formula H may be any one selected from Compound Group H. However, the compounds shown in Compound Group H are only examples, and the compound represented by Formula H is not limited to Compound Group H.

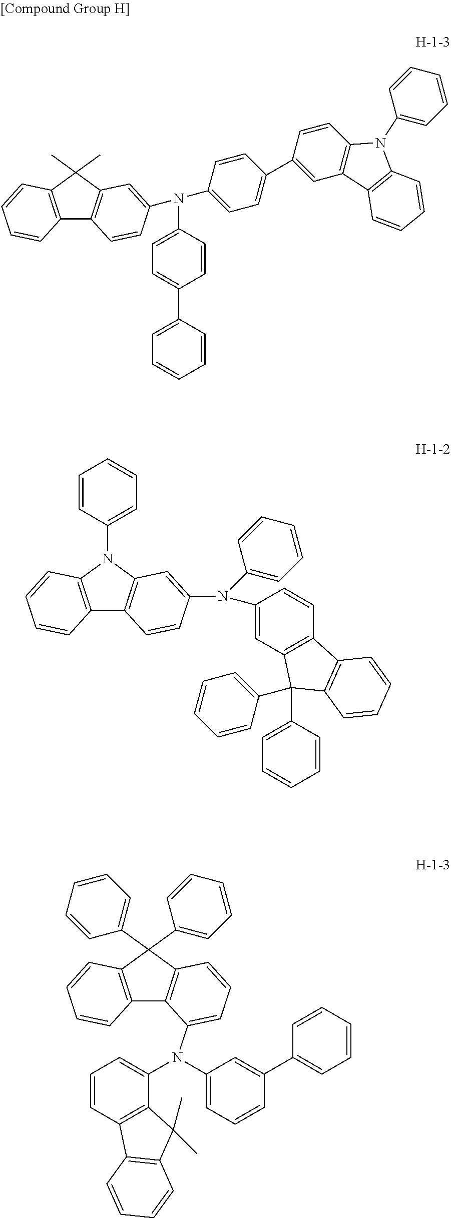 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

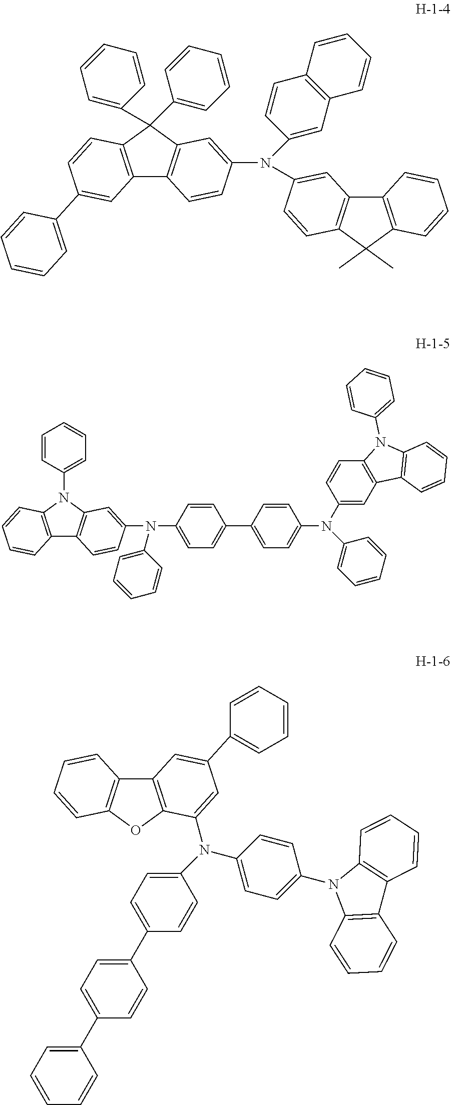 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

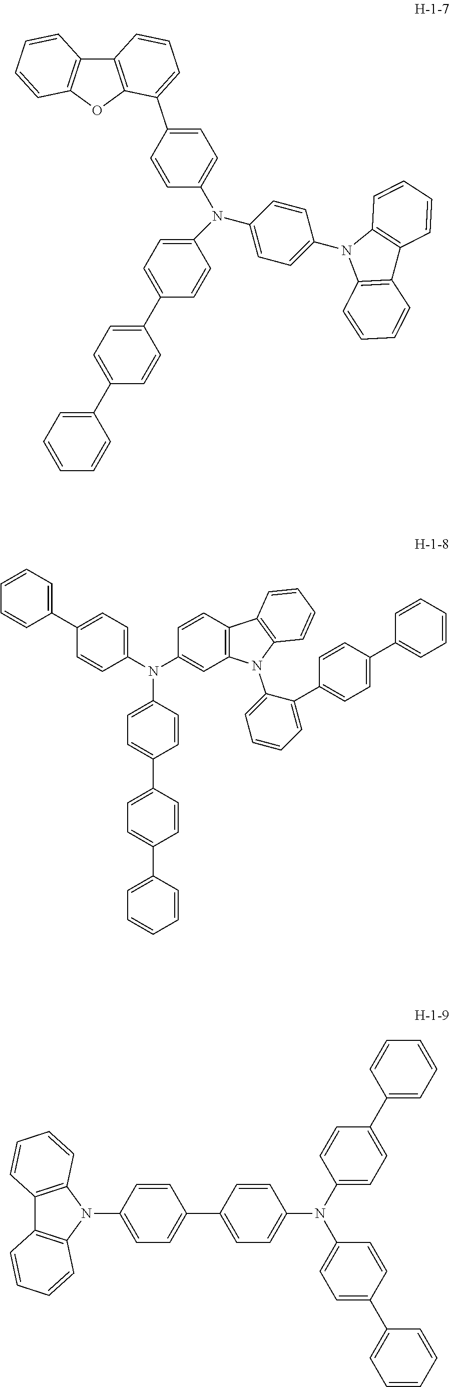 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

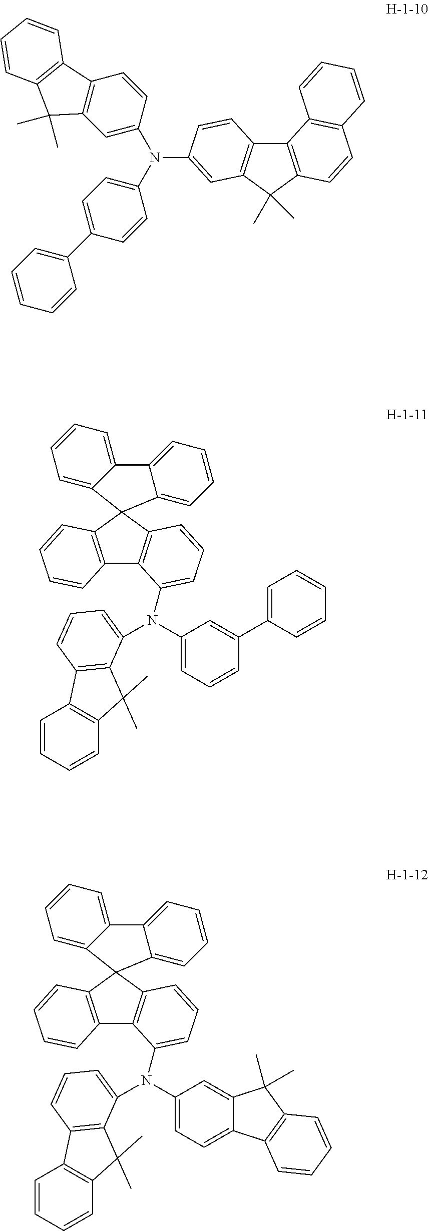 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21]

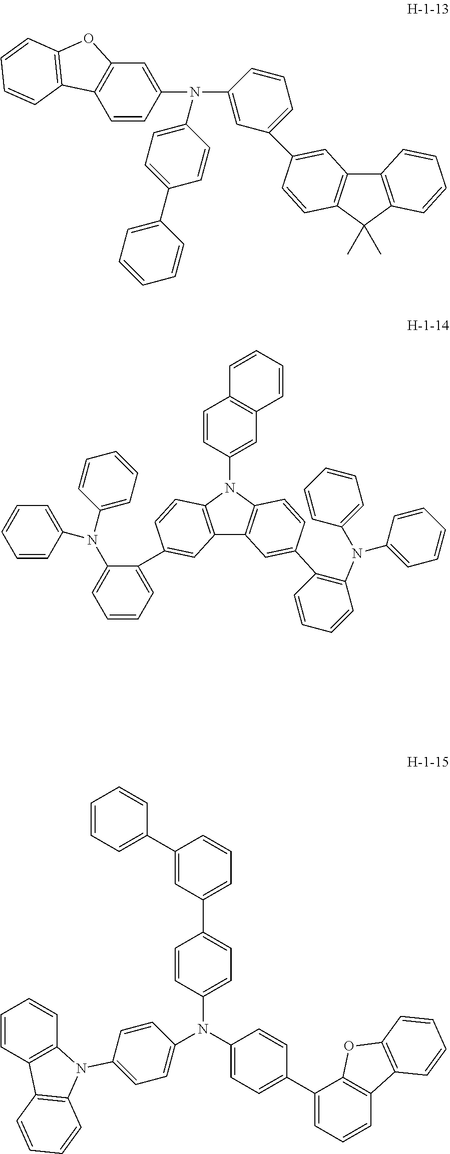 [SMILES: CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

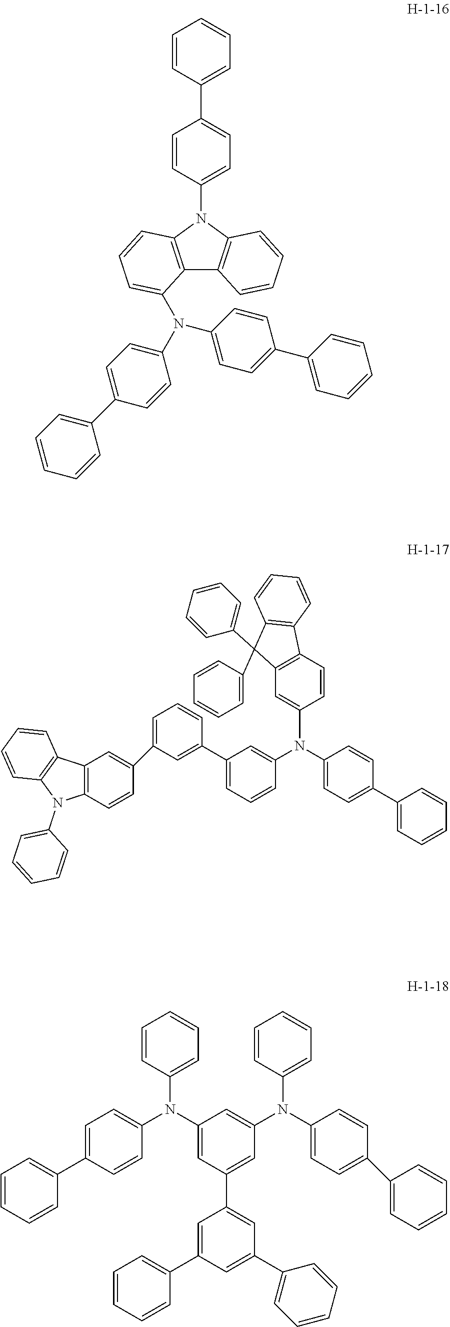 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

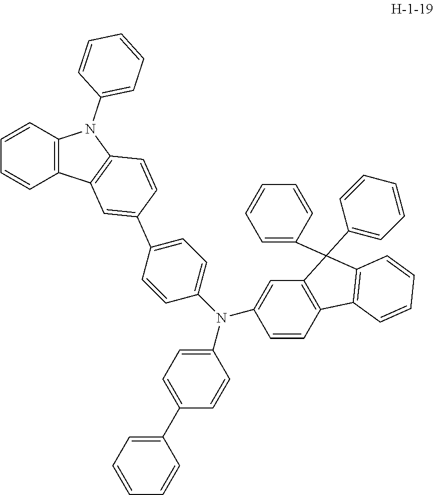 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a compound represented by Formula H-a. The compound represented by Formula H-a may be a monoamine compound.

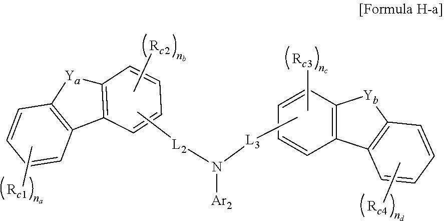

In Formula H-a, Yₐ and Y_{b} may each independently be C(R_{c5})(R_{c6}), N(R_{c7}), O, or S. Yₐ and Y_{b} may be the same as or different from each other. In an embodiment, Yₐ and Y_{b} may each independently be C(R_{c5})(R_{c6}). In another embodiment, any one of Yₐ and Y_{b} may be C(R_{c5})(R_{c6}), and the other of Yₐ and Y_{b} may be N(R_{c7}).

In Formula H-a, Ar₂ may be a substituted or unsubstituted aryl group of 6 to 30 ring-forming carbon atoms, or a substituted or unsubstituted heteroaryl group of 2 to 30 ring-forming carbon atoms. For example, Ar₂ may be a substituted or unsubstituted phenyl group, a substituted or unsubstituted biphenyl group, a substituted or unsubstituted naphthyl group, a substituted or unsubstituted fluorenyl group, or a substituted or unsubstituted terphenyl group.

In Formula H-a, L₂ and L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. For example, L₂ and L₃ may each independently be a direct linkage, a substituted or unsubstituted phenylene group, or a substituted or unsubstituted divalent biphenyl group.

In Formula H-a, R_{c1} to R_{c7} may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. For example, R_{c1} to R_{c7} may each independently be a hydrogen atom, a substituted or unsubstituted methyl group, or a substituted or unsubstituted phenyl group.

In Formula H-a, nₐ and n_{d} may each independently be an integer from 0 to 4, and n_{b} and n_{c} may each independently be an integer from 0 to 3.

The hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f:2′,3′-h] quinoxaline-2,3,6,7,10,11-hexacarbonitrile (HAT-CN), or the like.

The hole transport region HTR may include carbazole derivatives such as N-phenyl carbazole and polyvinyl carbazole, fluorene-based derivatives, N,N′-bis(3-methylphenyl)-N,N′-diphenyl-[1,1′-biphenyl]-4,4′-diamine (TPD), triphenylamine-based derivatives such as 4,4′,4″-tris(N-carbazolyl)triphenylamine (TCTA), N,N′-di(1-naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), etc.

The hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the compounds of the hole transport region in at least one of a hole injection layer HIL, a hole transport layer HTL, or an electron blocking layer EBL.

A thickness of the hole transport region HTR may be in a range of about 100 Å to about 10,000 Å. For example, the thickness of the hole transport region HTR may be in a range of about 100 Å to about 5,000 Å. In case that the hole transport region HTR includes a hole injection layer HIL, a thickness of the hole injection region HIL may be in a range of about 30 Å to about 1,000 Å. In case that the hole transport region HTR includes a hole transport layer HTL, a thickness of the hole transport layer HTL may be in a range of about 30 Å to about 1,000 Å. In case that the hole transport region HTR includes an electron blocking layer, a thickness of the electron blocking layer EBL may be in a range of about 10 Å to about 1,000 Å. If the thicknesses of the hole transport region HTR, the hole injection layer HIL, the hole transport layer HTL, and the electron blocking layer EBL satisfy the above-described ranges, satisfactory hole transport properties may be achieved without a substantial increase of driving voltage.

The hole transport region HTR may further include a charge generating material to increase conductivity, in addition to the above-described materials. The charge generating material may be dispersed uniformly or non-uniformly in the hole transport region HTR. The charge generating material may be, for example, a p-dopant. The p-dopant may include at least one of metal halide compounds, quinone derivatives, metal oxides, or cyano group-containing compounds, without limitation. For example, the p-dopant may include metal halide compounds such as CuI and RbI, quinone derivatives such as tetracyanoquinodimethane (TCNQ) and 2,3,5,6-tetrafluoro-7,7′,8,8-tetracyanoquinodimethane (F4-TCNQ), metal oxides such as tungsten oxide and molybdenum oxide, cyano group-containing compounds such as dipyrazino[2,3-f: 2′,3′-h] quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile, etc., without limitation.

As described above, the hole transport region HTR may further include at least one of a buffer layer (not shown) or an electron blocking layer EBL, in addition to the hole injection layer HIL and the hole transport layer HTL. The buffer layer (not shown) may compensate for a resonance distance according to a wavelength of light emitted from an emission layer EML and may increase light emitting efficiency. Materials which may be included in the hole transport region HTR may be used as materials included in the buffer layer (not shown). The electron blocking layer EBL may block the injection of electrons from an electron transport region ETR to a hole transport region HTR.

The emission layer EML is provided on the hole transport region HTR. The emission layer EML may have a thickness in a range of about 100 Å to about 1,000 Å. For example, the emission layer EML may have a thickness in a range of about 100 Å to about 300 Å. The emission layer EML may be a layer formed of a single material, a layer formed of different materials, or a multilayer structure having layers formed of different materials.

In the light emitting device ED of an embodiment, the emission layer EML may include multiple light emitting materials. The light emitting device ED of an embodiment may include at least one host and at least one dopant. For example, the light emitting device ED of an embodiment may include a first host and a second host, which are different from each other, and a first dopant. In another embodiment, the light emitting device ED of an embodiment may include a host, and a first dopant and a second dopant, which are different from each other.

In the description, the term “substituted or unsubstituted” may mean a group that is substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. Each of the substituents listed above may itself be substituted or unsubstituted. For example, a biphenyl group may be interpreted as an aryl group, or may be interpreted as a phenyl group substituted with a phenyl group.

In the description, the term “combined with an adjacent group to form a ring” may mean a group that is bonded to an adjacent group to form a substituted or unsubstituted hydrocarbon ring, or a substituted or unsubstituted heterocycle. The hydrocarbon ring may be an aliphatic hydrocarbon ring or an aromatic hydrocarbon ring. The heterocycle may be an aliphatic heterocycle or an aromatic heterocycle. The hydrocarbon ring and the heterocycle may each independently be monocyclic or polycyclic. A ring that is formed by the combination of adjacent groups may itself be combined with another ring to form a spiro structure.

In the description, the term “adjacent group” may mean a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. For example, in 4,5-dimethylphenanthrene, two methyl groups may be interpreted as “adjacent groups” to each other.

In the description, examples of a halogen atom may include a fluorine atom, a chlorine atom, a bromine atom, or an iodine atom.

In the description, an alkyl group may be a linear, a branched, or a cyclic type. The number of carbon atoms in the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the description, a hydrocarbon ring group may be any functional group or substituent derived from an aliphatic hydrocarbon ring. The hydrocarbon ring group may be a saturated hydrocarbon ring group of 5 to 20 ring-forming carbon atoms.

In the description, an aryl group may be any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

In the description, a heterocyclic group may be any functional group or substituent derived from a ring including one or more of B, O, N, P, Si, or S as heteroatoms. The heterocyclic group may be an aliphatic heterocyclic group or an aromatic heterocyclic group. The aromatic heterocyclic group may be a heteroaryl group. The aliphatic heterocyclic group and the aromatic heterocyclic group may each independently be monocyclic or polycyclic.

In the description, a heterocyclic group may include one or more of B, O, N, P, Si, or S as heteroatoms. If the heterocyclic group includes two or more heteroatoms, the two or more heteroatoms may be the same as or different from each other. The heterocyclic group may be a monocyclic heterocyclic group or a polycyclic heterocyclic group, and the heterocyclic group may be a heteroaryl group. The number of ring-forming carbon atoms in the heteroaryl group may be 2 to 30, 2 to 20, and 2 to 10.

In the description, a heteroaryl group may include one or more of B, O, N, P, Si, or S as heteroatoms. If the heteroaryl group includes two or more heteroatoms, the two or more heteroatoms may be the same as or different from each other. The heteroaryl group may be monocyclic or polycyclic. The number of ring-forming carbon atoms in the heteroaryl group may be 2 to 30, 2 to 20, or 2 to 10. Examples of the heteroaryl group may include thiophene, furan, pyrrole, imidazole, triazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the description, the same explanation on the above-described aryl group may be applied to an arylene group, except that the arylene group is a divalent group. The same explanation on the above-described heteroaryl group may be applied to a heteroarylene group, except that the heteroarylene group is a divalent group.

In the description, a silyl group may be an alkyl silyl group or an aryl silyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the description, a thio group may be an alkyl thio group or an aryl thio group. The thio group may be a sulfur atom bonded to an alkyl group or an aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the description, an oxy group may be an oxygen atom bonded to an alkyl group or an aryl group as defined above. The oxy group may be an alkoxy group or an aryl oxy group. The alkoxy group may be a linear, branched, or cyclic chain. The number of carbon atoms in the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc. However, embodiments are not limited thereto.

In the description, the number of carbon atoms in an amine group is not specifically limited, but may be 1 to 30. The amine group may be an alkyl amine group or an aryl amine group. Examples of the amine group may include a methylamine group, a dimethylamine group, a phenylamine group, a diphenylamine group, a naphthylamine group, a 9-methyl-anthracenylamine group, a triphenylamine group, etc., without limitation.

In the description, a direct linkage may be a single bond.

In the description, [symbol] or [symbol] each represents a bonding position to a neighboring atom.

In a light emitting device ED of an embodiment, the emission layer EML may include a compound represented by Formula 1 as a dopant. In the light emitting device ED according to an embodiment, the emission layer EML may include a fused polycyclic compound represented by Formula 1 as a first dopant.

The first dopant of an embodiment has a structure including at least one indolocarbazole group in a plate type resonance structure that includes a boron atom. In the description, an indolocarbazole group may be an aromatic heterocycle forming a fused structure of three benzene rings with a nitrogen atom as a center. In the first dopant of an embodiment, the indolocarbazole group may be bonded to a benzene ring which is connected with a boron atom at a central core. The boron atom and the indolocarbazole group may be bonded to a benzene ring at a para position to each other. The first dopant of an embodiment may have a structure in which an additional aromatic structure is fused to the boron atom and to heteroatoms, connected by a benzene ring, so that a conjugation length may be extended.

The indolocarbazole group may be combined with the central core at a carbon of position 5 or a carbon of position 10. For example, a carbon atom at a para position with respect to a nitrogen atom among the carbon atoms composing a benzene ring forming an indolocarbazole skeleton may be bonded to the benzene ring bonded to the boron atom. Accordingly, by increasing the electron donating properties of the indolocarbazole group and at the same time, by the suppression of quenching phenomenon due to intermolecular interaction by the reduction of symmetry of a whole molecule, the emission efficiency of a light emitting device may be further improved. The carbon number in the indolocarbazole group is shown in Formula a.

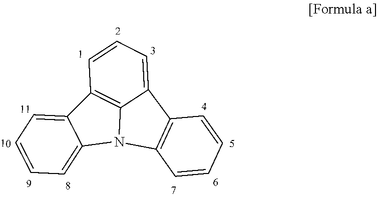 [SMILES: c1ccc2c(c1)c1cccc3c4ccccc4n2c13]

The first dopant of an embodiment may be represented by Formula 1.

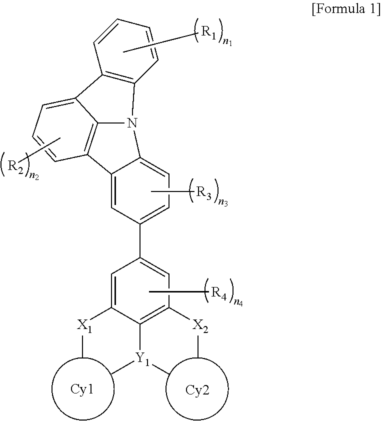

In Formula 1, X₁ to X₂ may each independently be N(R₅), O, or S. X₁ and X₂ may be the same as or different from each other. For example, X₁ and X₂ may each be N(R₅), may each be O, or may each be S. In another embodiment, any one of X₁ and X₂ may be N(R₅), and the other of X₁ and X₂ may be O or S. In still another embodiment, any one of X₁ and X₂ may be O, and the of X₁ and X₂ may be S.

In Formula 1, Y₁ may be B.

In Formula 1, Cy1 and Cy2 may each independently be a substituted or unsubstituted aromatic hydrocarbon ring of 6 to 30 ring-forming carbon atoms, or a substituted or unsubstituted aromatic heterocycle of 2 to 30 ring-forming carbon atoms, may be combined with an adjacent group to form a ring. For example, Cy1 may be combined with an adjacent X₁ group to form a ring, or Cy2 may be combined with adjacent an X₂ group to form a ring. In an embodiment, any one of X₁ and X₂ may be N(R₅), and any one of Cy1 and Cy2 may be bonded to N(R₅). In another embodiment, X₁ and X₂ may each independently be N(R₅), and Cy1 and Cy2 may be bonded to N(R₅) at each position among X₁ and X₂, wherein each R₅ group may be the same as or different from each other.

In Formula 1, R₁ to R₄ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. For example, R₁ to R₄ may each be a hydrogen atom.

In Formula 1, R₅ may be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. For example, R₅ may be a substituted or unsubstituted phenyl group, a substituted or unsubstituted biphenyl group, a substituted or unsubstituted terphenyl group, or a substituted or unsubstituted tetrahydronaphthyl group.

In Formula 1, n₁ may be an integer from 0 to 4. If n₁ is 0, the first dopant of an embodiment may not be substituted with R₁. A case in Formula 1 where n₁ is 4, and all of R₁ are hydrogen atoms may be the same as a case in Formula 1 where n₁ is 0. If n₁ is 2 or more, multiple R₁ groups may be all the same, or at least one R₁ group may be different.

In Formula 1, n₂ and n₃ may each independently be an integer from 0 to 3. If n₂ and n₃ are each 0, the first dopant of an embodiment may not be substituted with R₂ and R₃, respectively. A case in Formula 1 where n₂ and n₃ are each 3, and all of R₂ and all of R₃ are hydrogen atoms may be the same as a case in Formula 1 where n₂ and n₃ are each 0. If n₂ and n₃ are each 2 or more, multiple R₂ groups and multiple R₃ groups may be the same, respectively, or at least one R₂ group or R₃ group may be different.

In Formula 1, n₄ may be an integer from 0 to 2. If n₄ is 0, the first dopant of an embodiment may not be substituted with R₄. A case in Formula 1 where n₄ is 2, and all of R₄ are hydrogen atoms may be the same as a case Formula 1 where n₄ is 0. If n₄ is 2 or more, multiple R₄ groups may be all the same, or at least one R₄ group may be different.

In an embodiment, Cy1 and Cy2 in Formula 1 may each independently be a group represented by Formula 2.

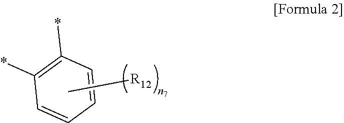

In Formula 2, R₁₂ may be a hydrogen atom, a deuterium atom, a halogen atom, a cyano group, a substituted or unsubstituted amine group, a substituted or unsubstituted oxy group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. For example, R₁₂ may be a hydrogen atom, a deuterium atom, a halogen atom, a substituted or unsubstituted alkyl group of 1 to 20 carbon atoms, a substituted or unsubstituted phenyl group, a substituted or unsubstituted amine group, a substituted or unsubstituted oxy group, a substituted or unsubstituted carbazole group, or a substituted or unsubstituted indolocarbazole group. In an embodiment, multiple R₁₂ groups may be provided, and adjacent multiple R₁₂ groups may be combined with each other to form a ring. In another embodiment, if R₁₂ is disposed adjacent to X₁ or X₂ of Formula 1, R₁₂ may be combined with X₁ or X₂ to form a ring.

In Formula 2, n₇ may be an integer from 0 to 4. In Formula 2, if n₇ is 0, the first dopant of an embodiment may not be substituted with R₁₂. A case in Formula 2 where n₇ is 4, and all of R₁₂ are hydrogen atoms may be the same as a case in Formula 2 where n₇ is 0. If n₇ is 2 or more, multiple R₁₂ groups may be all the same, or at least one R₁₂ group may be different.

In Formula 2, [symbol] are bonding sites to X₁ and Y₁ of Formula 1, or are bonding sites to X₂ and Y₁ of Formula 1.

The first dopant of an embodiment has a plate type skeleton structure with a boron atom as a center and has a structure including at least one indolocarbazole group in the plate type structure. The indolocarbazole has electron donating properties due to the unshared electron pair of a nitrogen atom positioned at a center, and may be introduced as a donor into a central core including boron. The first dopant of an embodiment includes at least one indolocarbazole group bonded to a benzene ring bonded to the boron atom, and the indolocarbazole group is substituted so that a nitrogen atom is bonded to the benzene ring at a para position with respect to the boron atom. Accordingly, the electron donating properties may increase. Since the indolocarbazole group is bonded to the central core through a carbon-carbon bond, chemical stability may be expected in a whole molecule.

In an indolocarbazole group having a fused structure of three benzene rings with one nitrogen atom as a center, an extinction coefficient is high, and if introduced into the first dopant of an embodiment, emission efficiency may increase. For example, by introducing a substituent having a high extinction coefficient, the light absorption of a compound itself may increase, efficient energy transfer from a host may be achieved, and the emission efficiency of a light emitting device may be improved. Since the first dopant of an embodiment includes an indolocarbazole group as a donor, high fluorescence quantum efficiency may be shown. For example, in a molecule of the first dopant of an embodiment, less non-radiative attenuation may arise, and thus, the emission efficiency properties of a light emitting device may increase even further.

Since the first dopant of an embodiment has a structure in which heteroatoms are substituted at ortho positions to the boron atom with a benzene ring as a center, a structure where an aromatic structure is additionally fused, i.e., a structure represented by Formula 2, may be included in a plate type structure. In a hydrocarbon ring compound having a conjugated structure like pyrene, orbitals may be distributed between a carbon-carbon bond. The first dopant has a structure in which three aromatic rings are fused with a boron atom having electron withdrawing properties as a center, and by introducing a nitrogen atom, an oxygen atom, or a sulfur atom, having electron donating properties, as constituent elements of a fused ring, the presence of orbitals in the atoms themselves not between the carbon-carbon bond may be induced. Since the first dopant has a structure in which an electron donating indolocarbazole group is connected with the fused structure, the spatial overlap of the highest occupied molecular orbital (HOMO) and the lowest unoccupied molecular orbital (LUMO) may be minimized. Accordingly, the dopant of an embodiment has a low ΔE_{ST} value, a polycyclic aromatic ring structure may be stabilized, a wavelength range suitable as a blue light emitting material may be selected, and if applied to a light emitting device ED, the efficiency of the light emitting device ED may be improved.

In an embodiment, if Cy1 and Cy2 are each a group represented by Formula 2, then Cy1 and Cy2 may each independently be a group represented by Formula 2-1 or Formula 2-2. Formula 2-1 represents a case of Formula 2 where bonding positions of R₁₂ and the type of a substituent are specified. Formula 2-2 represents a case of Formula 2 where multiple R₁₂ are provided and are combined with each other to form an additional ring. In an embodiment, both Cy1 and Cy2 may have a structure represented by Formula 2-1, or both may have a structure represented by Formula 2-2. In another embodiment, any one of Cy1 and Cy2 may have a structure represented by Formula 2-1, and the other of Cy1 and Cy2 may have a structure represented by Formula 2-2.

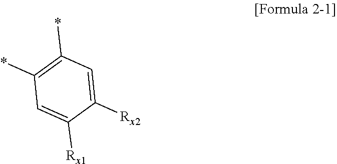

In Formula 2-1, Rₓ₁ and Rₓ₂ may each independently be a hydrogen atom, a deuterium atom, a halogen atom, a substituted or unsubstituted alkyl group of 1 to 20 carbon atoms, a substituted or unsubstituted phenyl group, a substituted or unsubstituted amine group, a substituted or unsubstituted carbazole group, or a substituted or unsubstituted indolocarbazole group, or may be combined with an adjacent group to form a ring.

In Formula 2-1, [symbol] are bonding sites to X₁ and Y₁ of Formula 1, or are bonding sites to X₂ and Y1 of Formula 1.

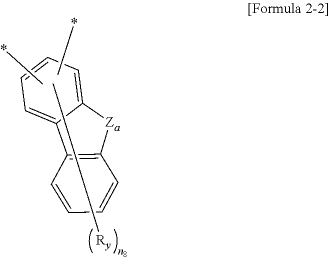

In Formula 2-2, Zₐ may be N(R₁₃) or O.

In Formula 2-2, R_{y} may be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula 2-2, Ria may be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula 2-2, n₈ may be an integer from 0 to 6. In Formula 2-2, if n₈ is 0, the first dopant of an embodiment may not be substituted with R_{y}. A case in Formula 2 where n₈ is 6, and all of R_{y} are hydrogen atoms may be the same as a case in Formula 2-2 where n₈ is 0. If n₈ is 2 or more, multiple R_{y} groups may be all the same, or at least one R_{y} group may be different.

In Formula 2-2, [symbol] are bonding sites to X₁ and Y₁ of Formula 1, or are bonding sites to X₂ and Y₁ of Formula 1.

In an embodiment, in the case where Cy1 is a group represented by Formula 2-2, Cy1 may be bonded to Y₁ of Formula 1 at a carbon at a para position with respect to Zₐ, and may be bonded to X₁ of Formula 1 at a carbon at a meta position with respect to Zₐ. In another embodiment, Cy1 may be bonded to X₁ of Formula 1 at a carbon at a para position with respect to Zₐ, and may be bonded to Y₁ of Formula 1 at a carbon at a meta position with respect to Zₐ.

In an embodiment, in the case where Cy2 is a group represented by Formula 2-2, Cy2 may be bonded to Y₁ of Formula 1 at a carbon at a para position with respect to Zₐ, and may be bonded to X₂ of Formula 1 at a carbon at a meta position with respect to Zₐ. In another embodiment, Cy2 may be bonded to X₂ of Formula 1 at a carbon at a para position with respect to Zₐ, and may be bonded to Y₁ of Formula 1 at a carbon at a meta position with respect to Zₐ.

In an embodiment, Rig of Formula 2 may be a substituted or unsubstituted alkyl group of 1 to 10 carbon atoms, a substituted or unsubstituted amine group, a substituted or unsubstituted oxy group, or a group represented by any one of Formula 3-1 to Formula 3-4. Formula 3-1 to Formula 3-4 represent cases of Formula 2 where the type of R₁₂ group is specified. Formula 3-1 represents a case where R₁₂ is a substituted or unsubstituted phenyl group. Formula 3-2 represents a case where R₁₂ is a substituted or unsubstituted carbazole group, and the nitrogen atom at position 9 of the carbazole group is bonded to the phenyl group of Formula 2. Formula 3-3 represents a case where R₁₂ is a substituted or unsubstituted heteroaryl group. Formula 3-4 represents a case where R₁₂ is a substituted or unsubstituted indolocarbazole group. In an embodiment, a case where at least one of Cy1 or Cy2 is a group represented by Formula 2, n₇ is 1, and R₁₂ of Formula 2 is a group represented by Formula 3-4, may mean that the first dopant represented by Formula 1 includes at least two indolocarbazole groups in a molecular structure. A case where Cy1 and Cy2 are each a group represented by Formula 2, n₇ is 1, and Rig of Formula 2 is represented by Formula 3-4, may mean that the first dopant represented by Formula 1 includes at least three indolocarbazole groups in a molecular structure.

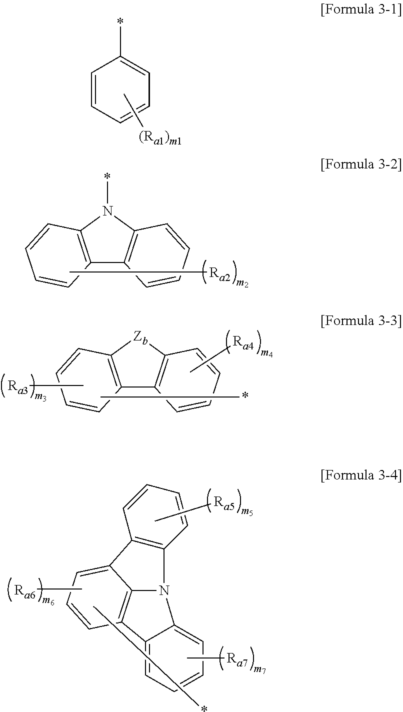

In Formula 3-3, Z_{b} may be N(R₁₄) or O.

In Formula 3-1 to Formula 3-4, Rₐ₁ to Rₐ₇ and R₁₄ may each independently be a hydrogen atom, a deuterium atom, a halogen atom, a cyano group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Rₐ₁ to Rₐ₇ and R₁₄ may each independently be a hydrogen atom, a deuterium atom, a cyano group, a t-butyl group, or an unsubstituted phenyl group.

In Formula 3-1 to Formula 3-4, m₁ may be an integer from 0 to 5, m₂ may be an integer from 0 to 8, m₃ to m₅ and m₇ may each independently be an integer from 0 to 4, and m₆ may be an integer from 0 to 3. In Formula 3-1 to Formula 3-4, a sum of m₃ and m₄ may be equal to or less than 7, and a sum of m₆ and m₇ may be equal to or less than 6.

If m₁ is 0, the first dopant of an embodiment may not be substituted with Rₐ₁. A case in Formula 3-1 where m₁ is 5, and all of Rₐ₁ are hydrogen atoms may be the same as a case in Formula 3-1 where m₁ is 0. If m₁ is 2 or more, multiple Rₐ₁ groups may be all the same, or at least one Rₐ₁ group may be different.

If m₂ is 0, the first dopant of an embodiment may not be substituted with Rₐ₂. A case in Formula 3-2 where m₂ is 8, and all of Rₐ₂ are hydrogen atoms may be the same as a case in Formula 3-2 where m₂ is 0. If m₁ is 2 or more, multiple Rₐ₇ groups may be all the same, or at least one Rₐ₂ group may be different.

If m₃ to m₅, and m₇ are 0, the first dopant of an embodiment may not be substituted with Rₐ₃ to Rₐ₅, and Rₐ₇, respectively. A case where each of m₃ to m₅, and m₇ is 4, and Rₐ₃ to Rₐ₅, and Rₐ₇ are all hydrogen atoms may be the same as a case in Formula 3-3 and Formula 3-4 where each of m₃ to m₅, and m₇ are each 0. If each of m₃ to m₅, and m₇ is 2 or more, multiple groups of Rₐ₃ to Rₐ₅, and Rₐ₇ may be all the same, or at least one group among Rₐ₃ to Rₐ₅, and Rₐ₇ may be different.

If m₆ is 0, the first dopant of an embodiment may not be substituted with Rₐ₆. A case in Formula 3-4 where m₆ is 3, and all of Rₐ₆ are hydrogen atoms may be the same as a case in Formula 3-4 where m₆ is 0. If m₆ is 2 or more, multiple Rₐ₆ groups may be all the same, or at least one Rₐ₆ group may be different.

In an embodiment, the first dopant represented by Formula 1 may be represented by any one of Formula 4-1 or Formula 4-2.

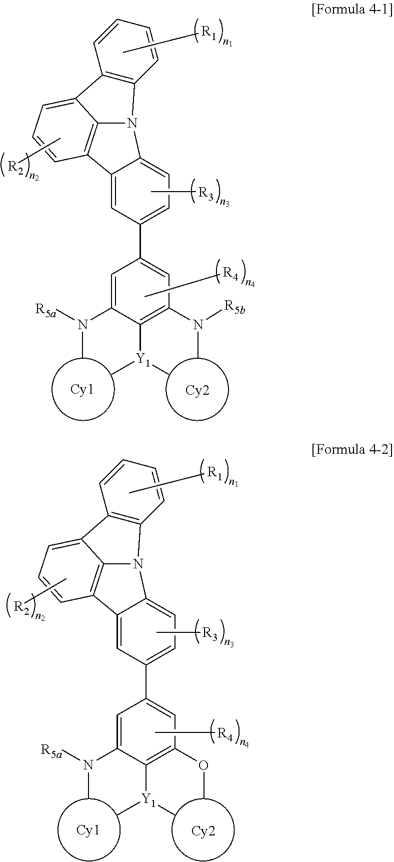

Formula 4-1 and Formula 4-2 each represent Formula 1 where X₁ and X₂ are specified. Formula 4-1 represents a case where X₁ and X₂ are each N(R₅). Formula 4-2 represents a case where X₁ is N(R₅) and X₂ is O.

In Formula 4-1 and Formula 4-2, R₅ₐ and R_{5b} may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. For example, R₅ₐ and R_{5b} may each independently be a substituted or unsubstituted phenyl group, a substituted or unsubstituted biphenyl group, a substituted or unsubstituted terphenyl group, or a substituted or unsubstituted tetrahydronaphthyl group. As another example, R₅ₐ may be combined with a substituent included in Cy1 to form a ring, and R_{5b} may be combined with a substituent included in Cy2 to form a ring.

In Formula 4-1 and Formula 4-2, Cy1, Cy2, Y₁, R₁ to R₄, and n₁ to n₄ are the same as defined in Formula 1.

In an embodiment, the first dopant represented by Formula 1 may be represented by any one of Formula 5-1 to Formula 5-3.

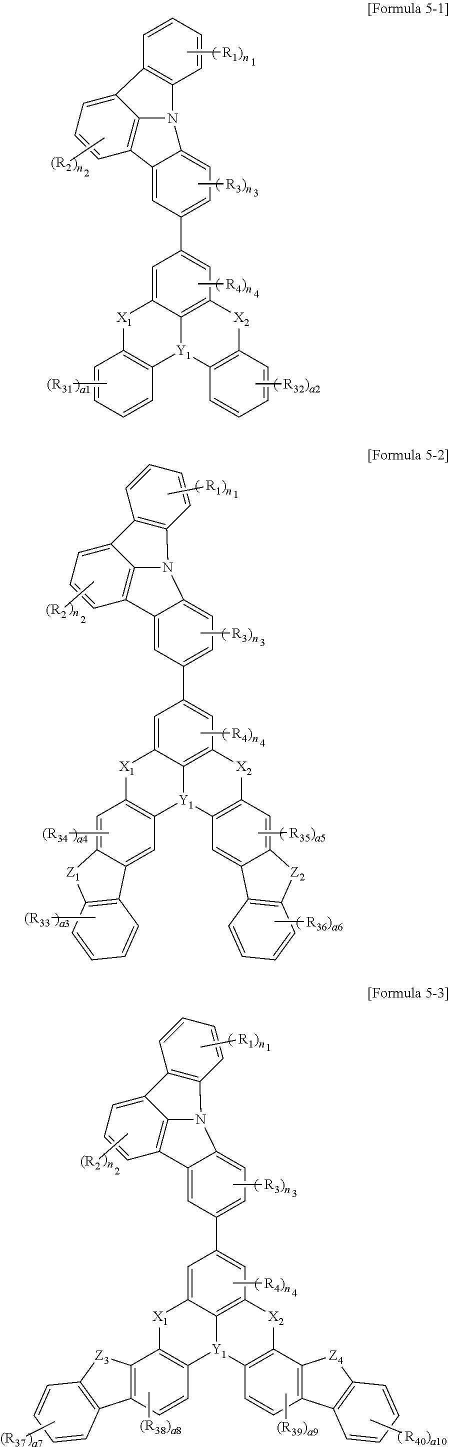

In Formula 5-1 to Formula 5-3, Z₁ to Z₄ may each independently be N(R₄₁) or 0.

In Formula 5-1 to Formula 5-3, R₃₁ to R₄₀ may each independently be a hydrogen atom, a deuterium atom, a halogen atom, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₃₁ to R₄₀ may each independently be a hydrogen atom, a deuterium atom, a substituted or unsubstituted t-butyl group, a substituted or unsubstituted phenyl group, a substituted or unsubstituted carbazole group, or a substituted or unsubstituted dibenzofuran group.

In Formula 5-1 to Formula 5-3, R₄₁ may be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₄₁ may be a substituted or unsubstituted phenyl group.

In Formula 5-1 to Formula 5-3, a1 to a3, a6, a7, and a10 may each independently be an integer from 0 to 4, and a4, a5, a8, and a9 may each independently be an integer from 0 to 2.

If each of a1 to a3, a6, a7, and a10 is 0, the first dopant according to an embodiment may not be substituted with each of R₃₁ to R₃₃, R₃₆, R₃₇, and R₂₀, respectively. A case where a1 to a3, a6, a7, and a10 are each 4, and R₃₁ to R₃₃, R₃₆, R₃₇, and R₂₀ are all hydrogen atoms, respectively, may be the same as a case where a1 to a3, a6, a7, and a10 are each 0. If each of a1 to a3, a6, a7, and a10 is 2 or more, multiple groups of R₃₁ to R₃₃, R₃₆, R₃₇, and R₂₀ may be all the same, or at least one group among R₃₁ to R₃₃, R₃₆, R₃₇, and R₂₀ may be different.

If each of a4, a5, a8, and a9 is 0, the first dopant according to an embodiment may not be substituted with each of R₃₄, R₃₅, R₃₈, and R₃₉, respectively. A case where a4, a5, a8, and a9 are each 2, and R₃₄, R₃₅, R₃₈, and R₃₉ are all hydrogen atoms, respectively, may be the same as a case where a1 to a4, a5, a8, and a9 are each 0. If each of a4, a5, a8, and a9 is 2 or more, multiple groups of R₃₄, R₃₅, R₃₈, and R₃₉ may be all the same, or at least one group among R₃₄, R₃₅, R₃₈, and R₃₉ may be different.

In Formula 5-1 to Formula 5-3, X₁, X₂, Y₁, R₁ to R₄, and n₁ to n₄ are the same as defined in Formula 1.

In an embodiment, if each of X₁ and X₂ in Formula 1 is N(R₅), then R₅ may be a group represented by any one of Formula 6-1 to Formula 6-4.

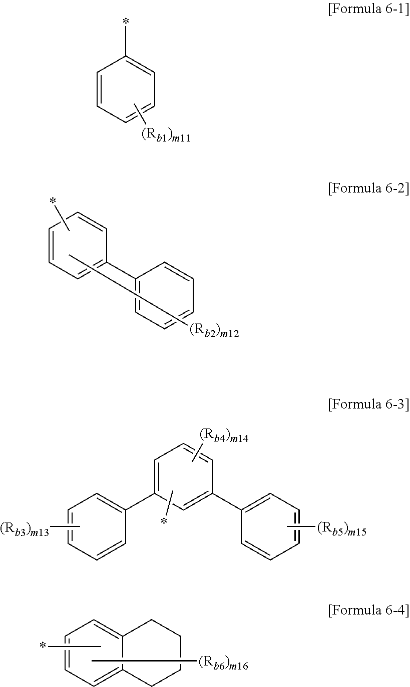

In Formula 6-1 to Formula 6-4, R_{b1} to R_{b6} may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R_{b1} to R_{b6} may each independently be a hydrogen atom, a methyl group, a t-butyl group, or a substituted or unsubstituted phenyl group with a t-butyl group. In Formula 6-1 to Formula 6-4, if each of X₁ and X₂ in Formula 1 is N(R₅), then [symbol] is a bonding site to the nitrogen atom of N(R₅).

In Formula 6-1 to Formula 6-4, m₁₁, m₁₃, and m₁₅ may each independently be an integer from 0 to 5, m₁₂ may be an integer from 0 to 9, m₁₄ may be an integer from 0 to 3, and m₁₆ may be an integer from 0 to 11.

If m₁₁, m₁₃, and m₁₅ are 0, the first dopant according to an embodiment may not be substituted with R_{b1}, R_{b3}, and R_{b5}, respectively. A case where m₁₁, m₁₃, and m₁₅ are each 5, and R_{b1}, R_{b3}, and R_{b5} are all hydrogen atoms, respectively, may be the same as a case where mu, m₁₃, and m₁₅ are each 0. If each of m₁₁, m₁₃, and m₁₅ is 2 or more, multiple groups of R_{b1}, R_{b3}, and R_{b5} may be all the same, or at least one group among R_{b1}, R_{b3}, and R_{b5} may be different.

If m₁₆ is 0, the first dopant according to an embodiment may not be substituted with R_{b2}. A case in Formula 6-2 where m₁₂ is 9, and R₂ are all hydrogen atoms may be the same as a case in Formula 6-2 where m₁₂ is 0. If m₁₂ is 2 or more, multiple R_{b2} groups may be all the same, or at least one R_{b2} group may be different.

If m₁₄ is 0, the first dopant according to an embodiment may not be substituted with R_{b4}. A case in Formula 6-3 where m₁₄ is 3, and R_{b4} are all hydrogen atoms may be the same as a case in Formula 6-3 where m₁₄ is 0. If m₁₄ is 2 or more, multiple R_{b4} groups may be all the same, or at least one R_{b4} group may be different.

If m₁₆ is 0, the first dopant according to an embodiment may not be substituted with R_{b6}. A case in Formula 6-4 where m₁₆ is 11, and R_{b6} are all hydrogen atoms, may be the same as a case in Formula 6-4 where m₁₆ is 0. If m₁₆ is 2 or more, multiple groups R_{b6} may be all the same, or at least one R_{b6} group may be different.

In the light emitting device ED of an embodiment, the emission layer EML may include a host. The host may not emit light but may transfer energy to a dopant in the light emitting device ED. The emission layer EML may include one or more types of hosts. For example, the emission layer EML may include two different types of hosts. In the case where the emission layer EML includes two types of hosts, the two types of hosts may include a hole transport host and an electron transport host. However, embodiments are not limited thereto, and the emission layer EML may include one type of a host or a mixture of two or more different types of hosts.

In an embodiment, the emission layer EML may include two different hosts. The host may include a first host, and a second host which is different from the first host. The host may include a first host having a hole transport moiety and a second host having an electron transport moiety. In the light emitting device ED of an embodiment, the first host and the second host may form an exciplex.

In an embodiment, the host may include a first host represented by Formula H-1, and a second host represented by Formula H-2. The first host may be a hole transport host, and the second host may be an electron transport host.

The emission layer EML according to an embodiment may include a first host including a moiety derived from a carbazole group. The first host may be represented by Formula H-1.

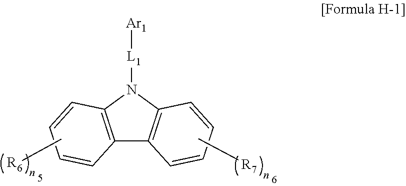

In Formula H-1, L₁ may be a direct linkage, a substituted or unsubstituted arylene group of 6 to 30 ring-forming carbon atoms, or a substituted or unsubstituted heteroarylene group of 2 to 30 ring-forming carbon atoms. In Formula H-1, Ar₁ may be a substituted or unsubstituted aryl group of 6 to 30 ring-forming carbon atoms, or a substituted or unsubstituted heteroaryl group of 2 to 30 ring-forming carbon atoms.

In Formula H-1, n₅ and n₆ may each independently be an integer from 0 to 4, and R₆ and R₇ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. If n₅ and n₆ are each 2 or more, multiple R₆ groups and multiple R₇ groups may be all the same, or at least one thereof may be different. For example, in Formula H-1, n₅ and n₆ may each be 0, so that the carbazole group of Formula H-1 may correspond to an unsubstituted carbazole group.

For example, in Formula H-1, L₁ may be a direct linkage, a phenylene group, a divalent biphenyl group, a divalent carbazole group, etc., but embodiments are not limited thereto. For example, in Formula H-1, Ar₁ may be a substituted or unsubstituted carbazole group, a substituted or unsubstituted dibenzofuran group, a substituted or unsubstituted dibenzothiophene group, a substituted or unsubstituted biphenyl group, etc., but embodiments are not limited thereto.

In the light emitting device ED of an embodiment, the emission layer EML may include a second host represented by Formula H-2.

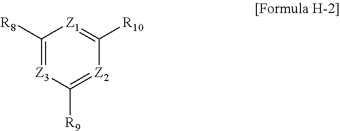

In Formula H-2, Z₁ to Z₃ may each independently be C(R₁₁) or N, and at least one of Z₁ to Z₃ may be N. For example, the second host represented by Formula H-2 may include a pyridine moiety, a pyrimidine moiety, or a triazine moiety.

In Formula H-2, R₈ to R₁₁ may each independently be a hydrogen atom, a deuterium atom, a cyano group, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms.

For example, in Formula H-2, R₈ to R₁₁ may be each independently a substituted or unsubstituted phenyl group, a substituted or unsubstituted carbazole group, etc., but embodiments are not limited thereto.

If the emission layer EML of the light emitting device ED of an embodiment includes the first host represented by Formula H-1 and the second host represented by Formula H-2, simultaneously, excellent emission efficiency and long-life characteristics may be shown. For example, in the emission layer EML of the light emitting device ED of an embodiment, the host may include an exciplex formed by the first host represented by Formula H-1 and the second host represented by Formula H-2.

Among the first and second hosts included in the emission layer EML simultaneously, the first host may be a hole transport host, and the second host may be an electron transport host. The light emitting device ED of an embodiment includes both a first host having excellent hole transport properties and a second host having excellent electron transport properties, and may efficiently transfer energy to a dopant compound, which will be explained later.

The light emitting device ED of an embodiment may further include a second dopant in addition to the first dopant represented by Formula 1 in the emission layer EML. The emission layer EML may include an organometallic complex including platinum (Pt) as a central metal element and ligands bonded to the central metal element, as the second dopant. In the light emitting device ED of an embodiment, the emission layer EML may include a second dopant represented by Formula D-2.

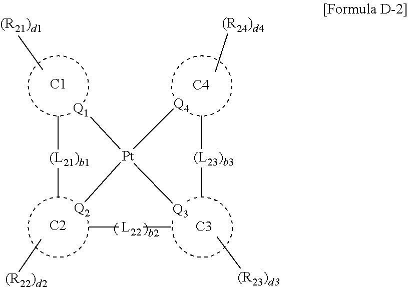

In Formula D-2, Q₁ to Q₄ may each independently be C or N.

In Formula D-2, C1 to C4 may each independently be a substituted or unsubstituted hydrocarbon ring of 5 to 30 ring-forming carbon atoms, or a substituted or unsubstituted heterocycle of 2 to 30 ring-forming carbon atoms.

In Formula D-2, L₂₁ to L₂₃ may each independently be a direct linkage,

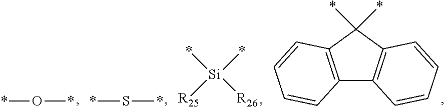

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms. In L₂₁ to L₂₃, [symbol] may be a bonding site to C1 to C4.

In Formula D-2, b1 to b3 may each independently be 0 or 1. If b1 is 0, C1 and C2 may not be connected to each other. If b2 is 0, C2 and C3 may not be connected to each other. If b3 is 0, C3 and C4 may not be connected to each other.

In Formula D-2, R₂₁ to R₂₆ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 1 to 30 ring-forming carbon atoms, or may be combined with an adjacent group to form a ring. For example, R₂₁ to R₂₆ may each independently be a methyl group or a t-butyl group.

In Formula D-2, d1 to d4 may each independently be an integer from 0 to 4. If each of d1 to d4 is 2 or more, multiple groups of R₂₁ to R₂₄ may be all the same, or at least one thereof may be different.

In Formula D-2, C1 to C4 may each independently be a substituted or unsubstituted hydrocarbon ring or a substituted or unsubstituted heterocycle, represented by any one of C-1 to C-4.

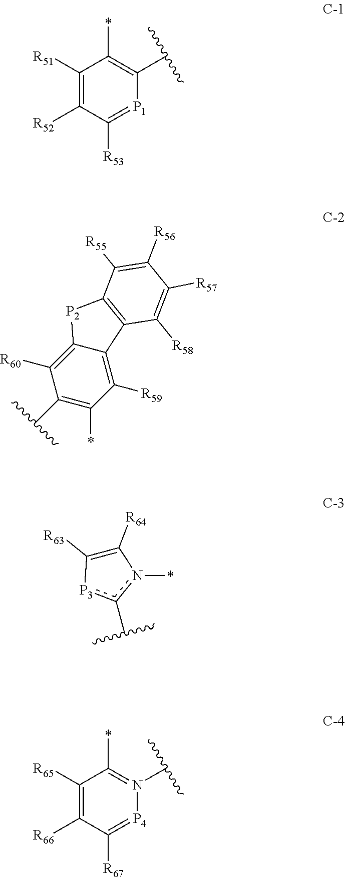

In C-1 to C-4, P₁ may be [symbol] or C(R₅₄), P₂ may be [symbol] or N(R₆₁), P₃ may be [symbol] or N(R₆₂), and P₄ may be [symbol] or C(R₆₈). In C-1 to C-4, R₅₁ to R₆₈ may each independently be a substituted or unsubstituted alkyl group of 1 to 20 carbon atoms, a substituted or unsubstituted aryl group of 6 to 30 ring-forming carbon atoms, or a substituted or unsubstituted heteroaryl group of 6 to 30 ring-forming carbon atoms, or may be combined with an adjacent group to form a ring.

In C-1 to C-4, [symbol] is a bonding site to a Pt central metal element, and [symbol] is a bonding site to a neighboring ring group (C1 to C4) or a linker (L₂₁ to L₂₄).

The second dopant represented by Formula D-2 may be a phosphorescence dopant.

In an embodiment, the first dopant may be a light emitting dopant that emits blue light, and the emission layer EML may emit fluorescence. For example, the emission layer EML may emit blue light of delayed fluorescence.

In an embodiment, the second dopant included in the emission layer EML may be a sensitizer. In the light emitting device ED of an embodiment, the second dopant included in the emission layer EML may function as a sensitizer and may transfer energy from the host to the first dopant which is a light-emitting dopant. For example, the second dopant may function as an auxiliary dopant, and may accelerate energy transfer to the first dopant, which is a light emitting dopant, to increase the light emitting ratio of the first dopant. Accordingly, the emission efficiency of the emission layer EML of an embodiment may be improved. When energy transfer to the first dopant increases, excitons formed in the emission layer EML may not accumulate in the emission layer EML but may emit light rapidly, thereby reducing the deterioration of a device. Accordingly, the life of the light emitting device ED of an embodiment may increase.

In the light emitting device ED of an embodiment, if the emission layer EML includes the first host, the second host, the first dopant, and the second dopant, an amount of the first dopant may be in a range of about 0.1 wt % to about 5 wt %, based on a total weight of the first host, the second host, the first dopant, and the second dopant. An amount of the second dopant may be in a range of about 5 wt % to about 25 wt %. For example, an amount of the second dopant may be in a range of about 10 wt % to about 15 wt %.

If the amounts of the first dopant and the second dopant satisfy the above-described ranges, the second dopant may efficiently transfer energy to the first dopant, and accordingly, the emission efficiency and device life of the light emitting device ED may increase.

In the emission layer EML, an amount of the first host and second host may be a remaining amount excluding the first dopant and the second dopant. For example, in the emission layer EML, the amount of the first host and second host may be in a range of about 70 wt % to about 94.9 wt %, based on the total weight of the first host, the second host, the first dopant, and the second dopant.

Among the total weight of the first host and second host, a weight ratio of the first host to the second host may be in a range of about 3:7 to about 7:3.

If the amount of the first host and second host satisfies the above-described ranges and ratios, charge balance properties in the emission layer EML may be improved, and the emission efficiency and device life may increase. If the amount of the first host and second host deviates from the above-described ratio range, charge balance in the emission layer EML may be lost, emission efficiency may be degraded, and the device may readily deteriorate.

If the first host, the second host, the first dopant, and the second dopant, included in the emission layer EML satisfy the above-described ranges and ratios, excellent emission efficiency and long life may be achieved.

The light emitting device ED of an embodiment includes all of the first host, the second host, the first dopant, and the second dopant, and the emission layer EML may include the combination of first and second hosts and first and second dopants. In the light emitting device ED of an embodiment, the emission layer EML may include two different hosts, a first dopant emitting delayed fluorescence, and a second dopant including an organometallic complex, simultaneously, and excellent emission efficiency properties may be shown.

In an embodiment, the first dopant represented by Formula 1 may be any one selected from Compound Group 1. The emission layer EML may include at least one selected from Compound Group 1 as the first dopant.

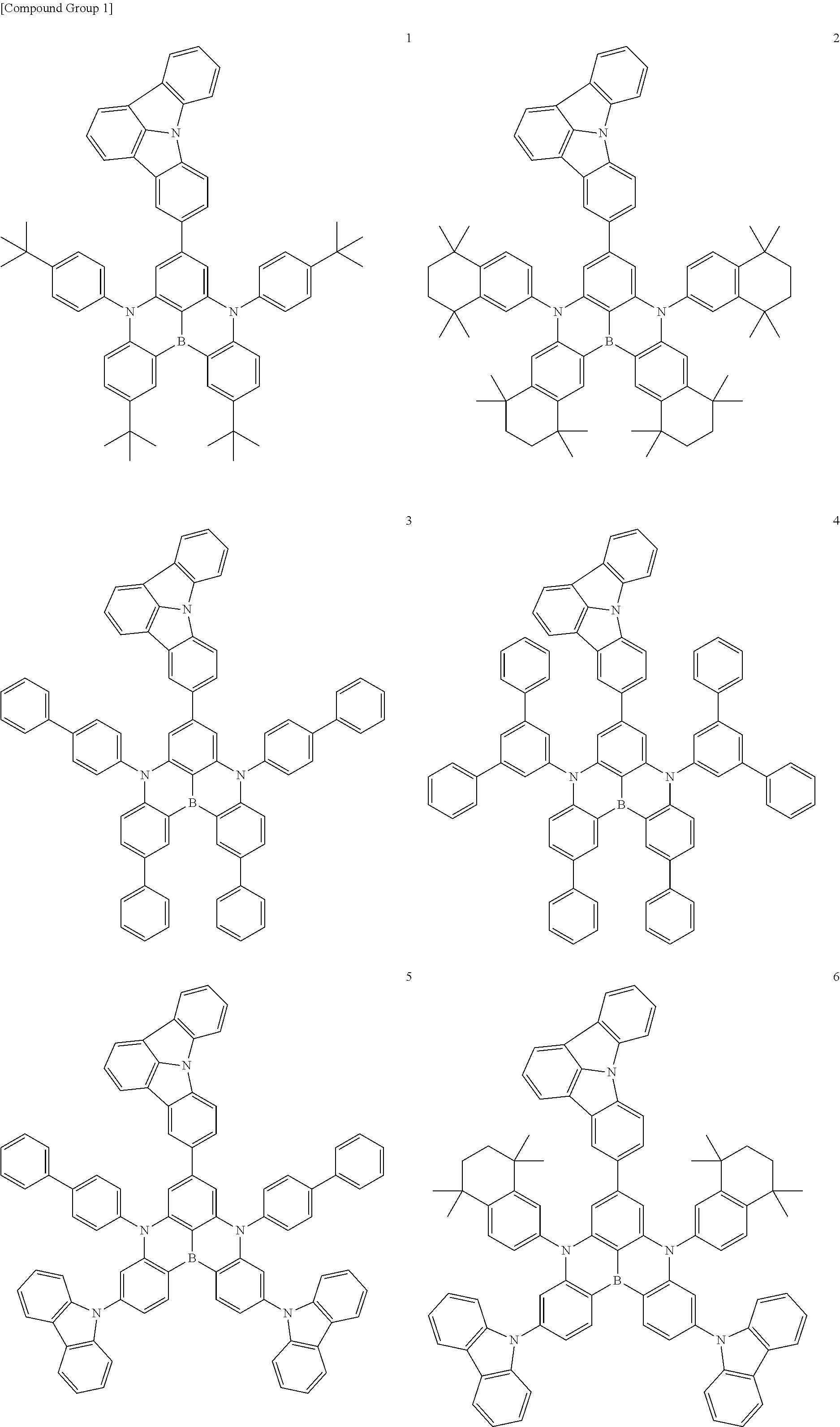

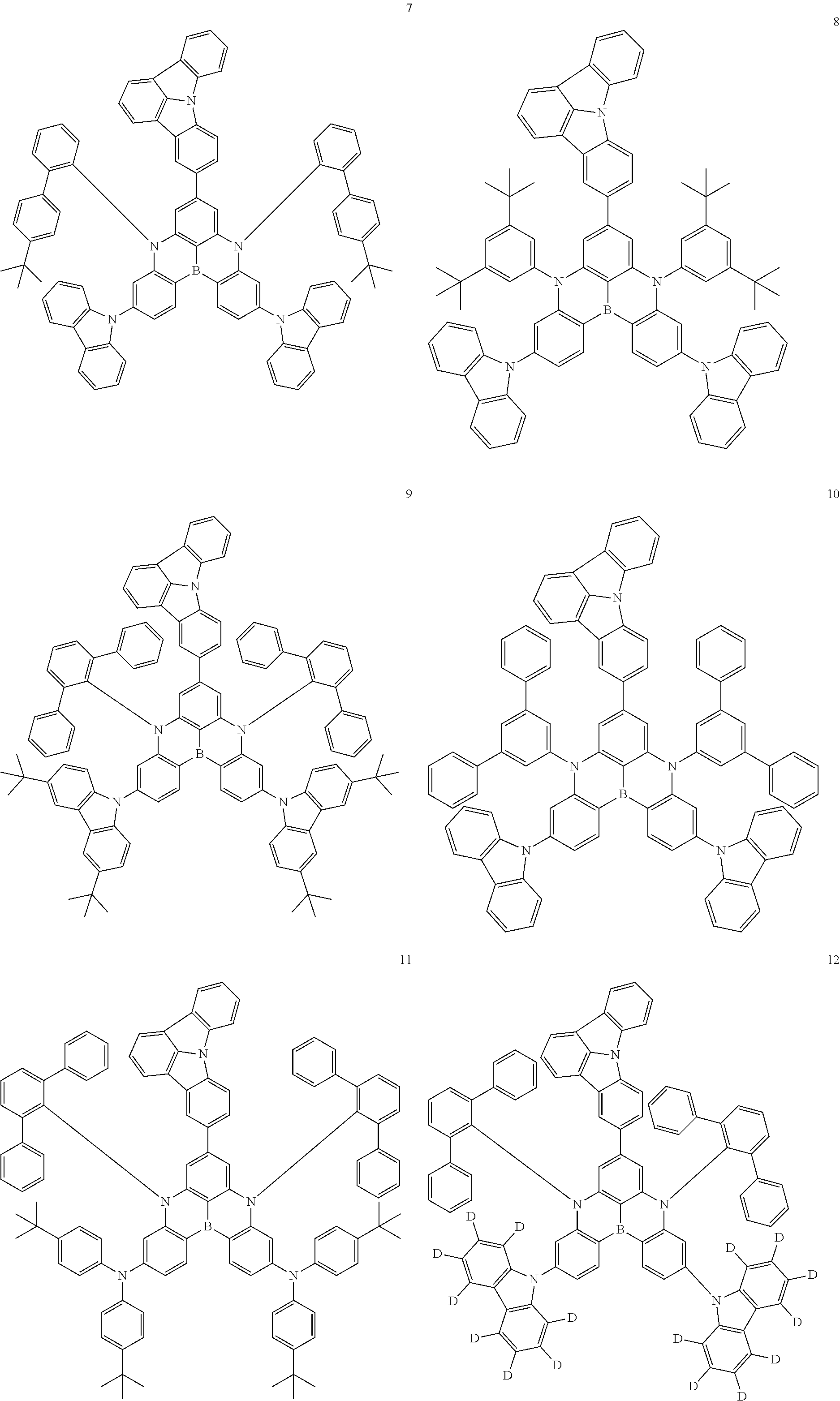

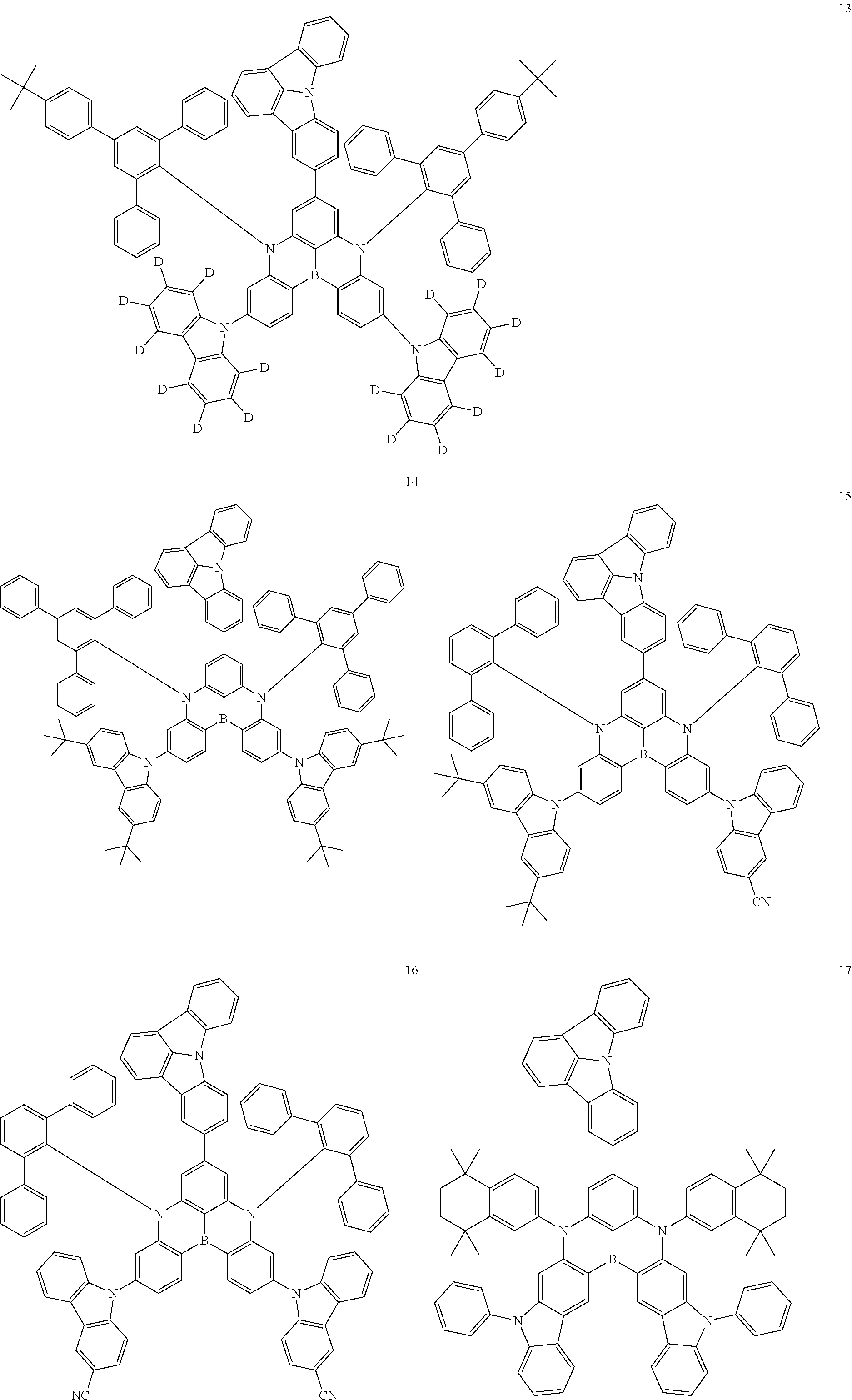

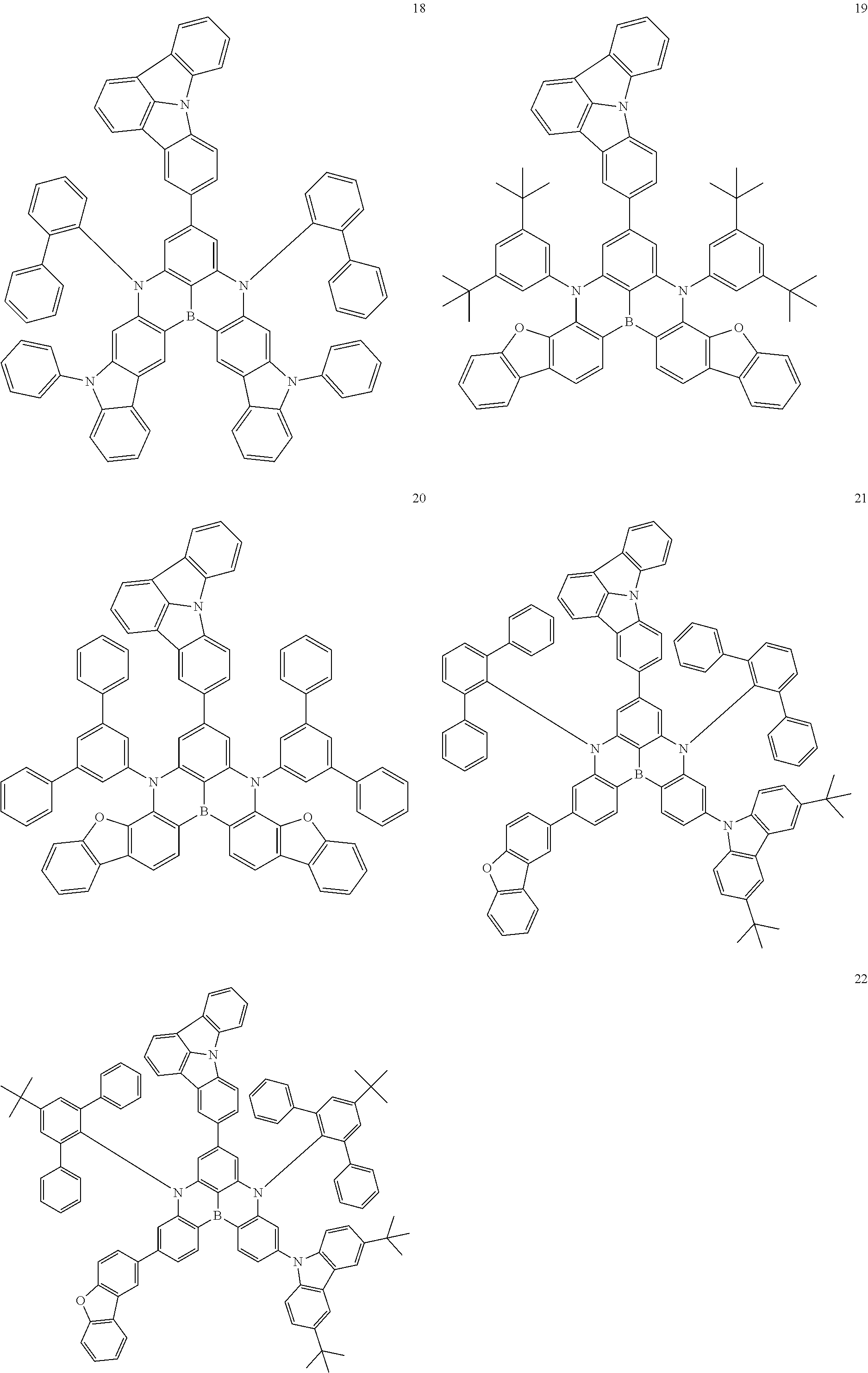

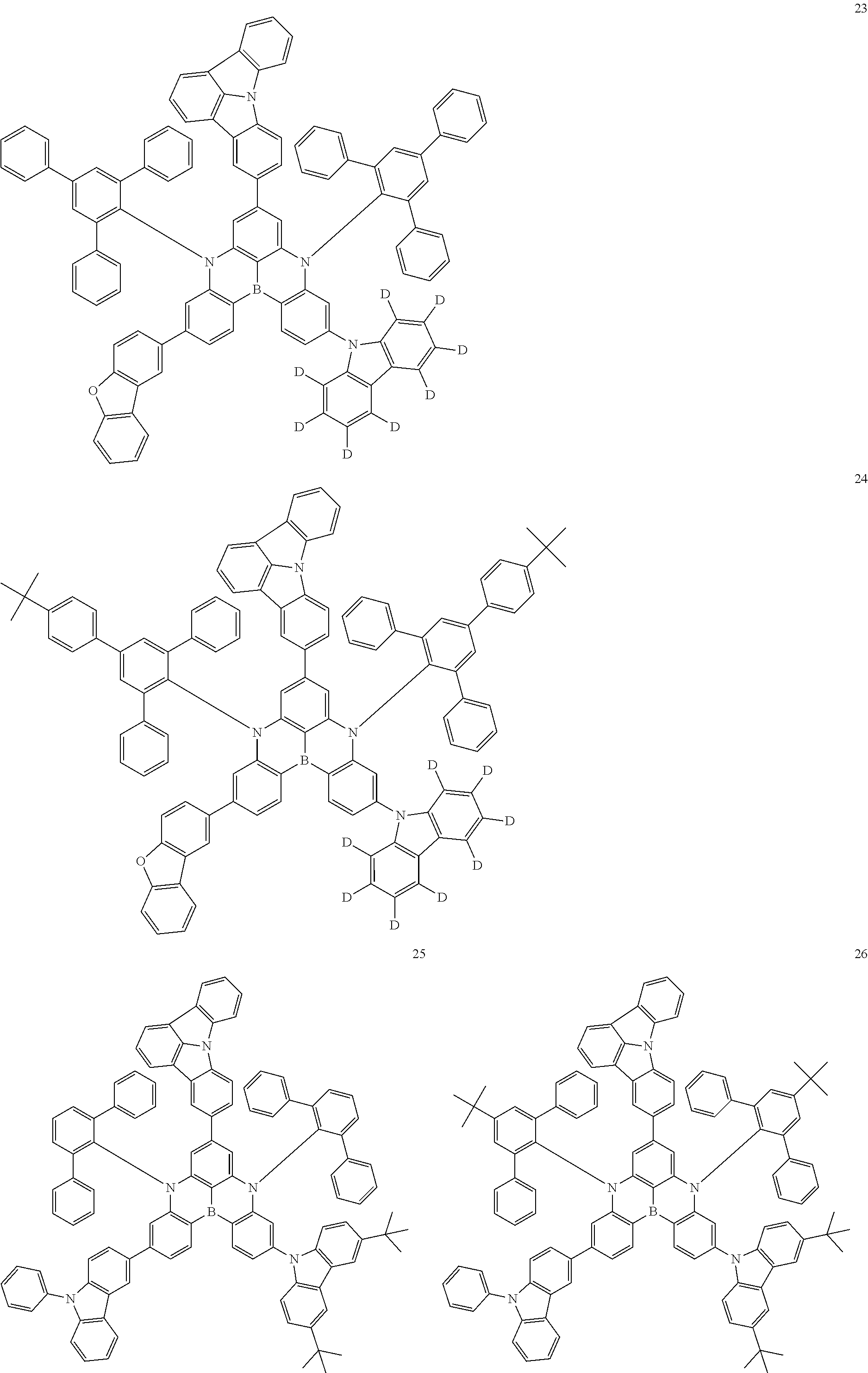

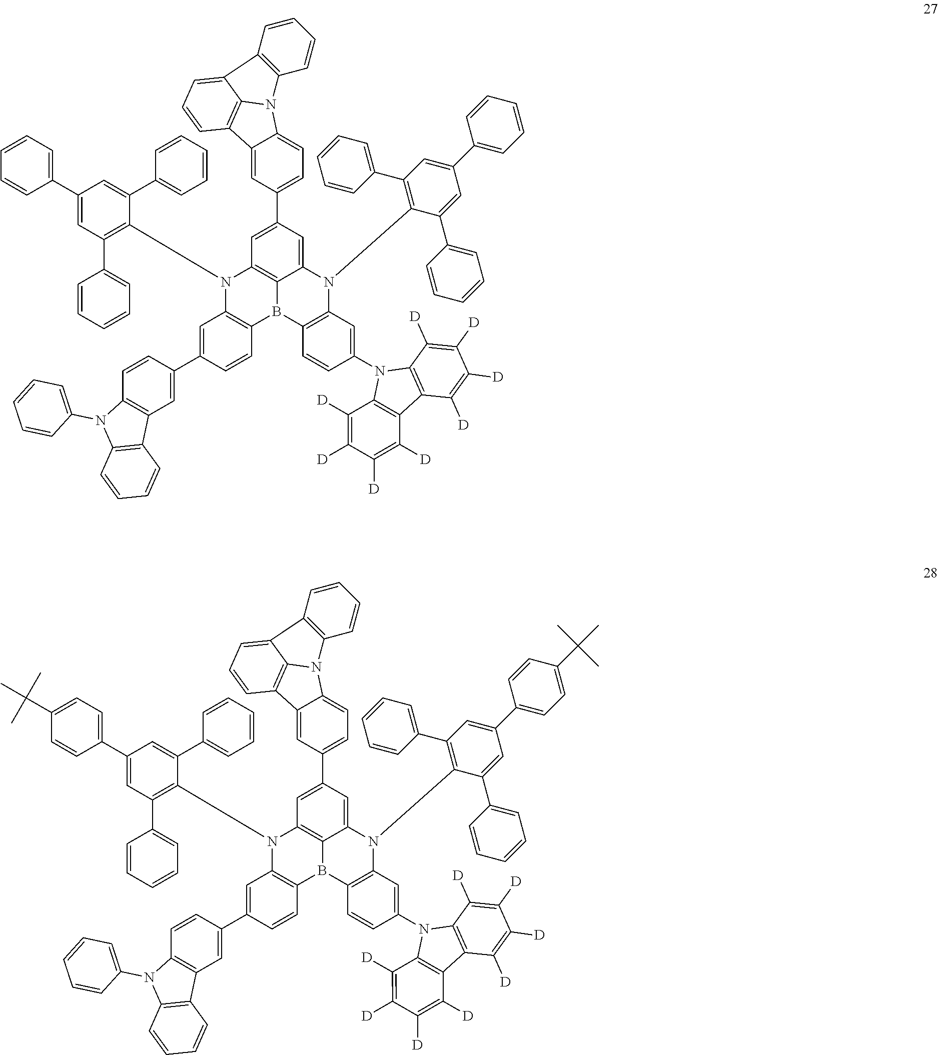

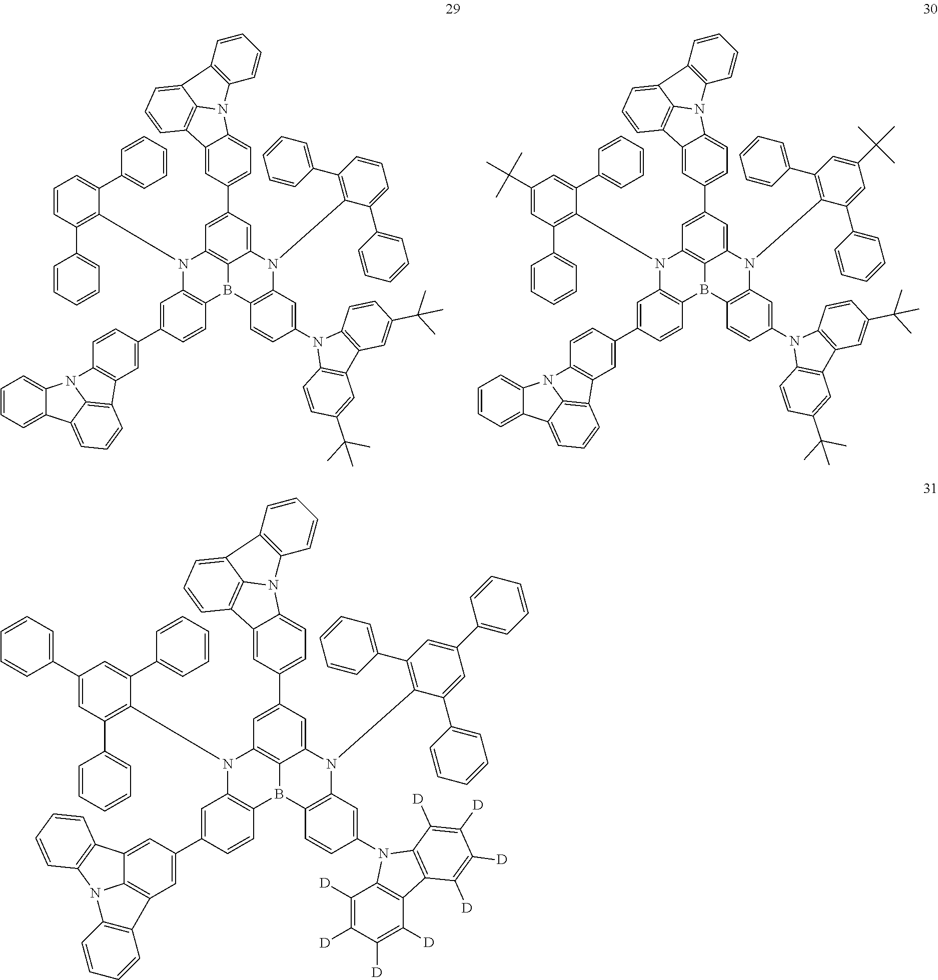

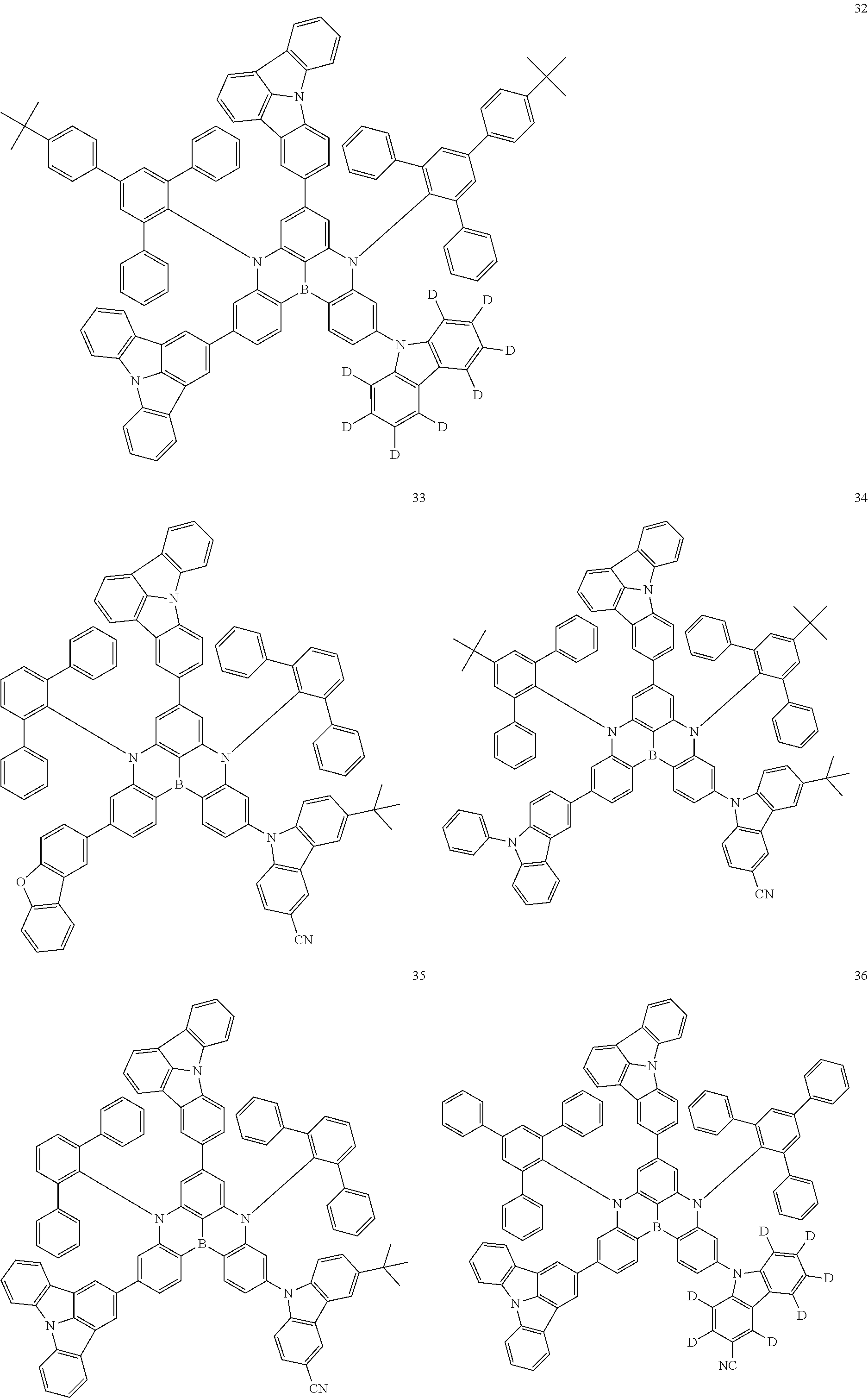

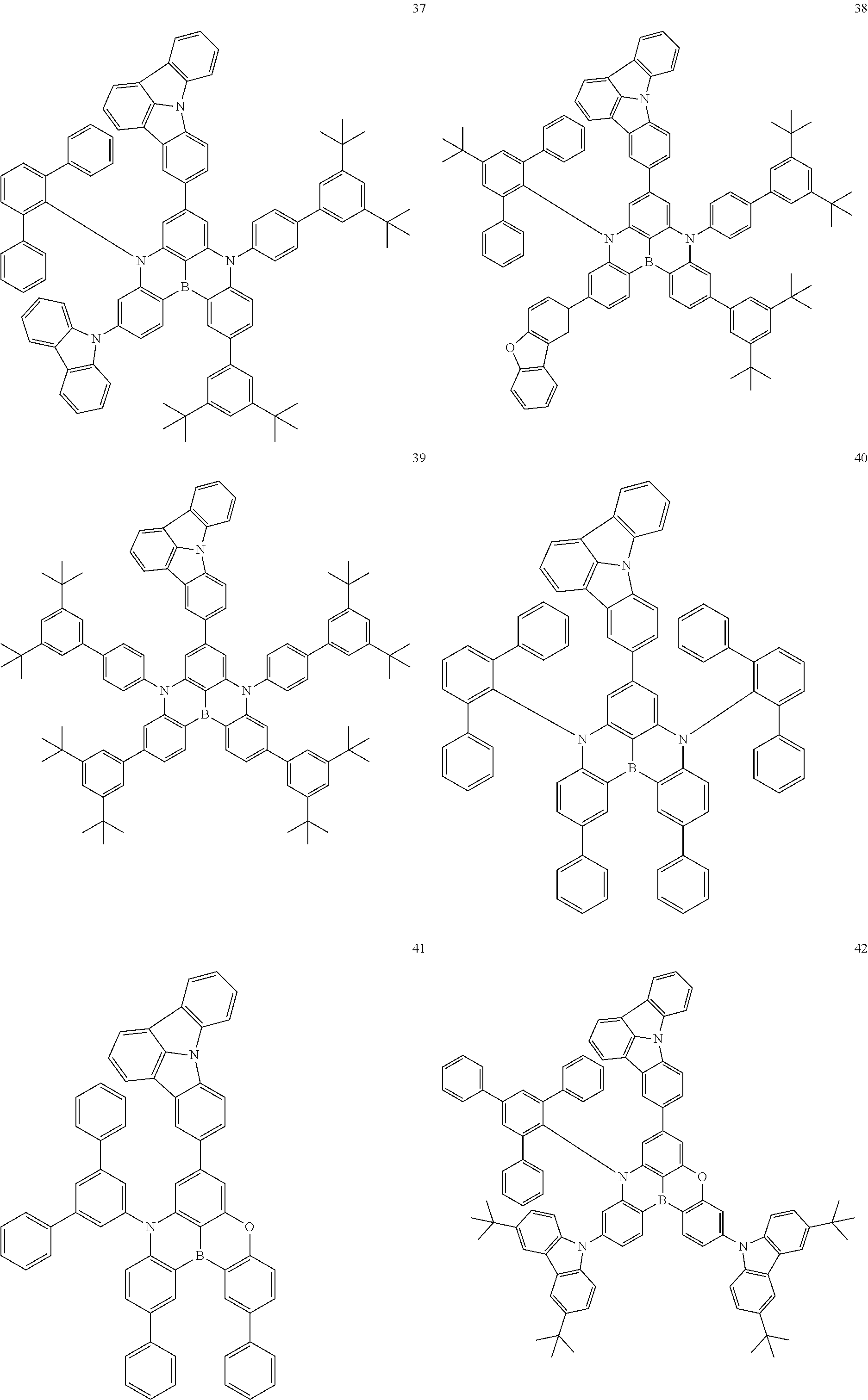

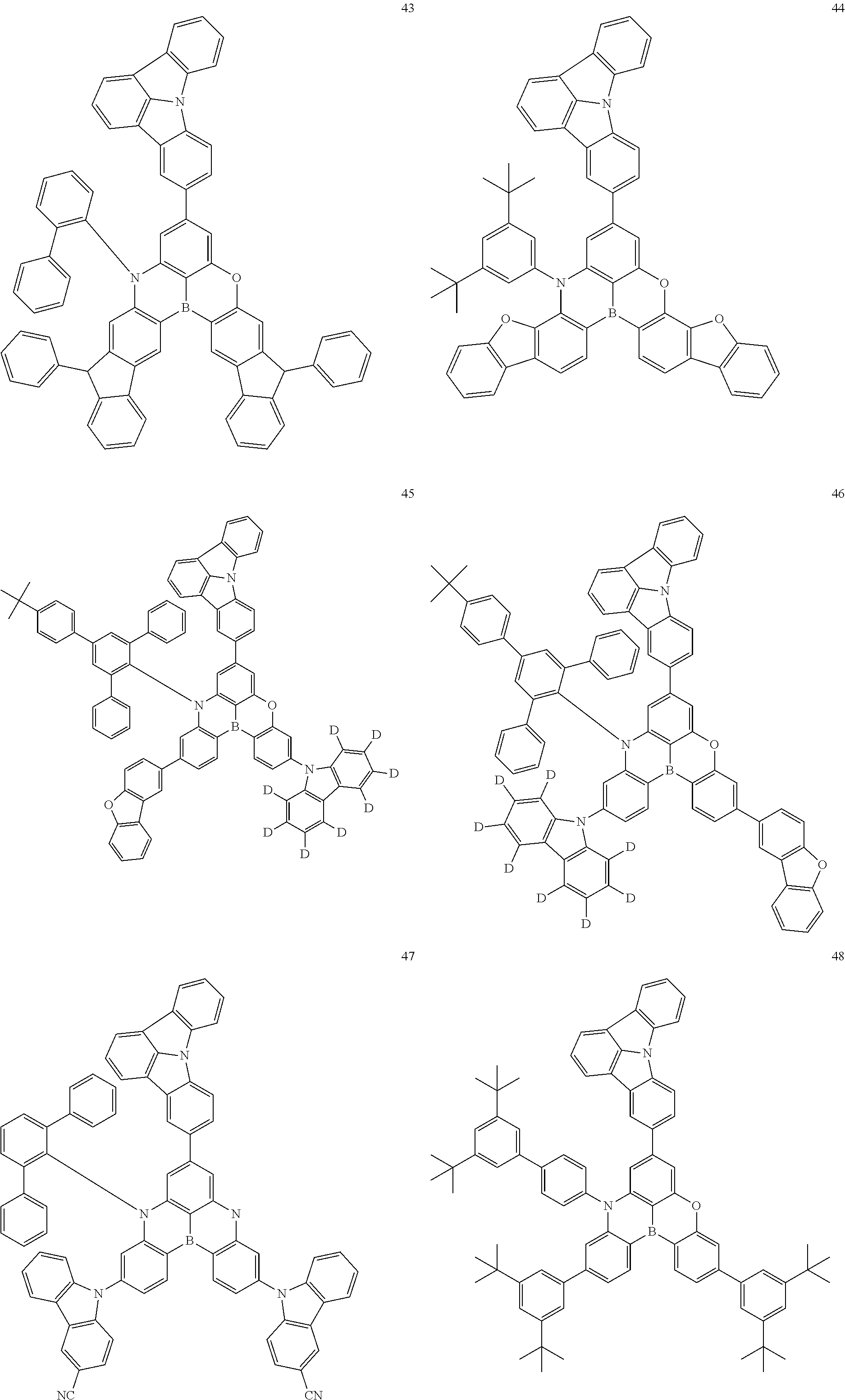

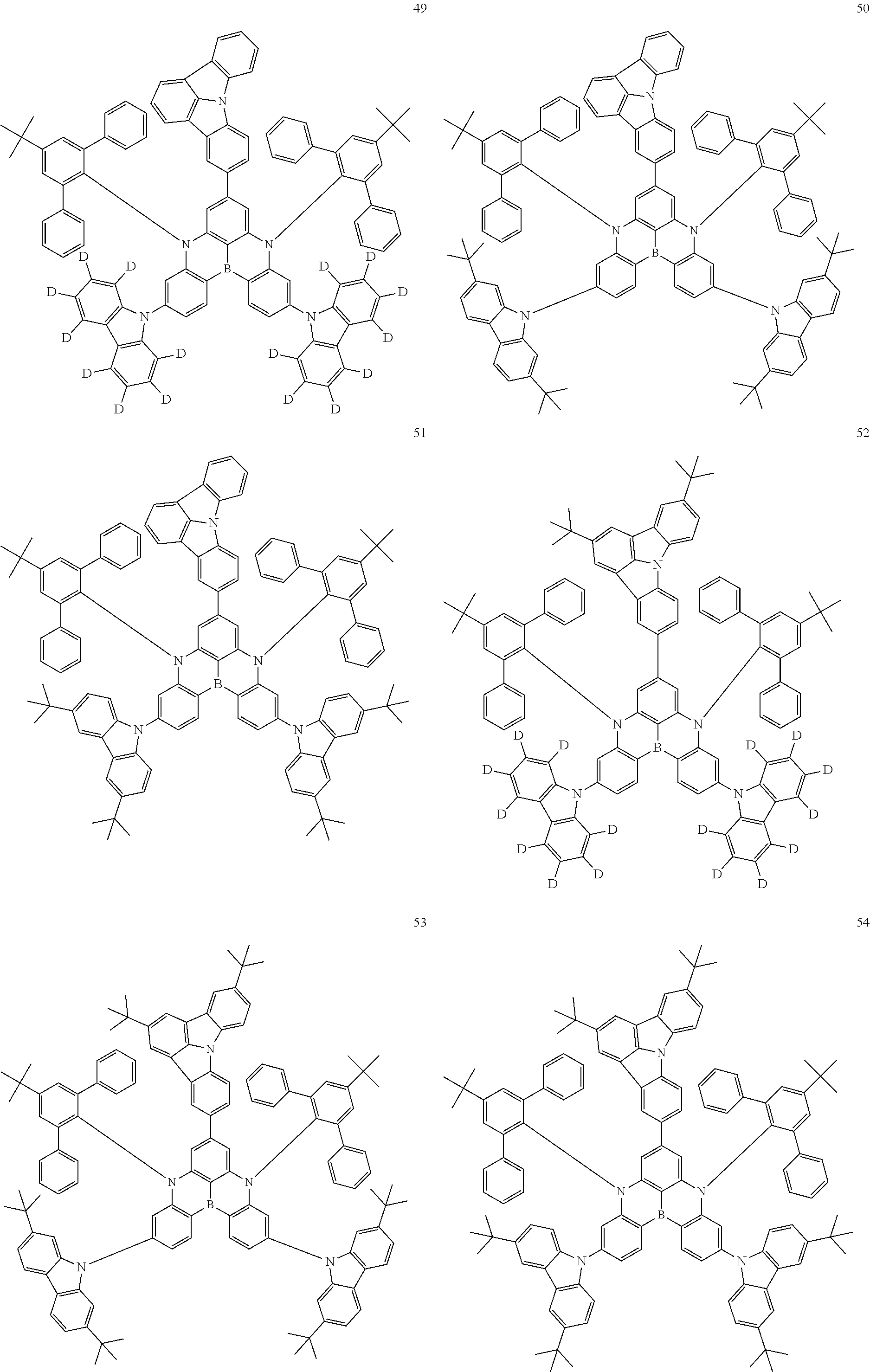

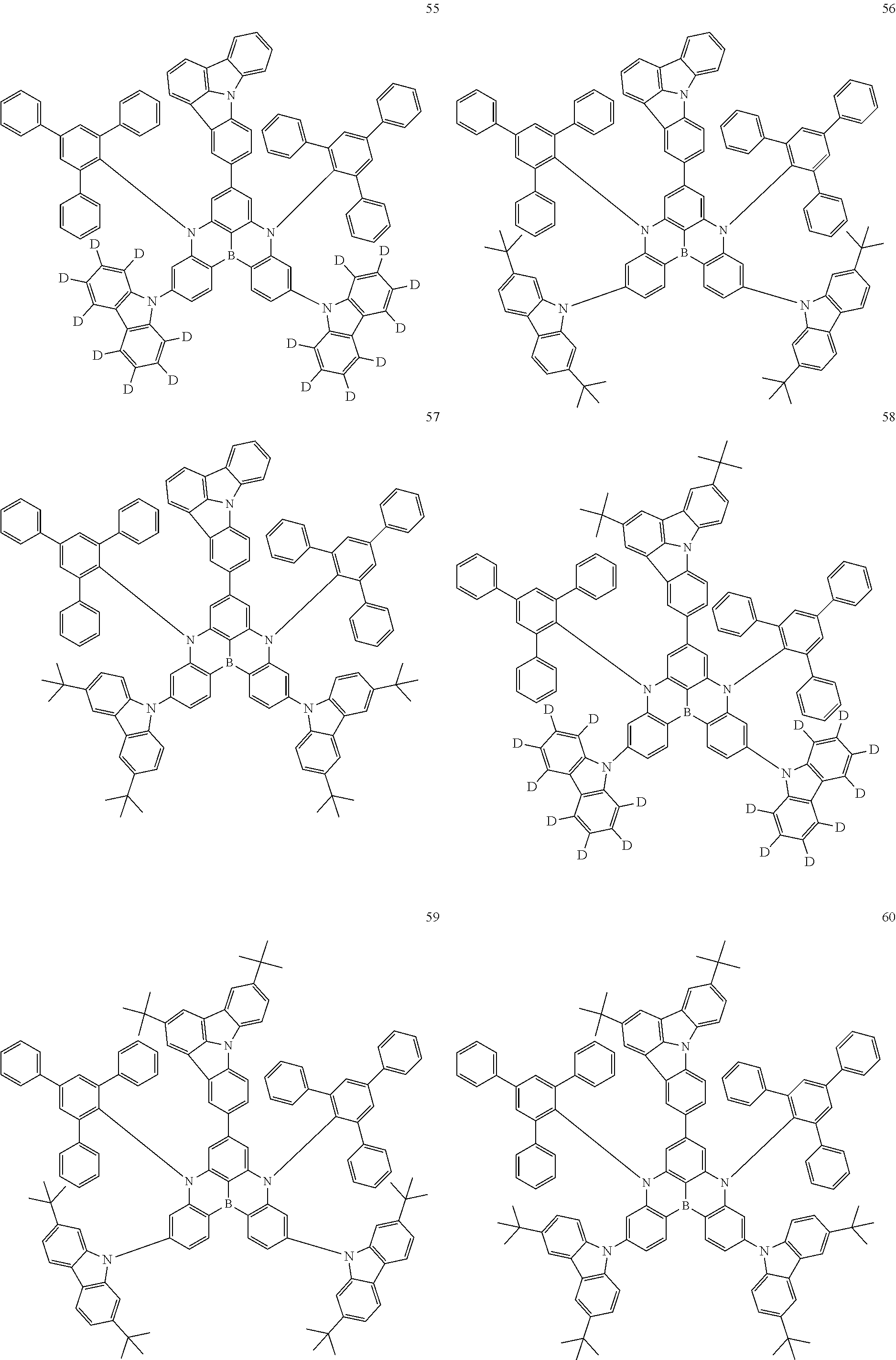

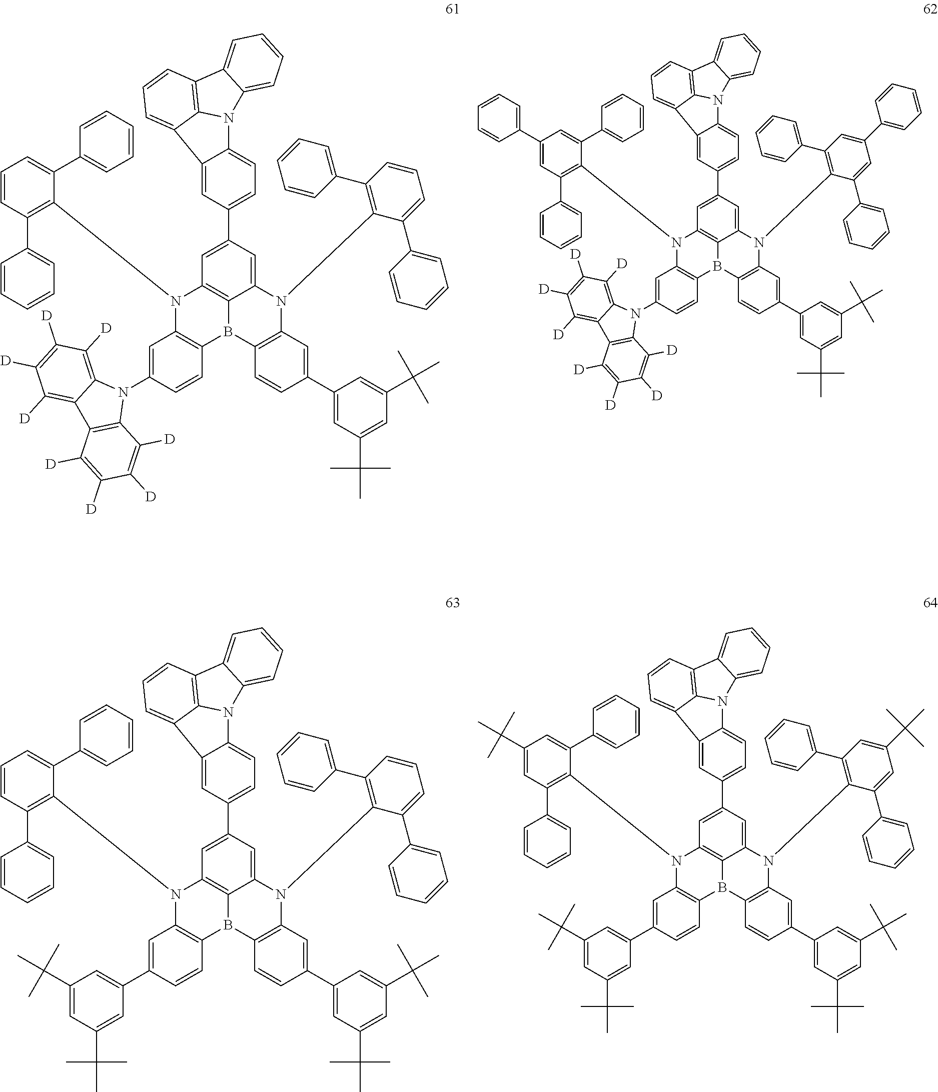

The light emission spectrum of the first dopant of an embodiment, represented by Formula 1, may have a full width at half maximum (FWHM) in a range of about 10 nm to about 50 nm. For example, the light emission spectrum of the first dopant may have a FWHM in a range of about 20 nm to about 40 nm. Since the light emission spectrum of the first dopant of an embodiment, represented by Formula 1, has a full width at half maximum in the range described above, emission efficiency may be improved when the first dopant is included in the light emitting device ED. If the first dopant is used as a material for a blue light emitting device, device life may be improved.

The first dopant of an embodiment, represented by Formula 1, may be a material for emitting thermally activated delayed fluorescence. The first dopant of an embodiment, represented by Formula 1, may be a thermally activated delayed fluorescence dopant having a difference (ΔE_{ST}) between a lowest triplet excitation energy level (T1 level) and a lowest singlet excitation energy level (S1 level) equal to or less than about 0.6 eV. For example, the first dopant of an embodiment, represented by Formula 1, may be a thermally activated delayed fluorescence dopant having a difference (ΔE_{ST}) between a lowest triplet excitation energy level (T1 level) and a lowest singlet excitation energy level (S1 level) equal to or less than about 0.2 eV.

The first dopant of an embodiment, represented by Formula 1, may be a light emitting material having a central emission wavelength in a range of about 430 nm to about 490 nm. For example, the first dopant of an embodiment, represented by Formula 1, may be a blue thermally activated delayed fluorescence (TADF) dopant. However, embodiments are not limited thereto. In case that the first dopant of an embodiment is included as a light emitting material, the first dopant may be used as a dopant emitting light in various wavelength regions including a red light emitting dopant, a green light emitting dopant, etc.

In the light emitting device ED of an embodiment, an emission layer EML may emit delayed fluorescence. For example, the emission layer EML may emit thermally activated delayed fluorescence (TADF).

The emission layer EML of the light emitting device ED may emit blue light. For example, the emission layer EML of the light emitting device ED may emit blue light in a wavelength region equal to or less than about 490 nm. For example, the emission layer EML including the first dopant of an embodiment, represented by Formula 1, may emit light having a central emission wavelength in a range of about 430 nm to about 490 nm. However, embodiments are not limited thereto. For example, the emission layer EML may emit green light or red light.

In an embodiment, the emission layer EML includes a host and a dopant and may include the first dopant as the light emitting dopant. For example, in the light emitting device ED of an embodiment, the emission layer EML may include a host for emitting delayed fluorescence and a dopant for emitting delayed fluorescence and may include the first dopant represented by Formula 1 as a dopant for emitting delayed fluorescence. The emission layer EML may include at least one compound selected from Compound Group 1 as a thermally activated delayed fluorescence dopant.

In an embodiment, the first host represented by Formula H-1 may be any one selected from Compound Group 2. The emission layer EML may include at least one selected from Compound Group 2 as the first host.

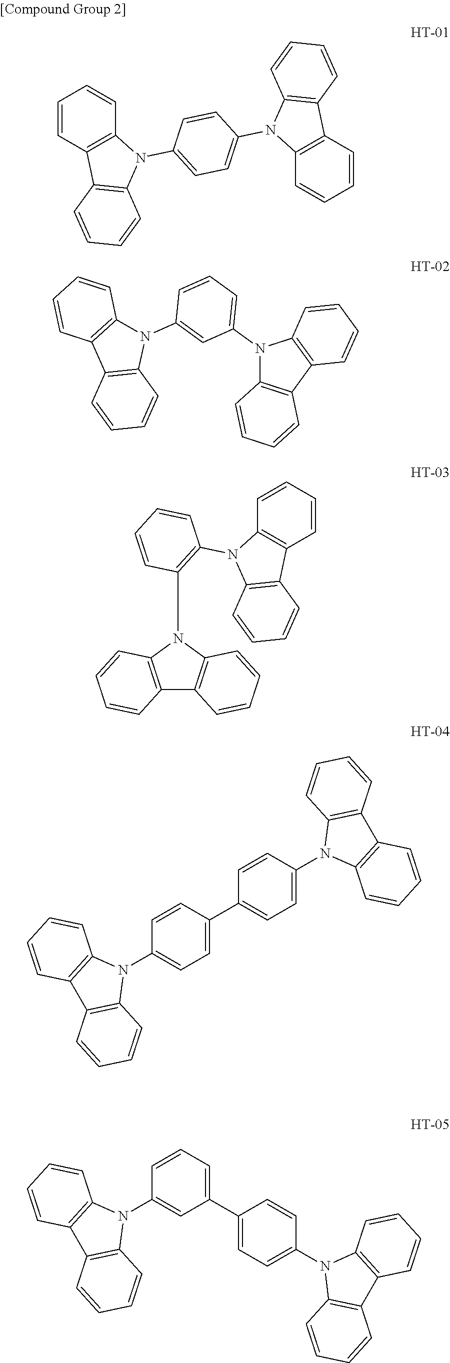

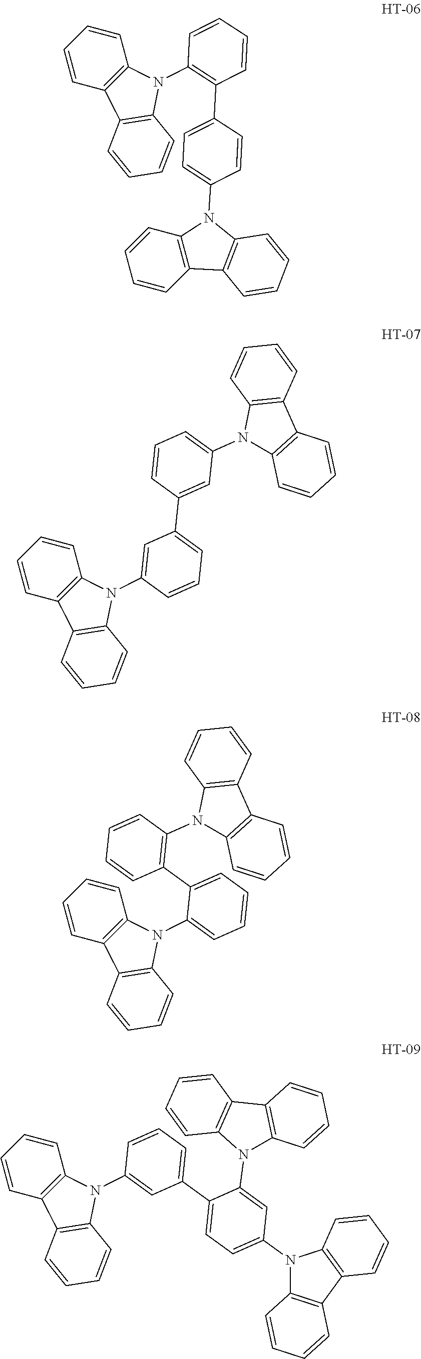 [SMILES: c1ccc(-n2c3ccccc3c3ccccc32)c(-c2ccc(-n3c4ccccc4c4ccccc43)cc2)c1 ; c1cc(-c2cccc(-n3c4ccccc4c4ccccc43)c2)cc(-n2c3ccccc3c3ccccc32)c1 ; c1ccc(-n2c3ccccc3c3ccccc32)c(-c2ccccc2-n2c3ccccc3c3ccccc32)c1 ; c1cc(-c2ccc(-n3c4ccccc4c4ccccc43)cc2-n2c3ccccc3c3ccccc32)cc(-n2c3ccccc3c3ccccc32)c1]

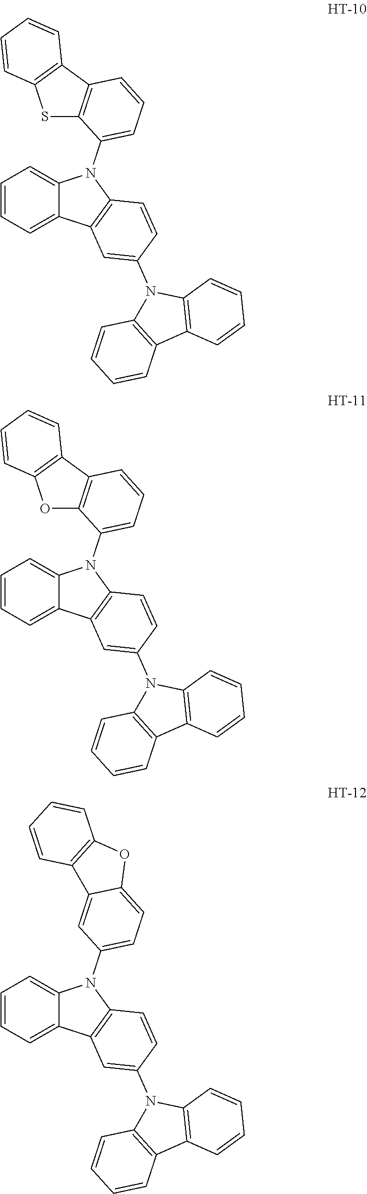

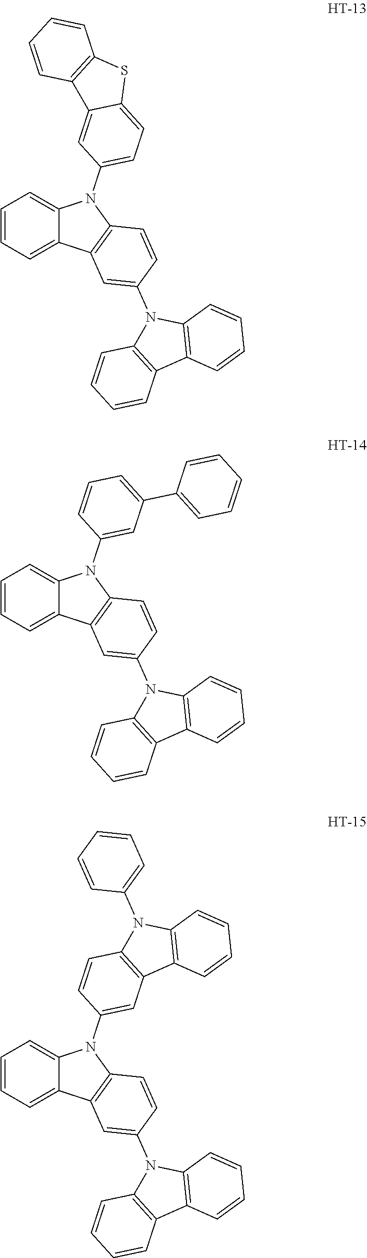 [SMILES: c1ccc2c(c1)sc1ccc(-n3c4ccccc4c4cc(-n5c6ccccc6c6ccccc65)ccc43)cc12 ; c1ccc(-c2cccc(-n3c4ccccc4c4cc(-n5c6ccccc6c6ccccc65)ccc43)c2)cc1 ; c1ccc(-n2c3ccccc3c3cc(-n4c5ccccc5c5cc(-n6c7ccccc7c7ccccc76)ccc54)ccc32)cc1]

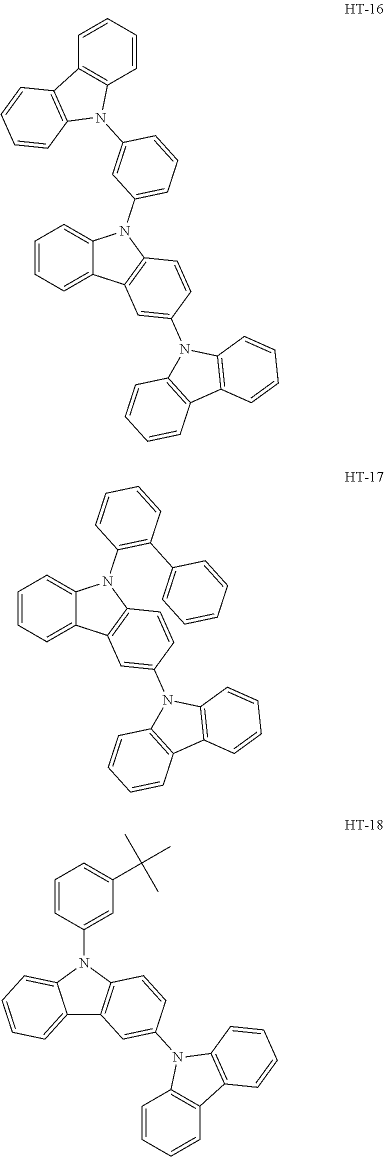

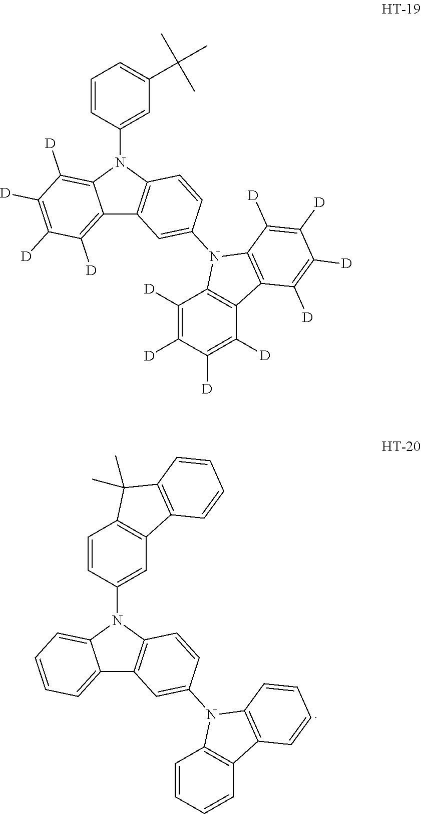 [SMILES: [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; CC1(C)c2ccccc2-c2cc(-n3c4ccccc4c4cc(-n5c6ccccc6c6ccccc65)ccc43)ccc21]

In an embodiment, the second host represented by Formula H-2 may be any one selected from Compound Group 3. The emission layer EML may include at least one selected from Compound Group 3 as the second host.

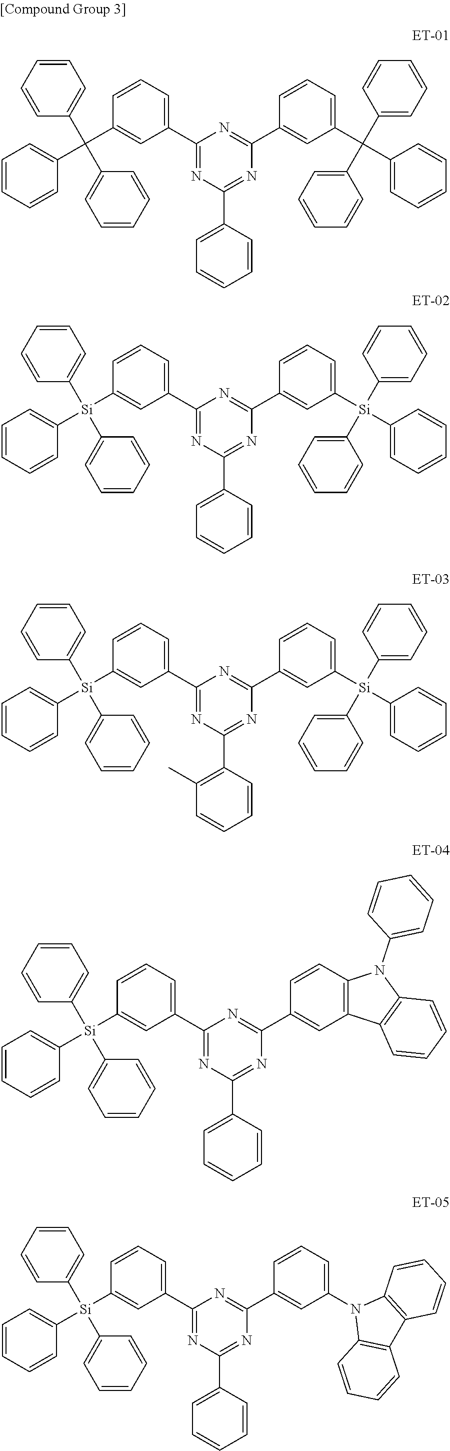

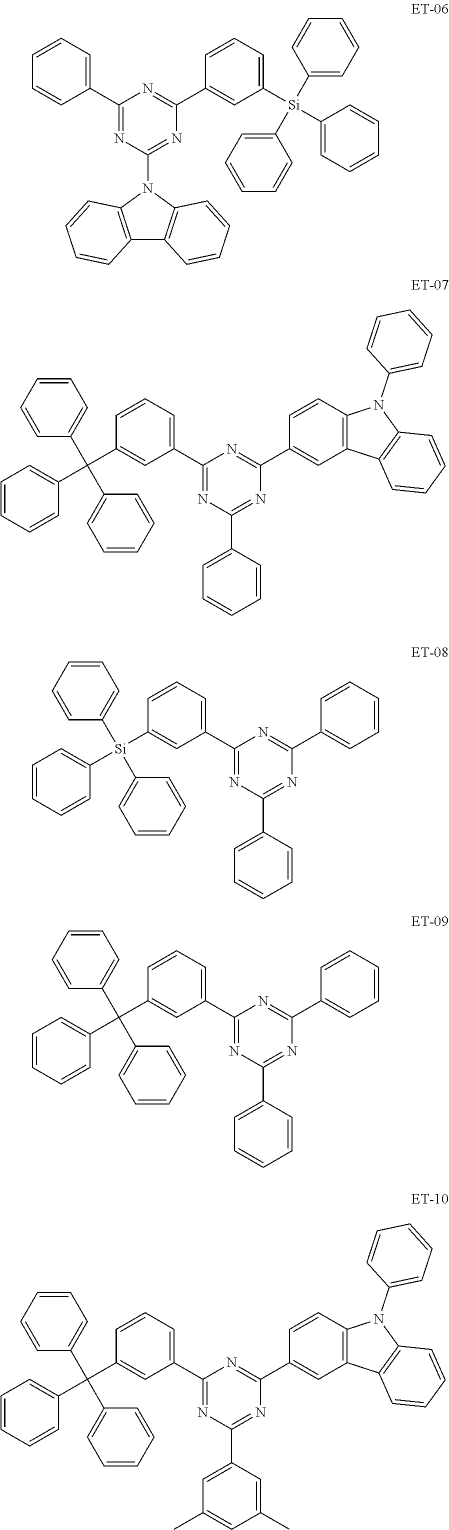 [SMILES: c1ccc(-c2nc(-c3cccc([Si](c4ccccc4)(c4ccccc4)c4ccccc4)c3)nc(-n3c4ccccc4c4ccccc43)n2)cc1 ; c1ccc(-c2nc(-c3cccc(C(c4ccccc4)(c4ccccc4)c4ccccc4)c3)nc(-c3ccc4c(c3)c3ccccc3n4-c3ccccc3)n2)cc1 ; c1ccc(-c2nc(-c3ccccc3)nc(-c3cccc([Si](c4ccccc4)(c4ccccc4)c4ccccc4)c3)n2)cc1 ; c1ccc(-c2nc(-c3ccccc3)nc(-c3cccc(C(c4ccccc4)(c4ccccc4)c4ccccc4)c3)n2)cc1 ; Cc1cc(C)cc(-c2nc(-c3cccc(C(c4ccccc4)(c4ccccc4)c4ccccc4)c3)nc(-c3ccc4c(c3)c3ccccc3n4-c3ccccc3)n2)c1]

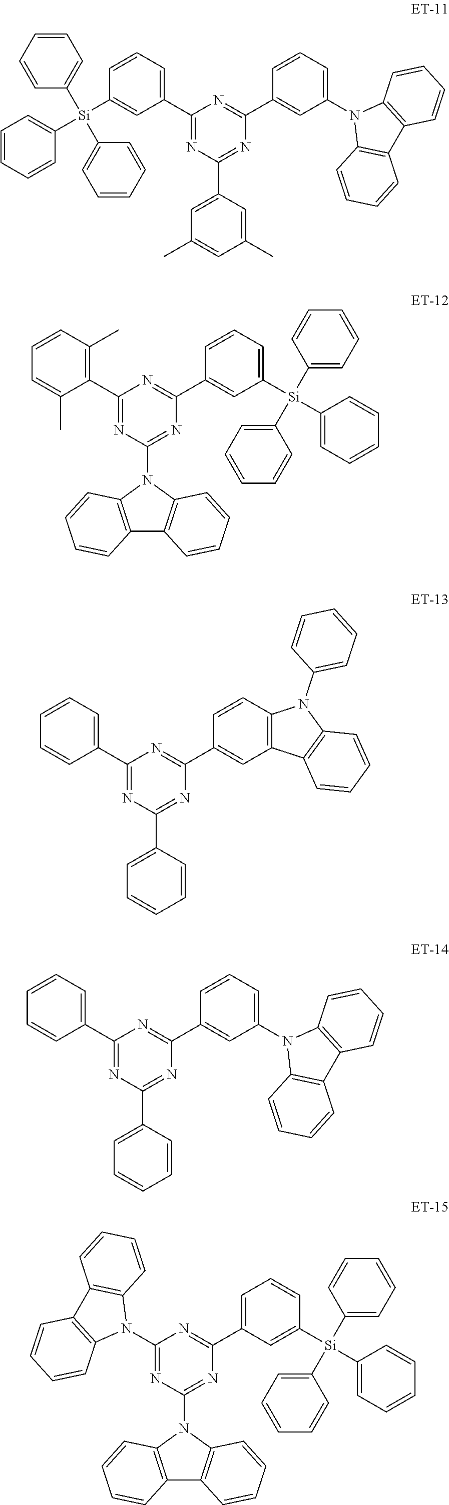 [SMILES: Cc1cc(C)cc(-c2nc(-c3cccc(-n4c5ccccc5c5ccccc54)c3)nc(-c3cccc([Si](c4ccccc4)(c4ccccc4)c4ccccc4)c3)n2)c1 ; Cc1cccc(C)c1-c1nc(-c2cccc([Si](c3ccccc3)(c3ccccc3)c3ccccc3)c2)nc(-n2c3ccccc3c3ccccc32)n1 ; c1ccc(-c2nc(-c3ccccc3)nc(-c3ccc4c(c3)c3ccccc3n4-c3ccccc3)n2)cc1 ; c1ccc(-c2nc(-c3ccccc3)nc(-c3cccc(-n4c5ccccc5c5ccccc54)c3)n2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1]

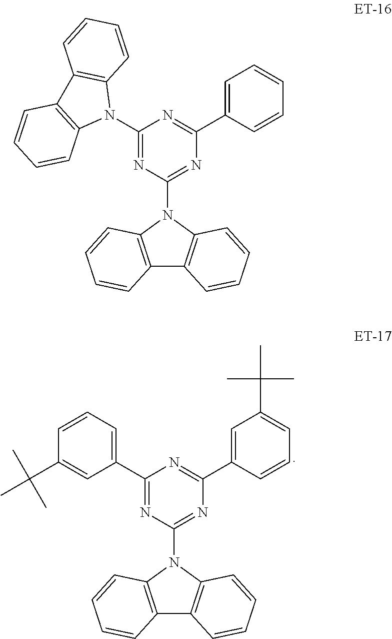 [SMILES: c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1]

In an embodiment, the emission layer EML may include at least one selected from Compound Group 4 as the second dopant.

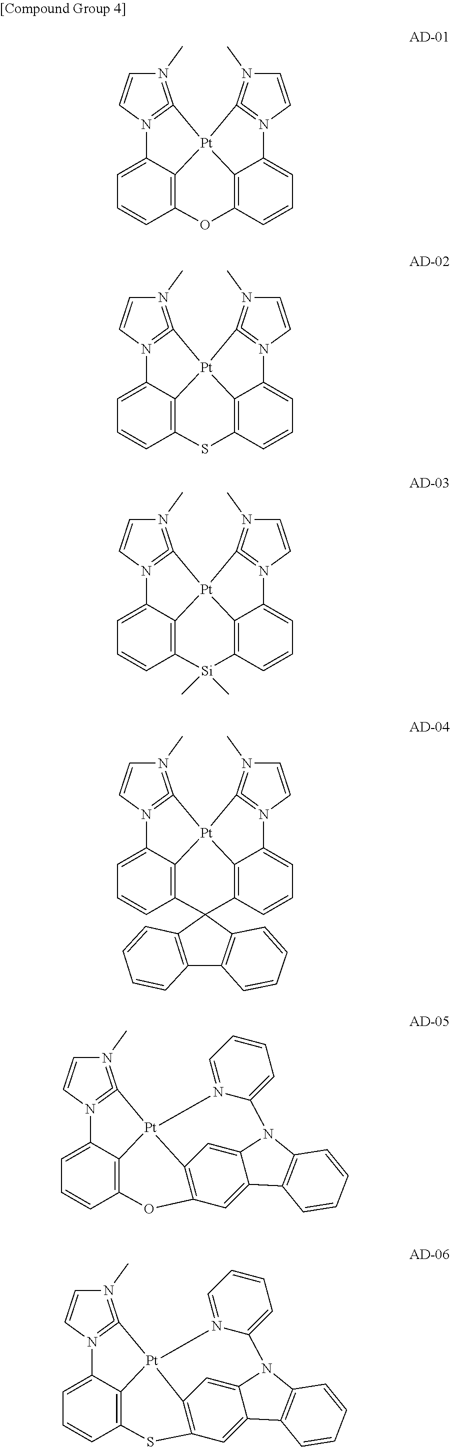

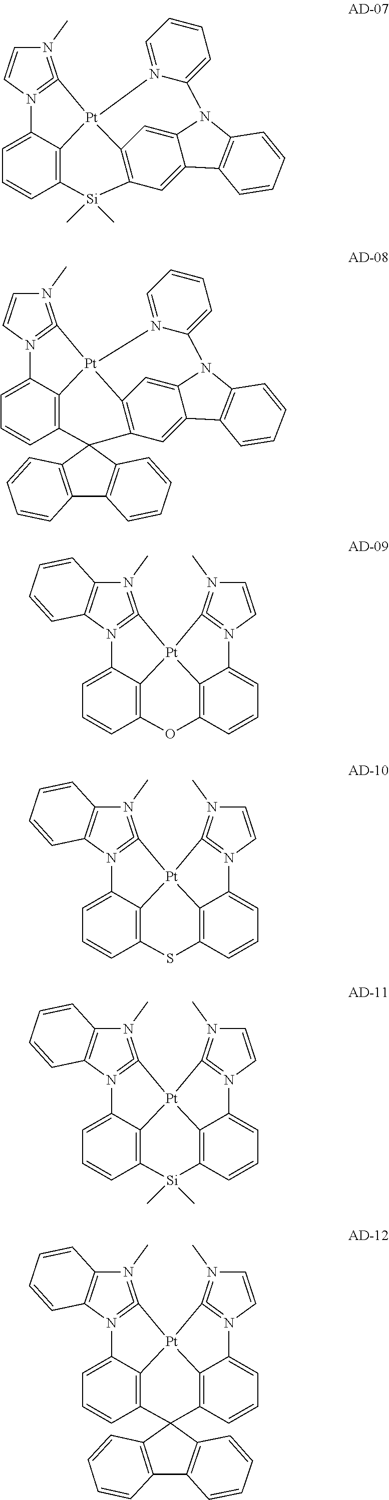

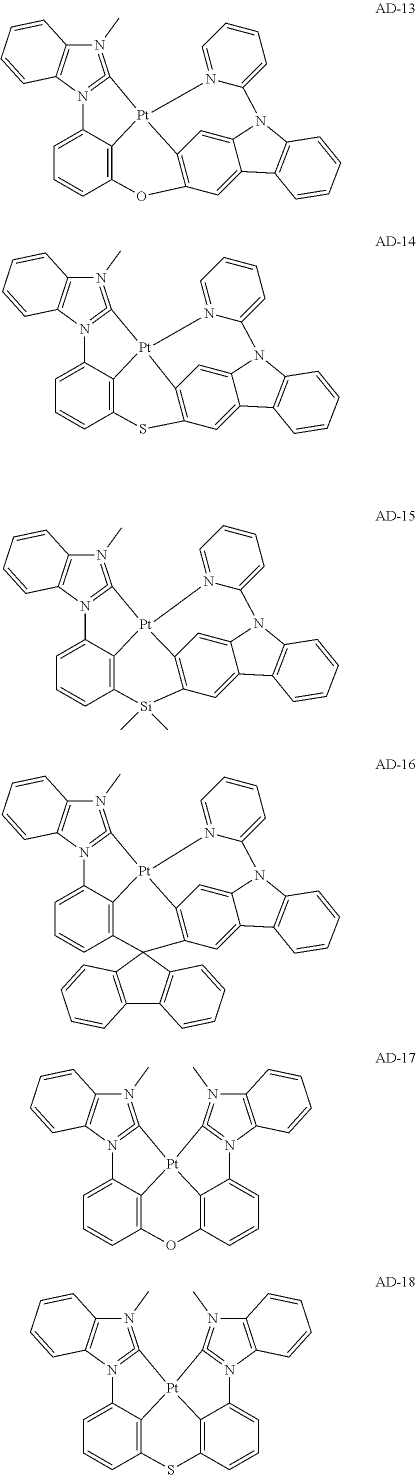

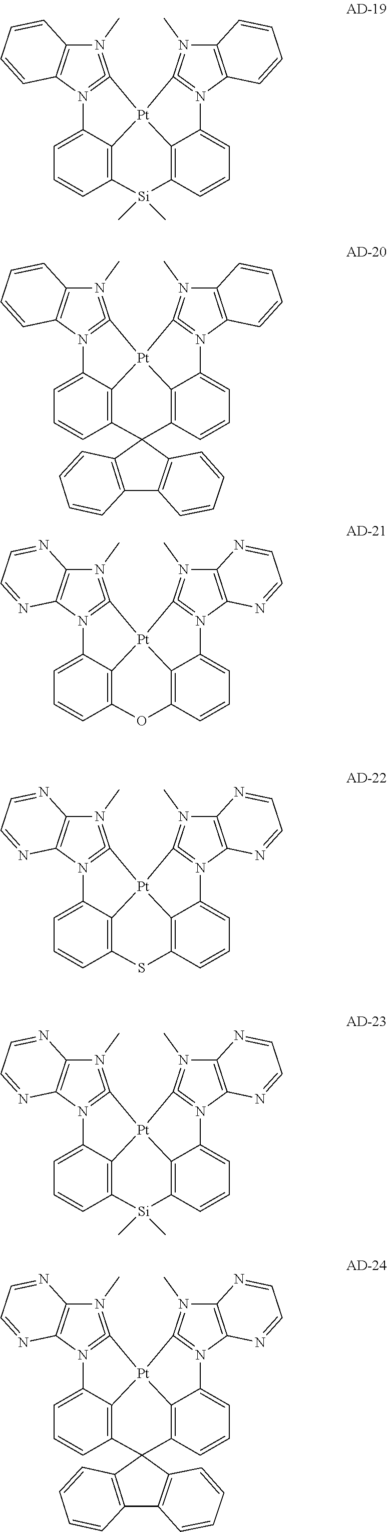

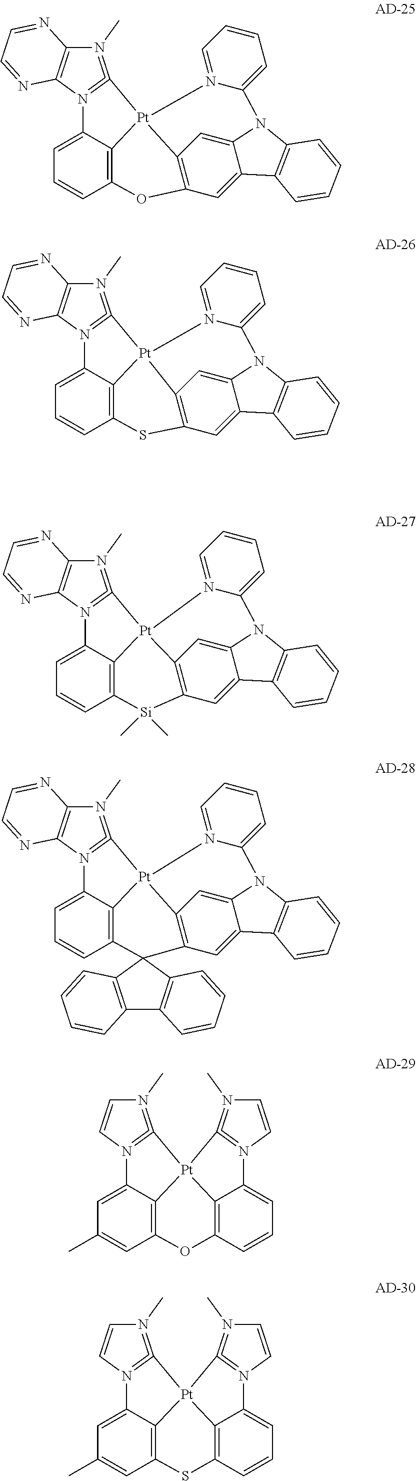

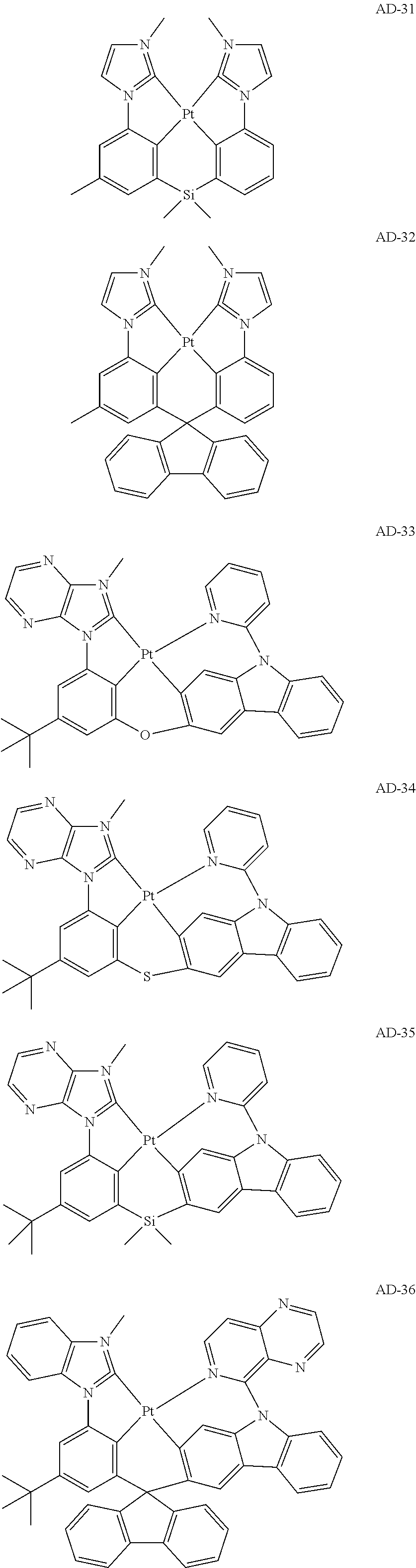

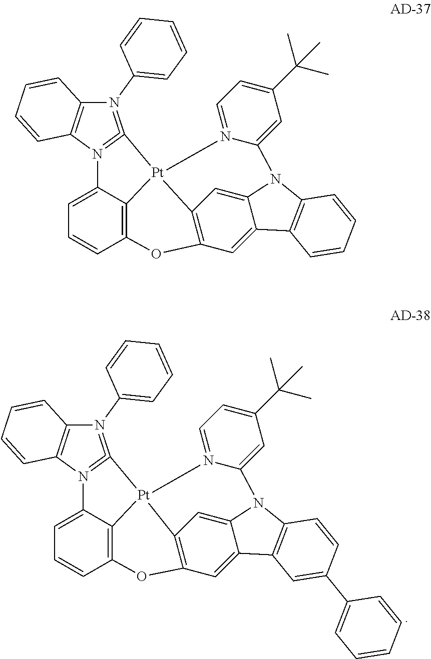

In an embodiment, the light emitting device ED may include multiple emission layers, which will be explained later. The light emitting device ED including the multiple emission layers may emit white light by providing the multiple emission layers as a stack. The light emitting device including the multiple emission layers may be a light emitting device with a tandem structure. If the light emitting device ED includes multiple emission layers, at least one emission layer EML may include the first host, the second host, the first dopant, and the second dopant as described above.

In the light emitting device ED of an embodiment, the emission layer EML may include anthracene derivatives, pyrene derivatives, fluoranthene derivatives, chrysene derivatives, dihydrobenzanthracene derivatives, or triphenylene derivatives. For example, the emission layer EML may include anthracene derivatives or pyrene derivatives.

In the light emitting devices ED of embodiments, as shown in FIG. 3 to FIG. 6, the emission layer EML may further include hosts and dopants of the related art in addition to the above-described host and dopant, and the emission layer EML may include a compound represented by Formula E-1. The compound represented by Formula E-1 may be used as a fluorescence host material.

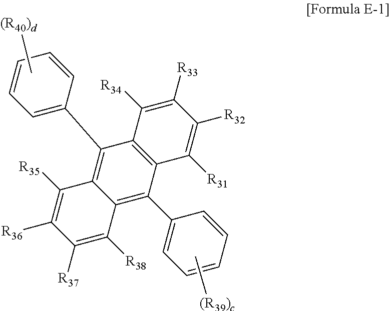

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. For example, R₃₁ to R₄₀ may be combined with an adjacent group to form a saturated hydrocarbon ring, an unsaturated hydrocarbon ring, a saturated heterocycle, or an unsaturated heterocycle.

In Formula E-1, c and d may each independently be an integer from 0 to 5.

The compound represented by Formula E-1 may be any one selected from Compound E1 to Compound E19.

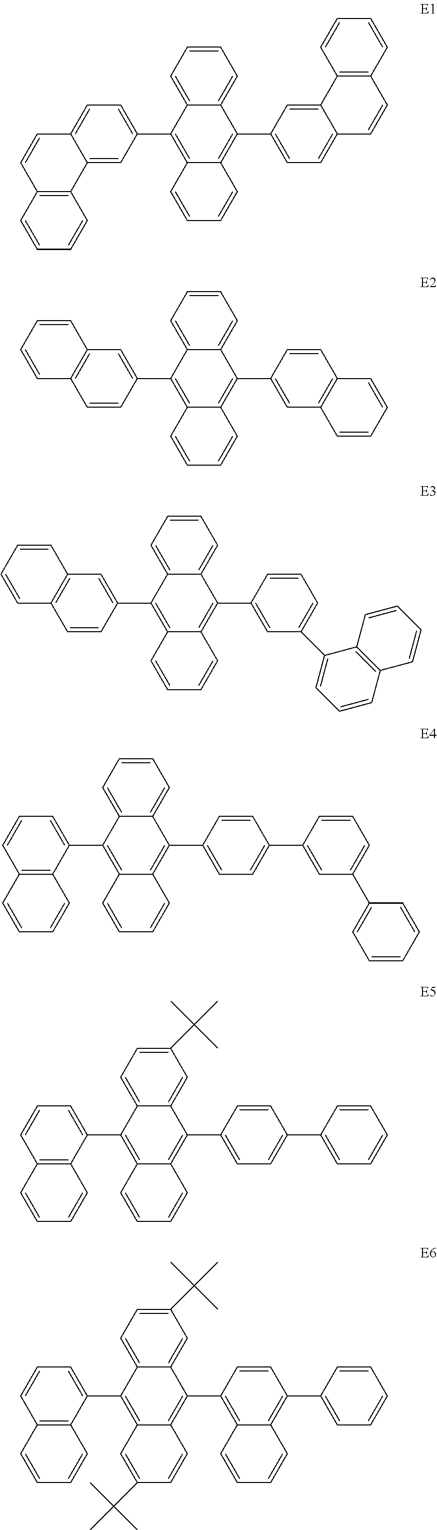

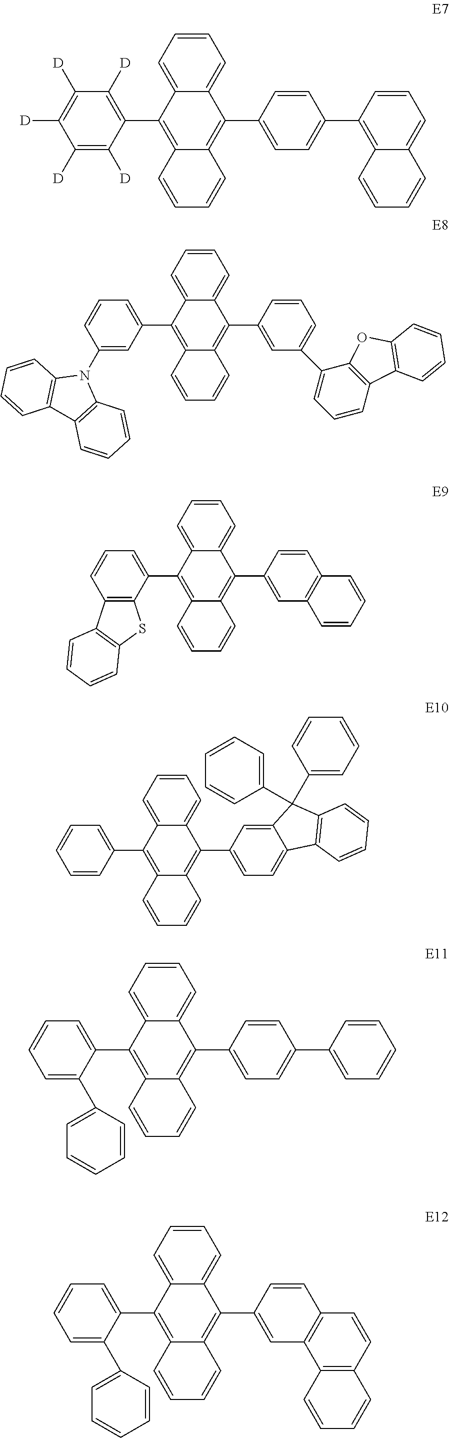

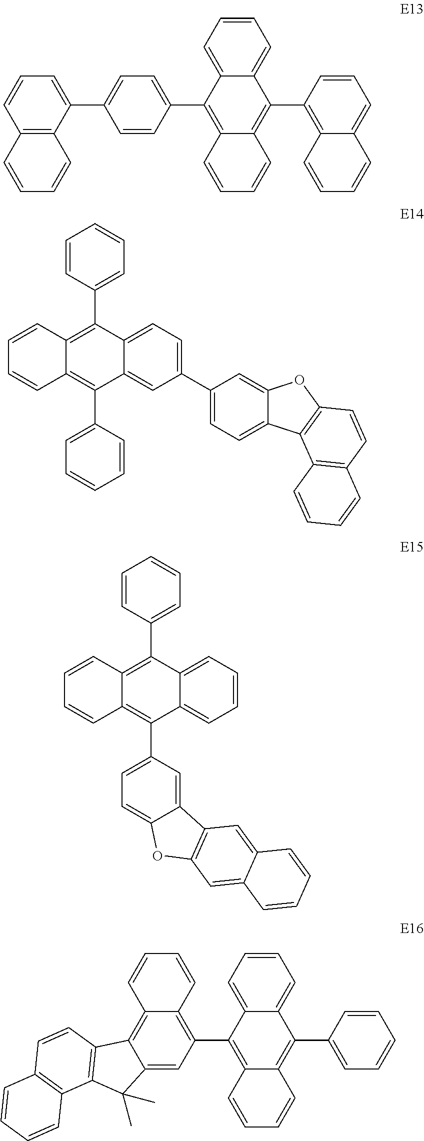

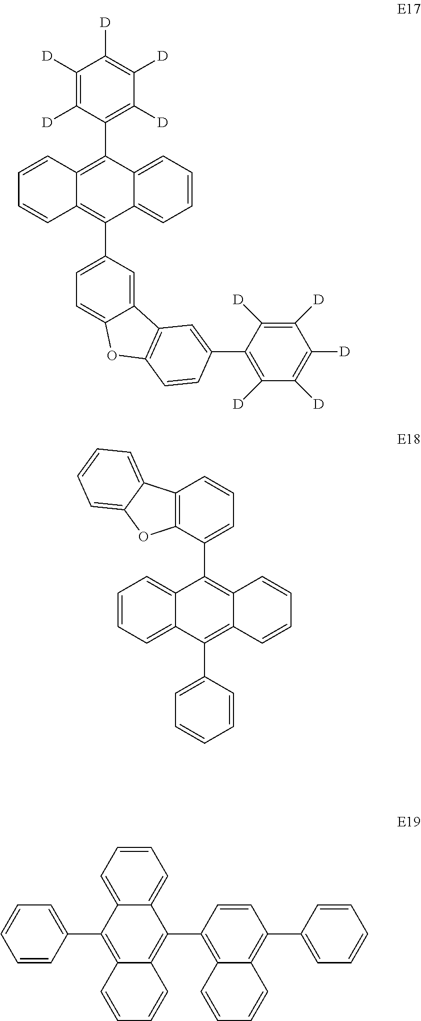

In an embodiment, the emission layer EML may further include a compound represented by Formula E-2a or Formula E-2b. The compound represented by Formula E-2a or Formula E-2b may be used as a phosphorescence host material.

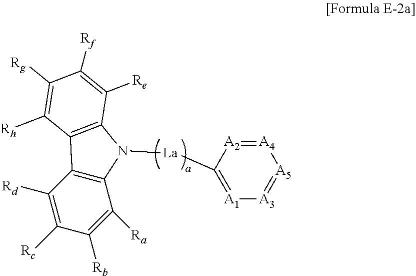

In Formula E-2a, a may be an integer from 0 to 10, and Lₐ may be a direct linkage, a substituted or unsubstituted arylene group of 6 to 30 ring-forming carbon atoms, or a substituted or unsubstituted heteroarylene group of 2 to 30 ring-forming carbon atoms. If a is 2 or more, multiple Lₐ groups may each independently be a substituted or unsubstituted arylene group of 6 to 30 ring-forming carbon atoms, or a substituted or unsubstituted heteroarylene group of 2 to 30 ring-forming carbon atoms.

In Formula E-2a, A₁ to A₅ may each independently be N or C(Rᵢ). In Formula E-2a, Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. For example, Rₐ to Rᵢ may be combined with an adjacent group to form a hydrocarbon ring or a heterocycle including N, O, S, etc. as a ring-forming atom.

In Formula E-2a, two or three of A₁ to A₅ may be N, and the remainder of A₁ to A₅ may be C(Rᵢ).

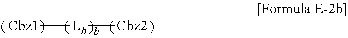

In Formula E-2b, Cbz1 and Cbz2 may each independently be an unsubstituted carbazole group, or a carbazole group substituted with an aryl group of 6 to 30 ring-forming carbon atoms. In Formula E-2b, L_{b} may be a direct linkage, a substituted or unsubstituted arylene group of 6 to 30 ring-forming carbon atoms, or a substituted or unsubstituted heteroarylene group of 2 to 30 ring-forming carbon atoms. In Formula E-2b, b may be an integer from 0 to 10, and if b is 2 or more, multiple L_{b} groups may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be any one selected from Compound Group E-2. However, the compounds shown in Compound Group E-2 are only examples, and the compound represented by Formula E-2a or Formula E-2b is not limited to Compound Group E-2.

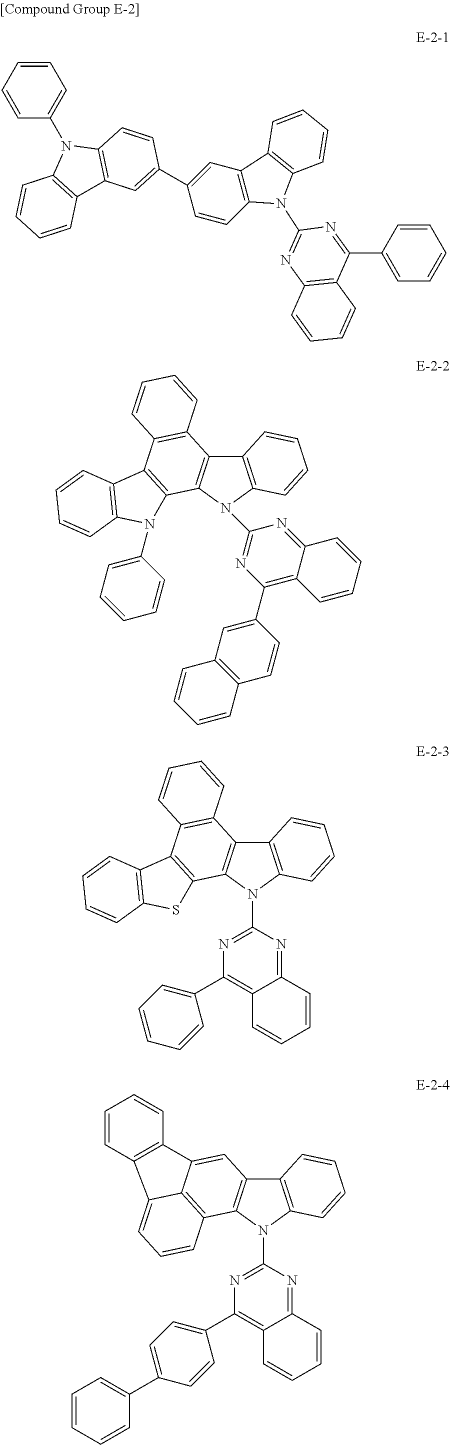

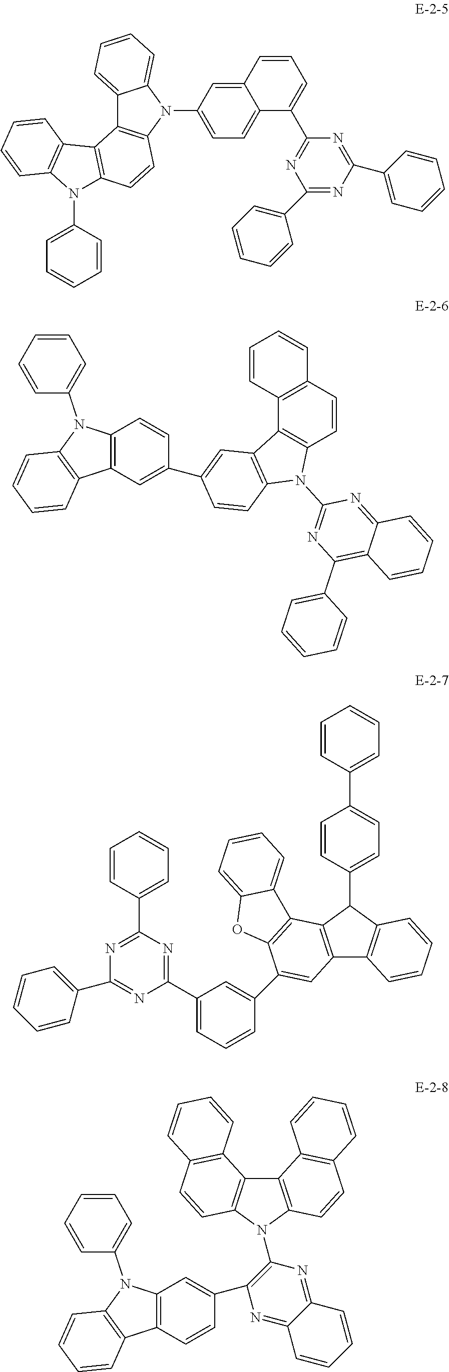 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(C3c4ccccc4-c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

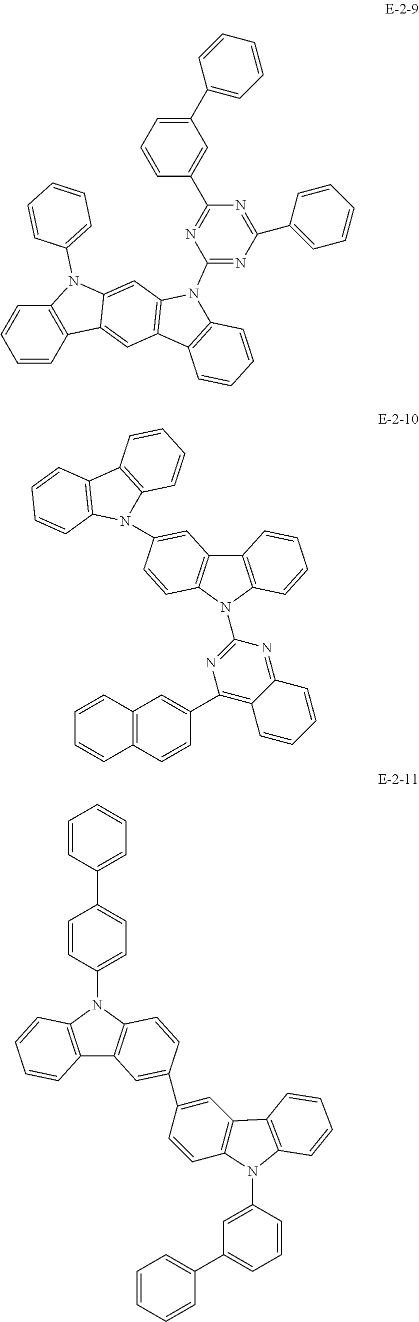

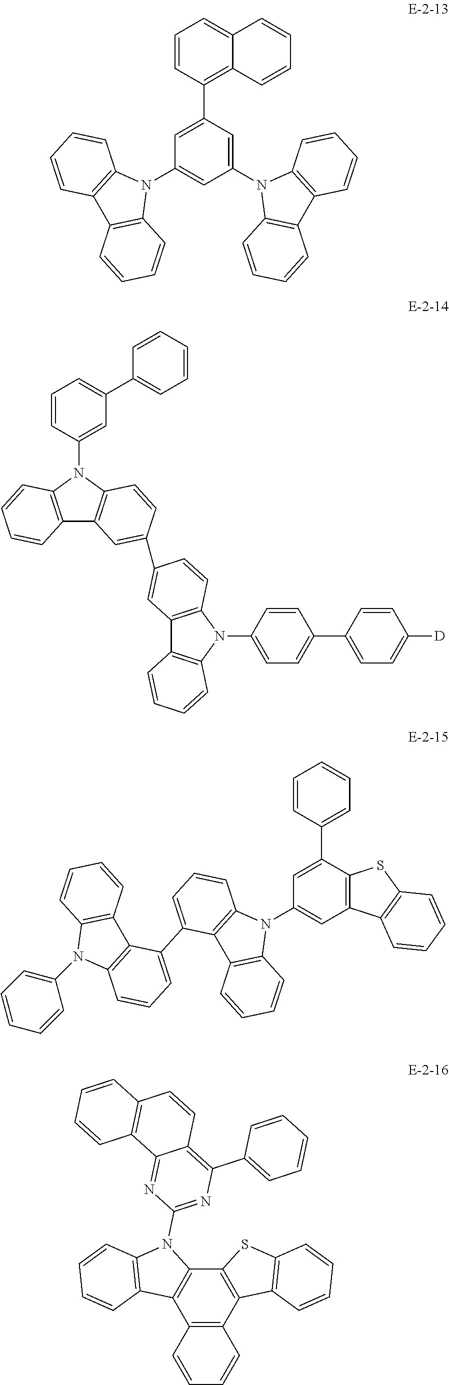 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2nc(-n3c4ccccc4c4c5ccccc5c5c6ccccc6sc5c43)nc3c2ccc2ccccc23)cc1]

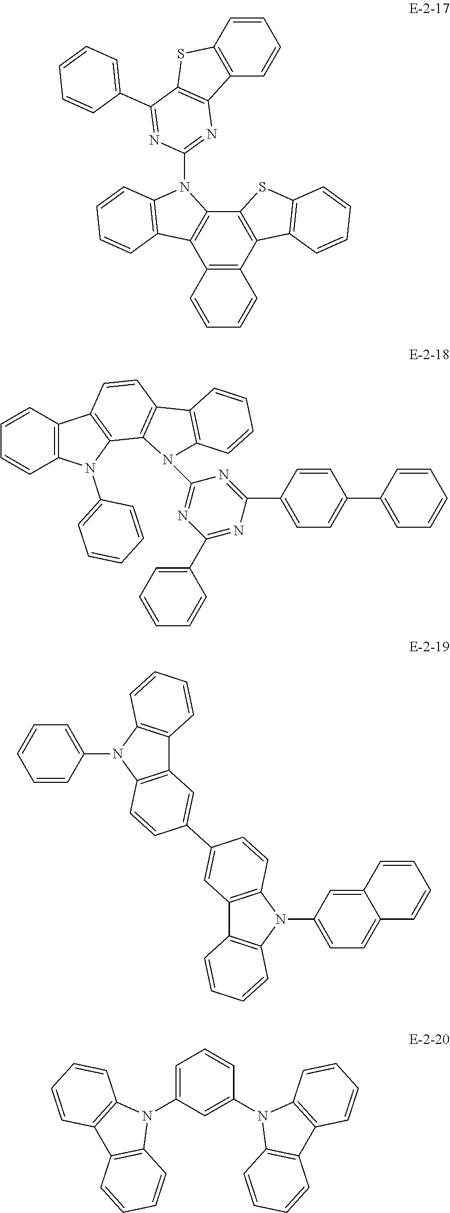

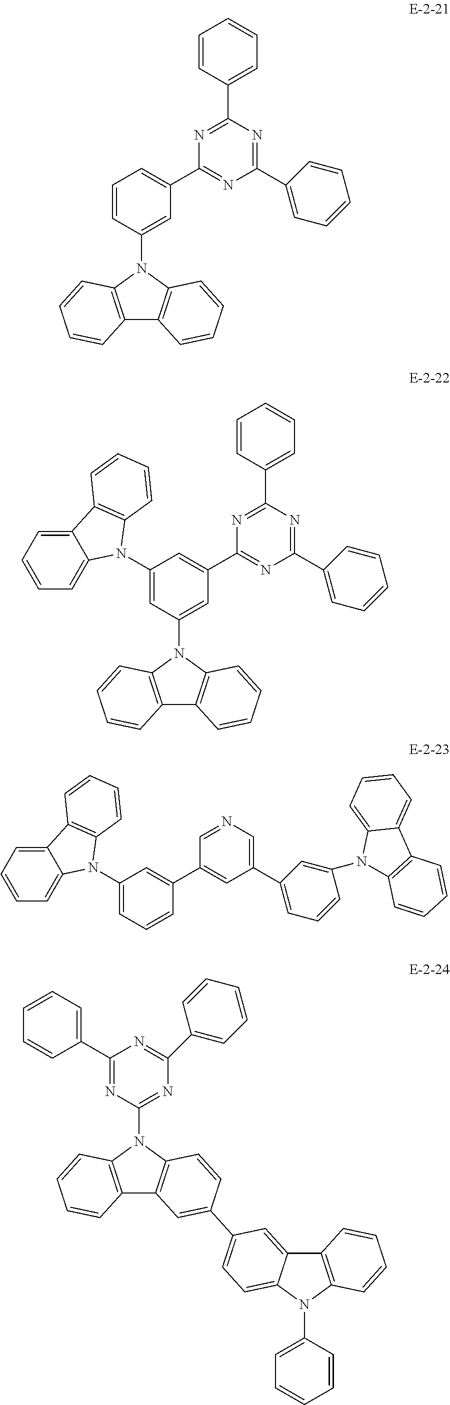 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material in the related art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(N-carbazolyl)-1,1′-biphenyl (CBP), 1,3-bis(carbazol yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, embodiments are not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as a host material.

The emission layer EML may further include a compound represented by Formula M-a or Formula M-b. The compound represented by Formula M-a or Formula M-b may be used as a phosphorescence dopant material.

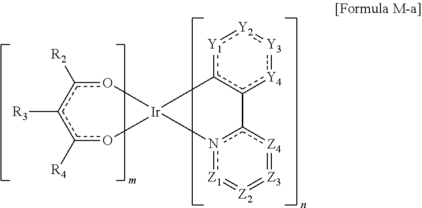

In Formula M-a, Y₁ to Y4 and Z₁ to Z₄ may each independently be C(R₁) or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, m may be 0 or 1, and n may be 2 or 3. In Formula M-a, if m is 0, n may be 3, and if m is 1, n may be 2.

The compound represented by Formula M-a may be used as a phosphorescence dopant.

The compound represented by Formula M-a may be any one selected from Compounds M-a1 to M-a25. However, Compounds M-a1 to M-a25 are only examples, and the compound represented by Formula M-a is not limited to Compounds M-a1 to M-a25.

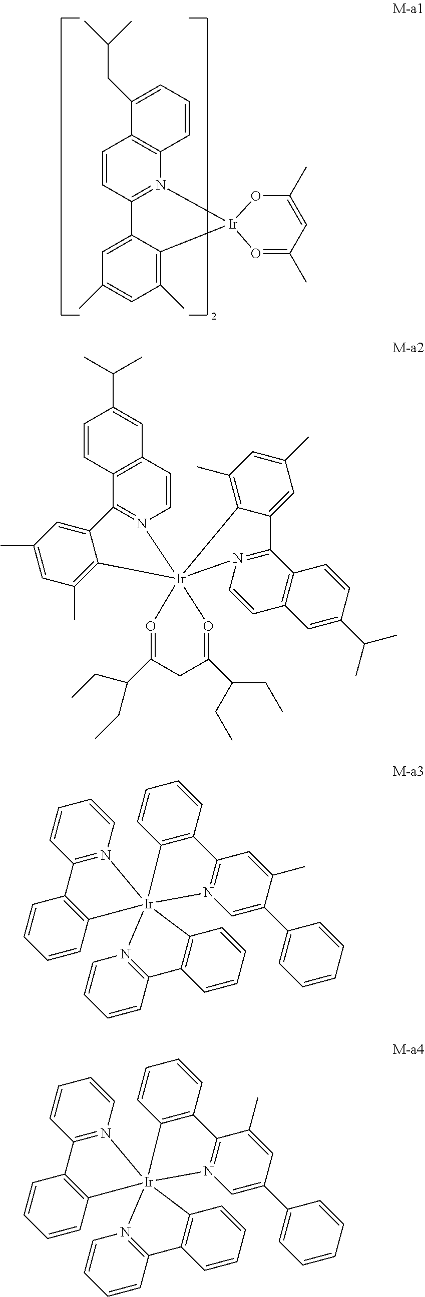

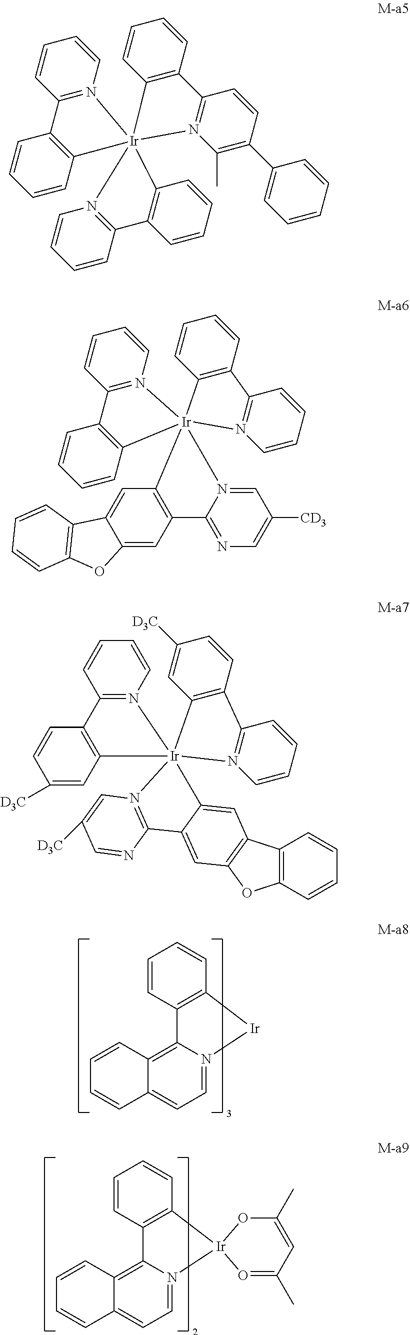

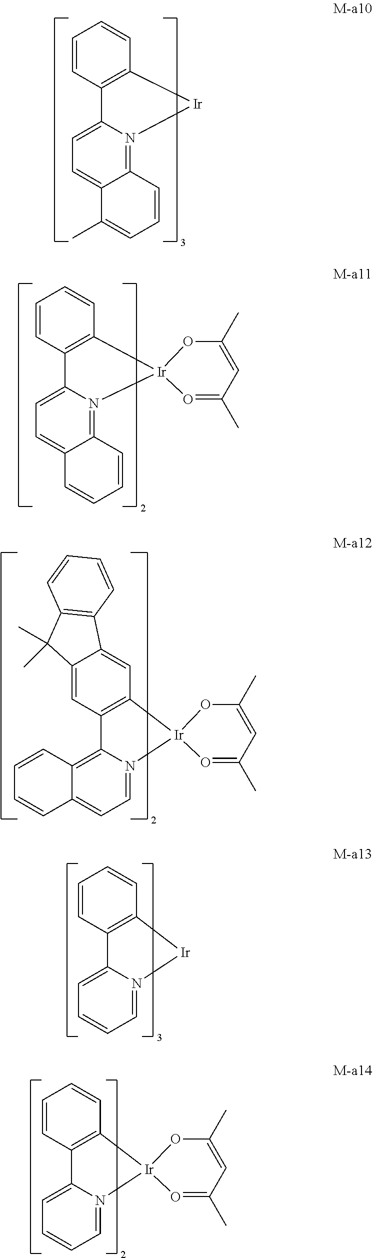

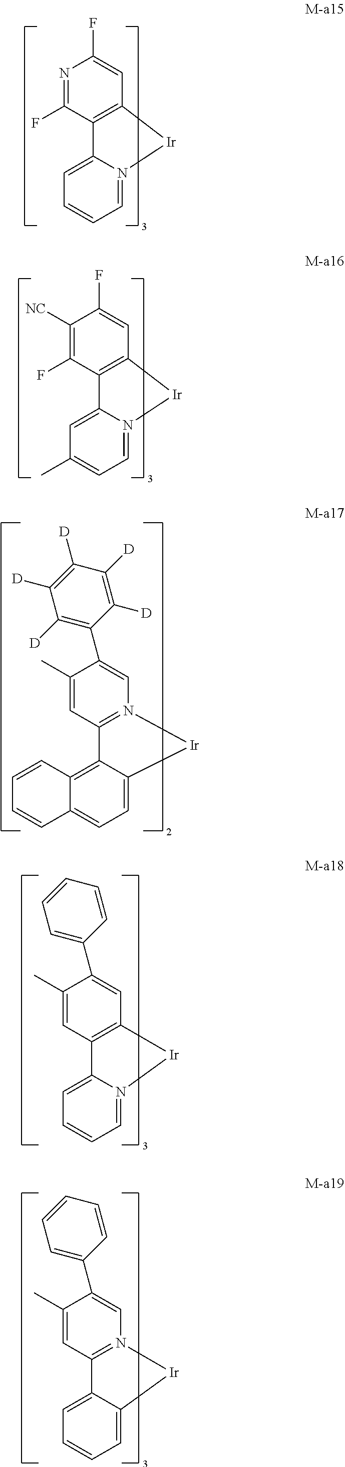

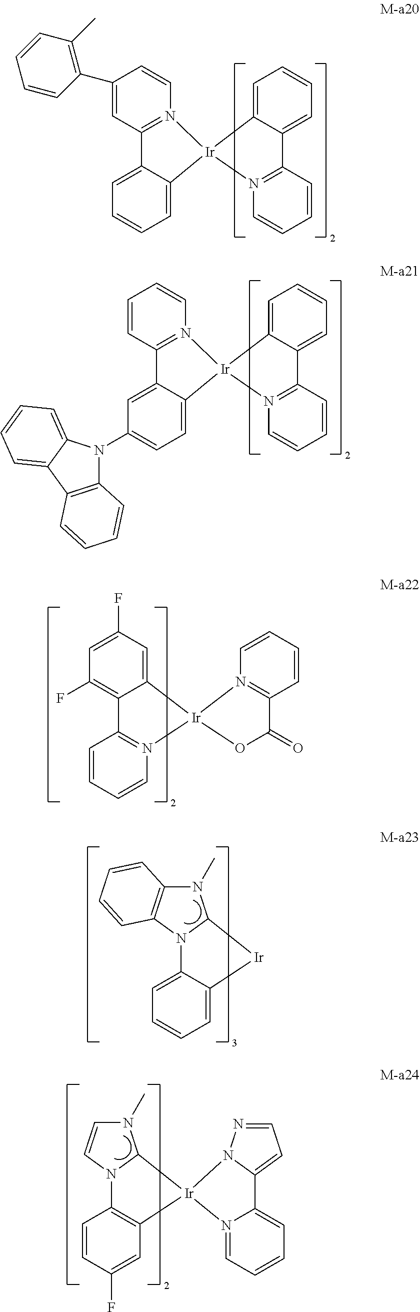

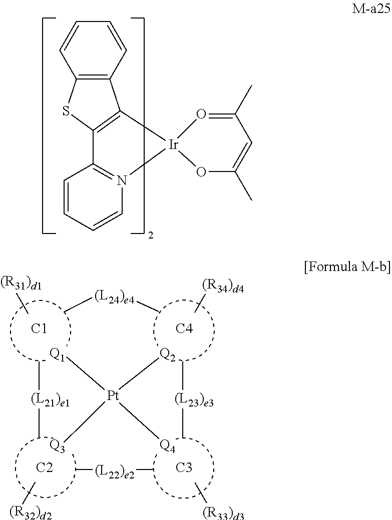

In Formula M-b, Q₁ to Q₄ may each independently be C or N, and C1 to C4 may each independently be a substituted or unsubstituted hydrocarbon ring of 5 to 30 ring-forming carbon atoms, or a substituted or unsubstituted heterocycle of 2 to 30 ring-forming carbon atoms. In Formula M-b, L₂₁ to L₂₄ may each independently be a direct linkage,

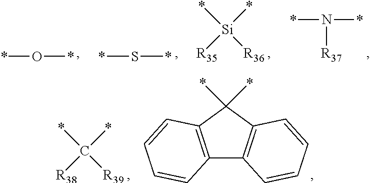

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and el to e4 may each independently be 0 or 1. In Formula M-b, R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring, and d1 to d4 may each independently be an integer from 0 to 4.

The compound represented by Formula M-b may be used as a blue phosphorescence dopant or a green phosphorescence dopant.

The compound represented by Formula M-b may be any one selected from Compounds M-b-1 to M-b-11. However, Compounds M-b-1 to M-b-11 are only examples, and the compound represented by Formula M-b is not limited to Compounds M-b-1 to M-b-11.

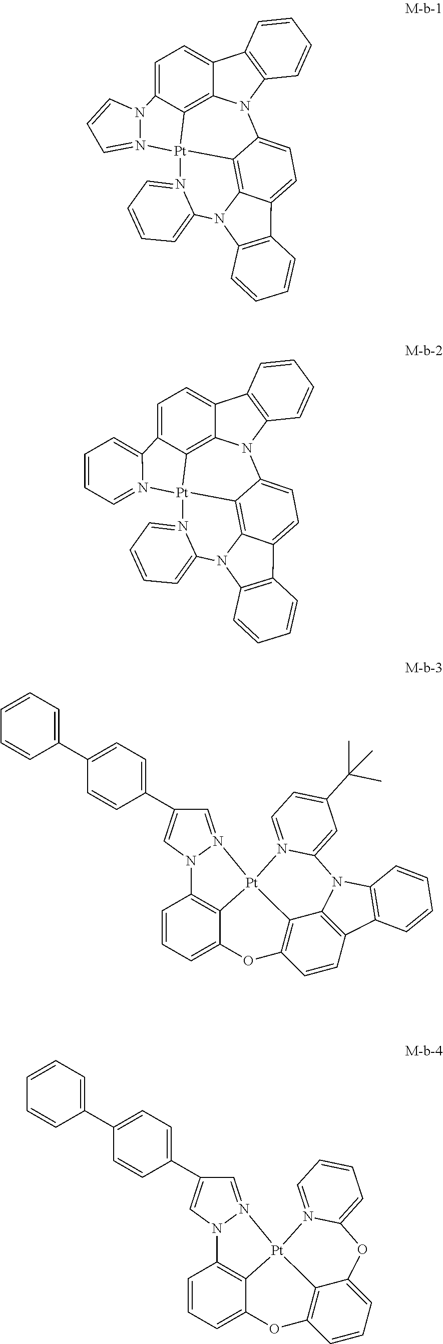

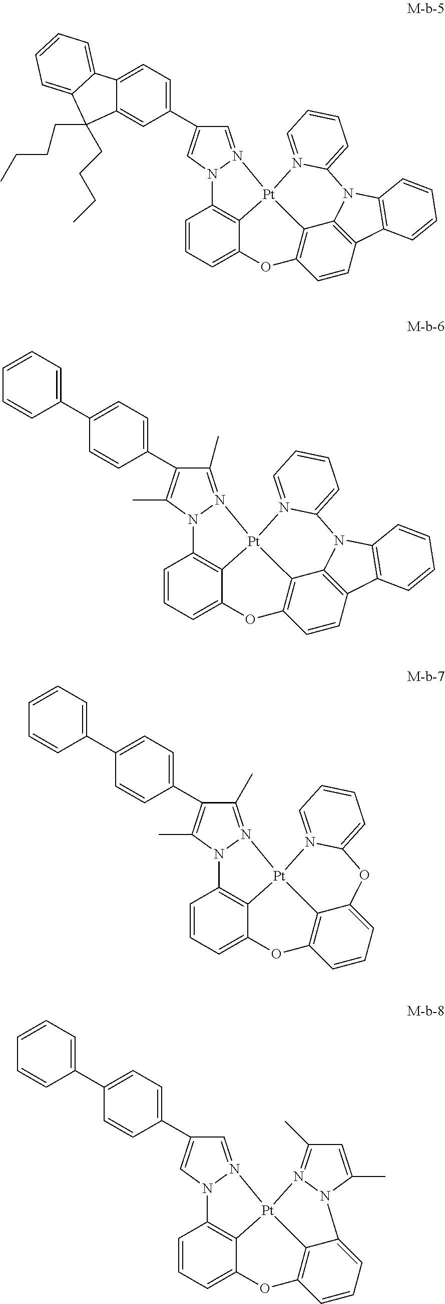

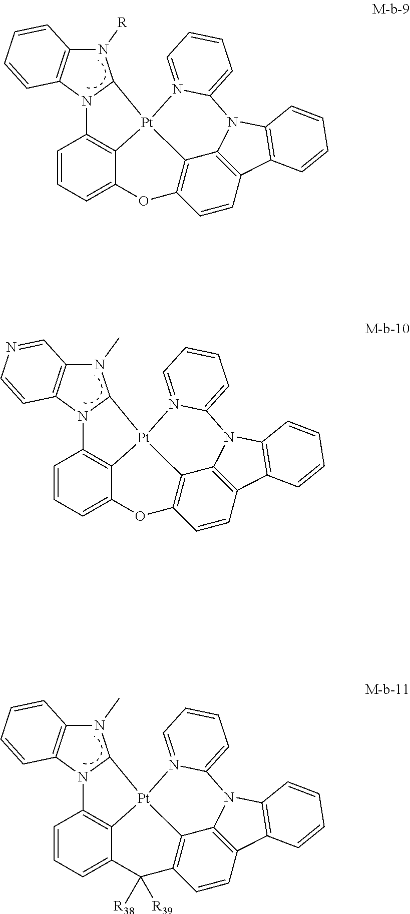

In Compounds M-b-1 to M-b-11, R, R₃₈, and R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may further include a compound represented by any one of Formula F-a to Formula F-c. The compounds represented by Formula F-a to Formula F-c may be used as fluorescence dopant materials.

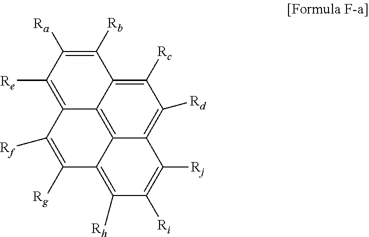

In Formula F-a, two of Rₐ to may each independently be substituted with a group represented by *—NAr₁Ar₂. The remainder of Rₐ to Rⱼ which are not substituted with the group represented by *—NAr₁Ar₂ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In the group represented by *—NAr₁Ar₂, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one of Ar₁ or Ar₂ may be a heteroaryl group including O or S as a ring-forming atom.

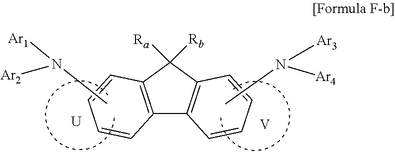

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may each independently be 0 or 1. For example, in Formula F-b, if the number of U or V is 1, a fused ring may be present at the part designated by U or V, and if the number of U or V is 0, a fused ring may not be present at the part designated by U or V. If the number of U is 0 and the number of V is 1, or if the number of U is 1 and the number of V is 0, a fused ring having the fluorene core of Formula F-b may be a polycyclic compound with four rings. If the number of both U and V is 0, a fused ring having the fluorene core of Formula F-b may be a polycyclic compound with three rings. If the number of both U and V is 1, a fused ring having the fluorene core of Formula F-b may be a polycyclic compound with five rings.

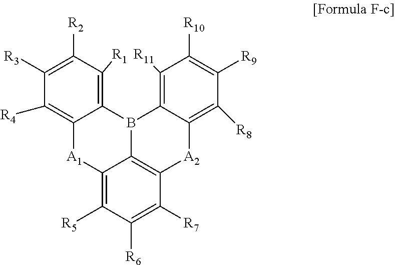

In Formula F-c, A₁ and A₂ may each independently be 0, S, Se, or N(Rₘ), and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In Formula F-c, R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula F-c, A₁ and A₂ may each independently be combined with the substituents of an adjacent ring to form a fused ring. For example, if A₁ and A₂ are each independently N(Rₘ), A₁ may be combined with R₄ or R₅ to form a ring. For example, A₂ may be combined with R₇ or R₈ to form a ring.

In an embodiment, the emission layer EML may include a dopant material of the related art, such as styryl derivatives (for example, 1,4-bis[2-(3-N-ethylcarbazoryl)vinyl]benzene (BCzVB), 4-(di-p-tolylamino)-4′-[(di-p-tolylamino)styryl] stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), etc.

The emission layer EML may include a phosphorescence dopant material of the related art. For example, the phosphorescence dopant may be a metal complex including iridium (Ir), platinum (Pt), osmium (Os), gold (Au), titanium (T1), zirconium (Zr), hafnium (Hf), europium (Eu), terbium (Tb), or thulium (Tm). For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be used as the phosphorescence dopant. However, embodiments are not limited thereto.

The emission layer EML may include a quantum dot. The quantum dot may be selected from a Group II-VI compound, a Group III-VI compound, a Group compound, a Group III-V compound, a Group III-II-V compound, a Group IV-VI compound, a Group IV element, a Group IV compound, or a combination thereof.

The Group II-VI compound may be selected from: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof; or any combination thereof.

The Group III-VI compound may include: a binary compound such as In₂S₃, and In₂Se₃; a ternary compound such as InGaS₃, and InGaSe₃; or any combination thereof.

The Group compound may be selected from: a ternary compound selected from the group consisting of AgInS, AgInS₂, CuInS, CuInS₂, AgGaS₂, CuGaS₂, CuGaO₂, AgGaO₂, AgAlO₂ and mixtures thereof; a quaternary compound such as AgInGaS₂, and CuInGaS₂; or any combination thereof.

The Group III-V compound may be selected from: a binary compound selected from the group consisting of GaN, GaP, GaAs, GaSb, AlN, AlP, AlAs, AlSb, InN, InP, InAs, InSb, and mixtures thereof; a ternary compound selected from the group consisting of GaNP, GaNAs, GaNSb, GaPAs, GaPSb, AlNP, AINAs, AlNSb, AlPAs, AlPSb, InGaP, InAlP, InNP, InNAs, InNSb, InPAs, InPSb, and mixtures thereof; a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof; or any combination thereof. The Group III-V compound may further include a Group II metal. For example, InZnP, etc. may be selected as a Group III-II-V compound.

The Group IV-VI compound may be selected from: a binary compound selected from the group consisting of SnS, SnSe, SnTe, PbS, PbSe, PbTe, and mixtures thereof; a ternary compound selected from the group consisting of SnSeS, SnSeTe, SnSTe, PbSeS, PbSeTe, PbSTe, SnPbS, SnPbSe, SnPbTe, and mixtures thereof; a quaternary compound selected from the group consisting of SnPbSSe, SnPbSeTe, SnPbSTe, and mixtures thereof; or any combination thereof. The Group IV element may be selected from the group consisting of Si, Ge, and a mixture thereof. The Group IV compound may be a binary compound selected from the group consisting of SiC, SiGe, and a mixture thereof.

A binary compound, a ternary compound, or a quaternary compound may be present in a particle at a uniform concentration or may be present at a partially different concentration distribution state. In an embodiment, the quantum dot may have a core/shell structure in which one quantum dot surrounds another quantum dot. A quantum dot having a core/shell structure may have a concentration gradient at an interface between the core that the shell in which the concentration of an element that is present in the shell decreases toward the center.

In embodiments, the quantum dot may have a core-shell structure including a core including a nanocrystal and a shell surrounding the core. The shell of the quantum dot may function as a protection layer that prevents the chemical deformation of the core to maintain semiconductor properties and/or may function as a charging layer that imparts the quantum dot with electrophoretic properties. The shell may be a single layer or a multilayer. Examples of the shell of the quantum dot may include a metal oxide, a non-metal oxide, a semiconductor compound, or combinations thereof.

For example, the metal oxide or the non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO, or a ternary compound such as MgAl₂O₄, CoFe₂O₄, NiFe₂O₄ and CoMn₂O₄, but embodiments are not limited thereto.

The semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dot may have a full width of half maximum (FWHM) of an emission wavelength spectrum equal to or less than about 45 nm. For example, the quantum dot may have a FWHM of an emission wavelength spectrum equal to or less than about 40 nm. For example, the quantum dot may have a FWHM of an emission wavelength spectrum equal to or less than about 30 nm. Within these ranges, color purity or color reproducibility may be improved. Light emitted through the quantum dot may be emitted in all directions, so that light viewing angle properties may be improved.

The shape of the quantum dot may be one that is used in the related art, without specific limitation. For example, the quantum dot may have a spherical shape, a pyramidal shape, a multi-arm shape, or a cubic shape, or the quantum dot may be in the form of a nanoparticle, a nanotube, a nanowire, a nanofiber, a nanoplate particle, etc.

The quantum dot may control the color of emitted light according to a particle size thereof, and accordingly, the quantum dot may have various emission colors such as blue, red, or green.

In the light emitting devices ED according to embodiments as shown in FIG. 3 to FIG. 6, the electron transport region ETR is provided on the emission layer EML. The electron transport region ETR may include at least one of a hole blocking layer HBL, an electron transport layer ETL, or an electron injection layer EIL. However, embodiments are not limited thereto.

The electron transport region ETR may be a layer formed of a single material, a layer formed of different materials, or a multilayer structure having layers formed of different materials.

For example, the electron transport region ETR may have a single layer structure of an electron injection layer EIL or an electron transport layer ETL, or a single layer structure formed of an electron injection material and an electron transport material. In other embodiments, the electron transport region ETR may have a single layer structure formed of different materials, or may have a structure in which an electron transport layer ETL/electron injection layer EIL, or a hole blocking layer HBL/electron transport layer ETL/electron injection layer EIL, are stacked in its respective stated order from the emission layer EML, but embodiments are not limited thereto. A thickness of the electron transport region ETR may be, for example, in a range of about 1,000 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-1.

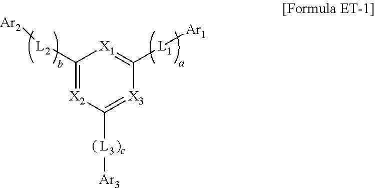

In Formula ET-1, at least one of X₁ to X₃ may be N, and the remainder of X₁ to X₃ may be C(Rₐ). Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In Formula ET-1, Ar₁ to Ara may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, a to c may each independently be an integer from 0 to 10. In Formula ET-1, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. If a to c are 2 or more, L₁ to L₃ may each independently be a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, embodiments are not limited thereto, and the electron transport region ETR may include, for example, diphenyl[4-(triphenylsilyl)phenyl]phosphine oxide (TSPO1), tris(8-hydroxyquinolinato)aluminum (Alq₃), 1,3,5-tri[(3-pyridyl)-phen-3-yl]benzene, 2,4,6-tris(3 (pyri din-3-yl)biphenyl-3-yl)-1,3,5-triazine, 2-(4-(N-phenylbenzoimidazolyl-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1, O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), and mixtures thereof, without limitation.

The electron transport region ETR may include at least one of Compounds ET1 to ET36.

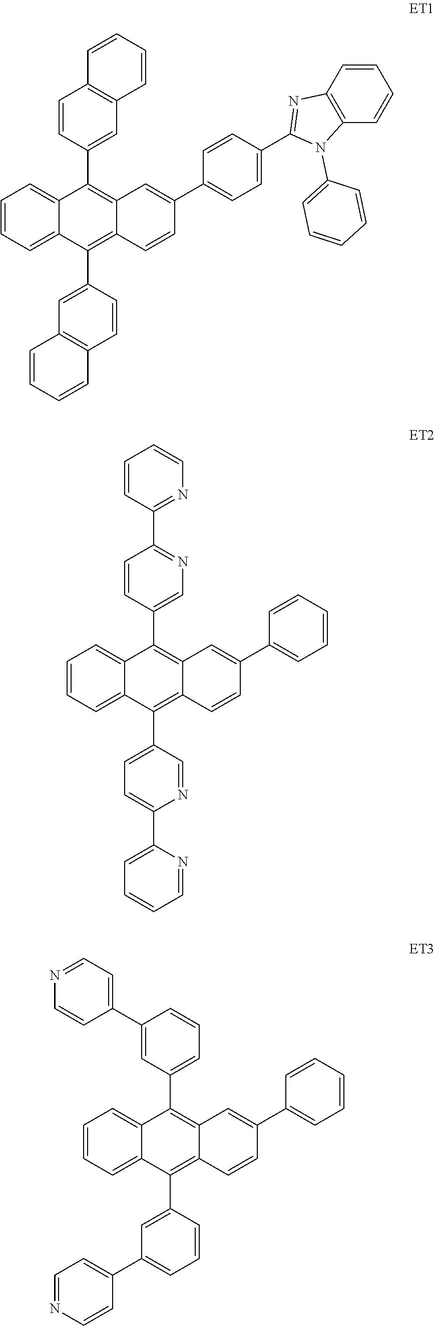 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

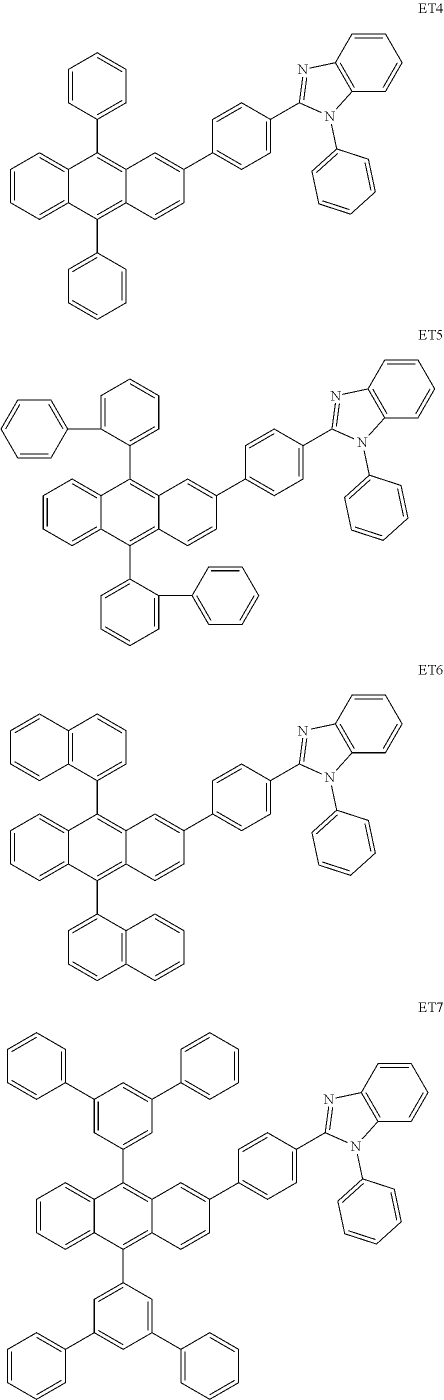 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

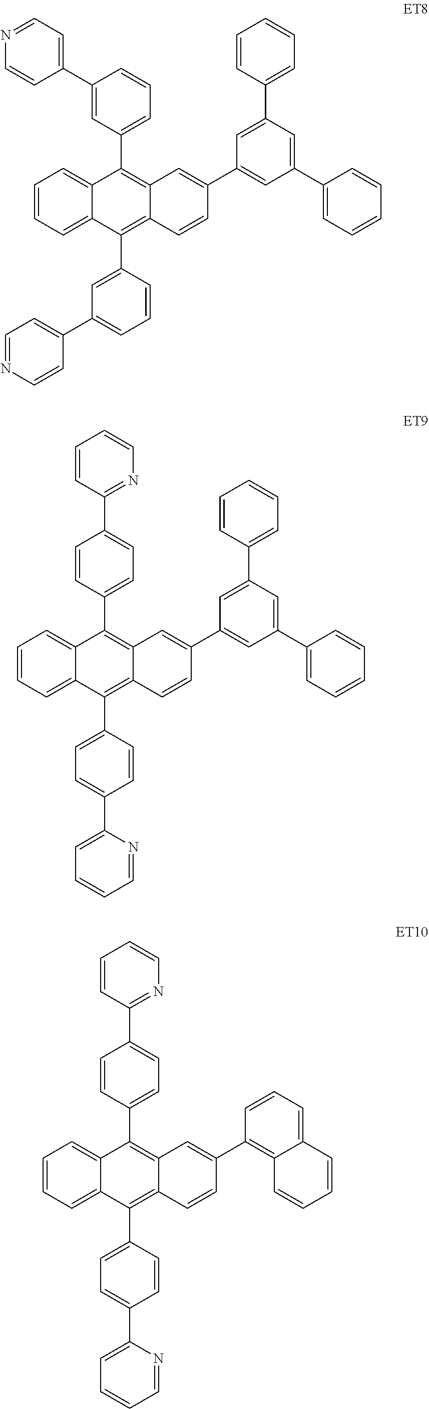 [SMILES: c1ccc(-c2cc(-c3ccccc3)cc(-c3ccc4c(-c5cccc(-c6ccncc6)c5)c5ccccc5c(-c5cccc(-c6ccncc6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

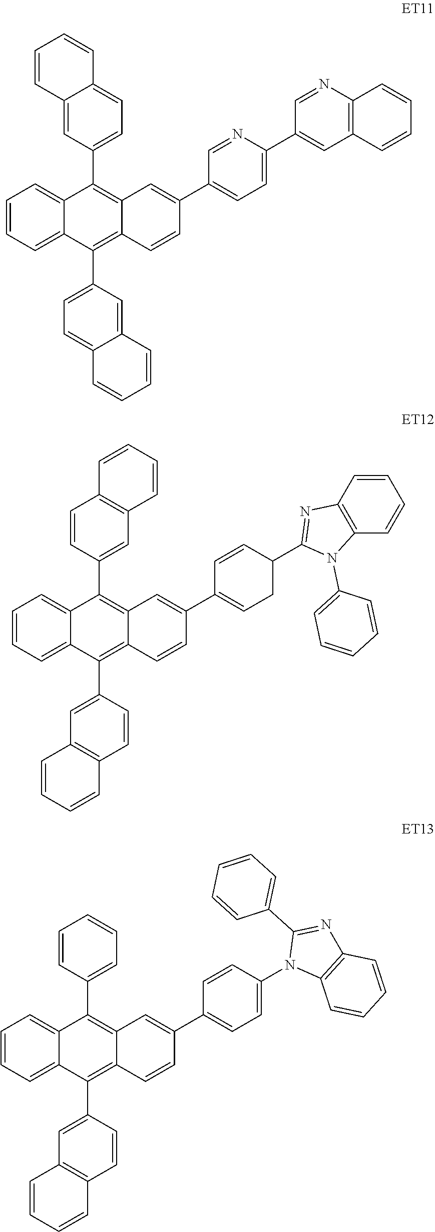

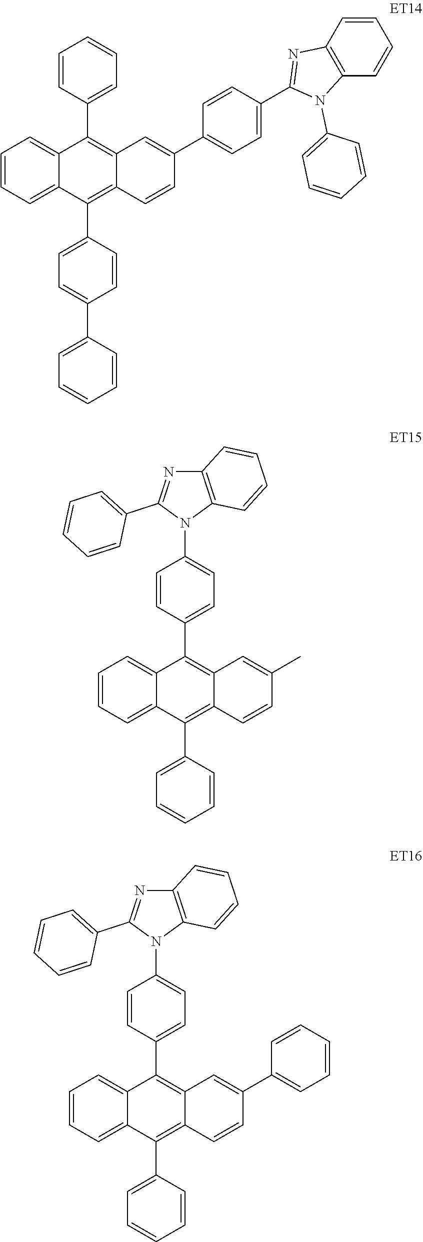

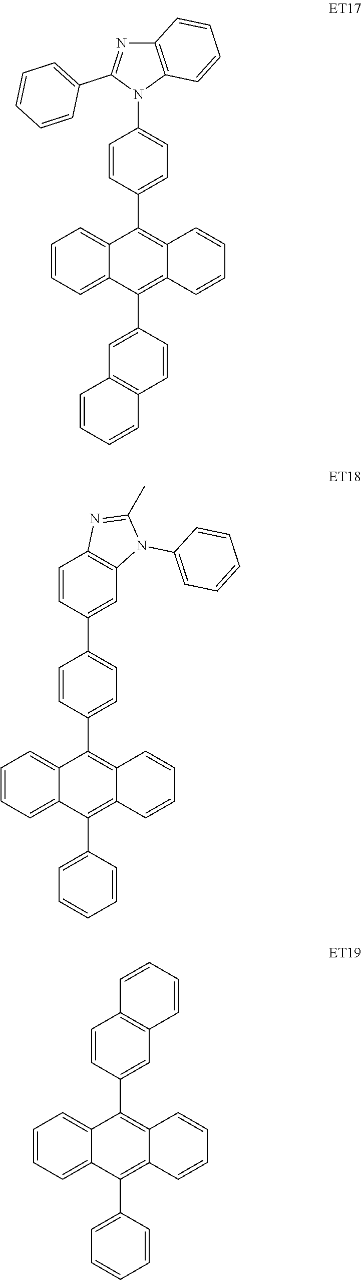

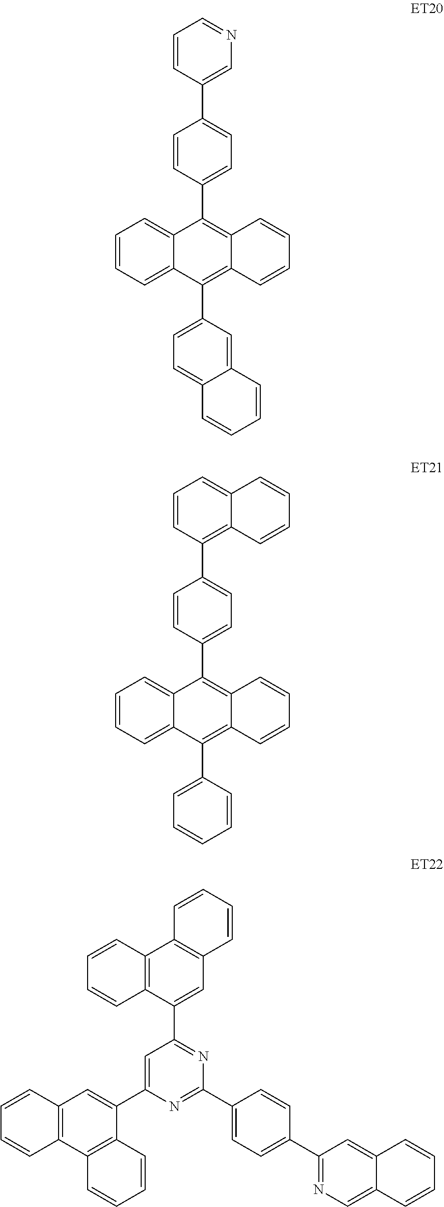

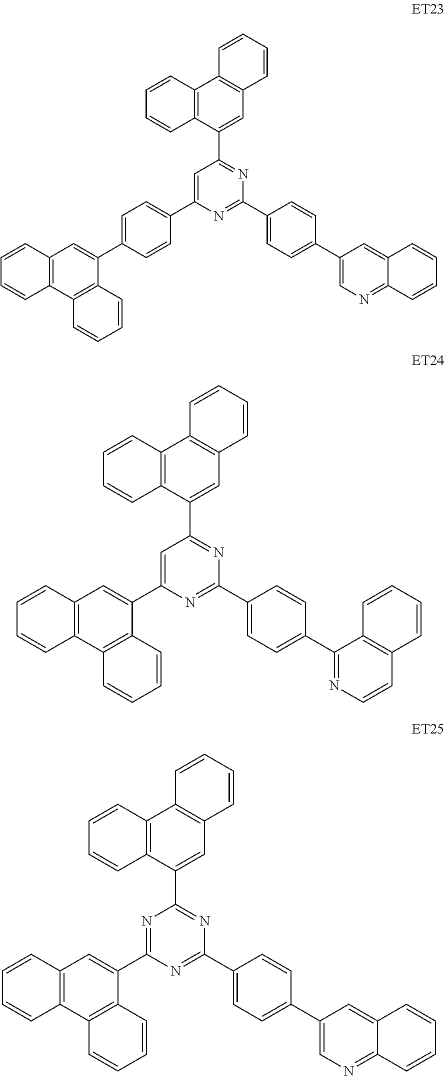 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

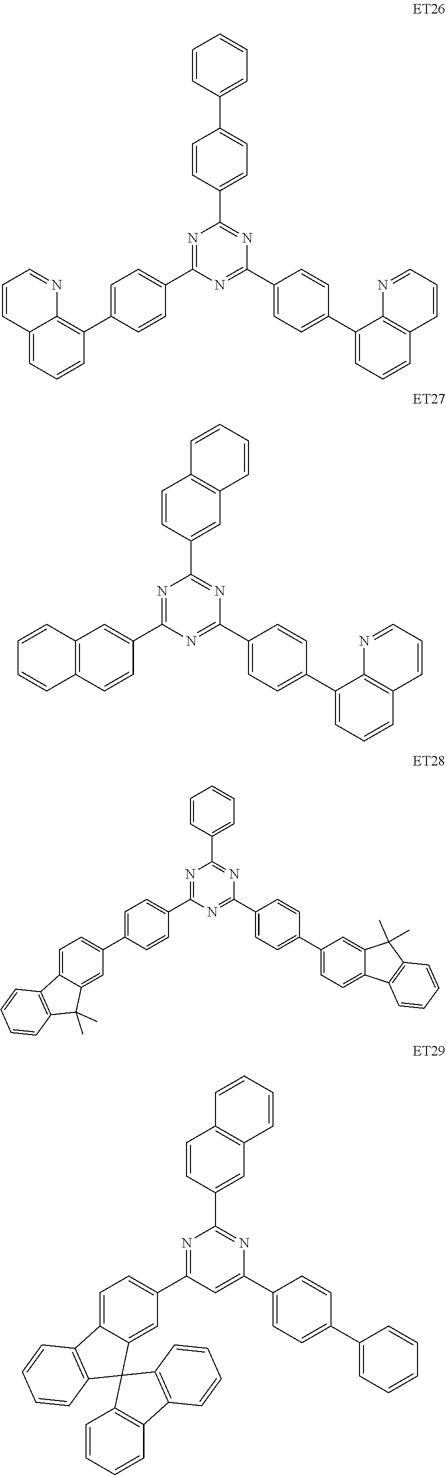 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

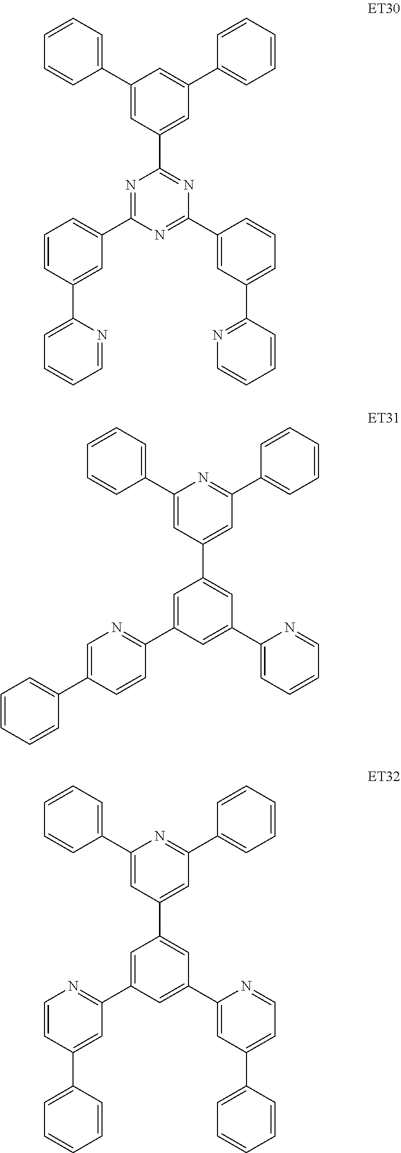 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

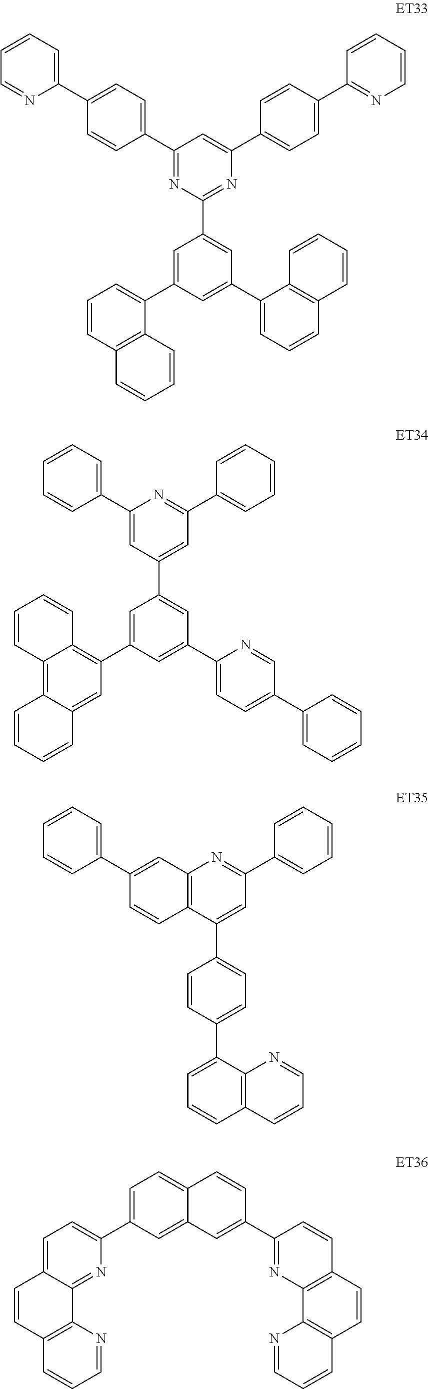 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccccc4)nc3c2)cc1 ; c1cnc2c(c1)ccc1ccc(-c3ccc4ccc(-c5ccc6ccc7cccnc7c6n5)cc4c3)nc12]

The electron transport region ETR may include a metal halide such as LiF, NaCl, CsF, RbCl, RbI, CuI, or KI, a lanthanide such as Yb, or a co-depositing material of the metal halide and the lanthanide. For example, the electron transport region ETR may include KI:Yb, RbI:Yb, LiF:Yb, etc., as the co-depositing material. The electron transport region ETR may include a metal oxide such as Li₂O and BaO, or 8-hydroxy-lithium quinolate (Liq). However, embodiments are not limited thereto. The electron transport region ETR may also be formed of a mixture material of an electron transport material and an insulating organo metal salt. The organo metal salt may be a material having an energy band gap equal to or greater than about 4 eV. For example, the organo metal salt may include metal acetates, metal benzoates, metal acetoacetates, metal acetylacetonates, or metal stearates.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, embodiments are not limited thereto.

The electron transport region ETR may include the compounds of the electron transport region in at least one of an electron injection layer EIL, an electron transport layer ETL, or a hole blocking layer HBL.

If the electron transport region ETR includes an electron transport layer ETL, a thickness of the electron transport layer ETL may be in a range of about 100 Å to about 1,000 Å. For example, the thickness of the electron transport layer ETL may be in a range of about 150 Å to about 500 Å. If the thickness of the electron transport layer ETL satisfies the above-described range, satisfactory electron transport properties may be obtained without a substantial increase of driving voltage. If the electron transport region ETR includes an electron injection layer EIL, a thickness of the electron injection layer EIL may be in a range of about 1 Å to about 100 Å. For example, the thickness of the electron injection layer EIL may be in a range of about 3 Å to about 90 Å. If the thickness of the electron injection layer EIL satisfies the above described range, satisfactory electron injection properties may be obtained without inducing a substantial increase of driving voltage.

The second electrode EL2 is provided on the electron transport region ETR. The second electrode EL2 may be a common electrode. The second electrode EL2 may be a cathode or an anode, but embodiments are not limited thereto. For example, if the first electrode EL1 is an anode, the second cathode EL2 may be a cathode, and if the first electrode EL1 is a cathode, the second electrode EL2 may be an anode.

The second electrode EL2 may be a transmissive electrode, a transflective electrode, or a reflective electrode. If the second electrode EL2 is a transmissive electrode, the second electrode EL2 may include a transparent metal oxide, for example, ITO, IZO, ZnO, ITZO, etc.

If the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, compounds thereof, or mixtures thereof (for example, AgMg, AgYb, or MgAg). In another embodiment, the second electrode EL2 may have a multilayered structure including a reflective layer or a transflective layer formed of the above-described materials and a transparent conductive layer formed of ITO, IZO, ZnO, ITZO, etc. For example, the second electrode EL2 may include the aforementioned metal materials, combinations of two or more metal materials selected from the aforementioned metal materials, or oxides of the aforementioned metal materials.

Although not shown in the drawings, the second electrode EL2 may be electrically connected to an auxiliary electrode. If the second electrode EL2 is electrically connected to the auxiliary electrode, the resistance of the second electrode EL2 may decrease.

In an embodiment, the light emitting device ED may further include a capping layer CPL disposed on the second electrode EL2. The capping layer CPL may be a multilayer or a single layer.

In an embodiment, the capping layer CPL may include an organic layer or an inorganic layer. For example, if the capping layer CPL includes an inorganic material, the inorganic material may include an alkali metal compound such as LiF, an alkaline earth metal compound such as MgF₂, SiON, SiNx, SiOy, etc.

For example, if the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol sol-9-yl) triphenylamine (TCTA), etc., or may include an epoxy resin, or acrylate such as methacrylate. For example, the capping layer CPL may include at least one of Compounds P1 to P5, but embodiments are not limited thereto.

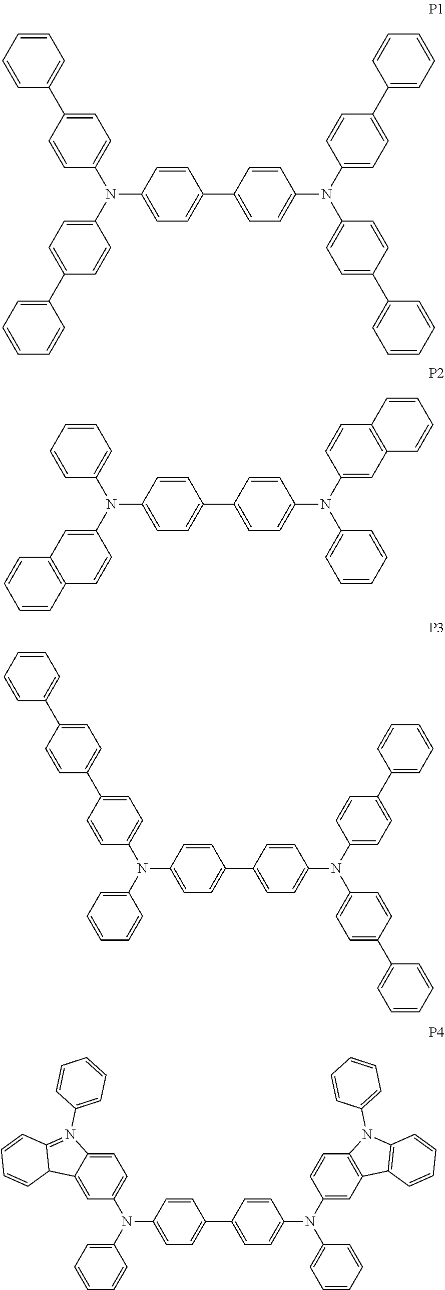

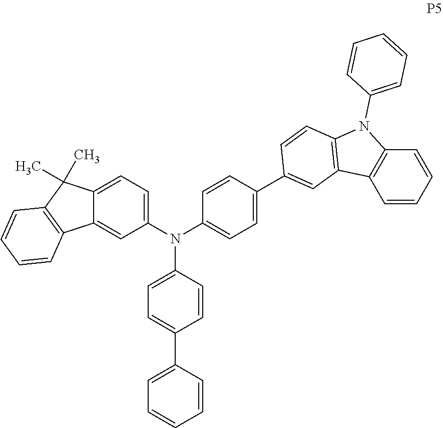 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, the refractive index of the capping layer CPL may be equal to or greater than about 1.6 with respect to light in a wavelength range of about 550 nm to about 660 nm.

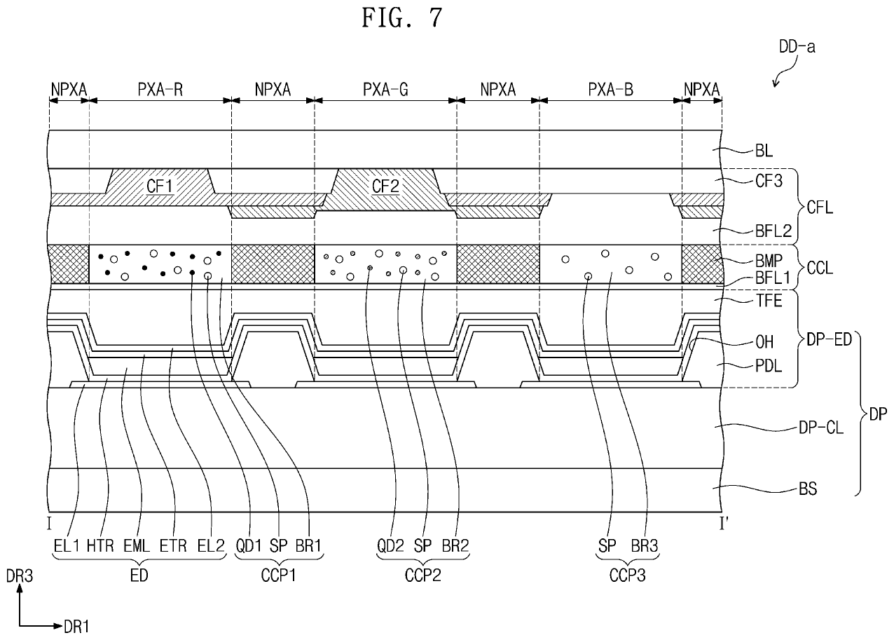

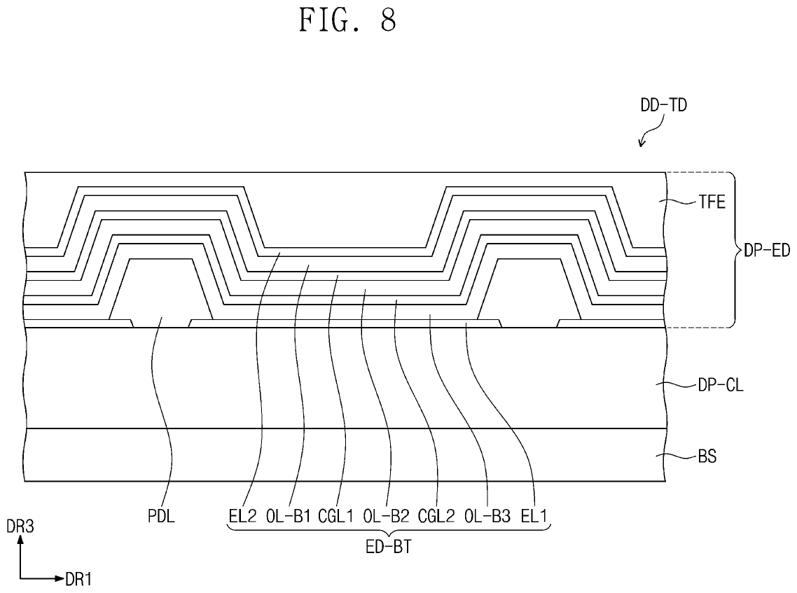

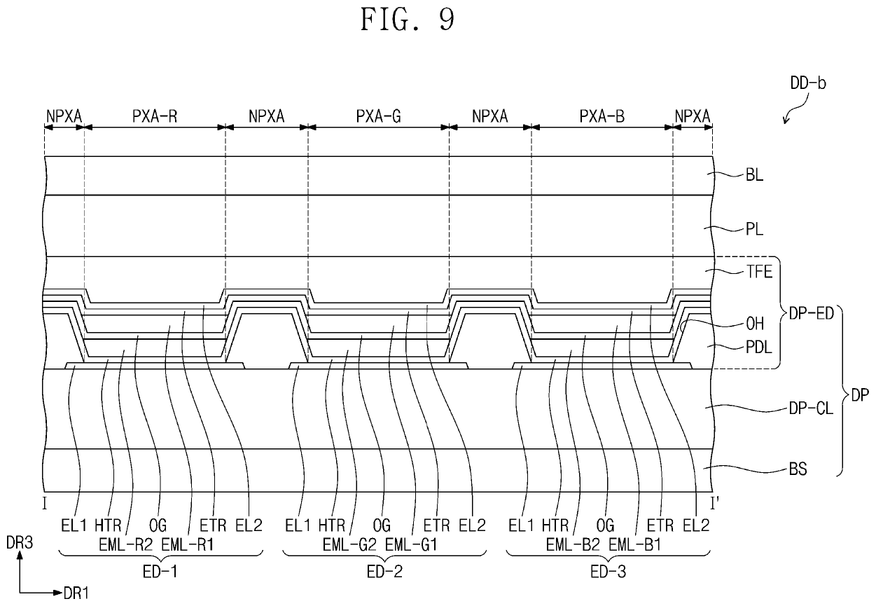

FIG. 7 and FIG. 8 are each a schematic cross-sectional view of a display apparatus according to embodiments. In the explanation on the display apparatuses of embodiments according to FIG. 7 and FIG. 8, the features which overlap with the explanation on FIG. 1 to FIG. 6 will not be explained again, and the different features will be explained.

Referring to FIG. 7, the display apparatus DD according to an embodiment may include a display panel DP including a display device layer DP-ED, a light controlling layer CCL disposed on the display panel DP, and a color filter layer CFL.

In an embodiment shown in FIG. 7, the display panel DP includes a base layer BS, a circuit layer DP-CL provided on the base layer BS, and a display device layer DP-ED, and the display device layer DP-ED may include a light emitting device ED.

The light emitting device ED may include a first electrode ELL a hole transport region HTR disposed on the first electrode ELL an emission layer EML disposed on the hole transport region HTR, an electron transport region ETR disposed on the emission layer EML, and a second electrode EL2 disposed on the electron transport region ETR. A structure of the light emitting device ED shown in FIG. 7 may be the same as a structure of a light emitting device according to FIG. 3 to FIG. 6.

Referring to FIG. 7, the emission layer EML may be disposed in an opening OH defined in a pixel definition layer PDL. For example, the emission layer EML which is separated by the pixel definition layer PDL and correspondingly provided to each of the luminous areas PXA-R, PXA-G, and PXA-B may emit light in a same wavelength region. In the display apparatus DD of an embodiment, the emission layer EML may emit blue light. Although not shown in the drawings, in an embodiment, the emission layer EML may be provided as a common layer for all luminous areas PXA-R, PXA-G, and PXA-B.

The light controlling layer CCL may be disposed on the display panel DP. The light controlling layer CCL may include a light converter. The light converter may be a quantum dot or a phosphor. The light converter may transform the wavelength of a provided light and emit the resulting light. For example, the light controlling layer CCL may be a layer including a quantum dot or a layer including a phosphor.

The light controlling layer CCL may include light controlling parts CCP1, CCP2, and CCP3. The light controlling parts CCP1, CCP2, and CCP3 may be separated from one another.

Referring to FIG. 7, a partition pattern BMP may be disposed between the separated light controlling parts CCP1, CCP2, and CCP3, but embodiments are not limited thereto. In FIG. 8, the partition pattern BMP is shown so that it does not overlap the light controlling parts CCP1, CCP2, and CCP3, but at least a portion of the edges of the light controlling parts CCP1, CCP2, and CCP3 may overlap the partition pattern BMP.

The light controlling layer CCL may include a first light controlling part CCP1 including a first quantum dot QD1 converting first color light provided from the light emitting device ED into second color light, a second light controlling part CCP2 including a second quantum dot QD2 converting first color light provided from the light emitting device ED into third color light, and a third light controlling part CCP3 transmitting first color light provided from the light emitting device ED.

In an embodiment, the first light controlling part CCP1 may provide red light which is the second color light, and the second light controlling part CCP2 may provide green light which is the third color light. The third color controlling part CCP3 may transmit and provide blue light which is the first color light provided from the light emitting device ED. For example, the first quantum dot QD1 may be a red quantum dot, and the second quantum dot QD2 may be a green quantum dot. The same explanation as provided above with respect to quantum dots may be applied to the quantum dots QD1 and QD2.

The light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but may include the scatterer SP.

The scatterer SP may be an inorganic particle. For example, the scatterer SP may include at least one of TiO₂, ZnO, Al₂O₃, SiO₂, or hollow silica. The scatterer SP may include at least one of TiO₂, ZnO, Al₂O₃, SiO₂, or hollow silica, or may be a mixture of two or more materials selected from TiO₂, ZnO, Al₂O₃, SiO₂, and hollow silica.

The first light controlling part CCP1, the second light controlling part CCP2, and the third light controlling part CCP3 may each include base resins BR1, BR2, and BR3 for dispersing the quantum dots QD1 and QD2 and the scatterer SP. In an embodiment, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2, and BR3 may each be a medium in which the quantum dots QD1 and QD2 and the scatterer SP are dispersed, and may be composed of various resin compositions which may be generally referred to as a binder. For example, the base resins BR1, BR2, and BR3 may each independently be acrylic resins, urethane-based resins, silicone-based resins, epoxy-based resins, etc. The base resins BR1, BR2, and BR3 may each be transparent resins. In an embodiment, the first base resin BR1, the second base resin BR2, and the third base resin BR3 may be the same as or different from each other.

The light controlling layer CCL may include a barrier layer BFL1. The barrier layer BFL1 may block the penetration of moisture and/or oxygen (hereinafter, will be referred to as “humidity/oxygen”). The barrier layer BFL1 may be disposed on the light controlling parts CCP1, CCP2, and CCP3 to block the exposure of the light controlling parts CCP1, CCP2, and CCP3 to humidity/oxygen. The barrier layer BFL1 may cover the light controlling parts CCP1, CCP2, and CCP3. A barrier layer BFL2 may be provided between the light controlling parts CCP1, CCP2, and CCP3 and a color filter layer CFL.

The barrier layers BFL1 and BFL2 may include at least one inorganic layer. For example, the barrier layers BFL1 and BFL2 may each include an inorganic material. For example, the barrier layers BFL1 and BFL2 may each independently include silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, or a metal thin film securing light transmittance. The barrier layers BFL1 and BFL2 may each further include an organic layer. The barrier layers BFL1 and BFL2 may be composed of a single layer or of multiple layers.

In the display apparatus DD of an embodiment, the color filter layer CFL may be disposed on the light controlling layer CCL. In an embodiment, the color filter layer CFL may be disposed directly on the light controlling layer CCL. For example, the barrier layer BFL2 may be omitted.

The color filter layer CFL may include a light blocking part BM and filters CF1, CF2, and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2, and CF3 may include a polymer photosensitive resin and a pigment or dye. The first filter CF1 may include a red pigment or dye, the second filter CF2 may include a green pigment or dye, and the third filter CF3 may include a blue pigment or dye. However, embodiments are not limited thereto, and the third filter CF3 may not include a pigment or dye. The third filter CF3 may include a polymer photosensitive resin and may not include a pigment or dye. The third filter CF3 may be transparent. The third filter CF3 may be formed of a transparent photosensitive resin.

In an embodiment, the first filter CF1 and the second filter CF2 may each be yellow filters. The first filter CF1 and the second filter CF2 may be provided as one body without distinction.

The light blocking part BM may be a black matrix. The light blocking part BM may include an organic light blocking material or an inorganic light blocking material each including a black pigment or a black dye. The light blocking part BM may prevent light leakage and may distinguish boundaries between adjacent filters CF1, CF2, and CF3. In an embodiment, the light blocking part BM may be formed of a blue filter.

Each of the first to third filters CF1, CF2, and CF3 may be disposed corresponding to the red luminous area PXA-R, the green luminous area PXA-G, and the blue luminous area PXA-B, respectively.

A base substrate BL may be disposed on the color filter layer CFL. The base substrate BL may provide a base surface on which the color filter layer CFL, the light controlling layer CCL, etc. are disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

FIG. 8 is a schematic cross-sectional view showing a portion of the display apparatus according to an embodiment. In a display apparatus DD-TD of an embodiment, the light emitting device ED-BT may include multiple light emitting structures OL-B1, OL-B2, and OL-B3. The light emitting device ED-BT may include a first electrode EL1, an oppositely disposed second electrode EL2, and the light emitting structures OL-B1, OL-B2, and OL-B3 stacked in a thickness direction and provided between the first electrode EL1 and the second electrode EL2. Each of the light emitting structures OL-B1, OL-B2, and OL-B3 may include an emission layer EML (FIG. 7), and a hole transport region HTR and an electron transport region ETR disposed with the emission layer EML (FIG. 7) therebetween.

For example, the light emitting device ED-BT included in the display apparatus DD-TD of an embodiment may be a light emitting device having a tandem structure including multiple emission layers.

In an embodiment shown in FIG. 8, light emitted from each of the light emitting structures OL-B1, OL-B2, and OL-B3 may be all blue light. However, embodiments are not limited thereto, and the wavelength regions of light emitted from the light emitting structures OL-B1, OL-B2, and OL-B3 may be different from each other. For example, the light emitting device ED-BT including the light emitting structures OL-B1, OL-B2, and OL-B3 emitting light in different wavelength regions may emit white light.

Charge generating layers CGL1 and CGL2 may be disposed between neighboring light emitting structures OL-B1, OL-B2, and OL-B3. The charge generating layers CGL1 and CGL2 may each independently include a p-type charge generating layer and/or an n-type charge generating layer.

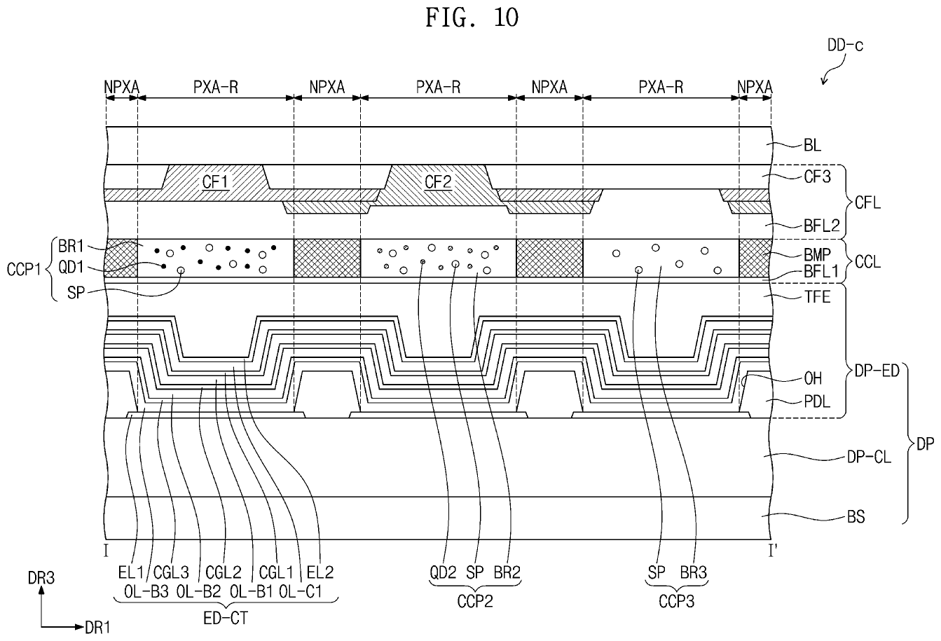

Referring to FIG. 9, a display apparatus DD-b according to an embodiment may include light emitting elements ED-1, ED-2, and ED-3, formed by stacking two emission layers. In comparison to the display apparatus DD of an embodiment shown in FIG. 2, an embodiment shown in FIG. 9 is different in that the first to third light emitting elements ED-1, ED-2, and ED-3 each include two emission layers stacked in a thickness direction. In the first to third light emitting elements ED-1, ED-2, and ED-3, two emission layers may emit light in a same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. The third light emitting element ED-3 may include a first blue emission layer EML-B1 and a second blue emission layer EML-B2. An emission auxiliary part OG may be disposed between the first red emission layer EML-R1 and the second red emission layer EML-R₂, between the first green emission layer EML-G1 and the second green emission layer EML-G2, and between the first blue emission layer EML-B1 and the second blue emission layer EML-B2.

The emission auxiliary part OG may be a single layer or a multilayer. The emission auxiliary part OG may include a charge generating layer. For example, the emission auxiliary part OG may include an electron transport region, a charge generating layer, and a hole transport region, stacked in that order. The emission auxiliary part OG may be provided as a common layer for all of the first to third light emitting elements ED-1, ED-2, and ED-3. However, embodiments are not limited thereto, and the emission auxiliary part OG may be patterned and provided in an opening OH defined in a pixel definition layer PDL.

The first red emission layer EML-R1, the first green emission layer EML-G1 and the first blue emission layer EML-B1 may be disposed between the electron transport region ETR and the emission auxiliary part OG. The second red emission layer EML-R2, the second green emission layer EML-G2 and the second blue emission layer EML-B2 may be disposed between the emission auxiliary part OG and the hole transport region HTR.

For example, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in that order. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in that order. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in that order.

An optical auxiliary layer PL may be disposed on a display device layer DP-ED. The optical auxiliary layer PL may include a polarization layer. The optical auxiliary layer PL may be disposed on a display panel DP and may control light reflected at the display panel DP from an external light. Although not shown in the drawings, in an embodiment, the optical auxiliary layer PL may be omitted from the display apparatus DD-b.

In comparison to FIG. 8 and FIG. 9, FIG. 10 shows a display apparatus DD-c in FIG. 10 that is different at least in that it includes four light emitting structures OL-B1, OL-B2, OL-B3, and OL-C1. A light emitting element ED-CT may include a first electrode EL1, an oppositely second electrode EL2, and first to fourth light emitting structures OL-B1, OL-B2, OL-B3, and OL-C1 stacked in a thickness direction between the first electrode EL1 and the second electrode EL2. Charge generating layers CGL1, CGL2, and CGL3 may be disposed between the first to fourth light emitting structures OL-B1, OL-B2, OL-B3, and OL-C1. Among the four light emitting structures, the first to third light emitting structures OL-B1, OL-B2, and OL-B3 may each emit blue light, and the fourth light emitting structure OL-C1 may emit green light. However, embodiments are not limited thereto, and the first to fourth light emitting structures OL-B1, OL-B2, OL-B3, and OL-C1 may each emit different wavelengths of light.

Charge generating layers CGL1, CGL2, and CGL3 may be disposed between neighboring light emitting structures OL-B1, OL-B2, OL-B3, and OL-C1. Charge generating layers CGL1, CGL2, and CGL3 may each independently include a p-type charge generating layer and/or an n-type charge generating layer.

The first dopant of an embodiment may be included in at least one of the light emitting structures OL-B1, OL-B2, OL-B3, and OL-C1 of the display apparatus DD-c.

The first dopant of an embodiment may be included in a functional layer other than the emission layer EML, as a material for a light emitting device ED. The light emitting device ED according to an embodiment may include the first dopant in at least one functional layer disposed between the first electrode EL1 and the second electrode EL2 or in a capping layer CPL disposed on the second electrode EL2.

The light emitting device ED according to an embodiment may optimize the combination of the first and second host and the first and second dopant in the emission layer as described above, and may show excellent emission efficiency properties. The light emitting device ED of an embodiment may show high efficiency and long-life characteristics in a blue wavelength region.

Hereinafter, the light emitting device according to an embodiment will be explained with reference to the Examples and the Comparative Examples. The Examples below are only provided as illustrations for understanding the disclosure, and the scope thereof is not limited thereto.

### EXAMPLES

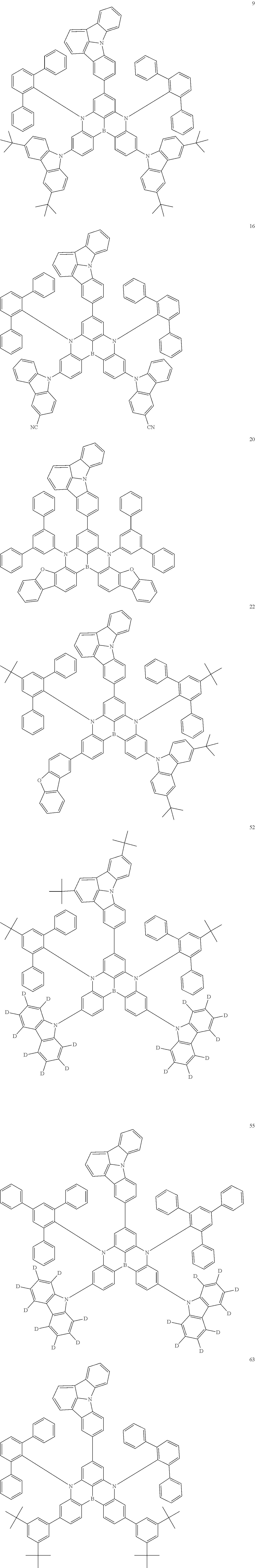

1. Synthesis of First Dopant

A synthesis method of the first dopant according to an embodiment will be explained by illustrating the synthesis methods of Compounds 9, 16, 20, 22, 52, 55 and 63. The synthesis methods of the first dopants explained hereinafter are only examples and the synthesis method of the first dopant according to an embodiment is not limited to the examples below.

### (1) Synthesis of Compound 9

### Synthesis of Intermediate 9-1

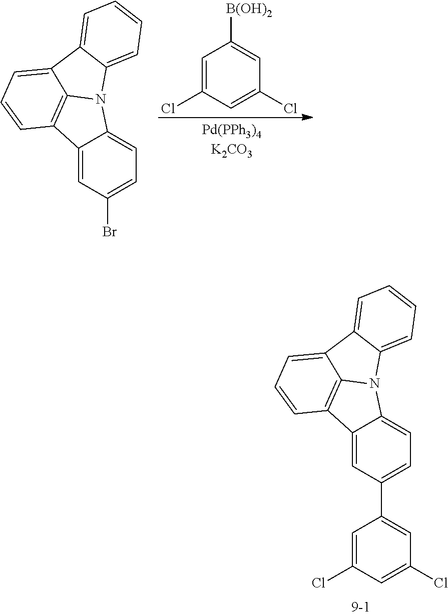 [SMILES: Brc1ccc2c(c1)c1cccc3c4ccccc4n2c31 ; OB(O)c1cc(Cl)cc(Cl)c1 ; Clc1cc(Cl)cc(-c2ccc3c(c2)c2cccc4c5ccccc5n3c42)c1]

5-bromoindolo[3,2,1-jk]carbazole (1 eq), (3,5-dichlorophenyl)boronic acid (1.1 eq), Pd(PPh₃)₄ (0.05 eq), and K₂CO₃ (3 eq) were dissolved in a mixture of water and THF in a ratio of 2:1, and stirred at about 80 degrees centigrade for about 12 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 9-1 (yield: 74%).

### Synthesis of Intermediate 9-2

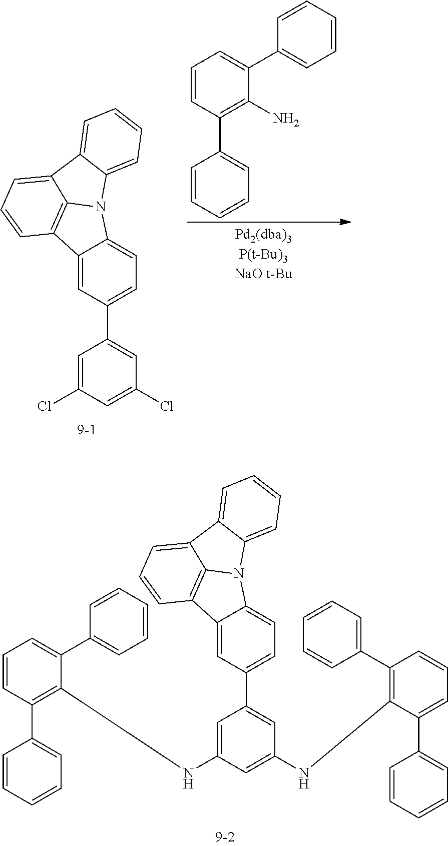 [SMILES: Clc1cc(Cl)cc(-c2ccc3c(c2)c2cccc4c5ccccc5n3c42)c1 ; Nc1c(-c2ccccc2)cccc1-c1ccccc1 ; c1ccc(-c2cccc(-c3ccccc3)c2Nc2cc(Nc3c(-c4ccccc4)cccc3-c3ccccc3)cc(-c3ccc4c(c3)c3cccc5c6ccccc6n4c53)c2)cc1]

Intermediate 9-1 (1 eq), [1,1′:3′,1″-terphenyl]-2′-amine (2 eq), tris(dibenzylideneacetone)dipalladium(0) (0.05 eq), tri-tert-butylphosphine (0.1 eq), and sodium tert-butoxide (3 eq) were dissolved in o-xylene and stirred at about 140 degrees centigrade for about 10 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 9-2 (yield: 78%).

### Synthesis of Intermediate 9-3

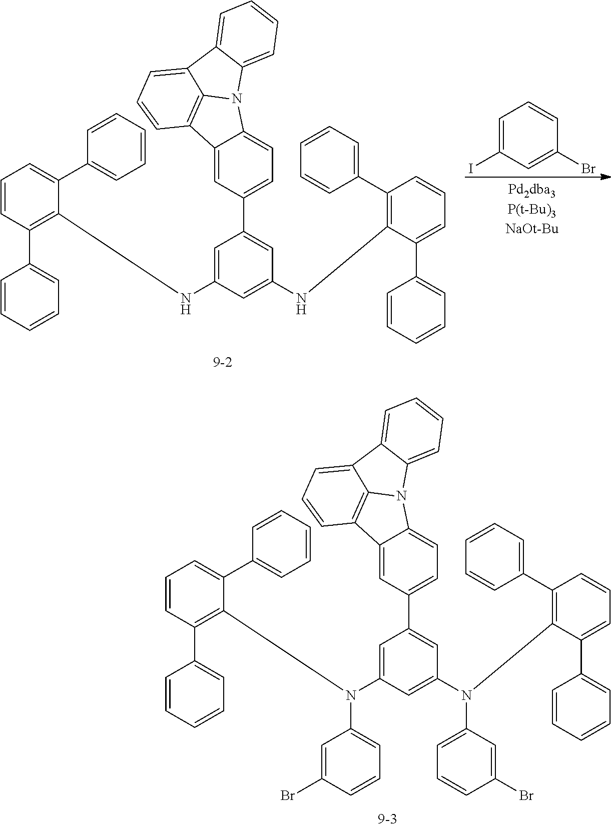

Intermediate 9-2 (1 eq), 1-bromo-3-iodobenzene (5 eq), tris(dibenzylideneacetone)dipalladium(0) (0.1 eq), tri-tert-butylphosphine (0.2 eq), sodium tert-butoxide (3 eq) were dissolved in o-xylene and stirred at about 160 degrees centigrade for about 48 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 9-3 (yield: 32%).

### Synthesis of Intermediate 9-4

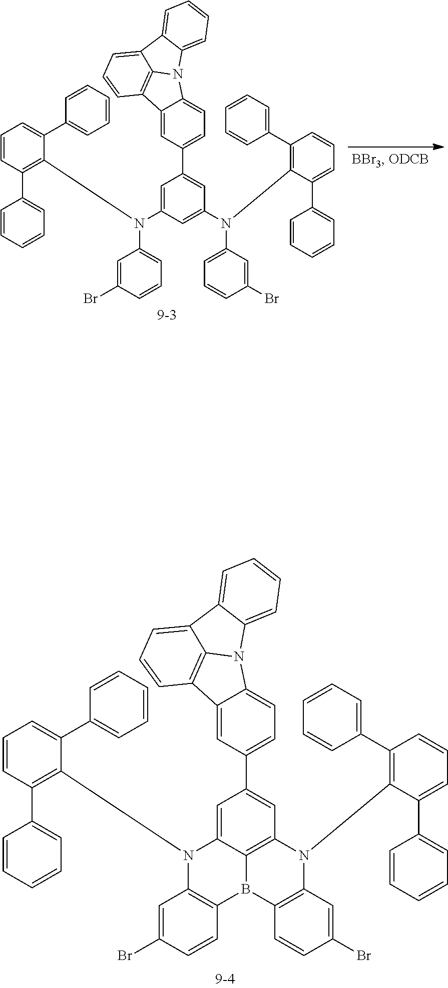 [SMILES: Brc1cccc(N(c2cc(-c3ccc4c(c3)c3cccc5c6ccccc6n4c53)cc(N(c3cccc(Br)c3)c3c(-c4ccccc4)cccc3-c3ccccc3)c2)c2c(-c3ccccc3)cccc2-c2ccccc2)c1 ; Brc1ccc2c(c1)N(c1c(-c3ccccc3)cccc1-c1ccccc1)c1cc(-c3ccc4c(c3)c3cccc5c6ccccc6n4c53)cc3c1B2c1ccc(Br)cc1N3c1c(-c2ccccc2)cccc1-c1ccccc1]

Intermediate 9-3 (1 eq) was dissolved in o-dichlorobenzene (ODCB) and cooled to about 0 degrees. BBr₃ (5 eq) was slowly added thereto dropwise, and the temperature was raised to about 180 degrees, followed by stirring for about 12 hours. After cooling, to a flask containing the reaction mixture, triethylamine was slowly added dropwise to terminate the reaction, and ethyl alcohol was added to the reaction product to precipitate. The precipitate was filtered to obtain a reaction product. The solid content thus obtained was separated by column chromatography using methylene chloride and n-hexane, and recrystallized using toluene and acetone to obtain Intermediate 9-4 (yield: 23%).

### Synthesis of Compound 9

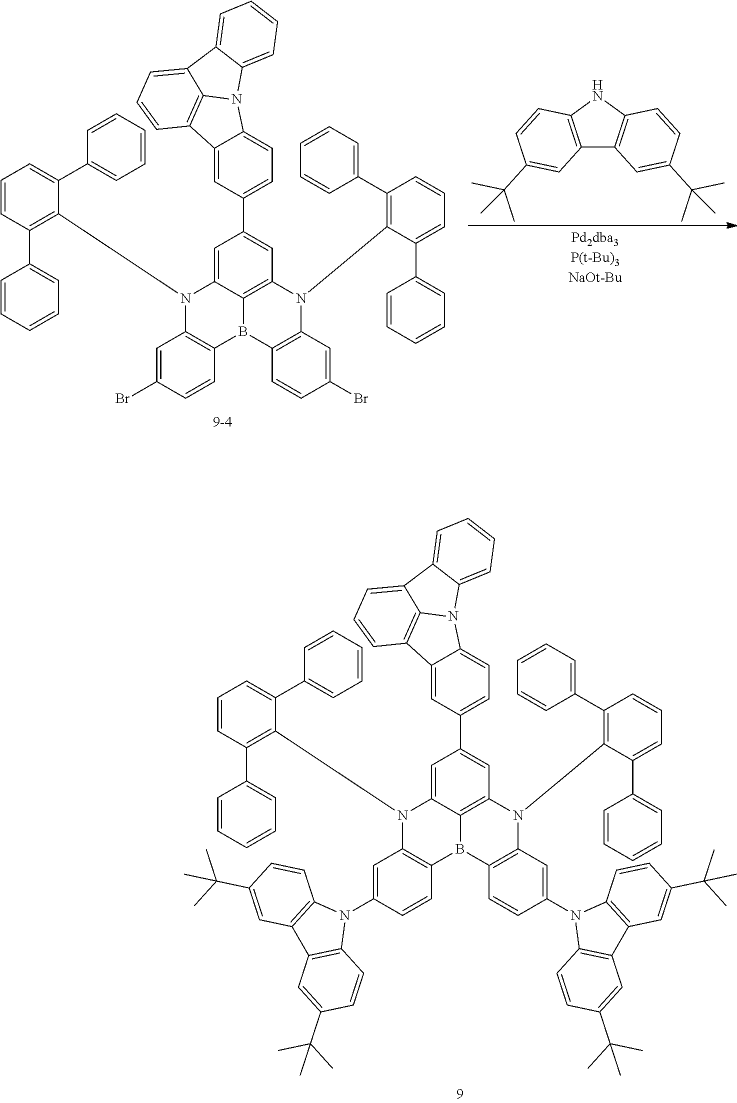

Intermediate 9-4 (1 eq), 3,6-di-tert-butyl-9H-carbazole (2.5 eq), tris(dibenzylideneacetone)dipalladium(0) (0.1 eq), tri-tert-butylphosphine (0.2 eq), and sodium tert-butoxide (3 eq) were dissolved in o-xylene and stirred at about 150 degrees centigrade for about 16 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Compound 9 (yield: 76%).

### (2) Synthesis of Compound 16

### Synthesis of Compound 16

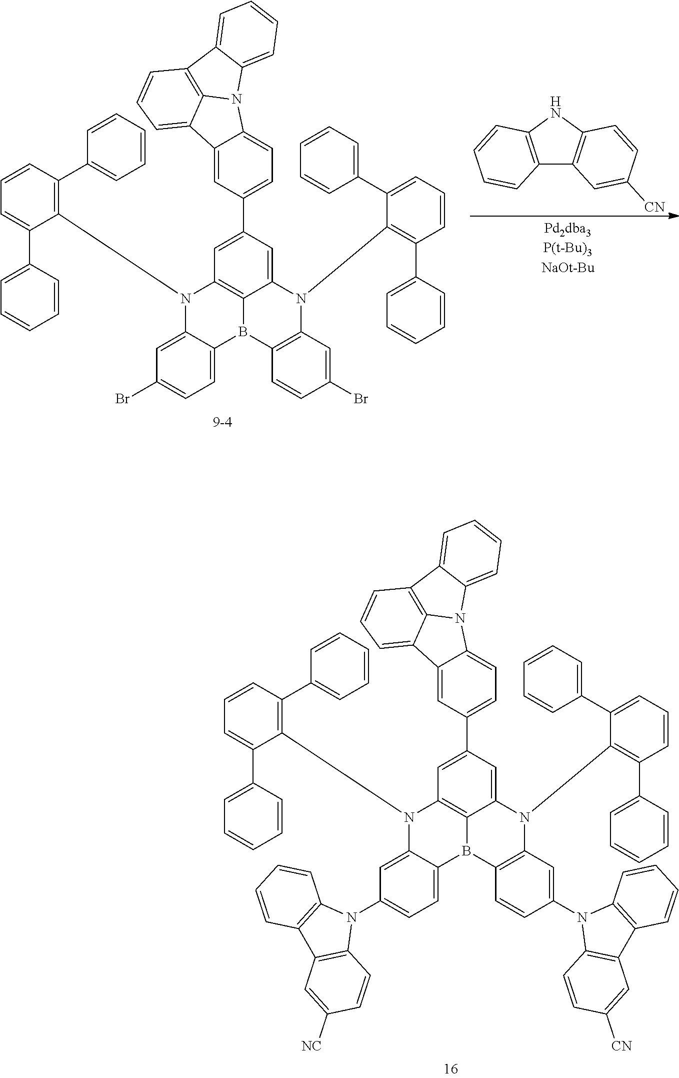

Intermediate 9-4 (1 eq), 9H-carbazole-3-carbonitrile (3 eq), tris(dibenzylideneacetone)dipalladium(0) (0.1 eq), tri-tert-butylphosphine (0.2 eq), and sodium tert-butoxide (3 eq) were dissolved in o-xylene and stirred at about 150 degrees centigrade for about 48 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Compound 16 (yield: 45%).

### (3) Synthesis of Compound 20

### Synthesis of Intermediate 20-1

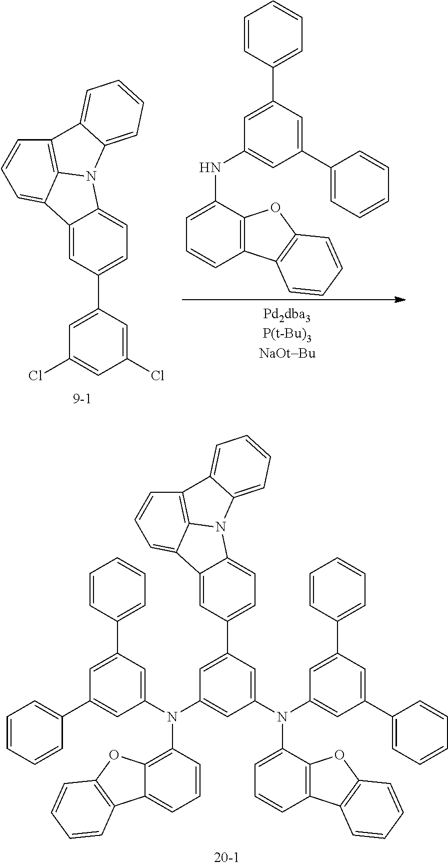 [SMILES: Clc1cc(Cl)cc(-c2ccc3c(c2)c2cccc4c5ccccc5n3c42)c1 ; c1ccc(-c2cc(Nc3cccc4c3oc3ccccc34)cc(-c3ccccc3)c2)cc1 ; c1ccc(-c2cc(-c3ccccc3)cc(N(c3cc(-c4ccc5c(c4)c4cccc6c7ccccc7n5c64)cc(N(c4cc(-c5ccccc5)cc(-c5ccccc5)c4)c4cccc5c4oc4ccccc45)c3)c3cccc4c3oc3ccccc34)c2)cc1]

Intermediate 9-1 (1 eq), N-([1,1′:3′,1″-terphenyl]-5′-yl)dibenzo[b,d]furan-4-amine (2 eq), tris(dibenzylideneacetone)dipalladium(0) (0.05 eq), tri-tert-butylphosphine (0.1 eq), and sodium tert-butoxide (3 eq) were dissolved in o-xylene and stirred at about 140 degrees centigrade for about 10 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 20-1 (yield: 66%).

### Synthesis of Compound 20

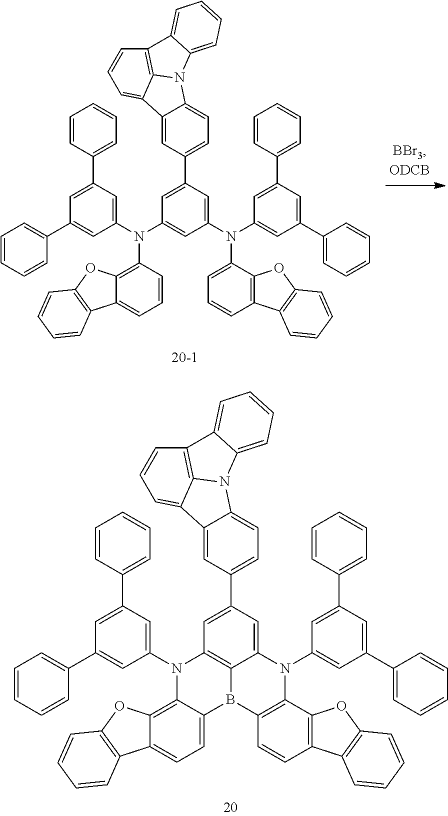 [SMILES: c1ccc(-c2cc(-c3ccccc3)cc(N(c3cc(-c4ccc5c(c4)c4cccc6c7ccccc7n5c64)cc(N(c4cc(-c5ccccc5)cc(-c5ccccc5)c4)c4cccc5c4oc4ccccc45)c3)c3cccc4c3oc3ccccc34)c2)cc1 ; c1ccc(-c2cc(-c3ccccc3)cc(N3c4cc(-c5ccc6c(c5)c5cccc7c8ccccc8n6c75)cc5c4B(c4ccc6c(oc7ccccc76)c43)c3ccc4c(oc6ccccc64)c3N5c3cc(-c4ccccc4)cc(-c4ccccc4)c3)c2)cc1]

Intermediate 20-1 (1 eq) was dissolved in o-dichlorobenzene (ODCB) and cooled to about 0 degrees centigrade. BBr₃ (5 eq) was slowly added thereto dropwise, and the temperature was raised to about 180 degrees, followed by stirring for about 12 hours. After cooling, to a flask containing the reaction mixture, triethylamine was slowly added dropwise to terminate the reaction, and ethyl alcohol was added to the reaction product to precipitate. The precipitate was filtered to obtain a reaction product. The solid content thus obtained was separated by column chromatography using methylene chloride and n-hexane, and recrystallized using toluene and acetone to obtain Compound 20 (yield: 18%).

### (4) Synthesis of Compound 22

### Synthesis of Intermediate 22-1

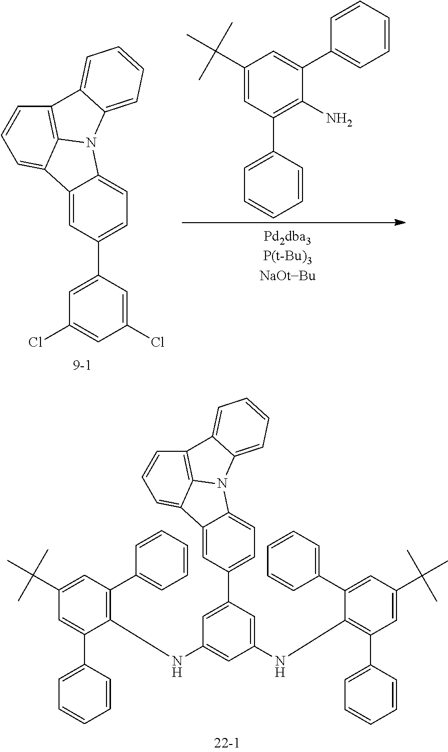 [SMILES: Clc1cc(Cl)cc(-c2ccc3c(c2)c2cccc4c5ccccc5n3c42)c1 ; CC(C)(C)c1cc(-c2ccccc2)c(N)c(-c2ccccc2)c1 ; CC(C)(C)c1cc(-c2ccccc2)c(Nc2cc(Nc3c(-c4ccccc4)cc(C(C)(C)C)cc3-c3ccccc3)cc(-c3ccc4c(c3)c3cccc5c6ccccc6n4c53)c2)c(-c2ccccc2)c1]

Intermediate 9-1 (1 eq), 5′-(tert-butyl)-[1,1′:3′,1″-terphenyl]-2′-amine (2 eq), tris(dibenzylideneacetone)dipalladium(0) (0.05 eq), tri-tert-butylphosphine (0.1 eq), and sodium tert-butoxide (3 eq) were dissolved in o-xylene and stirred at about 140 degrees centigrade for about 12 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 22-1 (yield: 75%).

### Synthesis of Intermediate 22-2

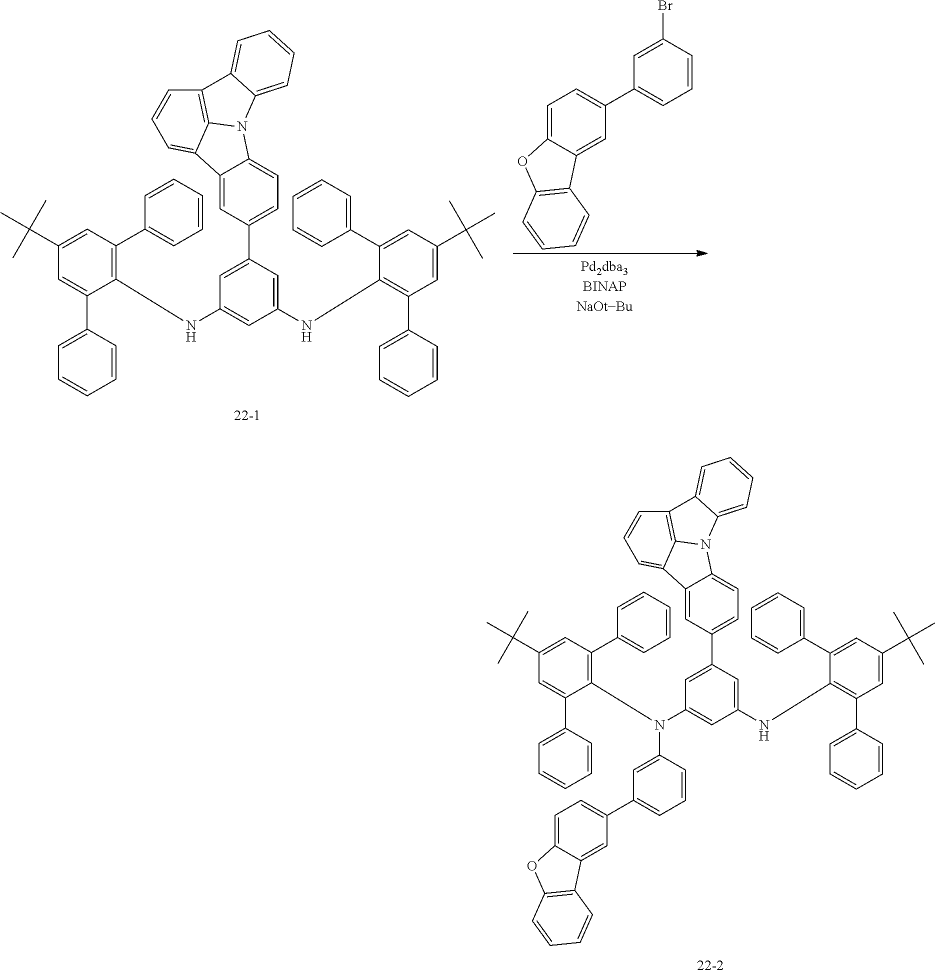

Intermediate 22-1 (1 eq), 2-(3-bromophenyl)dibenzo[b,d]furan (0.9 eq), tris(dibenzylideneacetone)dipalladium(0) (0.05 eq), BINAP (0.1 eq), and sodium tert-butoxide (3 eq) were dissolved in toluene and stirred at about 90 degrees centigrade for about 48 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 22-2 (yield: 42%).

### Synthesis of Intermediate 22-3

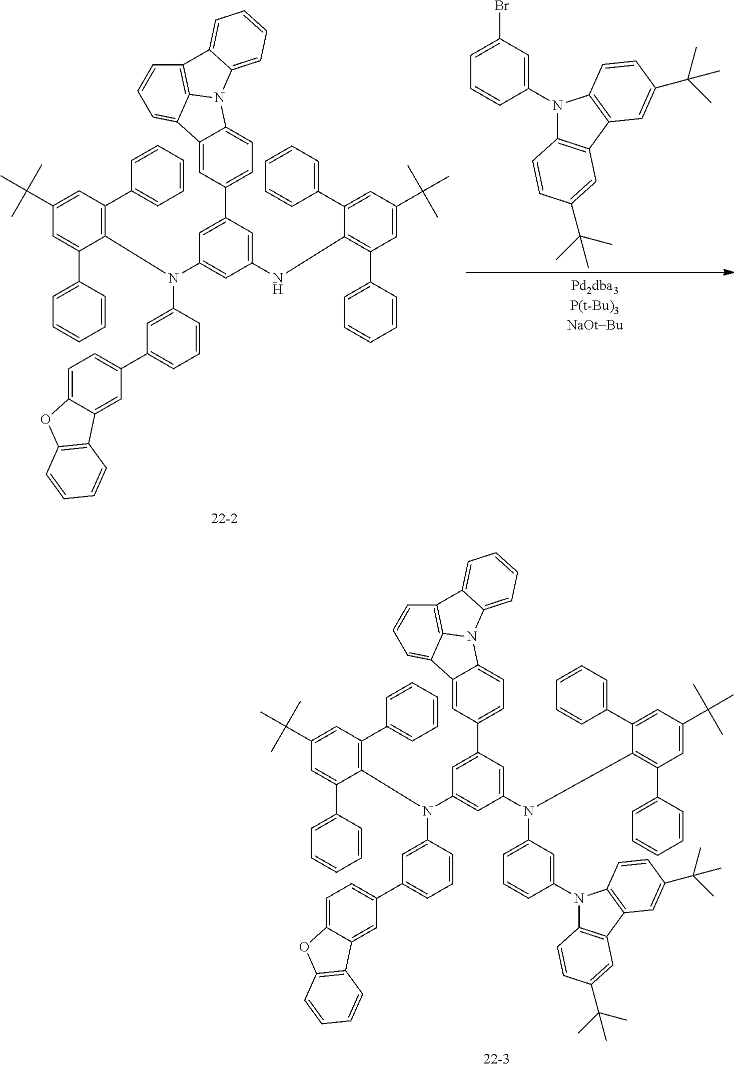

Intermediate 22-2 (1 eq), 9-(3-bromophenyl)-3,6-di-tert-butyl-9H-carbazole (2 eq), tris(dibenzylideneacetone)dipalladium(0) (0.1 eq), tri-tert-butylphosphine (0.1 eq), and sodium tert-butoxide (3 eq) were dissolved in o-xylene and stirred at about 140 degrees centigrade for about 36 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 22-3 (yield: 50%).

### Synthesis of Compound 22

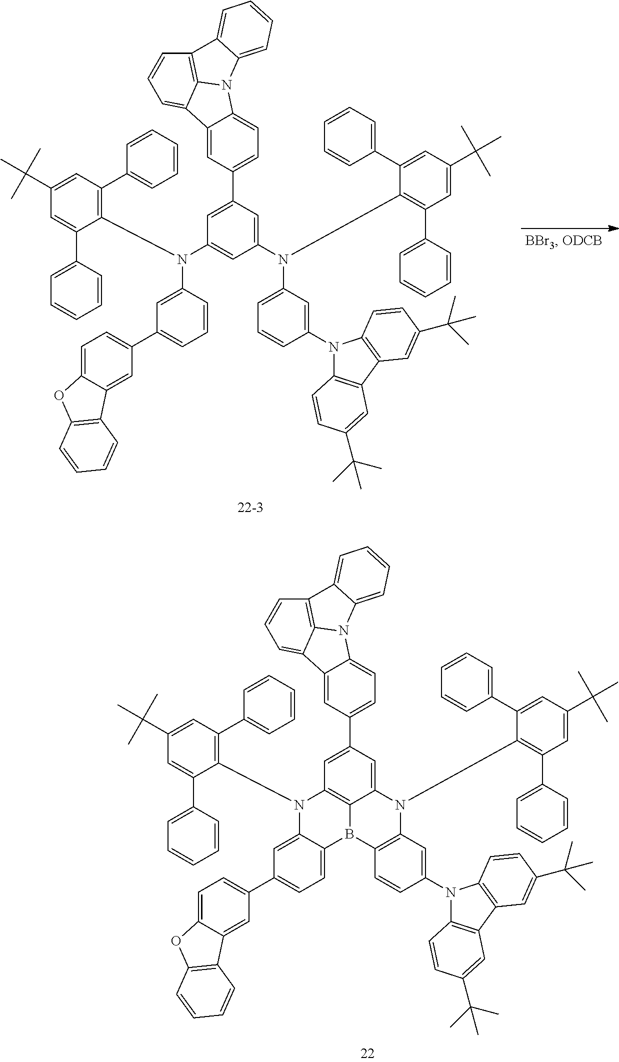

Intermediate 22-3 (1 eq) was dissolved in o-dichlorobenzene (ODCB) and cooled to about 0 degrees centigrade. BBr₃ (5 eq) was slowly added thereto dropwise, and the temperature was raised to about 180 degrees, followed by stirring for about 12 hours. After cooling, to a flask containing the reaction mixture, triethylamine was slowly added dropwise to terminate the reaction, and ethyl alcohol was added to the reaction product to precipitate. The precipitate was filtered to obtain a reaction product. The solid content thus obtained was separated by column chromatography using methylene chloride and n-hexane, and recrystallized using toluene and acetone to obtain Compound 22 (yield: 16%).

### (5) Synthesis of Compound 52

### Synthesis of Intermediate 52-1

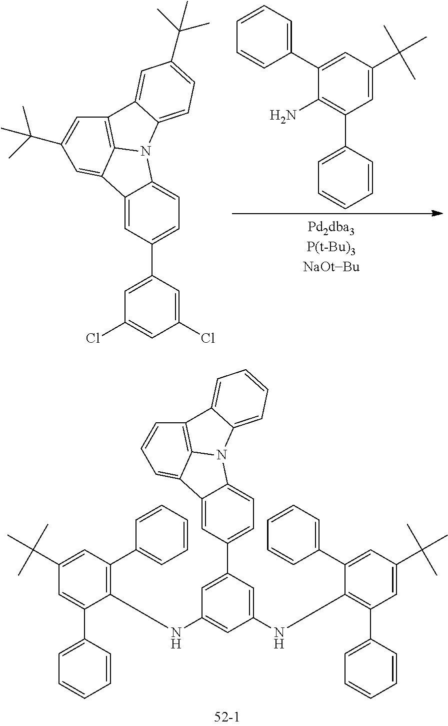

2,5-di-tert-butyl-11-(3, 5-dichlorophenyl)indolo[3,2,1-jk]carbazole (1 eq), 5′-(tert-butyl)-[1,1′:3′,1″-terphenyl]-2′-amine (2 eq), tris(dibenzylideneacetone)dipalladium(0) (0.05 eq), tri-tert-butylphosphine (0.1 eq), and sodium tert-butoxide (3 eq) were dissolved in o-xylene, and stirred at about 140 degrees centigrade for about 12 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 52-1 (yield: 62%).

### Synthesis of Intermediate 52-2

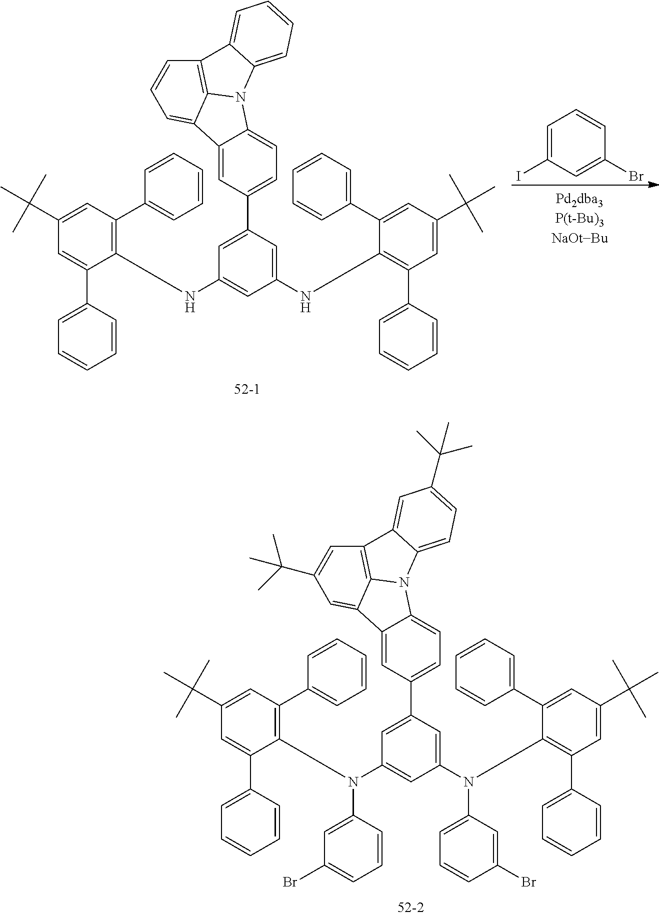

Intermediate 52-1 (1 eq), 1-bromo-3-iodobenzene (5 eq), tris(dibenzylideneacetone)dipalladium(0) (0.1 eq), tri-tert-butylphosphine (0.2 eq), and sodium tert-butoxide (3 eq) were dissolved in o-xylene and stirred at about 160 degrees centigrade for about 48 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 52-2 (yield: 41%).

### Synthesis of Intermediate 52-3

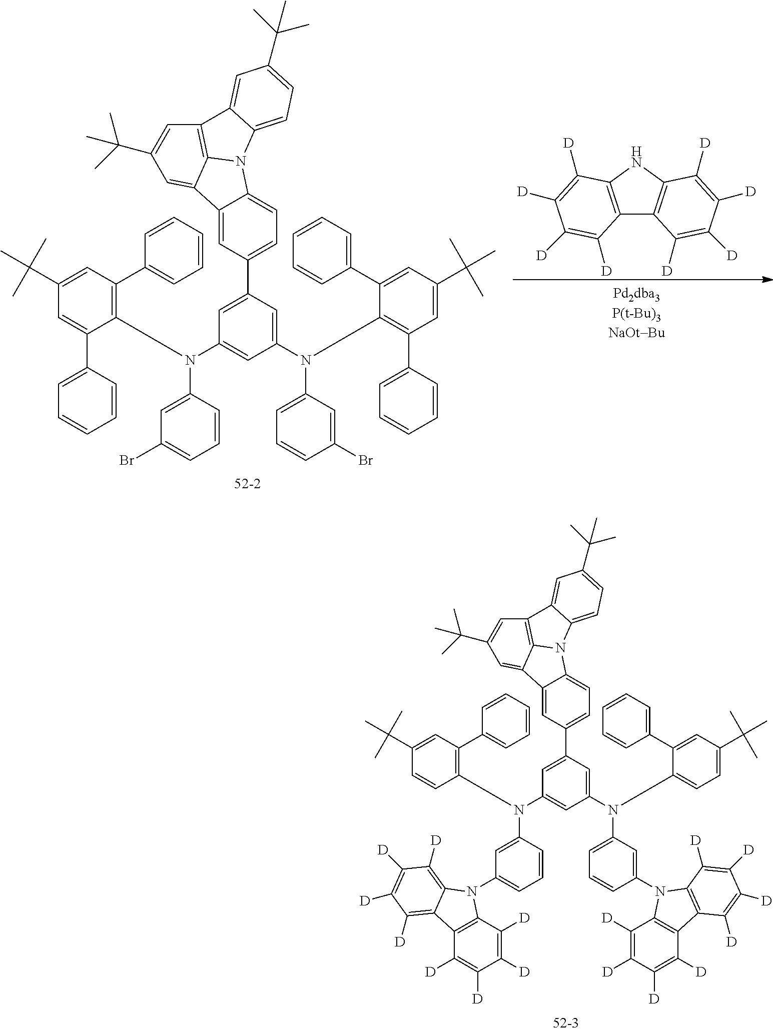

Intermediate 52-2 (1 eq), 9H-carbazole-1,2,3,4,5,6,7,8-d8 (2.5 eq), tris(dibenzylideneacetone)dipalladium(0) (0.1 eq), tri-tert-butylphosphine (0.2 eq), and sodium tert-butoxide (3 eq) were dissolved in o-xylene and stirred at about 150 degrees centigrade for about 24 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 52-3 (yield: 71%).

### Synthesis of Compound 52

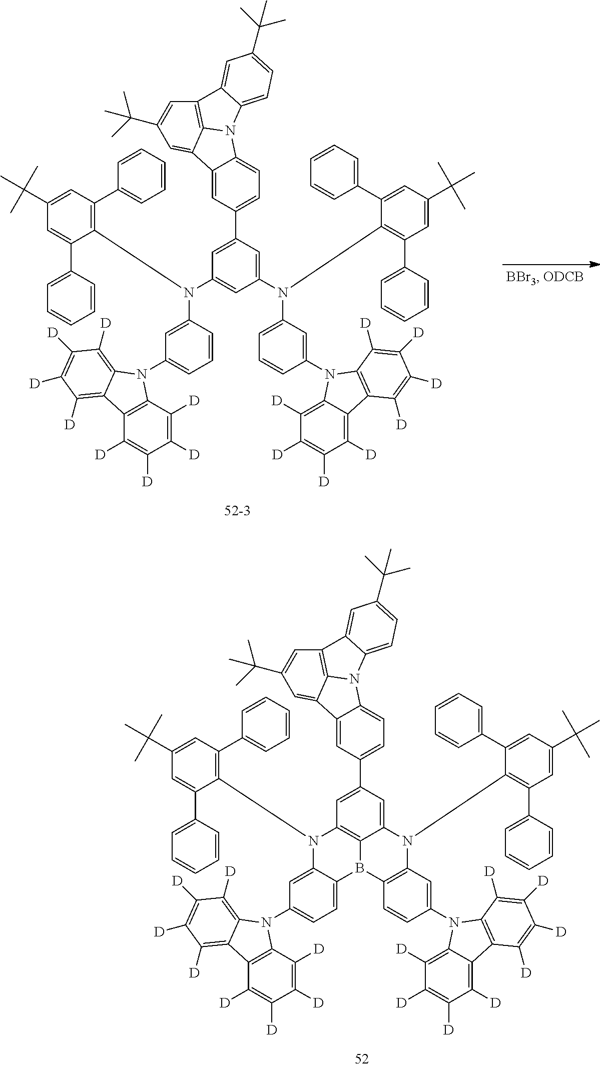

Intermediate 52-3 (1 eq) was dissolved in o-dichlorobenzene (ODCB) and cooled to about 0 degrees centigrade. BBr₃ (5 eq) was slowly added thereto dropwise, and the temperature was raised to about 180 degrees, followed by stirring for about 12 hours. After cooling, to a flask containing the reaction mixture, triethylamine was slowly added dropwise to terminate the reaction, and ethyl alcohol was added to the reaction product to precipitate. The precipitate was filtered to obtain a reaction product. The solid content thus obtained was separated by column chromatography using methylene chloride and n-hexane, and recrystallized using toluene and acetone to obtain Compound 52 (yield: 24%).

### (6) Synthesis of Compound 55

### Synthesis of Intermediate 55-1

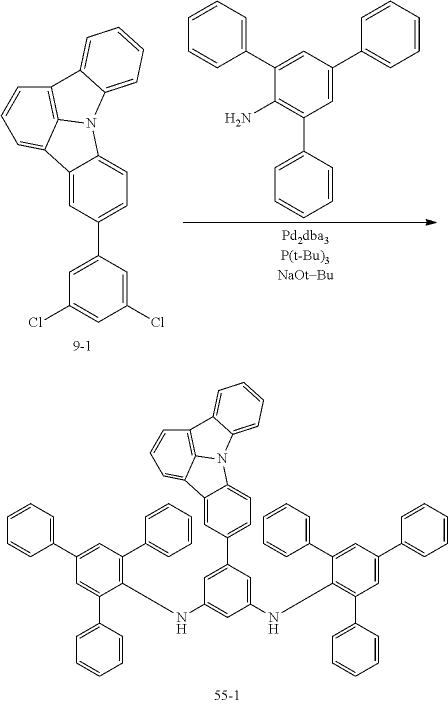 [SMILES: Clc1cc(Cl)cc(-c2ccc3c(c2)c2cccc4c5ccccc5n3c42)c1 ; Nc1c(-c2ccccc2)cc(-c2ccccc2)cc1-c1ccccc1 ; c1ccc(-c2cc(-c3ccccc3)c(Nc3cc(Nc4c(-c5ccccc5)cc(-c5ccccc5)cc4-c4ccccc4)cc(-c4ccc5c(c4)c4cccc6c7ccccc7n5c64)c3)c(-c3ccccc3)c2)cc1]

Intermediate 9-1 (1 eq), 5′-phenyl-[1,1′:3′,1″-terphenyl]-2′-amine (2 eq), tris(dibenzylideneacetone)dipalladium(0) (0.05 eq), tri-tert-butylphosphine (0.1 eq), and sodium tert-butoxide (3 eq) were dissolved in o-xylene, and stirred at about 140 degrees centigrade for about 12 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 55-1 (yield: 62%).

### Synthesis of Intermediate 55-2

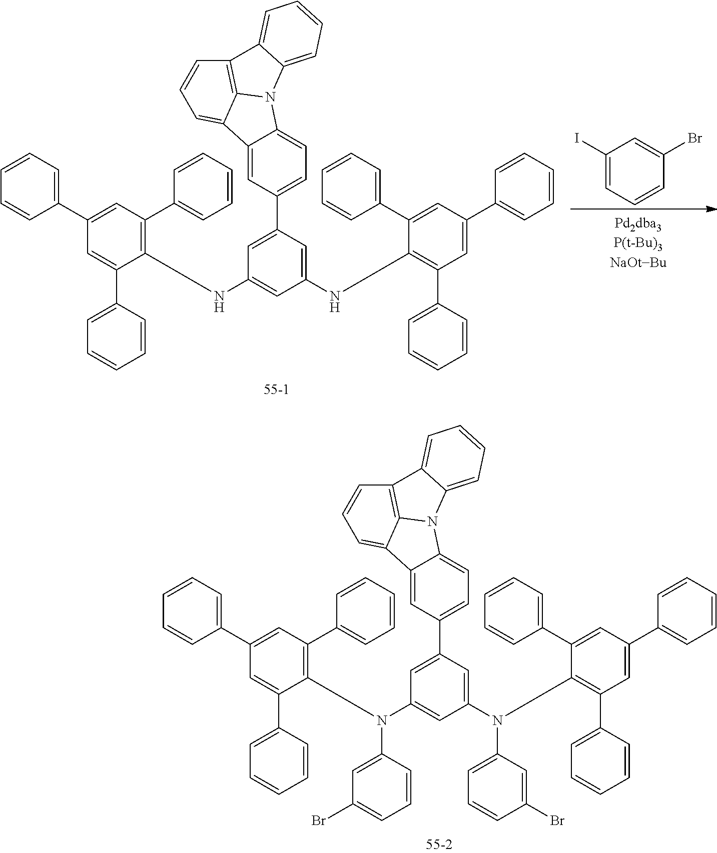

Intermediate 55-1 (1 eq), 1-bromo-3-iodobenzene (5 eq), tris(dibenzylideneacetone)dipalladium(0) (0.1 eq), tri-tert-butylphosphine (0.2 eq), and sodium tert-butoxide (3 eq) were dissolved in o-xylene and stirred at about 160 degrees for about 48 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 55-2 (yield: 50%).

### Synthesis of Intermediate 55-3

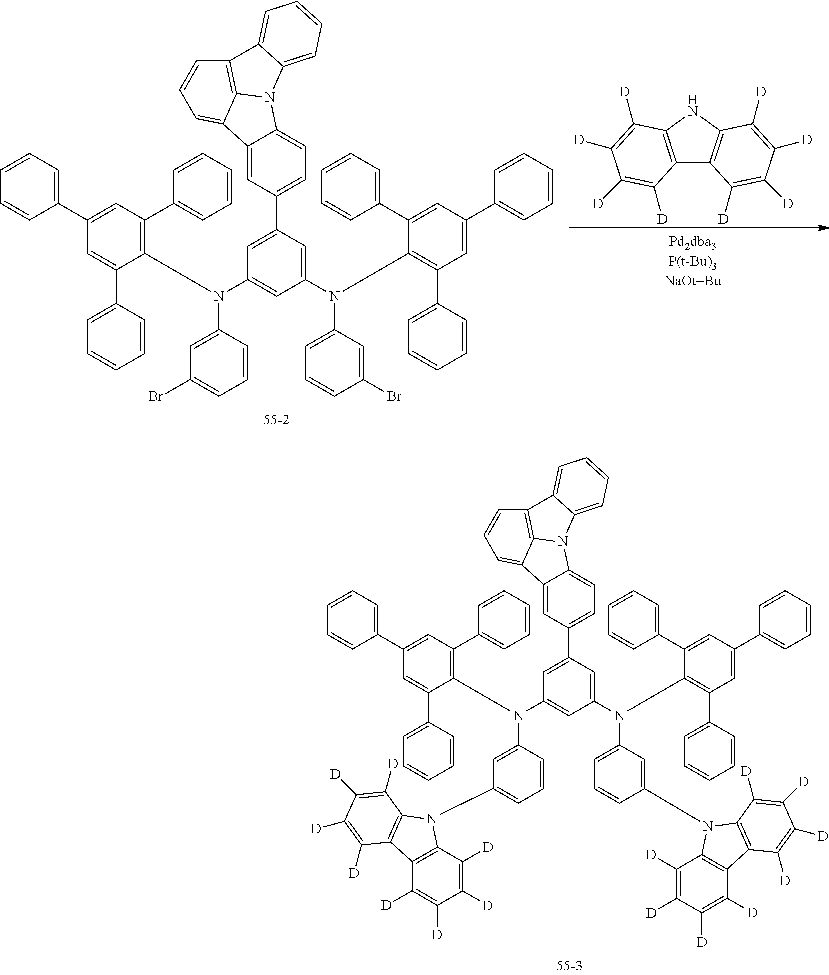

Intermediate 55-2 (1 eq), 9H-carbazole-1,2,3,4,5,6,7,8-d8 (2.5 eq), tris(dibenzylideneacetone)dipalladium(0) (0.1 eq), tri-tert-butylphosphine (0.2 eq), and sodium tert-butoxide (3 eq) were dissolved in o-xylene and stirred at about 150 degrees for about 24 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Intermediate 55-3 (yield: 65%).

### Synthesis of Compound 55

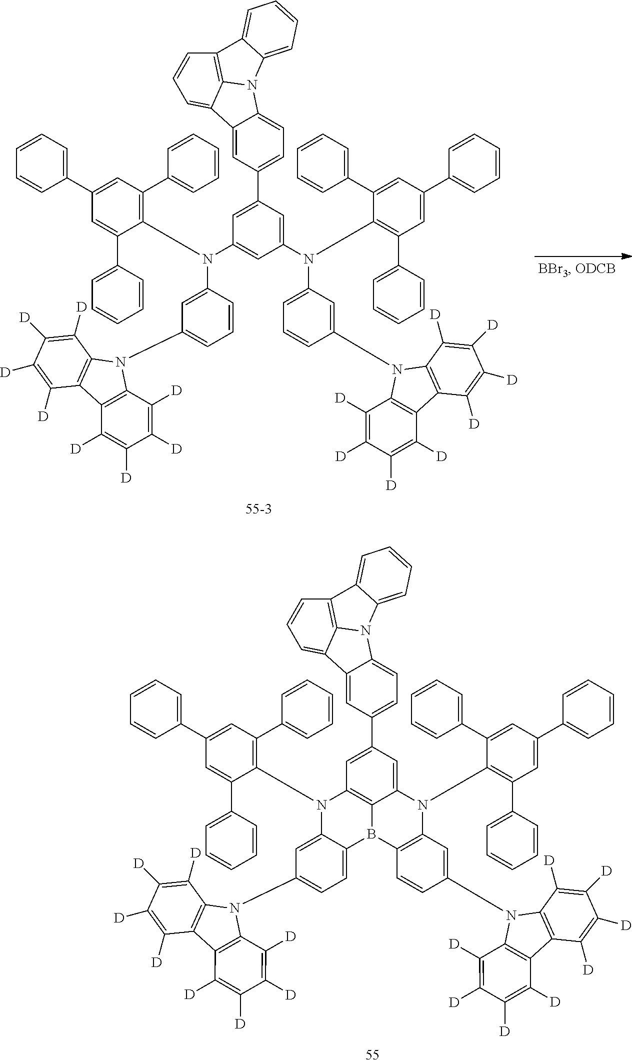

Intermediate 55-3 (1 eq) was dissolved in o-dichlorobenzene (ODCB) and cooled to about 0 degrees centigrade. BBr₃ (5 eq) was slowly added thereto dropwise, and the temperature was raised to about 180 degrees, followed by stirring for about 12 hours. After cooling, to a flask containing the reaction mixture, triethylamine was slowly added dropwise to terminate the reaction, and ethyl alcohol was added to the reaction product to precipitate. The precipitate was filtered to obtain a reaction product. The solid content thus obtained was separated by column chromatography using methylene chloride and n-hexane, and recrystallized using toluene and acetone to obtain Compound 55 (yield: 17%).

### (7) Synthesis of Compound 63

### Synthesis of Compound 63

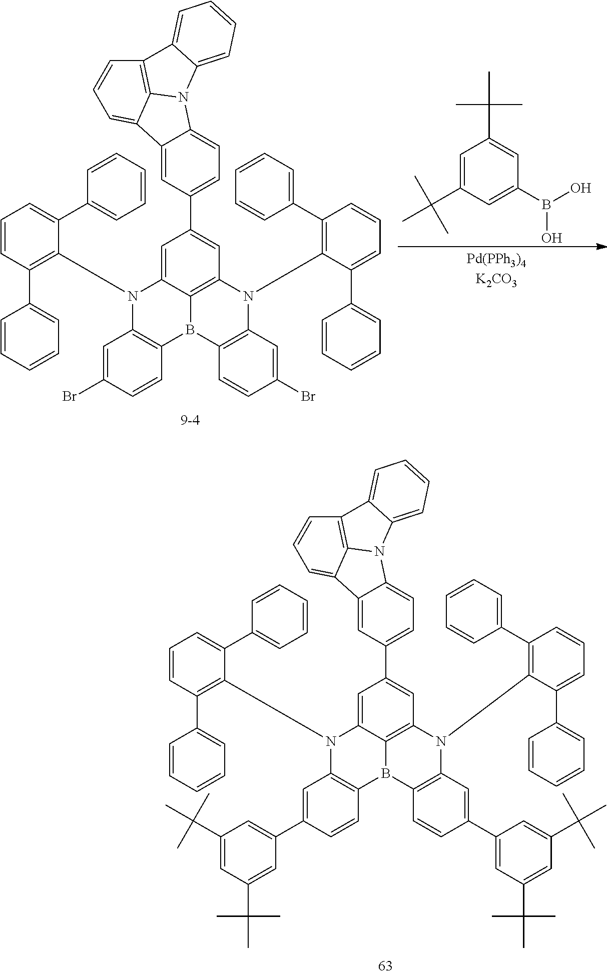

Intermediate 9-4 (1 eq), (3,5-di-tert-butylphenyl)boronic acid (3.5 eq), Pd(PPh₃)₄ (0.05 eq), and K₂CO₃ (3 eq) were dissolved in a mixture of water and THF in a ratio of 2:1, followed by stirring at about 80 degrees centigrade for about 48 hours. After cooling, the reaction product was washed with ethyl acetate and water three times, and an organic layer obtained by separating layers was dried with MgSO₄ and dried under a reduced pressure. The crude product thus obtained was separated by column chromatography using methylene chloride and n-hexane to obtain Compound 63 (yield: 70%).

¹H NMR and MS/FAB of the compounds synthesized in Synthesis Examples (1) to (7) are shown in Table 1. Referring to the aforementioned synthesis procedures and raw materials, synthesis methods of other compounds may be readily recognized by a person skilled in the art.

TABLE 1

| | NMR | Cal | Meas. |
|---|---|---|---|
| 9 | 8.54-8.48 (2H, d), 8.33-8.29 (1H, d), | 1518.813 | 1518.795 |
| | 8.24-8.19 (3H, m), 8.10-8.09 (4H, s), | | |
| | 8.07-8.04 (1H, d), 7.99-7.95 (1H, d), | | |
| | 7.74-7.72 (1H, m), 7.64-7.61 (2H, m), | | |
| | 7.60-7.55 (8H, d), 7.50-7.41 (13H, m), | | |
| | 7.40-7.31 (8H, m), 7.15-7.06 (6H, d), | | |
| | 6.89-6.77 (4H, m), 6.64-6.60 (2H, s), | | |
| | 1.57-1.42 (36H, m) | | |
| 16 | 9.21-9.14 (2H, d), 8.31-8.26 (1H, d), | 1344.401 | 1344.381 |
| | 8.22-8.10 (5H, m), 8.04-8.00 (2H, d), | | |
| | 7.63-7.55 (8H, d), 7.51-7.41 (8H, t) | | |
| | 7.99-7.95 (2H, d), 7.73-7.69 (1H, m), | | |
| | 7.64-7.61 (2H, m), 7.47-7.40 (9H, m), | | |
| | 7.36-7.31 (4H, t), 7.23-7.06 (8H, m) | | |
| | 6.85-6.77 (4H, m), 6.63-6.55 (2H, s) | | |
| 20 | 8.44-8.36 (2H, m), 8.28-8.19 (4H, m), | 1144.153 | 1144.02 |
| | 8.04-7.93 (6H, m) 7.69-7.55 (13H, d), | | |
| | 7.52-7.30 (17H, m), 6.92-6.81 (6H, s), | | |
| | 6.63-6.60 (2H, s) | | |
| 22 | 8.69-8.63 (1H, m), 8.56-8.50 (1H, m), | 1519.797 | 1519.688 |
| | 8.31-8.24 (4H, m), 8.10-8.05 (3H, m), | | |
| | 7.96-7.93 (3H, m), 7.68-7.56 (13H, m), | | |
| | 7.53-7.31 (20H, m), 7.13-7.06 (6H, d), | | |
| | 6.89-6.85 (2H, m), 6.64-6.60 (2H, s), | | |
| | 1.58-1.41 (36H, d) | | |
| 52 | 8.63-8.59 (2H, d), 8.28-8.13 (4H, d), | 1534.911 | 1534.901 |
| | 8.03-7.93 (2H, d), 7.73-7.55 (10H, m), | | |
| | 7.50-7.32 (12H, t), 7.12-7.06 (6H, d) | | |
| | 6.87-6.85 (2H, m), 6.63-6.60 (2H, s), | | |
| | 1.59-1.41 (36H, m) | | |
| 55 | 8.62-8.53 (2H, m), 8.29-8.19 (4H, m), | 1462.675 | 1462.575 |
| | 8.04-7.98 (2H, m), 7.74-7.71 (1H, m), | | |
| | 7.64-7.57 (10H, m), 7.54-7.38 (19H, m) | | |
| | 7.35-7.31 (4H, t), 7.15-7.06 (6H, d), | | |
| | 6.91-6.85 (2H, m), 6.66-6.60 (2H, s) | | |
| 63 | 8.66-8.57 (2H, d), 8.20-8.11 (5H, m), | 1340.619 | 1340.588 |
| | 7.82-7.74 (6H, m) 7.64-7.61 (2H, m), | | |
| | 7.63-7.55 (19H, m), 7.36-7.31 (4H, m), | | |
| | 7.17-7.07 (6H, m), 6.85-6.77 (2H, m), | | |
| | 6.63-6.55 (2H, s), 6.81-6.77 (2H, t), | | |
| | 1.39-1.35 (36H, s) | | |

2. Manufacture and evaluation of light emitting device including first dopant

(Manufacture of Light Emitting Device)

Light emitting devices of Examples 1 to 21 were manufactured using Compounds 9, 16, 20, 22, 52, 55 and 63 as the first dopant materials of an emission layer.

### Example Compounds

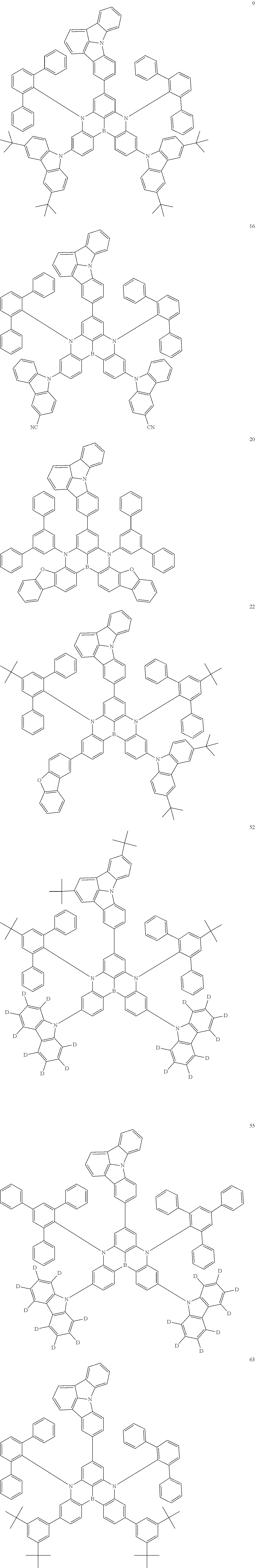

Comparative Compound X-1 and Comparative Compound X-2 were used for the manufacture of devices of Comparative Example 1 and Comparative Example 2.

[Comparative Compounds]

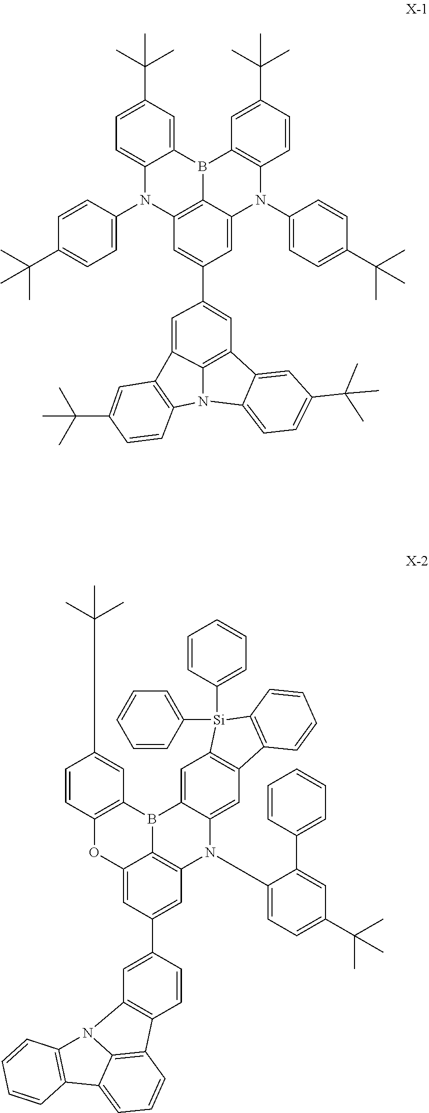

(Manufacture of Light Emitting Devices)

The light emitting devices of the Examples and the Comparative Examples were manufactured as follows. An ITO glass substrate was cut into a size of about 50 mm×50 mm×0.7 mm, washed by ultrasonic waves using isopropyl alcohol and distilled water for about 5 minutes each, and cleaned by irradiating ultraviolet rays for about 30 minutes and with ozone. After that, the ITO glass substrate was installed in a vacuum deposition apparatus. A hole injection layer HIL with a thickness of about 300 Å was formed of NPD, a hole transport layer HTL with a thickness of about 200 Å was formed of HT-1-1, and an emission auxiliary layer with a thickness of about 100 Å was formed of CzSi. A host compound of a mixture of a first host and a second host according to embodiments in a ratio of 1:1, a second dopant, and the Example Compound or Comparative Compound were co-deposited in a weight ratio of about 85:14:1 to form an emission layer EML with a thickness of about 200 Å, and a hole blocking layer with a thickness of about 200 Å was formed of TSPO1. An electron transport layer ETL with a thickness of about 300 Å was formed of TPBi, and an electron injection layer EIL with a thickness of about 10 Å was formed of LiF. A second electrode EL2 with a thickness of about 3000 Å was formed of Al. All layers were formed by a vapor deposition method.

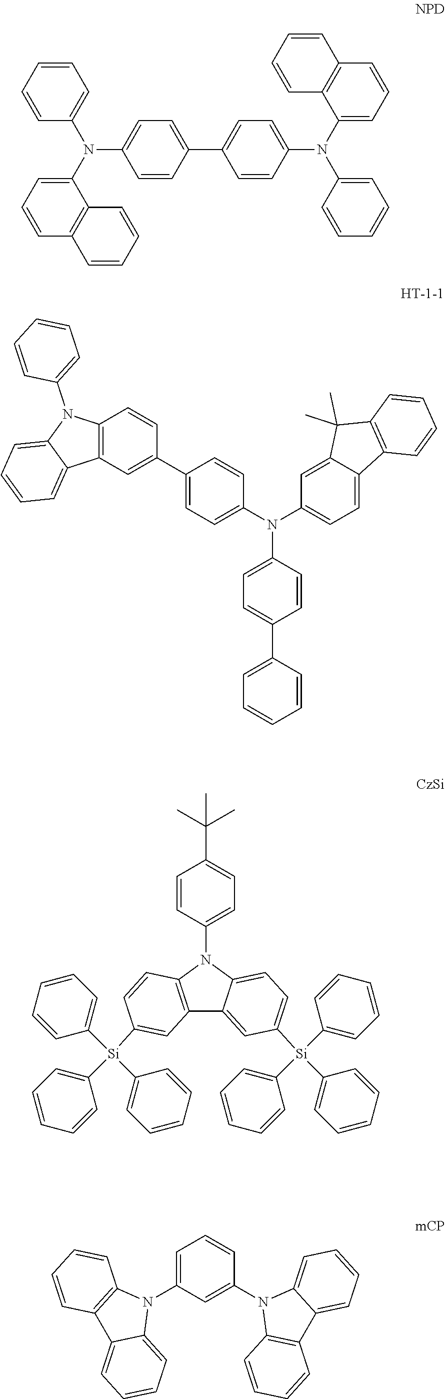 [SMILES: c1ccc(N(c2ccc(-c3ccc(N(c4ccccc4)c4cccc5ccccc45)cc3)cc2)c2cccc3ccccc23)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC(C)(C)c1ccc(-n2c3ccc([Si](c4ccccc4)(c4ccccc4)c4ccccc4)cc3c3cc([Si](c4ccccc4)(c4ccccc4)c4ccccc4)ccc32)cc1 ; c1cc(-n2c3ccccc3c3ccccc32)cc(-n2c3ccccc3c3ccccc32)c1]

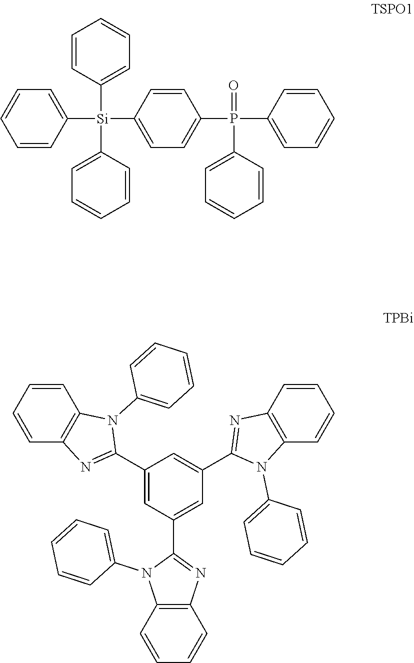 [SMILES: O=P(c1ccccc1)(c1ccccc1)c1ccc([Si](c2ccccc2)(c2ccccc2)c2ccccc2)cc1 ; c1ccc(-n2c(-c3cc(-c4nc5ccccc5n4-c4ccccc4)cc(-c4nc5ccccc5n4-c4ccccc4)c3)nc3ccccc32)cc1]

(Evaluation of Properties of Light Emitting Devices)

The evaluation of the properties of the light emitting devices was conducted using a light distribution measurement system. In order to evaluate the properties of the light emitting devices according to the Examples and Comparative Examples, a driving voltage, efficiency, and an emission wavelength were measured. In Table 2, a current density of about 10 mA/cm², and emission efficiency (cd/A) at a luminance of about 1000 cd/m² were measured for the light emitting devices manufactured.

TABLE 2

| Device | | | | Driving | Emission | Emission |
|---|---|---|---|---|---|---|
| manufacturing | First host/ | First | Second | voltage | efficiency | wavelength |
| example | second host | dopant | dopant | (V) | (Cd/A) | (nm) |
| Example 1 | HT-12/ET-15 | Compound 9 | AD-37 | 4.2 | 25.1 | 459 |
| Example 2 | HT-12/ET-15 | Compound 16 | AD-37 | 4.3 | 25.3 | 463 |
| Example 3 | HT-12/ET-15 | Compound 20 | AD-37 | 4.5 | 24.1 | 462 |
| Example 4 | HT-12/ET-15 | Compound 22 | AD-37 | 4.3 | 26.4 | 461 |
| Example 5 | HT-12/ET-15 | Compound 52 | AD-37 | 4.3 | 24.3 | 458 |
| Example 6 | HT-12/ET-15 | Compound 55 | AD-37 | 4.4 | 26.1 | 463 |
| Example 7 | HT-12/ET-15 | Compound 63 | AD-37 | 4.4 | 25.9 | 463 |
| Example 8 | HT-19/ET-16 | Compound 9 | AD-38 | 4.2 | 25.1 | 459 |
| Example 9 | HT-19/ET-16 | Compound 16 | AD-38 | 4.3 | 26.1 | 463 |
| Example 10 | HT-19/ET-16 | Compound 20 | AD-37 | 4.2 | 25.8 | 463 |
| Example 11 | HT-19/ET-16 | Compound 22 | AD-38 | 4.4 | 24.4 | 461 |
| Example 12 | HT-19/ET-16 | Compound 52 | AD-38 | 4.5 | 23.2 | 459 |
| Example 13 | HT-19/ET-16 | Compound 55 | AD-38 | 4.4 | 25.6 | 462 |
| Example 14 | HT-19/ET-16 | Compound 63 | AD-37 | 4.3 | 25.8 | 463 |
| Example 15 | HT-20/ET-16 | Compound 52 | AD-37 | 4.5 | 25.2 | 458 |
| Example 16 | HT-19/ET-15 | Compound 9 | AD-37 | 4.4 | 25.2 | 459 |
| Example 17 | HT-12/ET-17 | Compound 16 | AD-38 | 4.2 | 26.8 | 462 |
| Example 18 | HT-20/ET-15 | Compound 20 | AD-37 | 4.2 | 26.3 | 463 |
| Example 19 | HT-12/ET-16 | Compound 22 | AD-38 | 4.3 | 25.4 | 462 |
| Example 20 | HT-18/ET-17 | Compound 55 | AD-38 | 4.5 | 25.1 | 460 |
| Example 21 | HT-19/ET-17 | Compound 63 | AD-37 | 4.4 | 25.3 | 463 |
| Comparative | HT-12/ET-15 | Comparative | — | 5.1 | 16.5 | 465 |
| Example 1 | | Compound X-1 | | | | |
| Comparative | HT-19/ET-16 | Comparative | — | 5.2 | 15.9 | 464 |
| Example 2 | | Compound X-1 | | | | |
| Comparative | HT-12/ET-15 | Comparative | — | 5.5 | 12.1 | 455 |
| Example 3 | | Compound X-2 | | | | |
| Comparative | HT-12/ET-15 | Comparative | AD-37 | 4.8 | 19.9 | 464 |
| Example 4 | | Compound X-1 | | | | |
| Comparative | HT-12/ET-15 | Comparative | AD-37 | 5.3 | 15.4 | 459 |
| Example 5 | | Compound X-2 | | | | |

Referring to the results of Table 1, it could be confirmed that the Examples of the light emitting devices using the first dopant according to an embodiment as a light emitting material showed a reduced driving voltage and improved emission efficiency, while maintaining the light emitting wavelength of blue light when compared to the Comparative Examples. The first dopant according to an embodiment includes at least one indolocarbazole group as a substituent in a plate type skeleton structure with a boron atom as a center. The indolocarbazole group may be bonded to a benzene ring bonded to a boron atom at a central core, and the boron atom and the indolocarbazole group may be bonded at a para position to each other. The indolocarbazole group may be bonded to the central core at a carbon of position 5 or a carbon at position 10. Accordingly, the first dopant according to an embodiment has a high oscillator strength value and a small ΔE_{ST} value, through the increase of multiple resonance effects due to the increase of the electron donating properties of the indolocarbazole group, and improved delayed fluorescence emission properties may be expected. The first dopant according to an embodiment may have a strong bond structure through a carbon-carbon bond between the indolocarbazole group and the central core, and the chemical stability of the material itself could be improved. The first dopant of an embodiment includes an indolocarbazole group, and the light absorption of the compound itself could be increased, and accordingly, when the first dopant of an embodiment is used as a thermally activated delayed fluorescence dopant, energy transfer efficiency with a host material may be improved, and emission efficiency may be improved even further. The light emitting device of an embodiment includes the first dopant of an embodiment as the light emitting dopant of a thermally activated delayed fluorescence (TADF) emitting device, and high device efficiency, for example, in a blue wavelength region may be achieved.

It could be confirmed that Comparative Compound X-1 included in Comparative Example 1, Comparative Example 2, and Comparative Example 4 has a plate type skeleton structure with one boron atom as a center and include an indolocarbazole group as a substituent, but has a structure in which a carbon at position 2 of the indolocarbazole group is bonded to a central core, rather than a carbon at position 5 or a carbon at position 10. Thus, Comparative Compound X-1 included in Comparative Example 1, Comparative Example 2, and Comparative Example 4 showed a high driving voltage and degraded emission efficiency when compared to the Examples. In the case of Comparative Compound X-1, a carbon at position 2 of the indolocarbazole is bonded to the central core, and it is thought that the symmetry of a whole molecule increased, high crystallinity was achieved, the stability of a thin film was reduced, and the quenching phenomenon by the pi-pi interaction between adjacent molecules was generated to deteriorate the emission efficiency when compared to the Examples. In the cases of Comparative Example 1 and Comparative Example 2, it could be found that the second dopant of an embodiment was not included in an emission layer, and relatively low emission efficiency was shown when compared to Comparative Example 4 and the Examples.

Comparative Compound X-2 included in Comparative Example 3 and Comparative Example 5 has a plate type skeleton structure with one boron atom as a center and include one indolocarbazole group as a substituent, but has a structure in which the boron atom and the nitrogen atom of the indolocarbazole group are bonded at a meta position to each other, and it could be confirmed that a driving voltage was high, and emission efficiency was degraded when compared to the Examples. It was determined that such results were achieved because the boron atom and the nitrogen atom of the indolocarbazole group were bonded at a meta position to each other and the electron donating properties of the indolocarbazole group was reduced. In Comparative Example 3, the second dopant of an embodiment was not included in the emission layer, and relatively low emission efficiency was shown when compared to Comparative Example 5 and the Examples.

The light emitting device of an embodiment may show improved device properties of high efficiency.

The first dopant of an embodiment may be included in the emission layer of a light emitting device and may contribute to the increase of efficiency of the light emitting device.

Embodiments have been disclosed herein, and although terms are employed, they are used and are to be interpreted in a generic and descriptive sense only and not for purpose of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the following claims.

What is claimed is:

1. A light emitting device, comprising:
a first electrode;
a second electrode facing the first electrode; and
an emission layer disposed between the first electrode and the second electrode, wherein
the emission layer comprises:
a first host represented by Formula H-1;
a second host represented by Formula H-2; and
a first dopant represented by Formula 1: 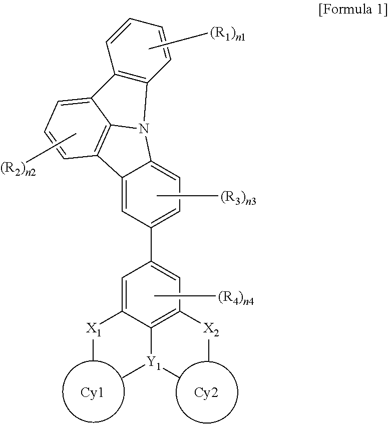
wherein in Formula 1,
X₁ and X₂ are each independently N(R₅), O, or S,
Y₁ is B,
Cy1 and Cy2 are each independently a substituted or unsubstituted aromatic hydrocarbon ring of 6 to 30 ring-forming carbon atoms, or a substituted or unsubstituted aromatic heterocycle of 2 to 30 ring-forming carbon atoms, or are combined with an adjacent group to form a ring,
R₁ to R₄ are each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or are combined with an adjacent group to form a ring,
R₅ is a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or is combined with an adjacent group to form a ring,
n₁ is an integer from 0 to 4,
n₂ and n₃ are each independently an integer from 0 to 3, and
n₄ is an integer from 0 to 2, 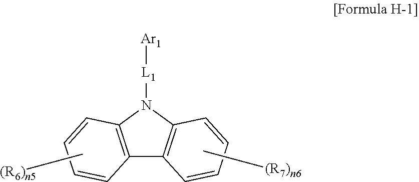
wherein in Formula H-1,
L₁ is a direct linkage, a substituted or unsubstituted arylene group of 6 to 30 ring-forming carbon atoms, or a substituted or unsubstituted heteroarylene group of 2 to 30 ring-forming carbon atoms,
Ar₁ is a substituted or unsubstituted aryl group of 6 to 30 ring-forming carbon atoms, or a substituted or unsubstituted heteroaryl group of 2 to 30 ring-forming carbon atoms,
R₆ and R₇ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and
n₅ and n₆ are each independently an integer from 0 to 4, 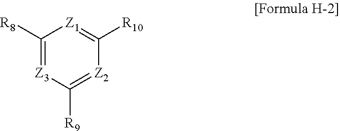
wherein in Formula H-2,
Z₁ to Z₃ are each independently C(R₁₁) or N,
at least one of Z₁ to Z₃ is N, and
R₈ to R₁₁ are each independently a hydrogen atom, a deuterium atom, a cyano group, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms.

2. The light emitting device of claim 1, wherein Cy1 and Cy2 are each independently a group represented by Formula 2: 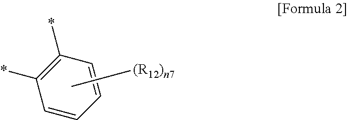
wherein in Formula 2,
R₁₂ is a hydrogen atom, a deuterium atom, a halogen atom, a cyano group, a substituted or unsubstituted amine group, a substituted or unsubstituted oxy group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or is combined with an adjacent group to form a ring,
n₇ is an integer from 0 to 4, and [symbol]
are bonding sites to X₁ and Y₁ of Formula 1, or are bonding sites to X₂ and Y₁ of Formula 1.

3. The light emitting device of claim 2, wherein
if Cy1 and Cy2 are each a group represented by Formula 2, then Cy1 and Cy2 are each independently a group represented by Formula 2-1 or Formula 2-2: 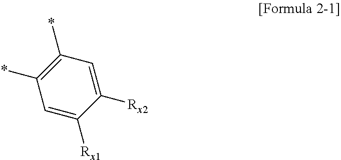
wherein in Formula 2-1,
Rₓ₁ and Rₓ₂ are each independently a hydrogen atom, a deuterium atom, a halogen atom, a substituted or unsubstituted alkyl group of 1 to 20 carbon atoms, a substituted or unsubstituted phenyl group, a substituted or unsubstituted amine group, a substituted or unsubstituted carbazole group, or a substituted or unsubstituted indolocarbazole group, or are combined with an adjacent group to form a ring, and [symbol]
are bonding sites to X₁ and Y₁ of Formula 1, or are bonding sites to X₂ and Y₁ of Formula 1, 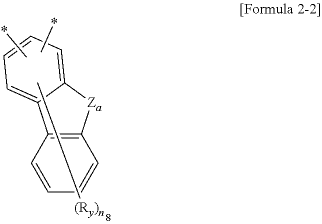
wherein in Formula 2-2,
Zₐ is N(R₁₃) or O,
R_{y} is a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or is combined with an adjacent group to form a ring,
R₁₃ is a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n₈ is an integer from 0 to 6, and [symbol]
are bonding sites to X₁ and Y₁ of Formula 1, or are bonding sites to X₂ and Y₁ of Formula 1.

4. The light emitting device of claim 2, wherein R₁₂ is a substituted or unsubstituted alkyl group of 1 to 10 carbon atoms, a substituted or unsubstituted amine group, a substituted or unsubstituted oxy group, or a group represented by one of Formula 3-1 to Formula 3-4: 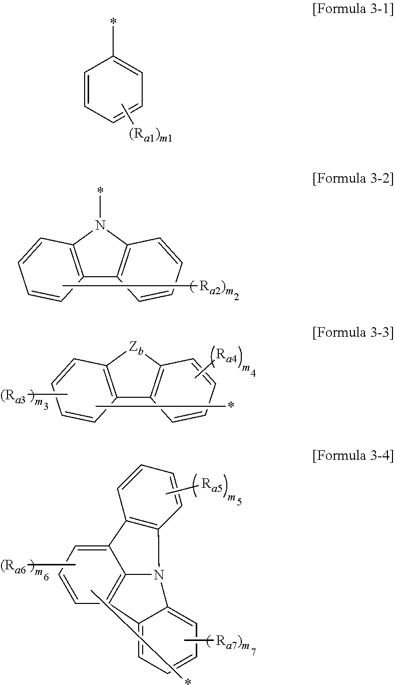
wherein in Formula 3-1 to Formula 3-4,
Z_{b} is N(R₁₄) or O,
Rₐ₁ to Rₐ₇ and R₁₄ are each independently a hydrogen atom, a deuterium atom, a halogen atom, a cyano group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
m₁ is an integer from 0 to 5,
m₂ is an integer from 0 to 8,
m₃ to m₅, and m₇ are each independently an integer from 0 to 4,
m₆ is an integer from 0 to 3,
a sum of m₃ and m₄ is equal to or less than 7, and
a sum of m₆ and m₇ is equal to or less than 6.

5. The light emitting device of claim 1, wherein the first dopant represented by Formula 1 is represented by one of Formula 4-1 or Formula 4-2: 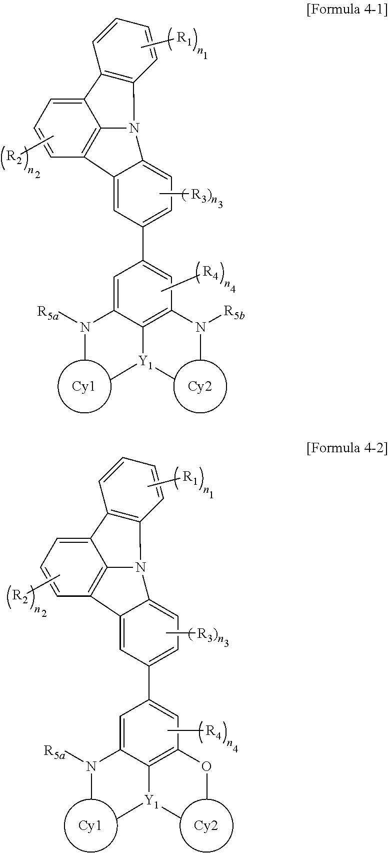
wherein in Formula 4-1 and Formula 4-2,
R₅ₐ and R_{5b} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or are combined with an adjacent group to form a ring, and
Cy1, Cy2, Y₁, R₁ to R₄, and n₁ to n₄ are the same as defined in Formula 1.

6. The light emitting device of claim 1, wherein the first dopant represented by Formula 1 is represented by one of Formula 5-1 to Formula 5-3: 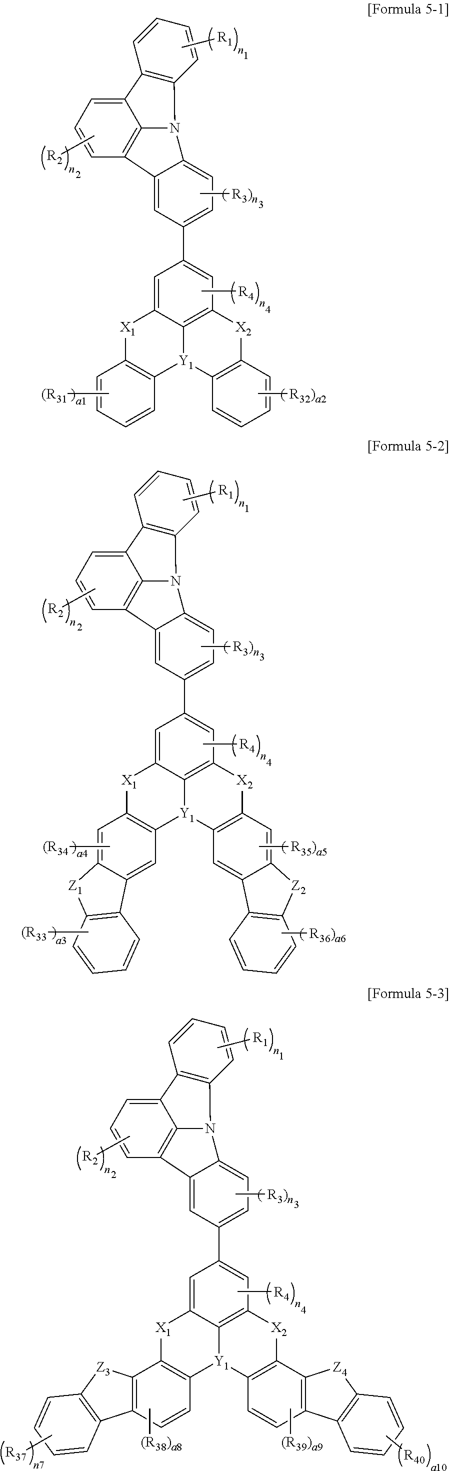
wherein in Formula 5-1 to Formula 5-3,
Z₁ to Z₄ are each independently N(R₄₁) or O,
R₃₁ to R₄₀ are each independently a hydrogen atom, a deuterium atom, a halogen atom, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
R₄₁ is a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
a1 to a3, a6, a7, and a10 are each independently an integer from 0 to 4,
a4, a5, a8, and a9 are each independently an integer from 0 to 2, and
X₁, X₂, Y₁, R₁ to R₄, and n₁ to n₄ are the same as defined in Formula 1.

7. The light emitting device of claim 1, wherein
if each of X₁ and X₂ in Formula 1 is N(R₅), then R₅ is a group represented by one of Formula 6-1 to Formula 6-4: 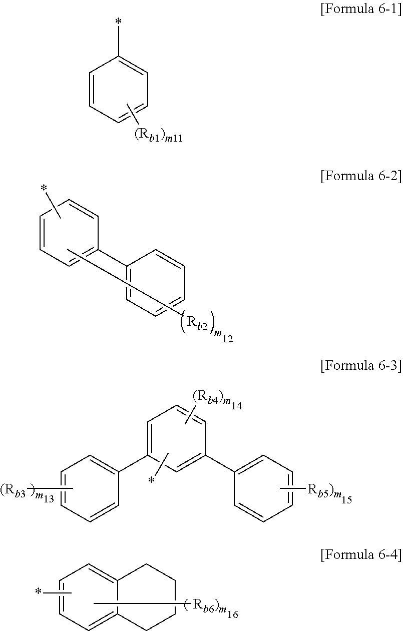
wherein in Formula 6-1 to Formula 6-4,
R_{b1} to R_{b6} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
m₁₁, m₁₃, and m₁₅ are each independently an integer from 0 to 5,
m₁₂ is an integer from 0 to 9,
m₁₄ is an integer from 0 to 3, and
m₁₆ is an integer from 0 to 11.

8. The light emitting device of claim 1, wherein the first dopant comprises at least one selected from Compound Group 1:

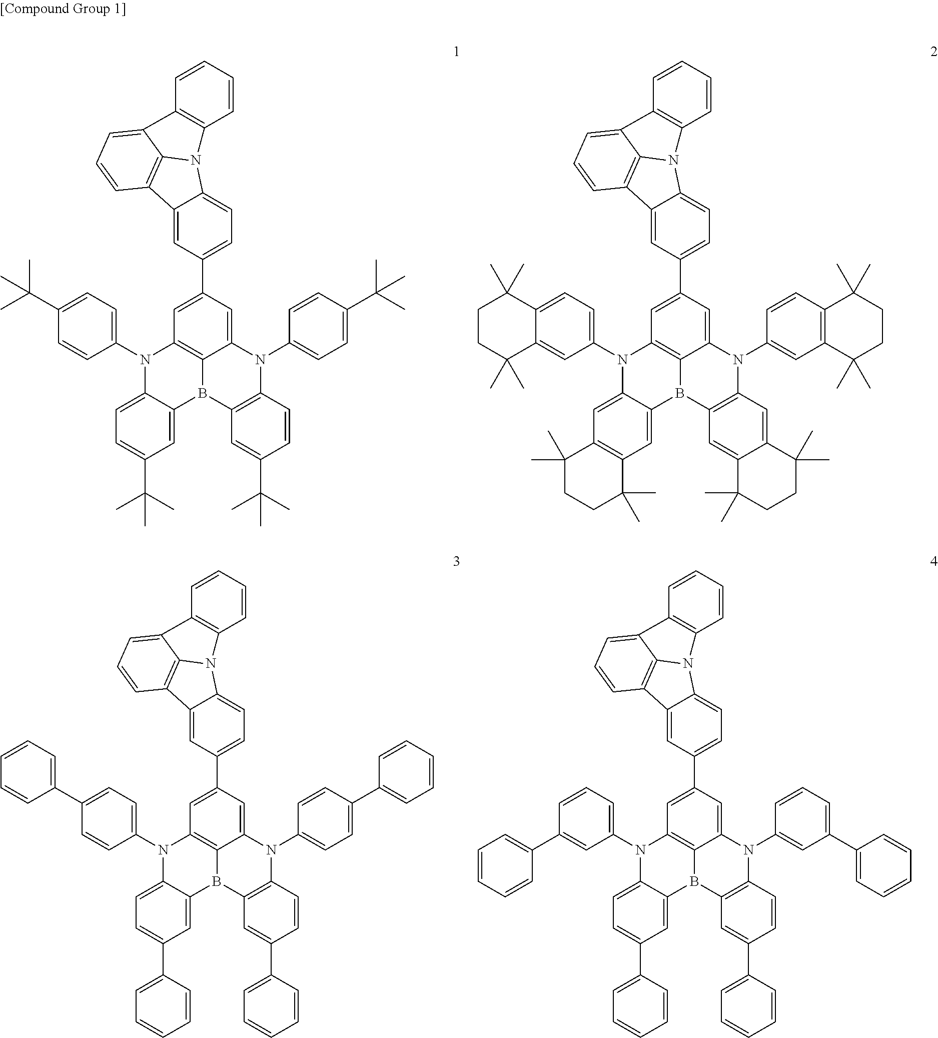

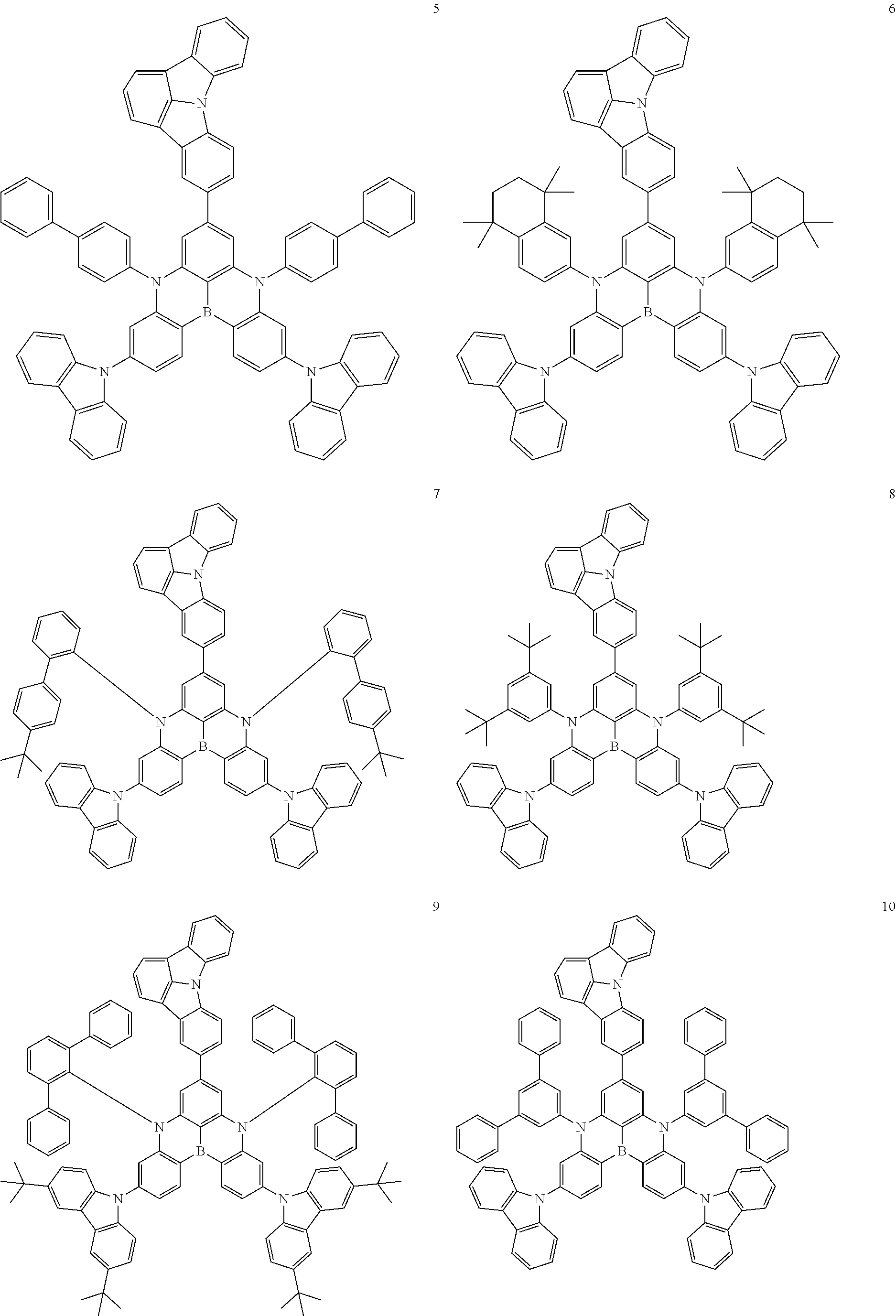

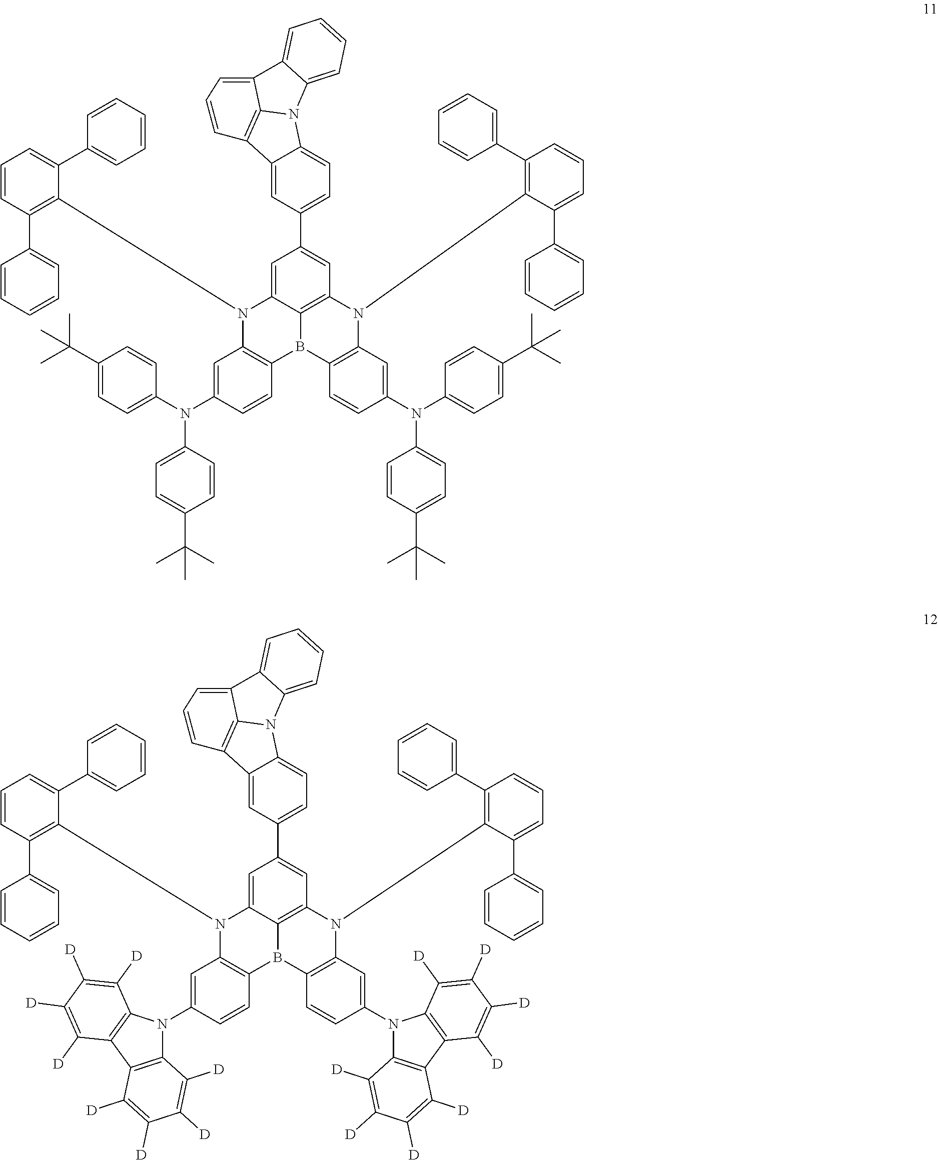 [SMILES: CC(C)(C)c1ccc(N(c2ccc(C(C)(C)C)cc2)c2ccc3c(c2)N(c2c(-c4ccccc4)cccc2-c2ccccc2)c2cc(-c4ccc5c(c4)c4cccc6c7ccccc7n5c64)cc4c2B3c2ccc(N(c3ccc(C(C)(C)C)cc3)c3ccc(C(C)(C)C)cc3)cc2N4c2c(-c3ccccc3)cccc2-c2ccccc2)cc1 ; [2H]c1c([2H])c([2H])c2c(c1[2H])c1c([2H])c([2H])c([2H])c([2H])c1n2-c1ccc2c(c1)N(c1c(-c3ccccc3)cccc1-c1ccccc1)c1cc(-c3ccc4c(c3)c3cccc5c6ccccc6n4c53)cc3c1B2c1ccc(-n2c4c([2H])c([2H])c([2H])c([2H])c4c4c([2H])c([2H])c([2H])c([2H])c42)cc1N3c1c(-c2ccccc2)cccc1-c1ccccc1]

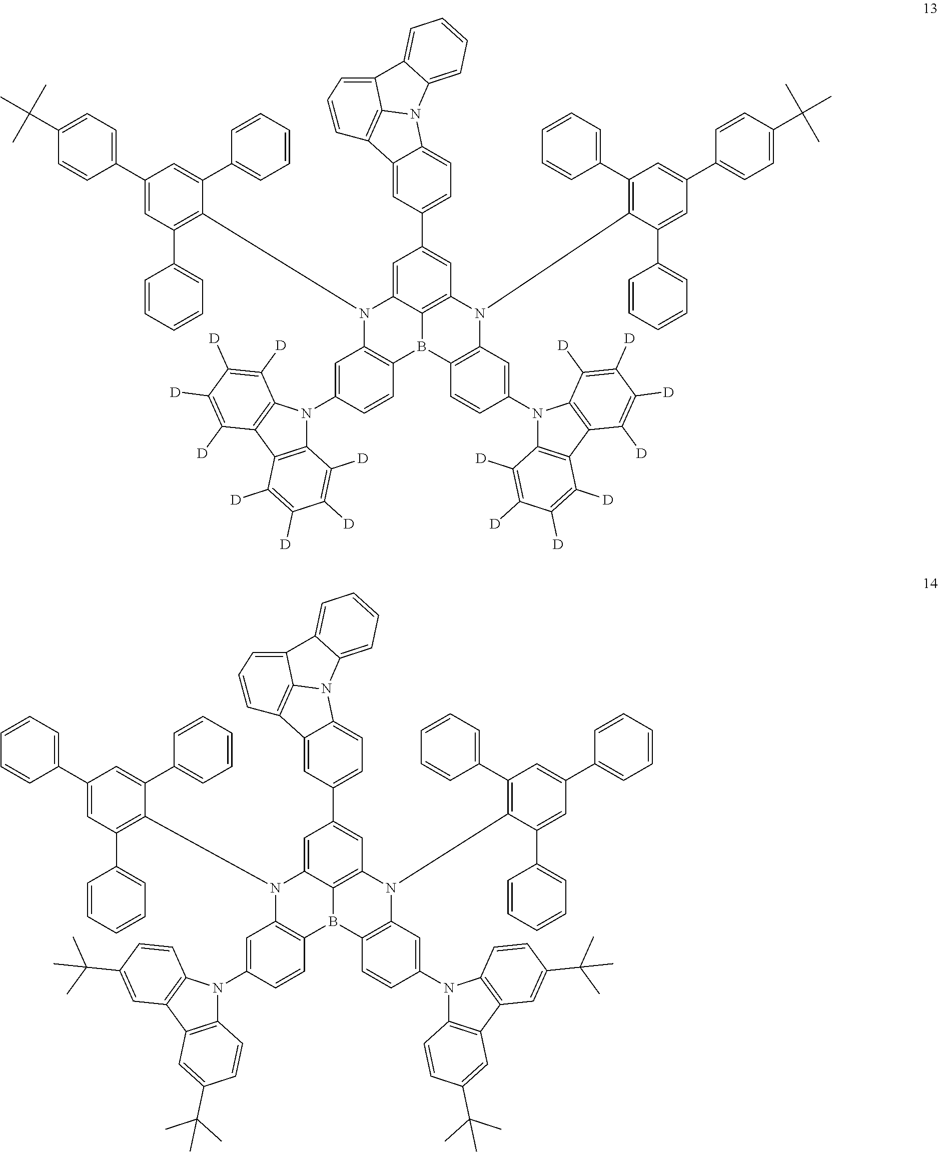

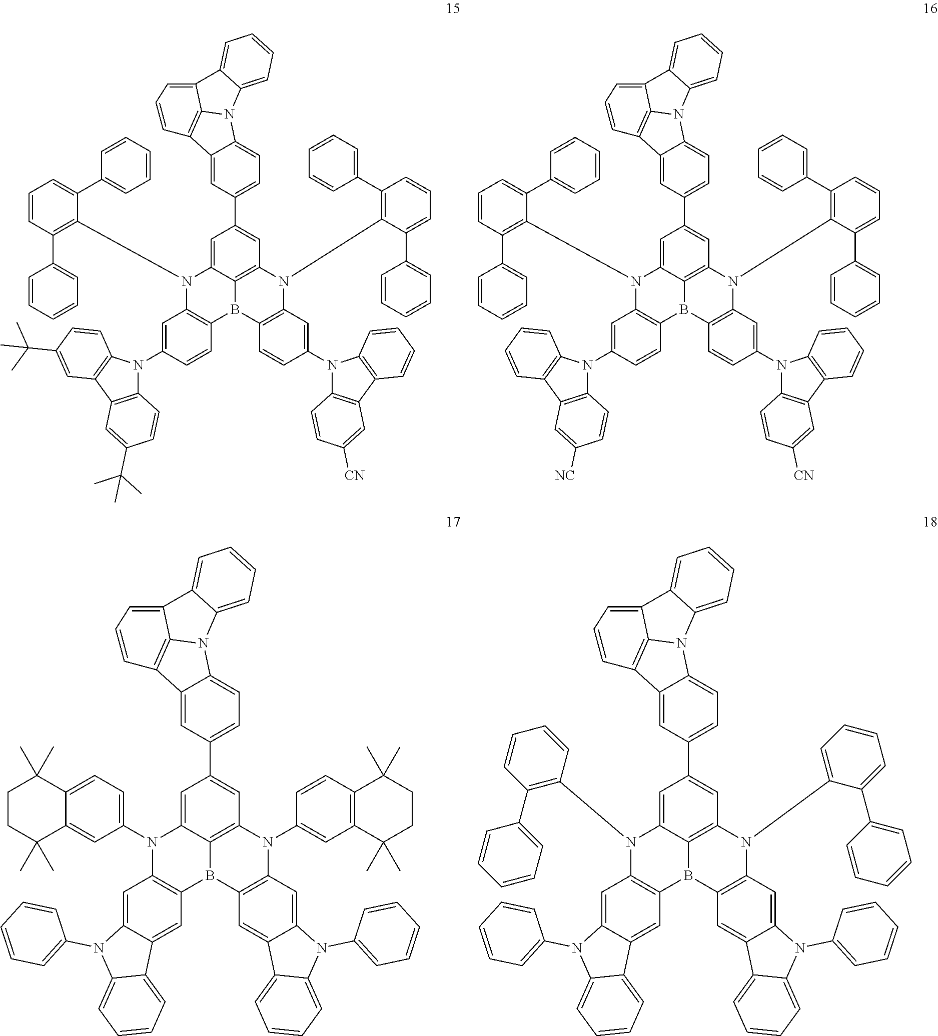

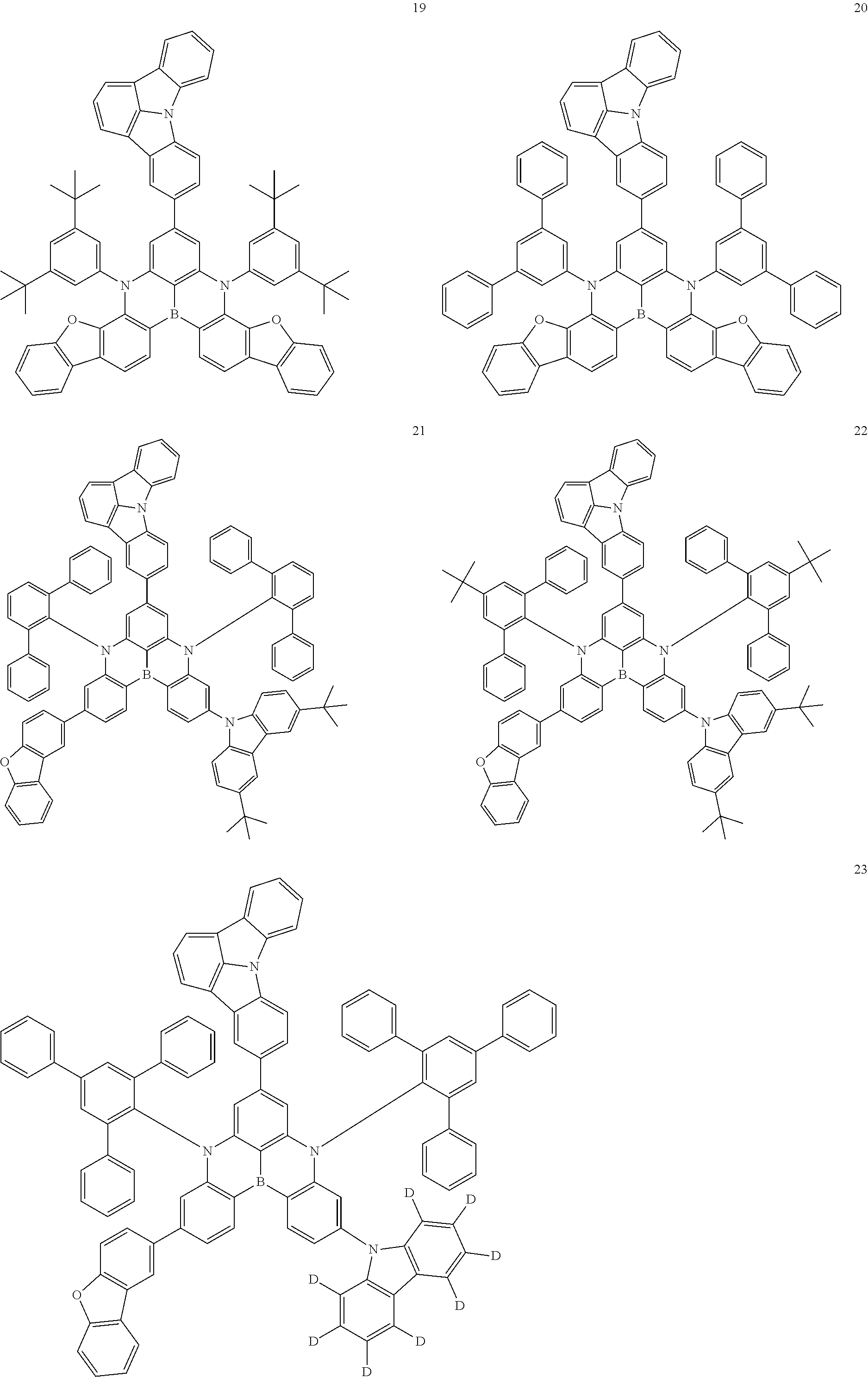

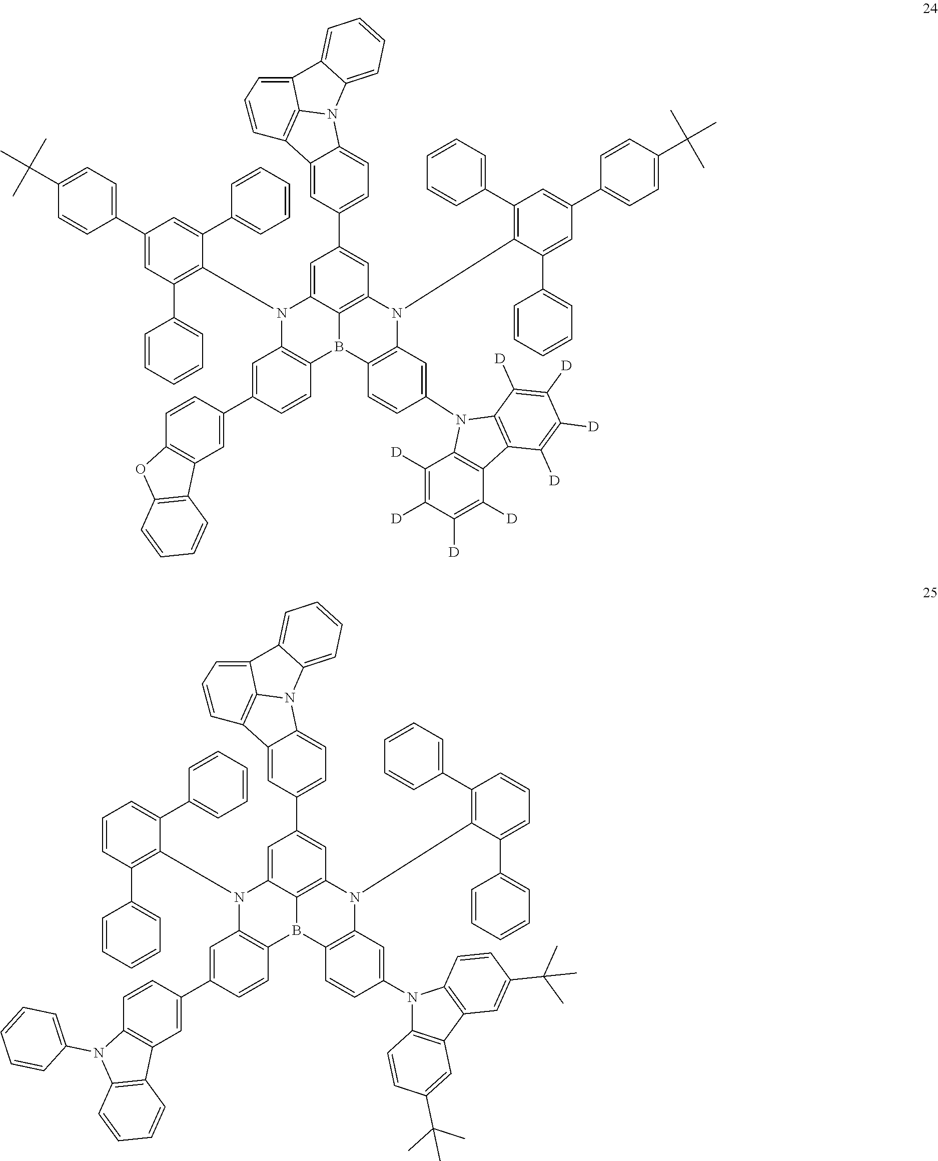 [SMILES: [2H]c1c([2H])c([2H])c2c(c1[2H])c1c([2H])c([2H])c([2H])c([2H])c1n2-c1ccc2c(c1)N(c1c(-c3ccccc3)cc(-c3ccc(C(C)(C)C)cc3)cc1-c1ccccc1)c1cc(-c3ccc4c(c3)c3cccc5c6ccccc6n4c53)cc3c1B2c1ccc(-c2ccc4oc5ccccc5c4c2)cc1N3c1c(-c2ccccc2)cc(-c2ccc(C(C)(C)C)cc2)cc1-c1ccccc1 ; CC(C)(C)c1ccc2c(c1)c1cc(C(C)(C)C)ccc1n2-c1ccc2c(c1)N(c1c(-c3ccccc3)cccc1-c1ccccc1)c1cc(-c3ccc4c(c3)c3cccc5c6ccccc6n4c53)cc3c1B2c1ccc(-c2ccc4c(c2)c2ccccc2n4-c2ccccc2)cc1N3c1c(-c2ccccc2)cccc1-c1ccccc1]

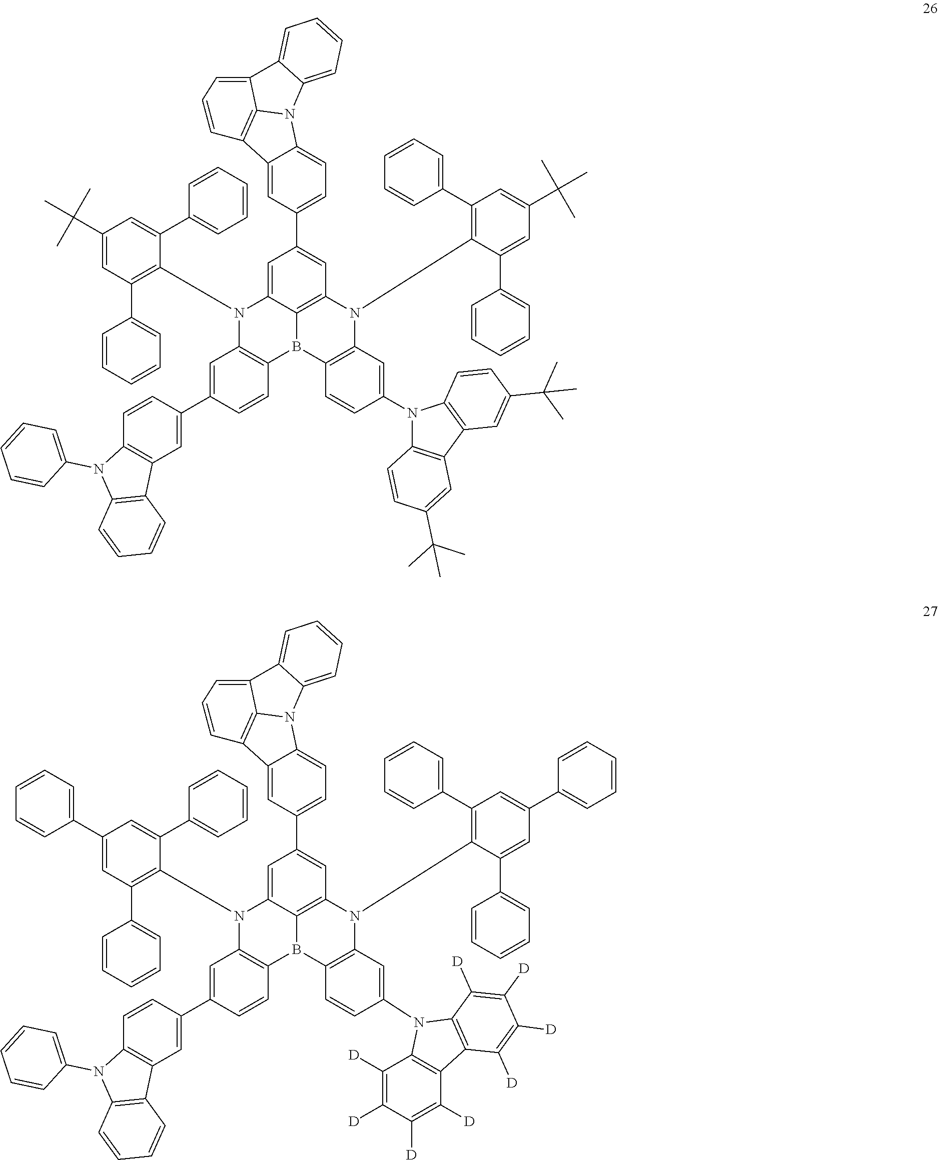 [SMILES: CC(C)(C)c1cc(-c2ccccc2)c(N2c3cc(-c4ccc5c(c4)c4ccccc4n5-c4ccccc4)ccc3B3c4ccc(-n5c6ccc(C(C)(C)C)cc6c6cc(C(C)(C)C)ccc65)cc4N(c4c(-c5ccccc5)cc(C(C)(C)C)cc4-c4ccccc4)c4cc(-c5ccc6c(c5)c5cccc7c8ccccc8n6c75)cc2c43)c(-c2ccccc2)c1 ; [2H]c1c([2H])c([2H])c2c(c1[2H])c1c([2H])c([2H])c([2H])c([2H])c1n2-c1ccc2c(c1)N(c1c(-c3ccccc3)cc(-c3ccccc3)cc1-c1ccccc1)c1cc(-c3ccc4c(c3)c3cccc5c6ccccc6n4c53)cc3c1B2c1ccc(-c2ccc4c(c2)c2ccccc2n4-c2ccccc2)cc1N3c1c(-c2ccccc2)cc(-c2ccccc2)cc1-c1ccccc1]

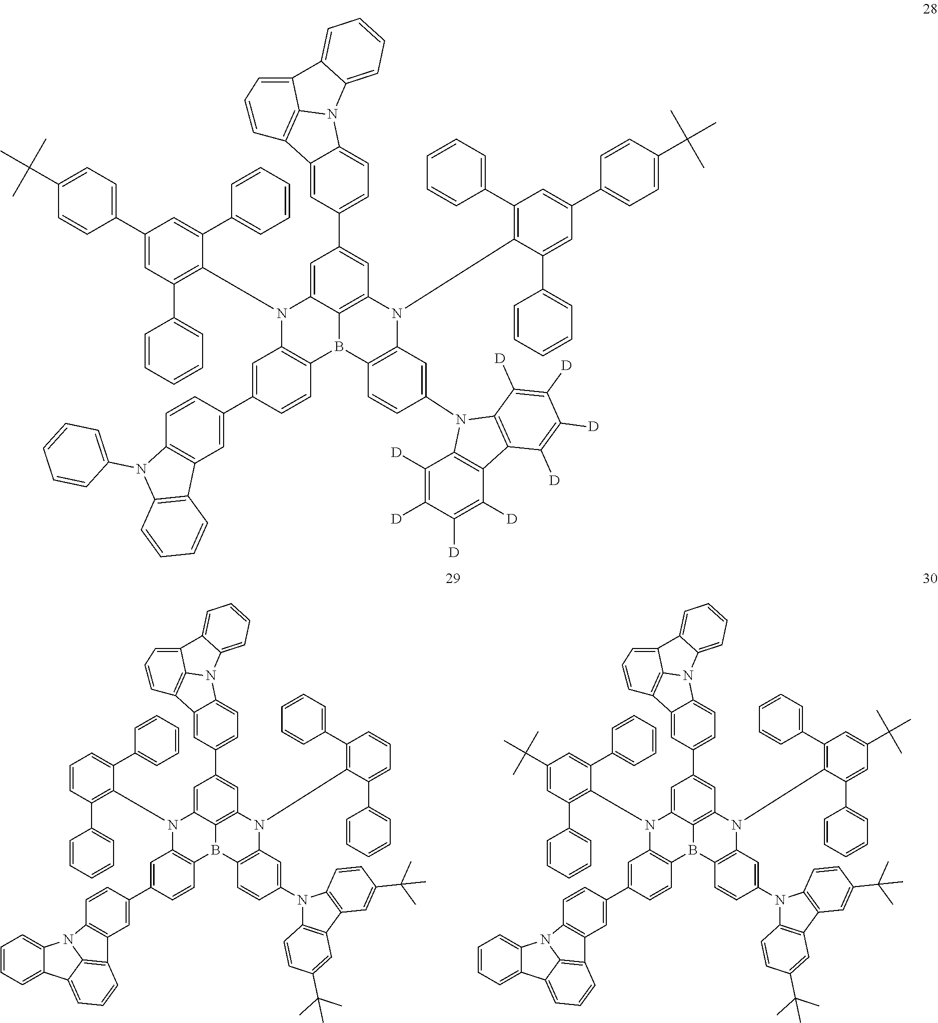

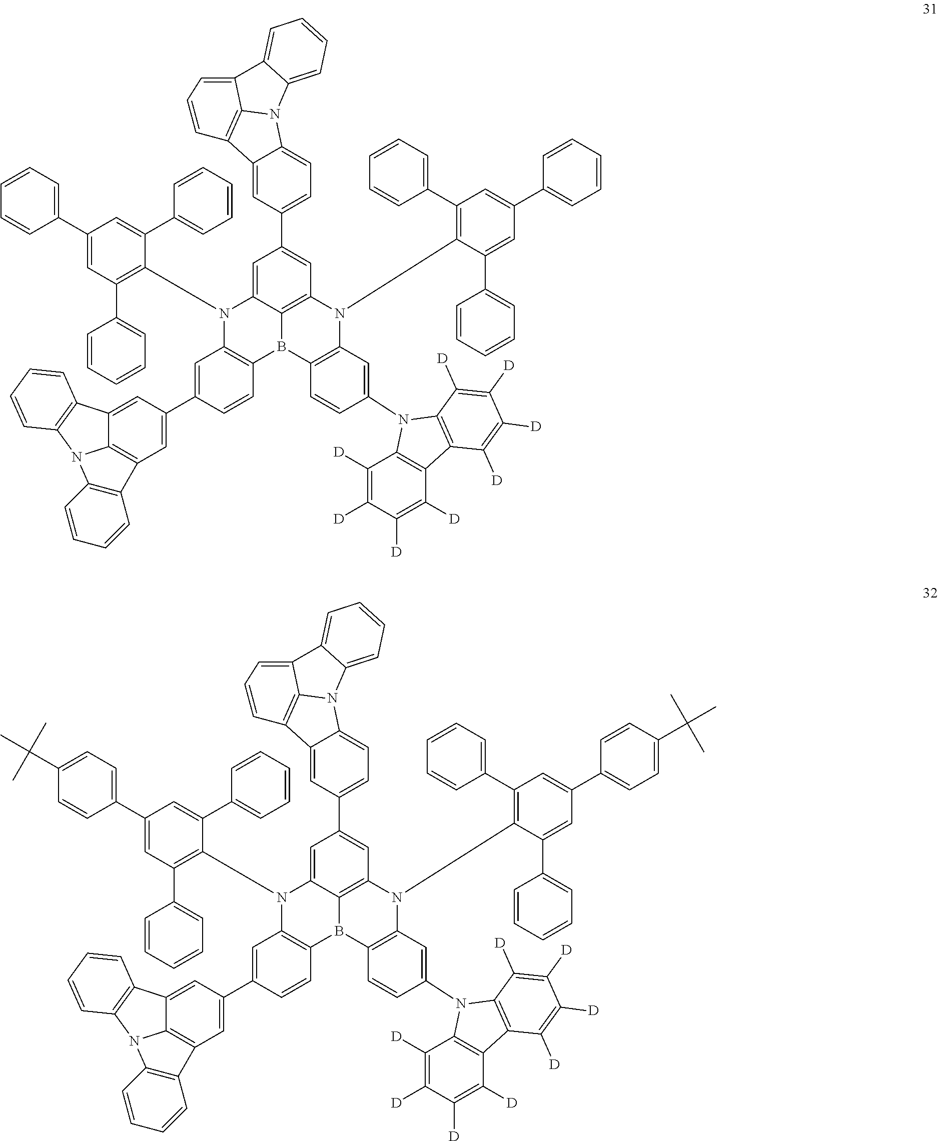

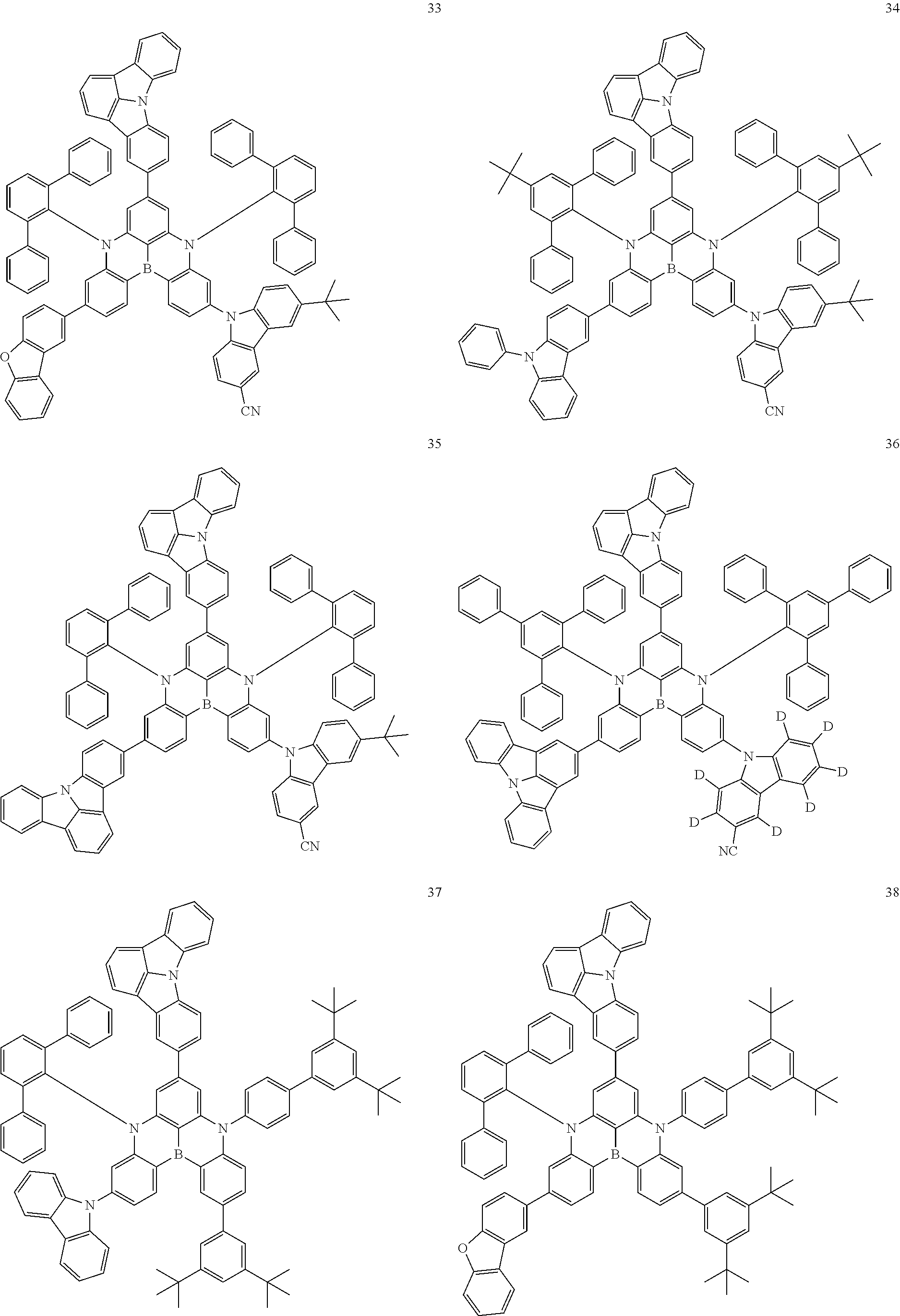

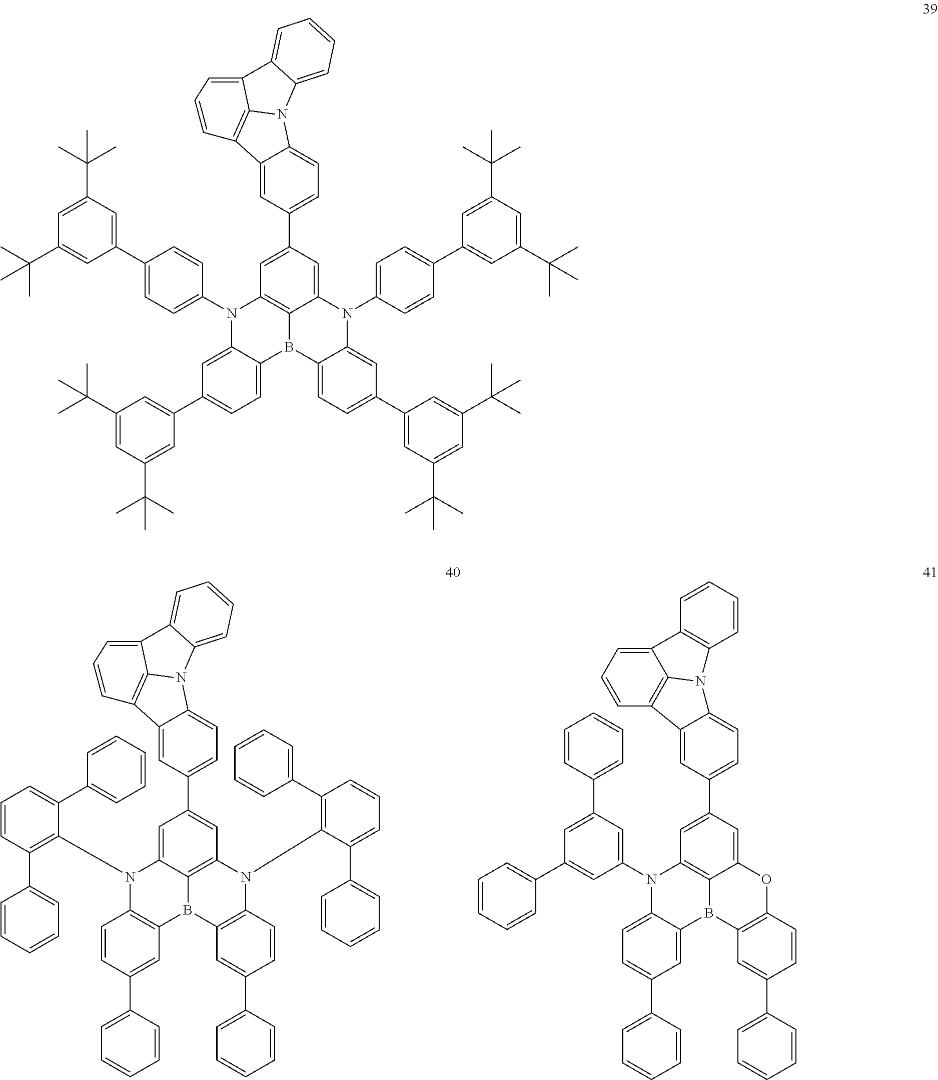 [SMILES: CC(C)(C)c1cc(-c2ccc(N3c4cc(-c5cc(C(C)(C)C)cc(C(C)(C)C)c5)ccc4B4c5ccc(-c6cc(C(C)(C)C)cc(C(C)(C)C)c6)cc5N(c5ccc(-c6cc(C(C)(C)C)cc(C(C)(C)C)c6)cc5)c5cc(-c6ccc7c(c6)c6cccc8c9ccccc9n7c86)cc3c54)cc2)cc(C(C)(C)C)c1 ; c1ccc(-c2ccc3c(c2)B2c4cc(-c5ccccc5)ccc4N(c4c(-c5ccccc5)cccc4-c4ccccc4)c4cc(-c5ccc6c(c5)c5cccc7c8ccccc8n6c75)cc(c42)N3c2c(-c3ccccc3)cccc2-c2ccccc2)cc1 ; c1ccc(-c2cc(-c3ccccc3)cc(N3c4ccc(-c5ccccc5)cc4B4c5cc(-c6ccccc6)ccc5Oc5cc(-c6ccc7c(c6)c6cccc8c9ccccc9n7c86)cc3c54)c2)cc1]

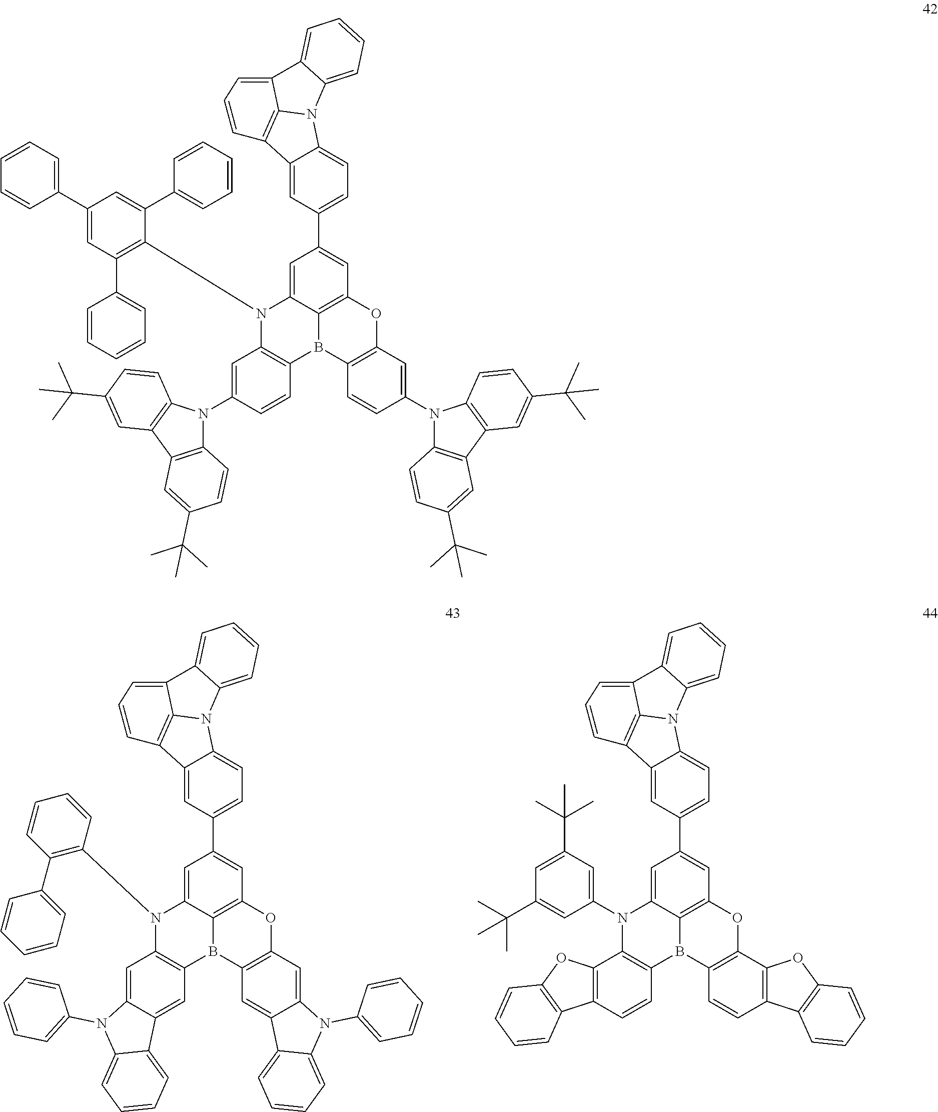

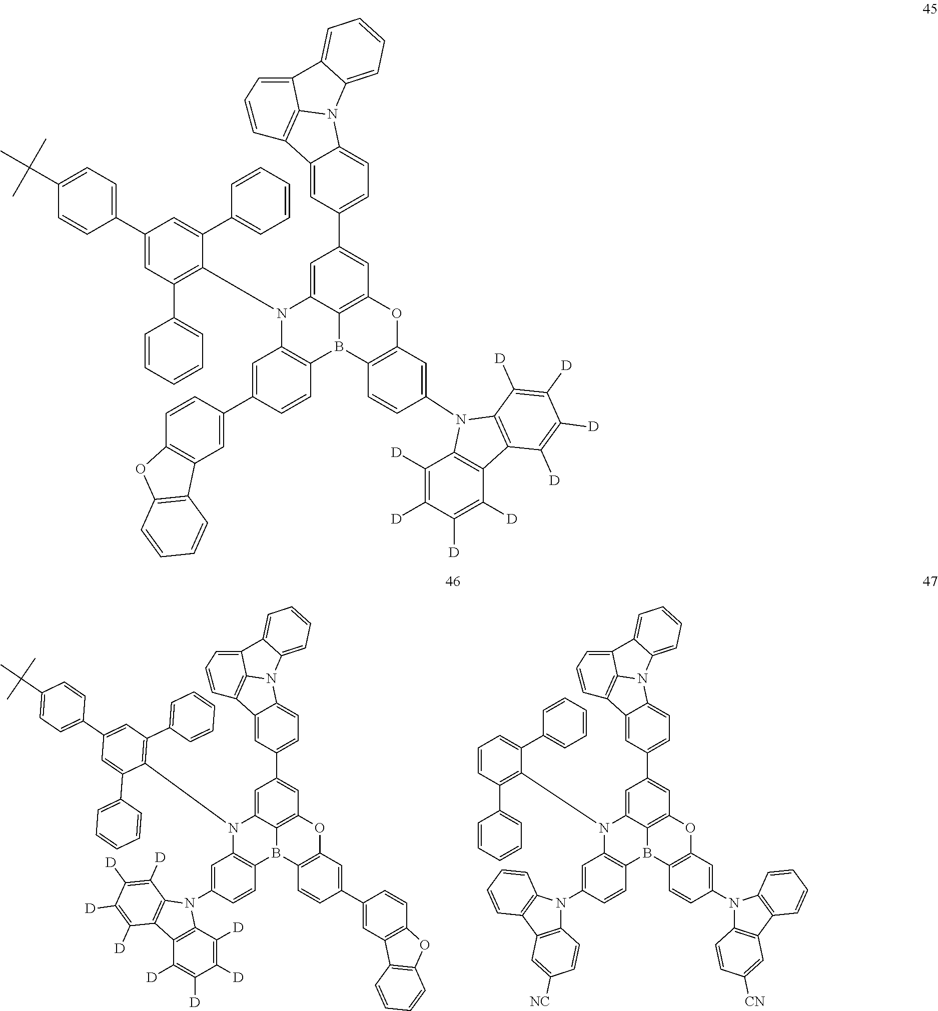

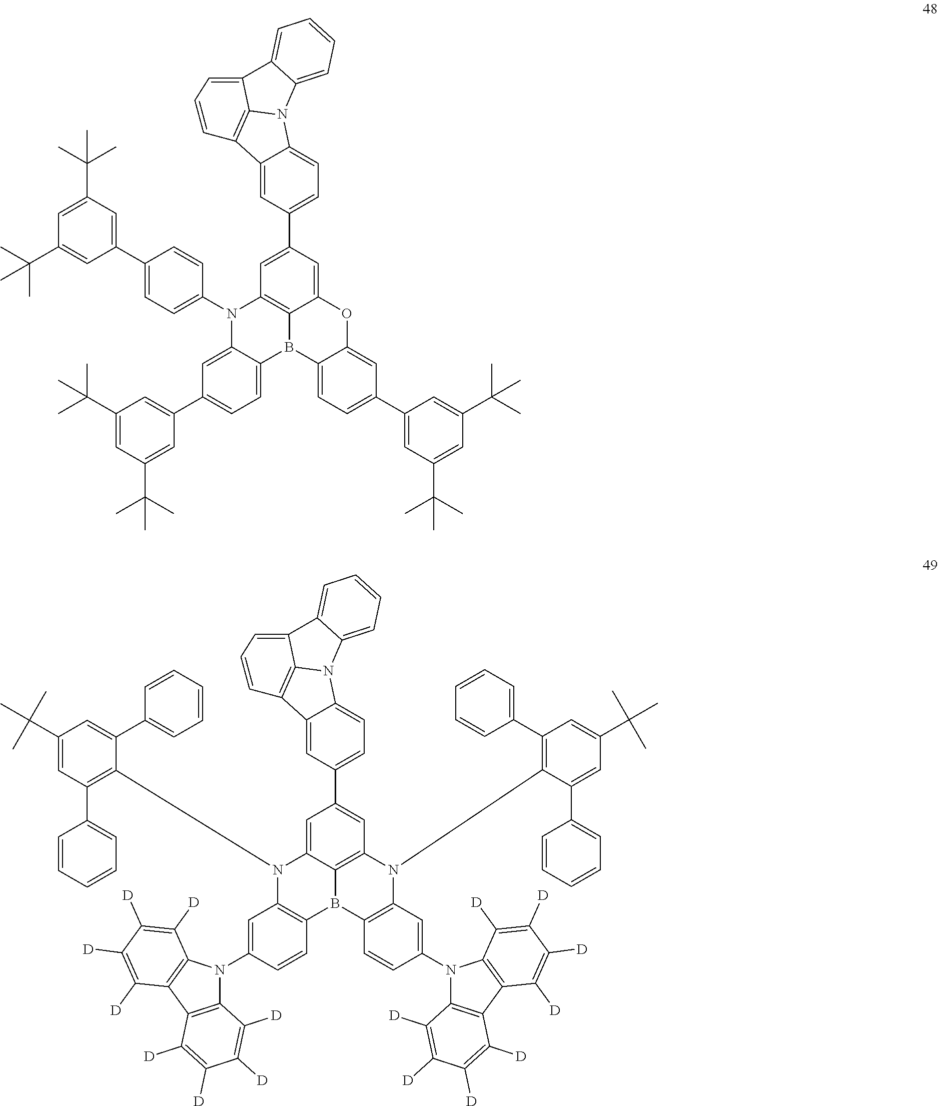

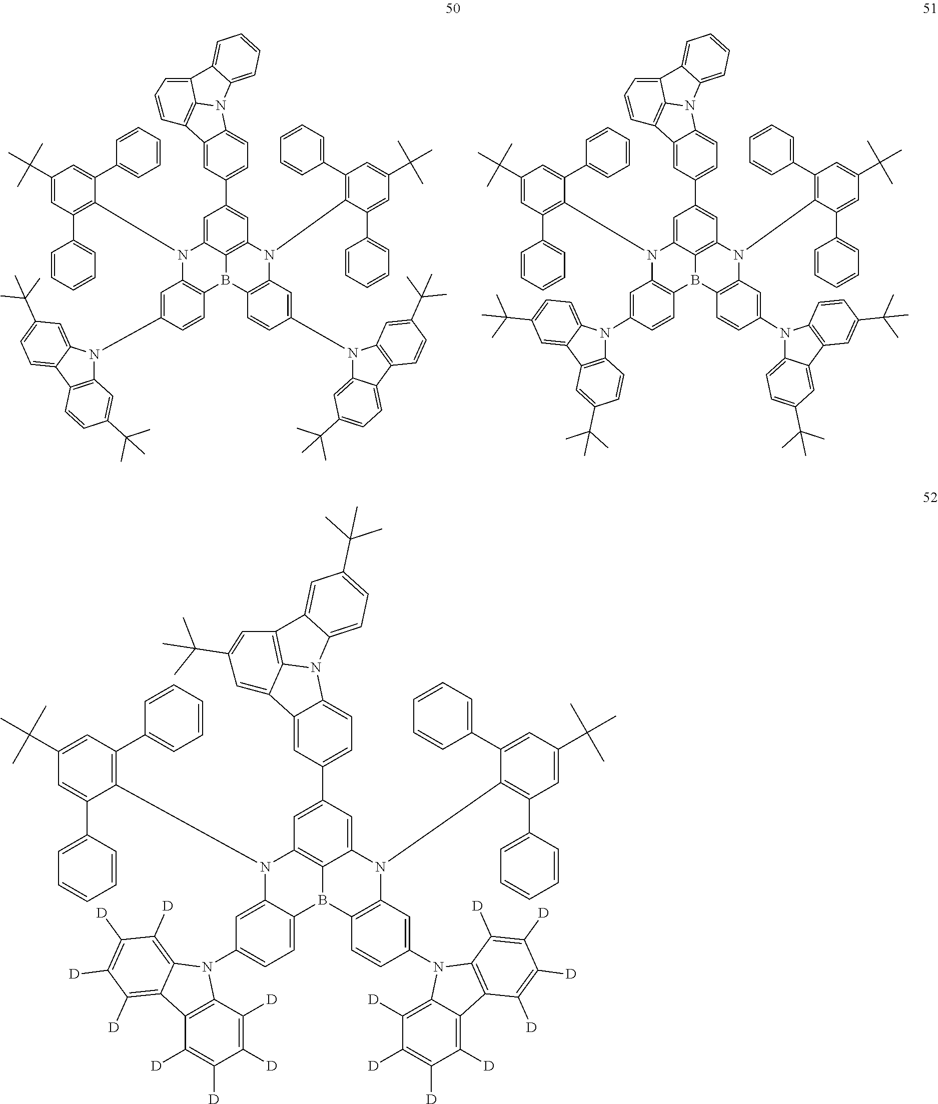

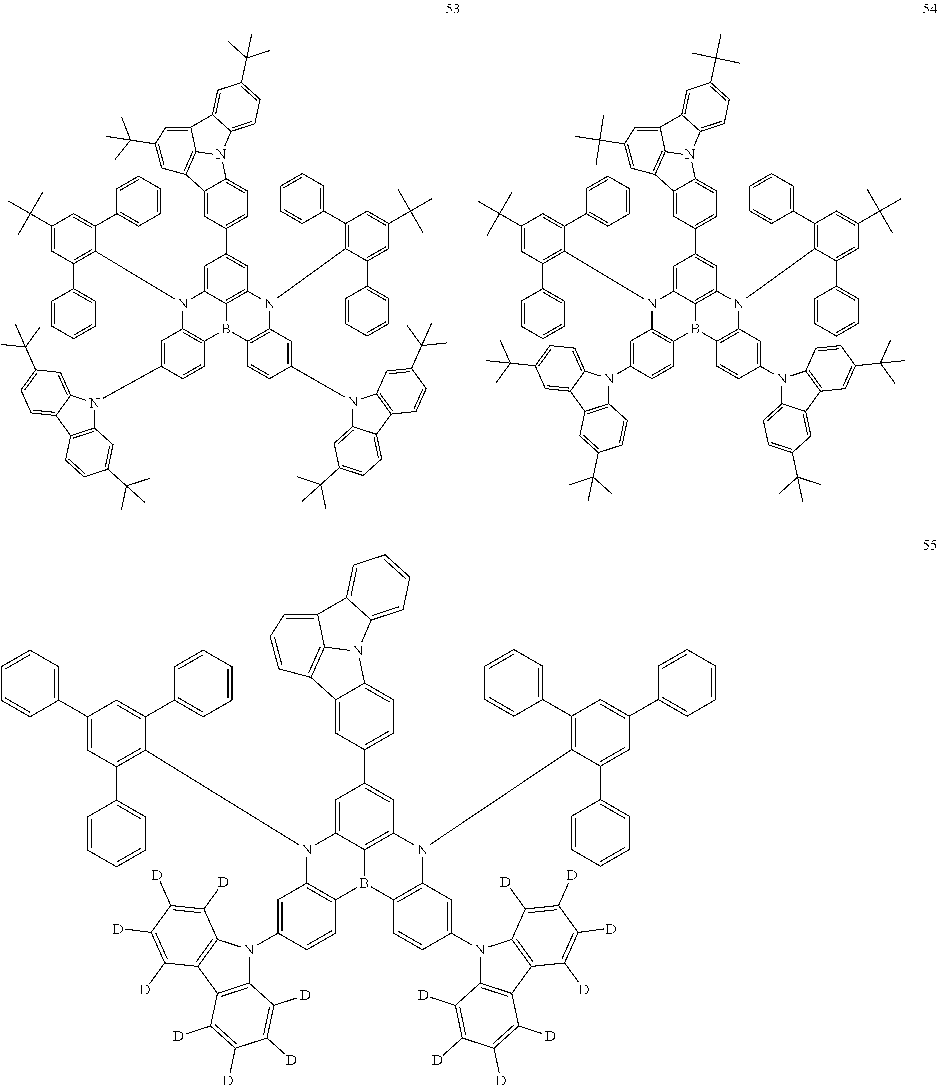

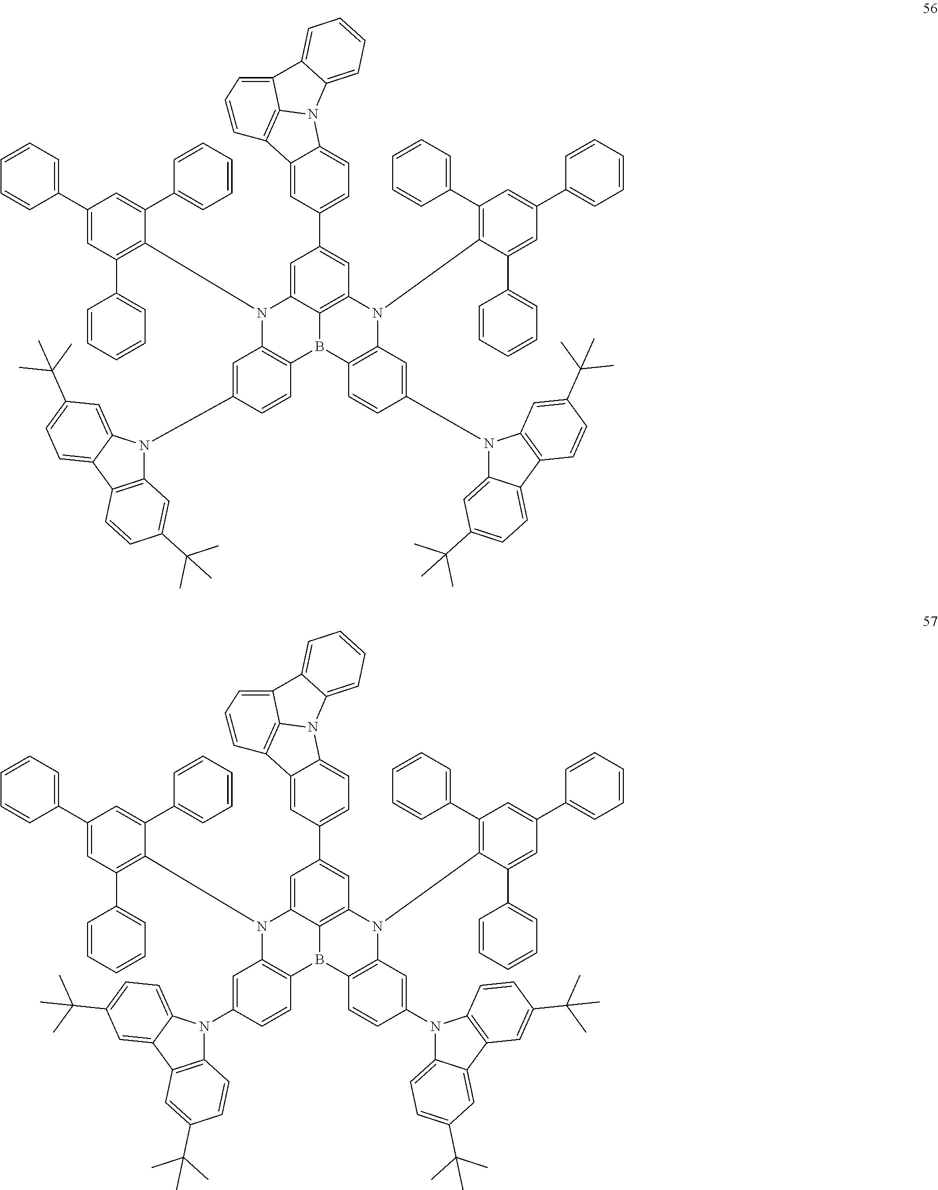 [SMILES: CC(C)(C)c1ccc2c3ccc(C(C)(C)C)cc3n(-c3ccc4c(c3)N(c3c(-c5ccccc5)cc(-c5ccccc5)cc3-c3ccccc3)c3cc(-c5ccc6c(c5)c5cccc7c8ccccc8n6c75)cc5c3B4c3ccc(-n4c6cc(C(C)(C)C)ccc6c6ccc(C(C)(C)C)cc64)cc3N5c3c(-c4ccccc4)cc(-c4ccccc4)cc3-c3ccccc3)c2c1 ; CC(C)(C)c1ccc2c(c1)c1cc(C(C)(C)C)ccc1n2-c1ccc2c(c1)N(c1c(-c3ccccc3)cc(-c3ccccc3)cc1-c1ccccc1)c1cc(-c3ccc4c(c3)c3cccc5c6ccccc6n4c53)cc3c1B2c1ccc(-n2c4ccc(C(C)(C)C)cc4c4cc(C(C)(C)C)ccc42)cc1N3c1c(-c2ccccc2)cc(-c2ccccc2)cc1-c1ccccc1]

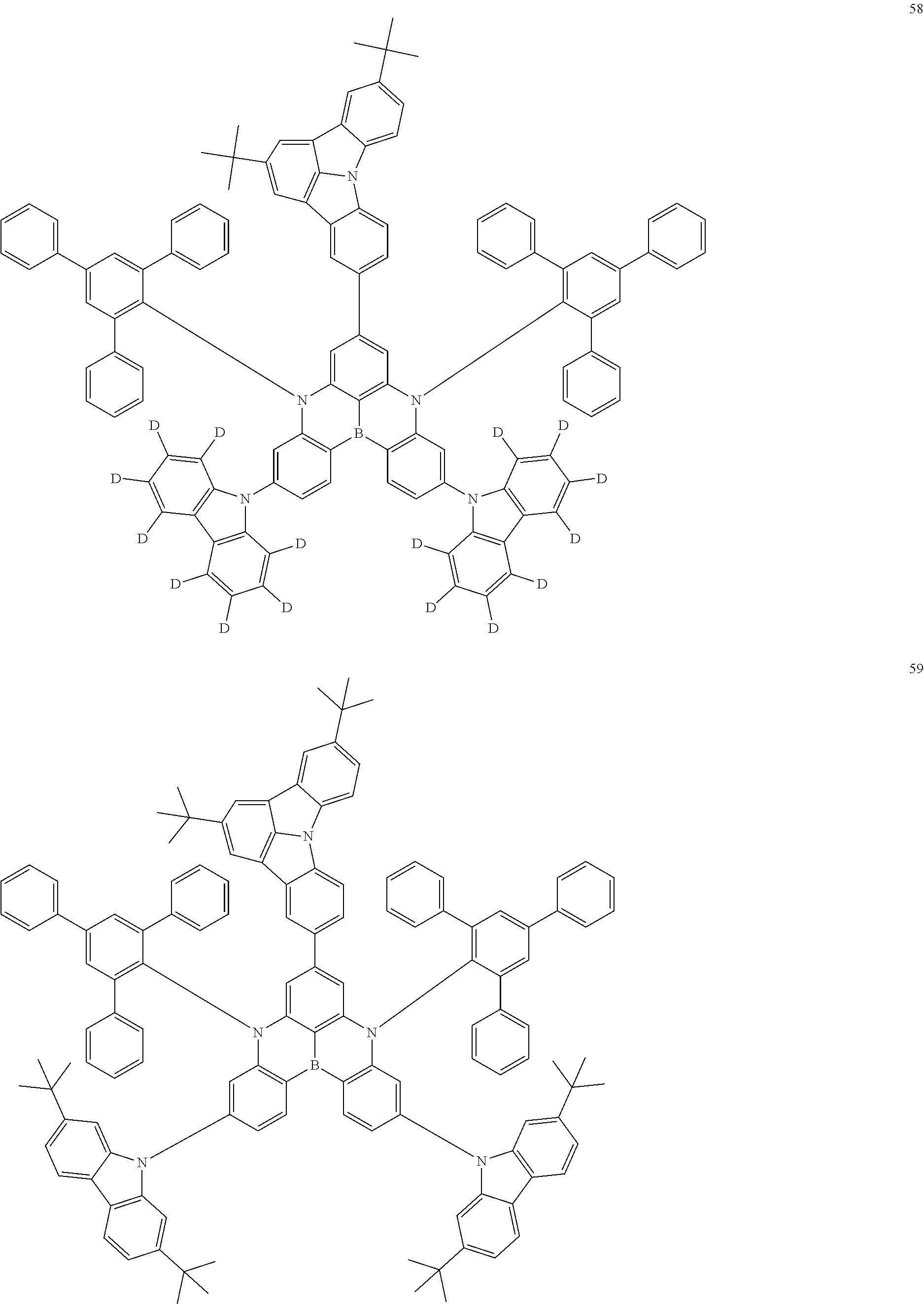

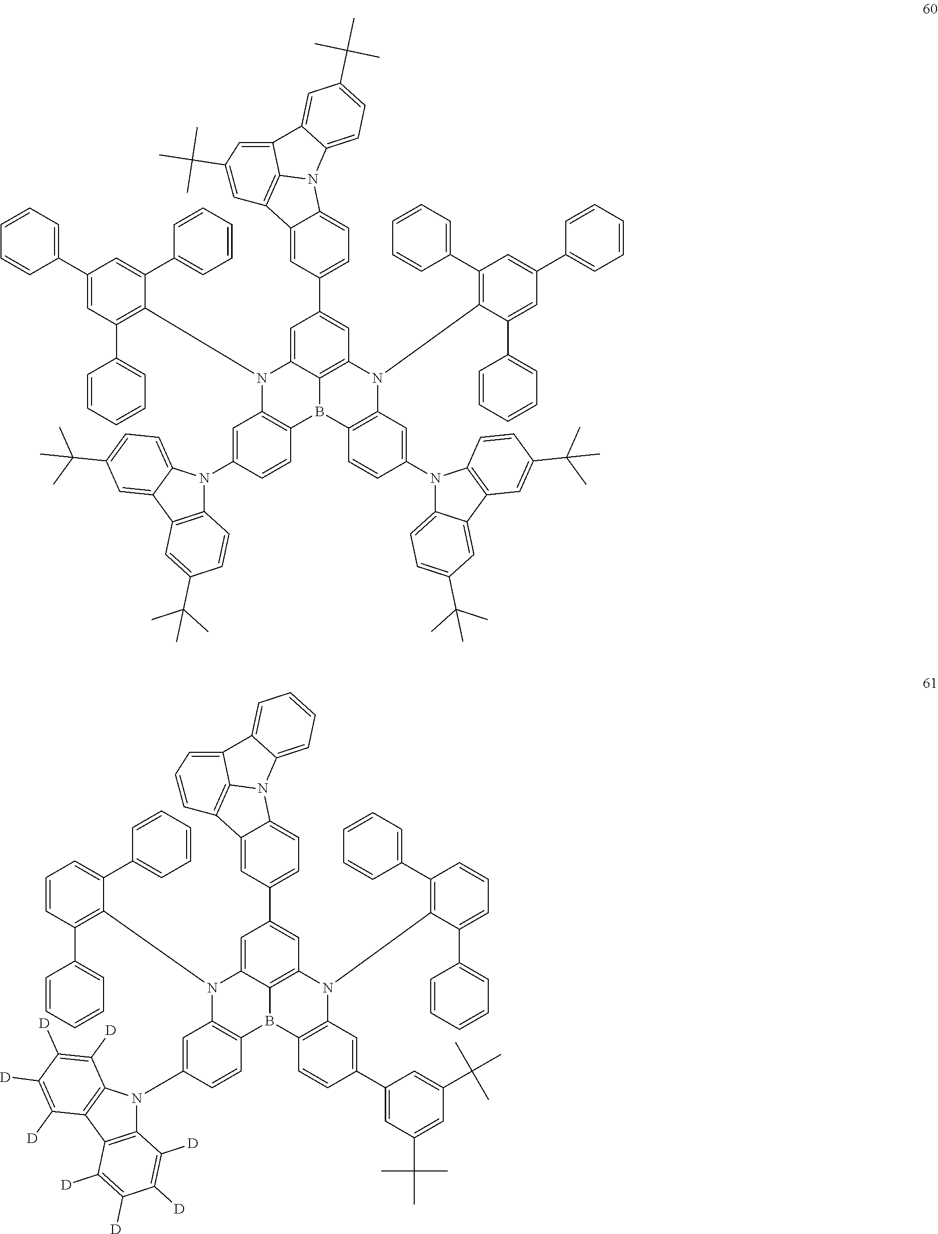 [SMILES: CC(C)(C)c1ccc2c(c1)c1cc(C(C)(C)C)ccc1n2-c1ccc2c(c1)N(c1c(-c3ccccc3)cc(-c3ccccc3)cc1-c1ccccc1)c1cc(-c3ccc4c(c3)c3cc(C(C)(C)C)cc5c6cc(C(C)(C)C)ccc6n4c35)cc3c1B2c1ccc(-n2c4ccc(C(C)(C)C)cc4c4cc(C(C)(C)C)ccc42)cc1N3c1c(-c2ccccc2)cc(-c2ccccc2)cc1-c1ccccc1 ; [2H]c1c([2H])c([2H])c2c(c1[2H])c1c([2H])c([2H])c([2H])c([2H])c1n2-c1ccc2c(c1)N(c1c(-c3ccccc3)cccc1-c1ccccc1)c1cc(-c3ccc4c(c3)c3cccc5c6ccccc6n4c53)cc3c1B2c1ccc(-c2cc(C(C)(C)C)cc(C(C)(C)C)c2)cc1N3c1c(-c2ccccc2)cccc1-c1ccccc1]

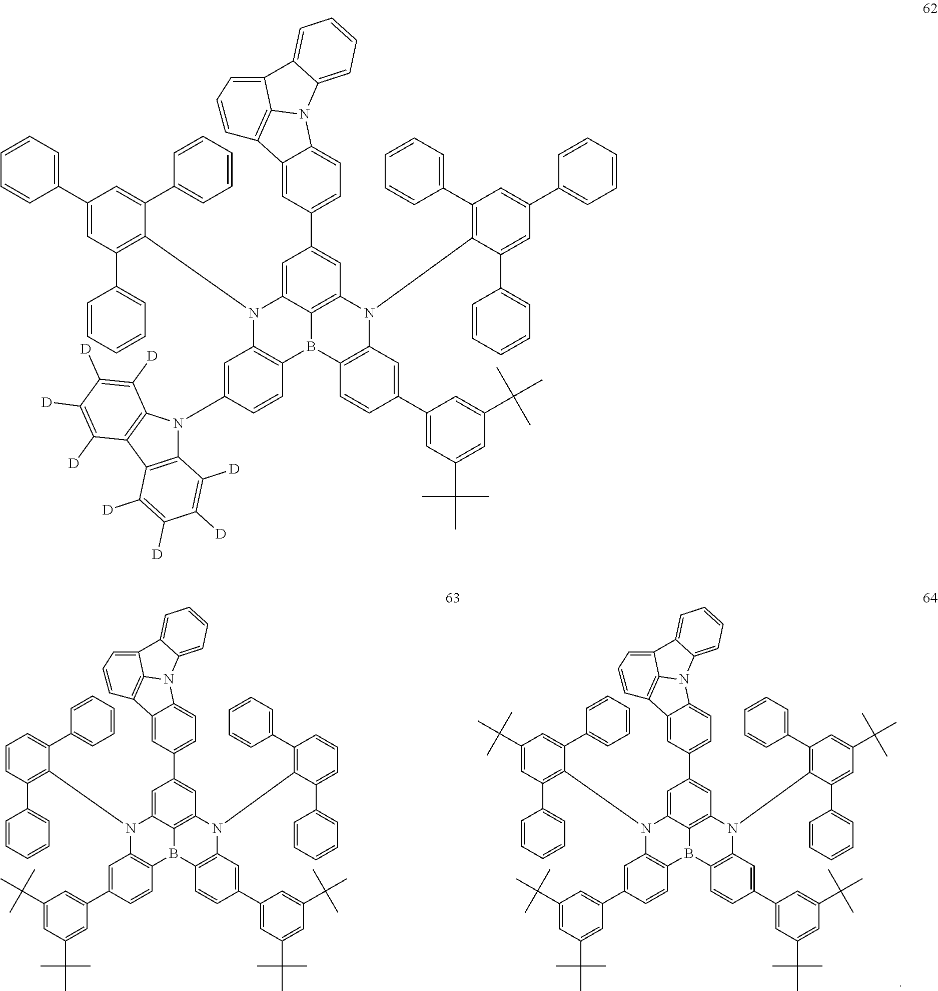

9. The light emitting device of claim 1, wherein the first host comprises at least one selected from Compound Group 2:

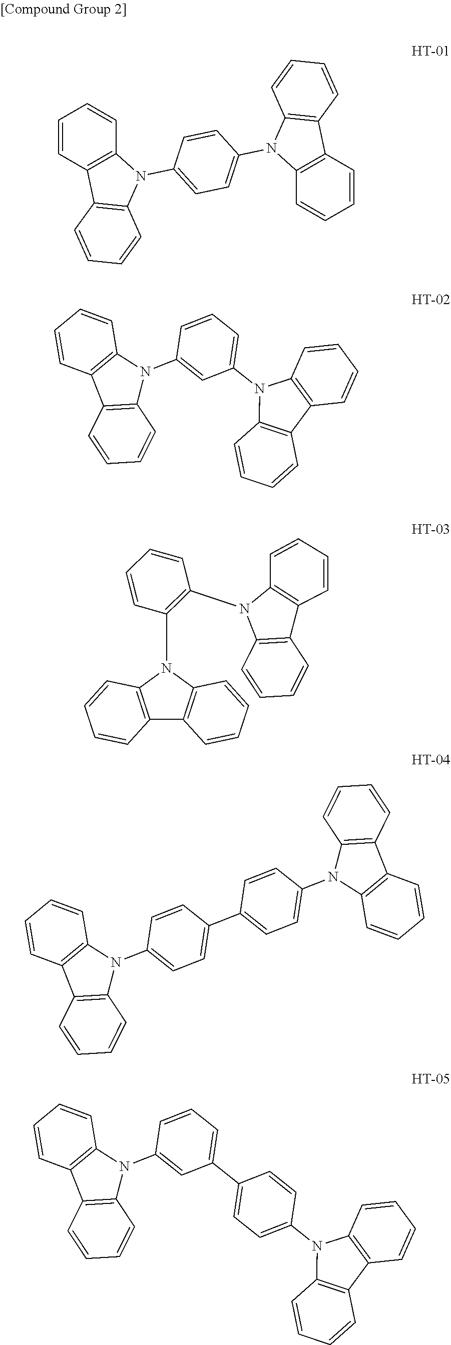 [SMILES: c1ccc2c(c1)c1ccccc1n2-c1ccc(-n2c3ccccc3c3ccccc32)cc1 ; c1cc(-n2c3ccccc3c3ccccc32)cc(-n2c3ccccc3c3ccccc32)c1 ; c1ccc(-n2c3ccccc3c3ccccc32)c(-n2c3ccccc3c3ccccc32)c1 ; c1ccc2c(c1)c1ccccc1n2-c1ccc(-c2ccc(-n3c4ccccc4c4ccccc43)cc2)cc1 ; c1cc(-c2ccc(-n3c4ccccc4c4ccccc43)cc2)cc(-n2c3ccccc3c3ccccc32)c1]

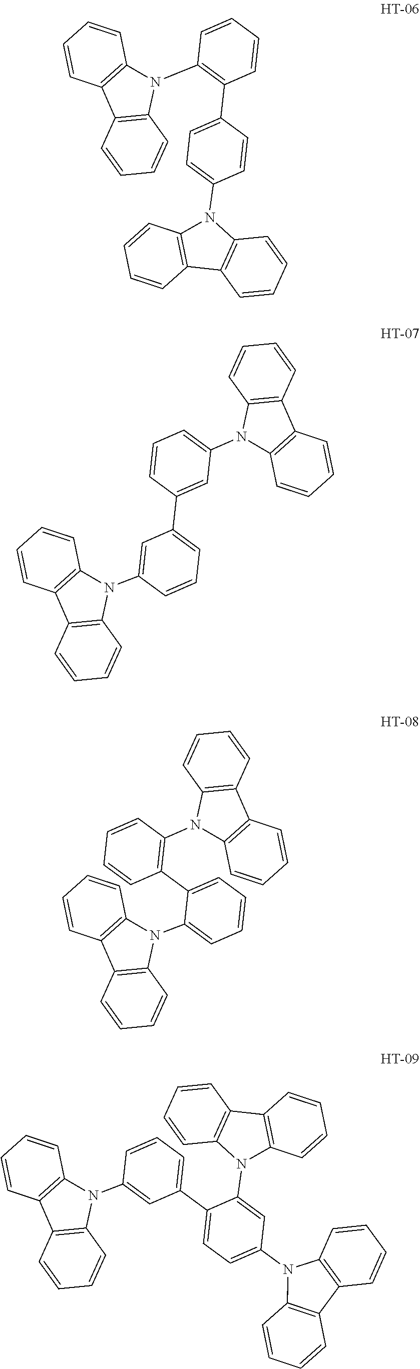 [SMILES: c1ccc(-n2c3ccccc3c3ccccc32)c(-c2ccc(-n3c4ccccc4c4ccccc43)cc2)c1 ; c1cc(-c2cccc(-n3c4ccccc4c4ccccc43)c2)cc(-n2c3ccccc3c3ccccc32)c1 ; c1ccc(-n2c3ccccc3c3ccccc32)c(-c2ccccc2-n2c3ccccc3c3ccccc32)c1 ; c1cc(-c2ccc(-n3c4ccccc4c4ccccc43)cc2-n2c3ccccc3c3ccccc32)cc(-n2c3ccccc3c3ccccc32)c1]

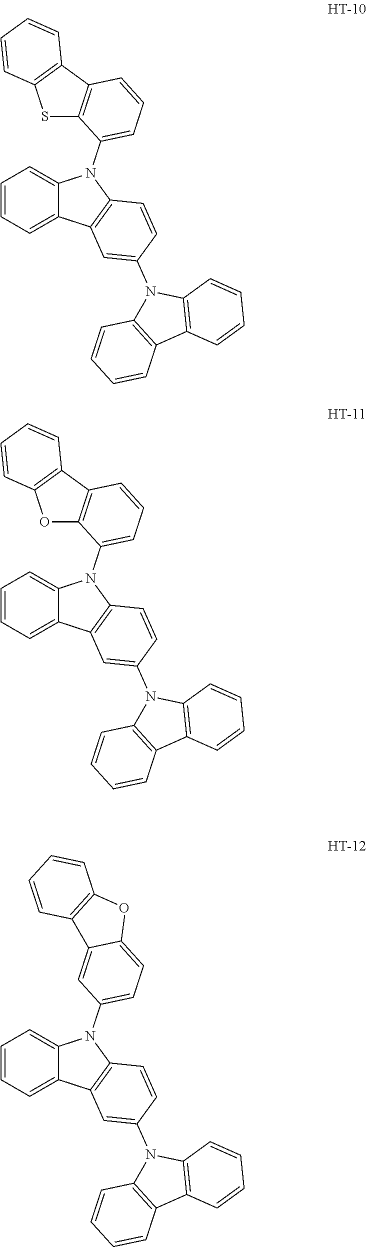 [SMILES: c1ccc2c(c1)sc1c(-n3c4ccccc4c4cc(-n5c6ccccc6c6ccccc65)ccc43)cccc12 ; c1ccc2c(c1)oc1c(-n3c4ccccc4c4cc(-n5c6ccccc6c6ccccc65)ccc43)cccc12 ; c1ccc2c(c1)oc1ccc(-n3c4ccccc4c4cc(-n5c6ccccc6c6ccccc65)ccc43)cc12]

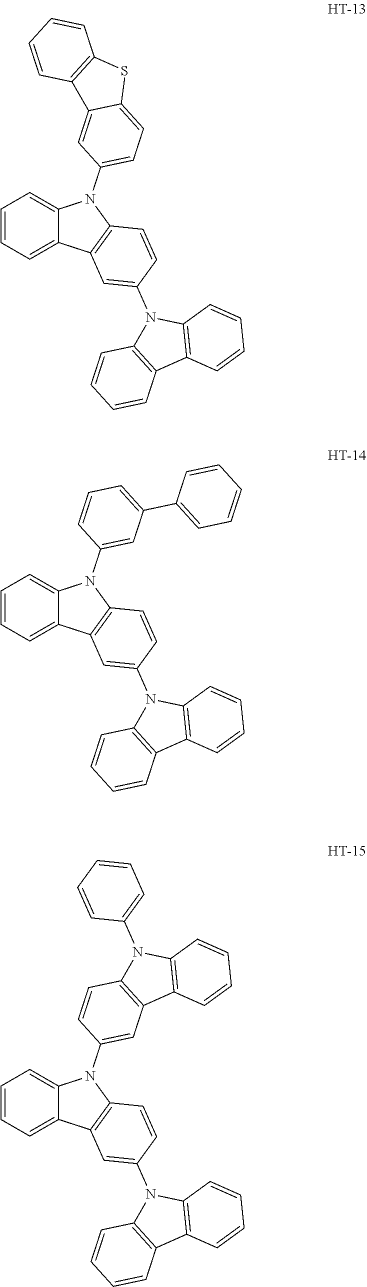 [SMILES: c1ccc2c(c1)sc1ccc(-n3c4ccccc4c4cc(-n5c6ccccc6c6ccccc65)ccc43)cc12 ; c1ccc(-c2cccc(-n3c4ccccc4c4cc(-n5c6ccccc6c6ccccc65)ccc43)c2)cc1 ; c1ccc(-n2c3ccccc3c3cc(-n4c5ccccc5c5cc(-n6c7ccccc7c7ccccc76)ccc54)ccc32)cc1]

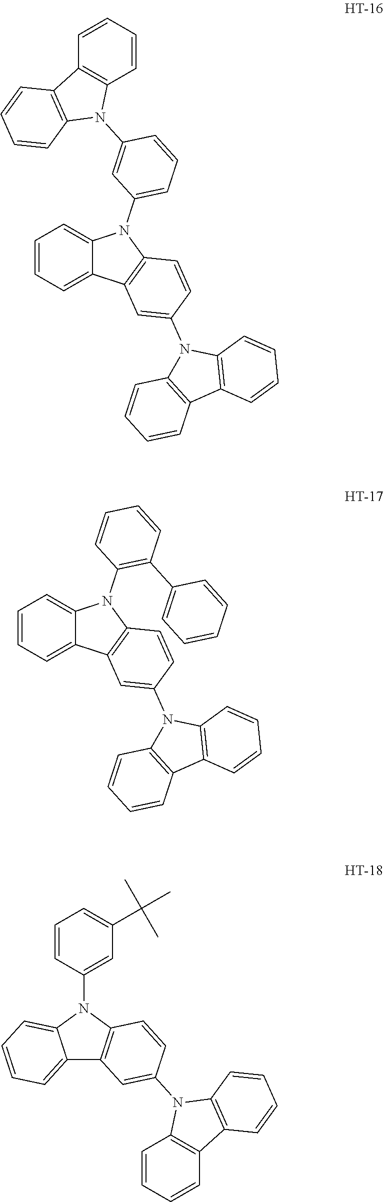 [SMILES: c1cc(-n2c3ccccc3c3ccccc32)cc(-n2c3ccccc3c3cc(-n4c5ccccc5c5ccccc54)ccc32)c1 ; c1ccc(-c2ccccc2-n2c3ccccc3c3cc(-n4c5ccccc5c5ccccc54)ccc32)cc1 ; CC(C)(C)c1cccc(-n2c3ccccc3c3cc(-n4c5ccccc5c5ccccc54)ccc32)c1]

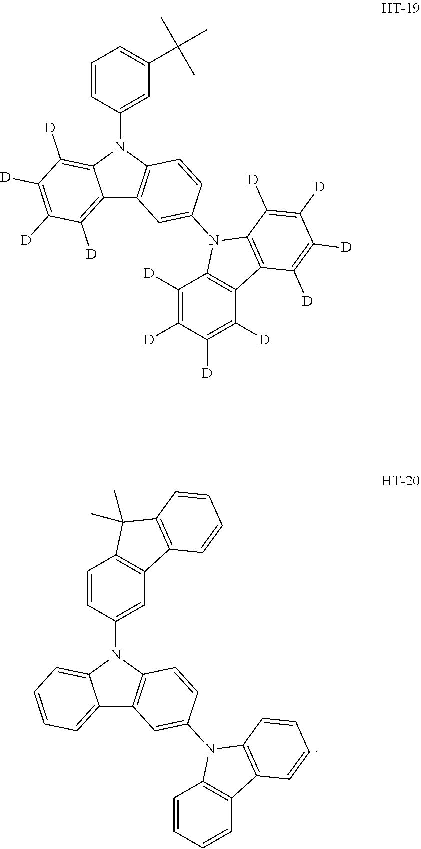 [SMILES: [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; CC1(C)c2ccccc2-c2cc(-n3c4ccccc4c4cc(-n5c6ccccc6c6ccccc65)ccc43)ccc21]

10. The light emitting device of claim 1, wherein the second host comprises at least one selected from Compound Group 3:

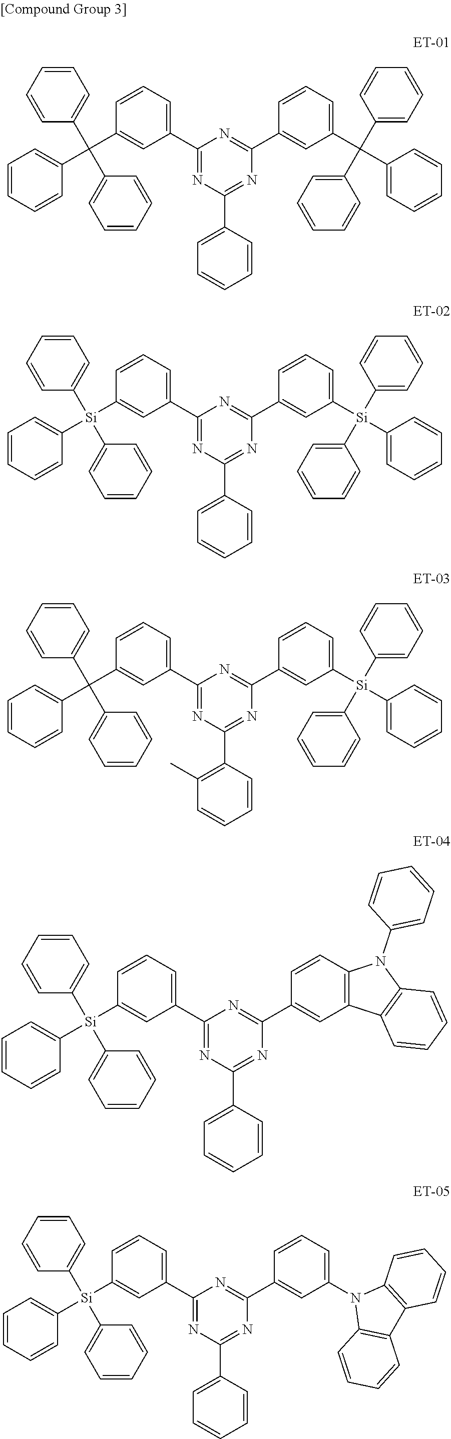

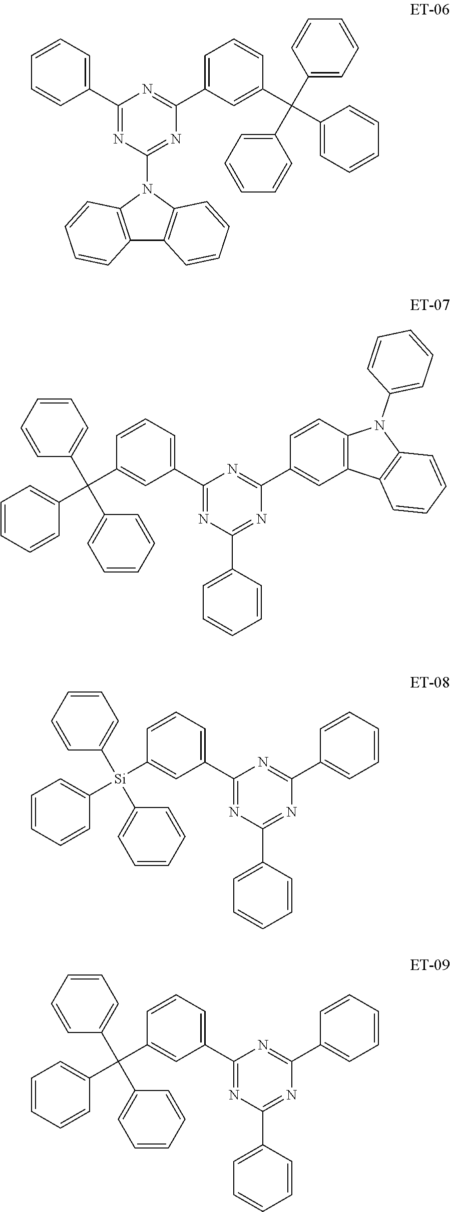 [SMILES: c1ccc(-c2nc(-c3cccc(C(c4ccccc4)(c4ccccc4)c4ccccc4)c3)nc(-n3c4ccccc4c4ccccc43)n2)cc1 ; c1ccc(-c2nc(-c3cccc(C(c4ccccc4)(c4ccccc4)c4ccccc4)c3)nc(-c3ccc4c(c3)c3ccccc3n4-c3ccccc3)n2)cc1 ; c1ccc(-c2nc(-c3ccccc3)nc(-c3cccc([Si](c4ccccc4)(c4ccccc4)c4ccccc4)c3)n2)cc1 ; c1ccc(-c2nc(-c3ccccc3)nc(-c3cccc(C(c4ccccc4)(c4ccccc4)c4ccccc4)c3)n2)cc1]

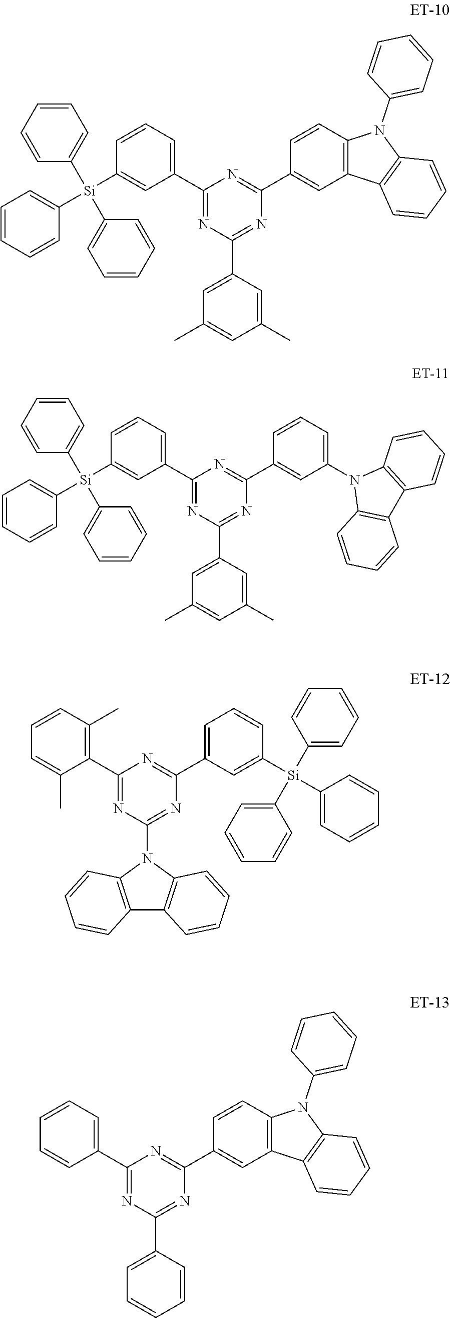 [SMILES: Cc1cc(C)cc(-c2nc(-c3cccc([Si](c4ccccc4)(c4ccccc4)c4ccccc4)c3)nc(-c3ccc4c(c3)c3ccccc3n4-c3ccccc3)n2)c1 ; Cc1cc(C)cc(-c2nc(-c3cccc(-n4c5ccccc5c5ccccc54)c3)nc(-c3cccc([Si](c4ccccc4)(c4ccccc4)c4ccccc4)c3)n2)c1 ; Cc1cccc(C)c1-c1nc(-c2cccc([Si](c3ccccc3)(c3ccccc3)c3ccccc3)c2)nc(-n2c3ccccc3c3ccccc32)n1 ; c1ccc(-c2nc(-c3ccccc3)nc(-c3ccc4c(c3)c3ccccc3n4-c3ccccc3)n2)cc1]

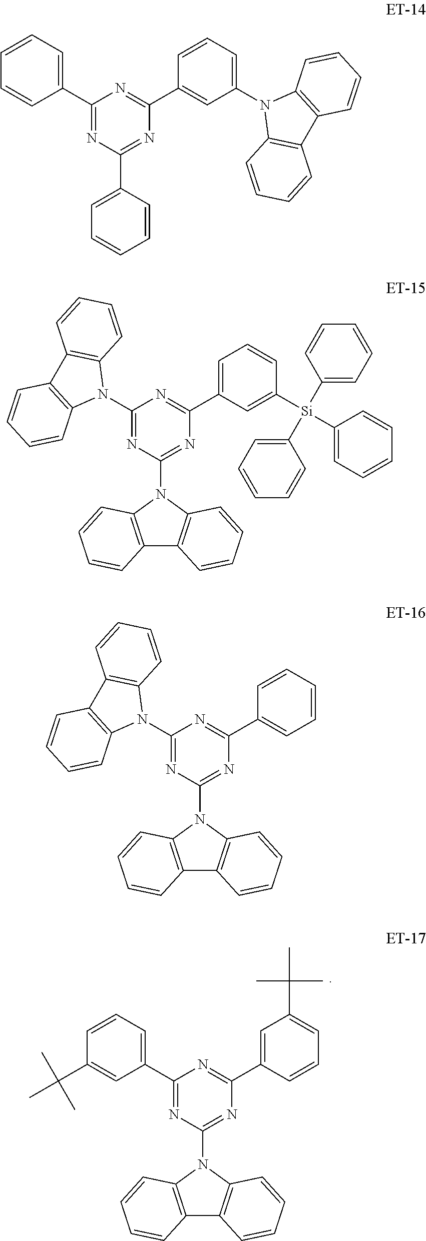 [SMILES: c1ccc(-c2nc(-c3ccccc3)nc(-c3cccc(-n4c5ccccc5c5ccccc54)c3)n2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1]

11. The light emitting device of claim 1, wherein the emission layer emits delayed fluorescence.

12. The light emitting device of claim 1, wherein the emission layer emits light having a central emission wavelength in a range of about 430 nm to about 490 nm.

13. The light emitting device of claim 1, wherein
the emission layer further comprises a second dopant which is different from the first dopant, and
the second dopant is represented by Formula D-2: 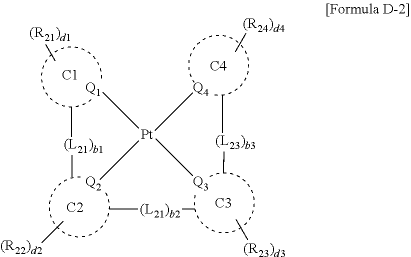
wherein in Formula D-2,
Q₁ to Q₄ are each independently C or N,
C1 to C4 are each independently a substituted or unsubstituted hydrocarbon ring of 5 to 30 ring-forming carbon atoms, or a substituted or unsubstituted heterocycle of 2 to 30 ring-forming carbon atoms,
L₂₁ to L₂₃ are each independently a direct linkage, 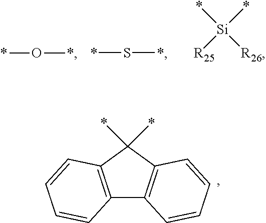
a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms,
b1 to b3 are each independently 0 or 1,
R₂₁ to R₂₆ are each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 1 to 30 ring-forming carbon atoms, or are combined with an adjacent group to form a ring, and
d1 to d4 are each independently an integer from 0 to 4.

14. The light emitting device of claim 13, wherein the second dopant comprises at least one selected from Compound Group 4:

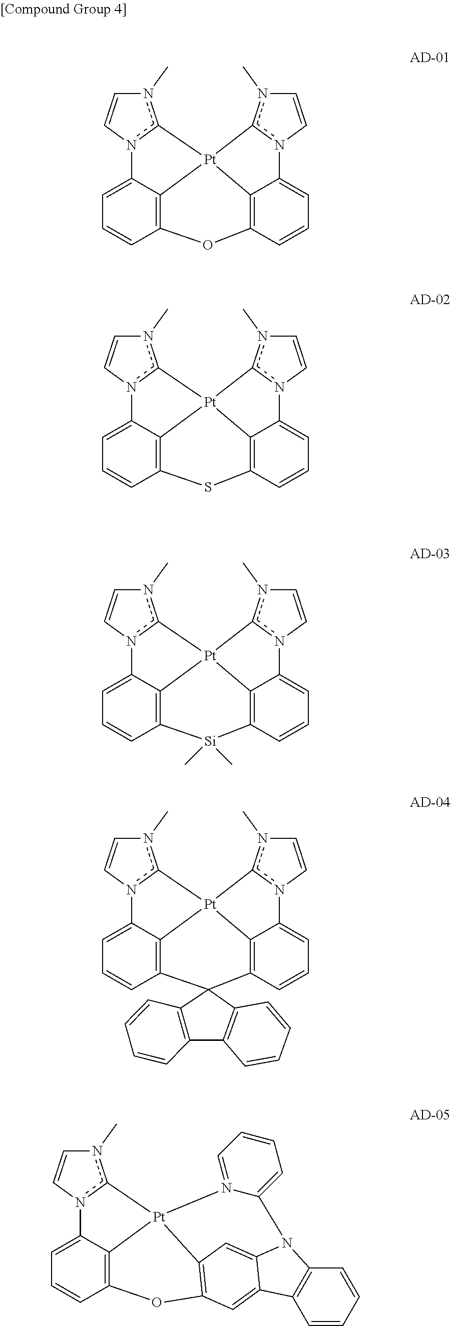

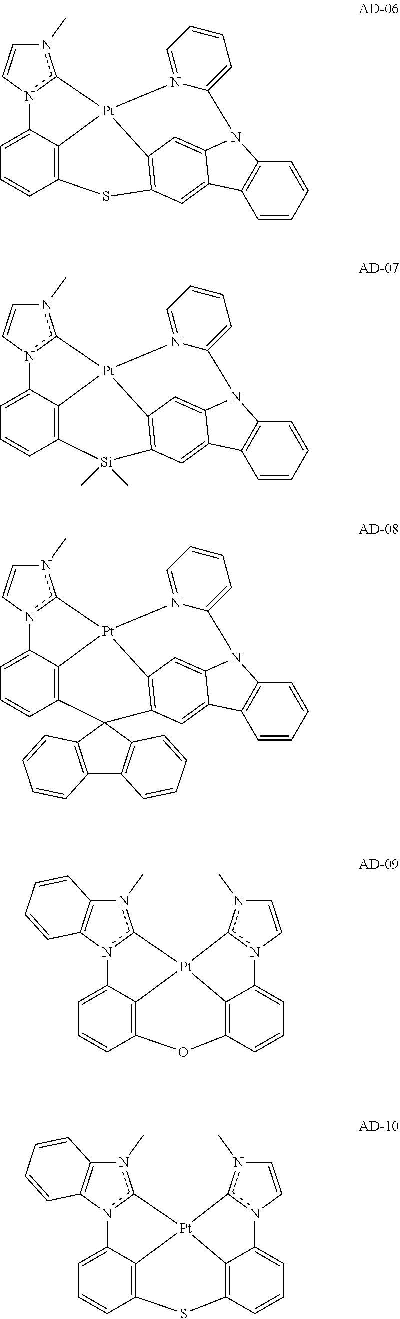

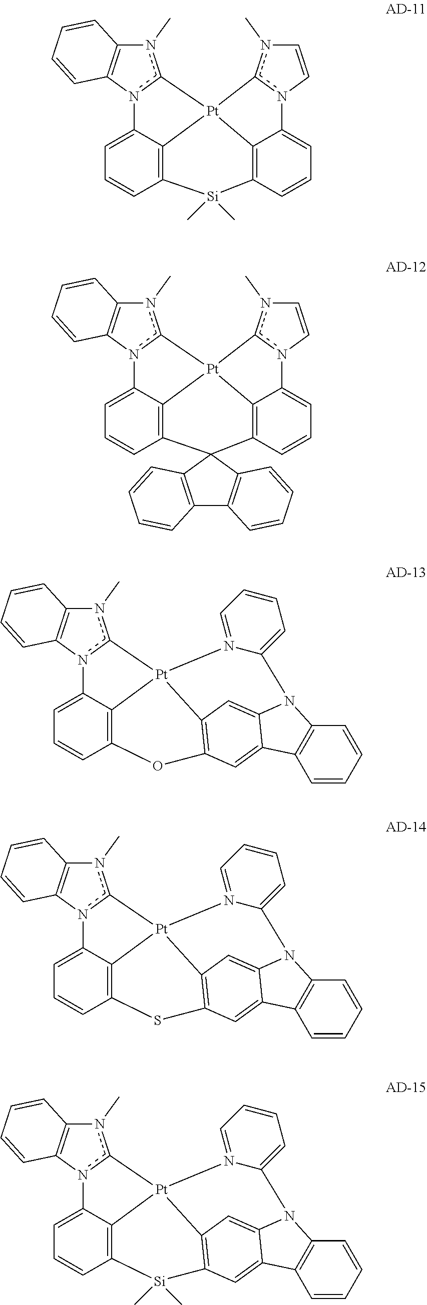

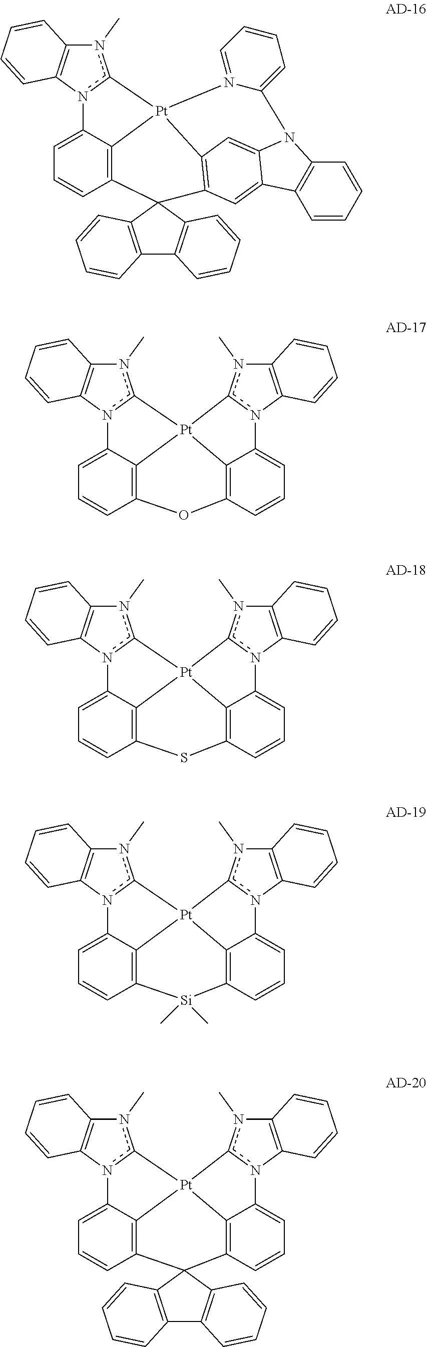

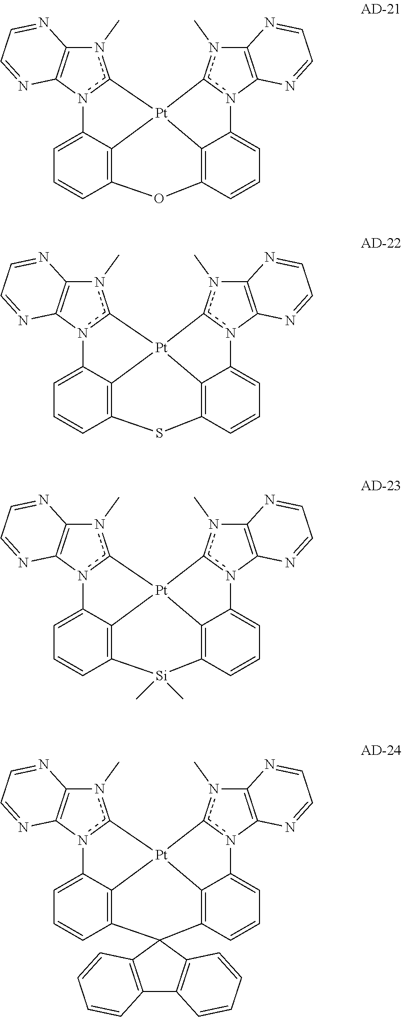

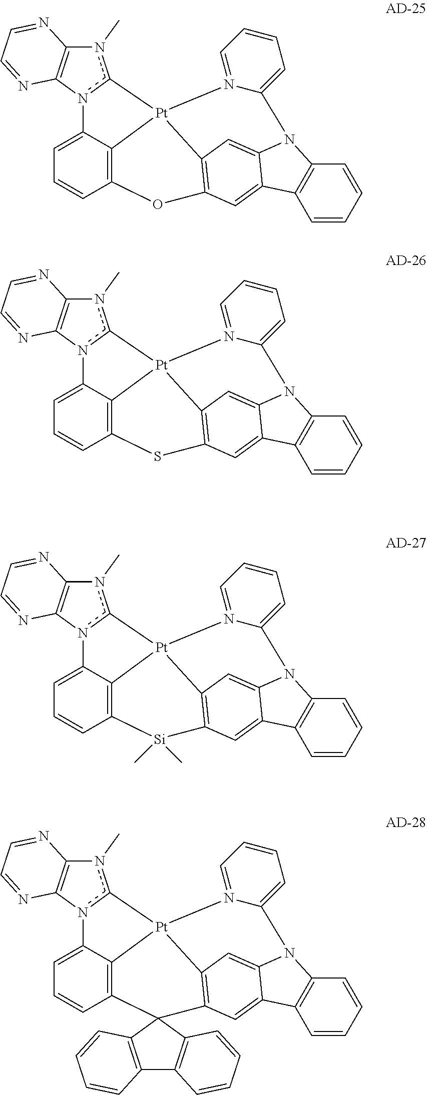

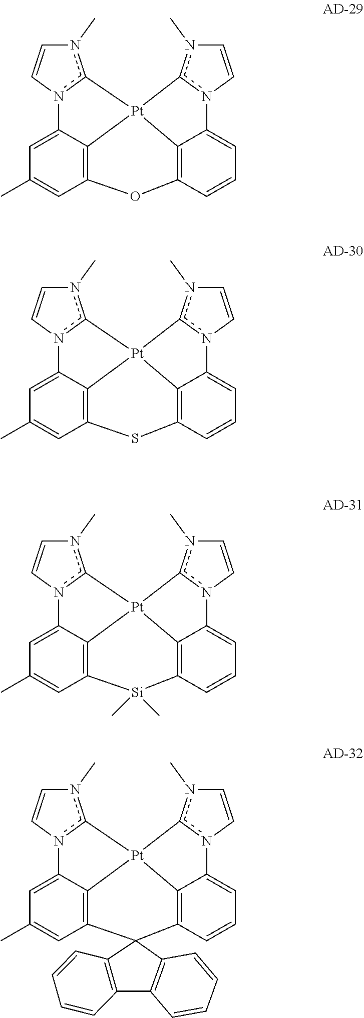

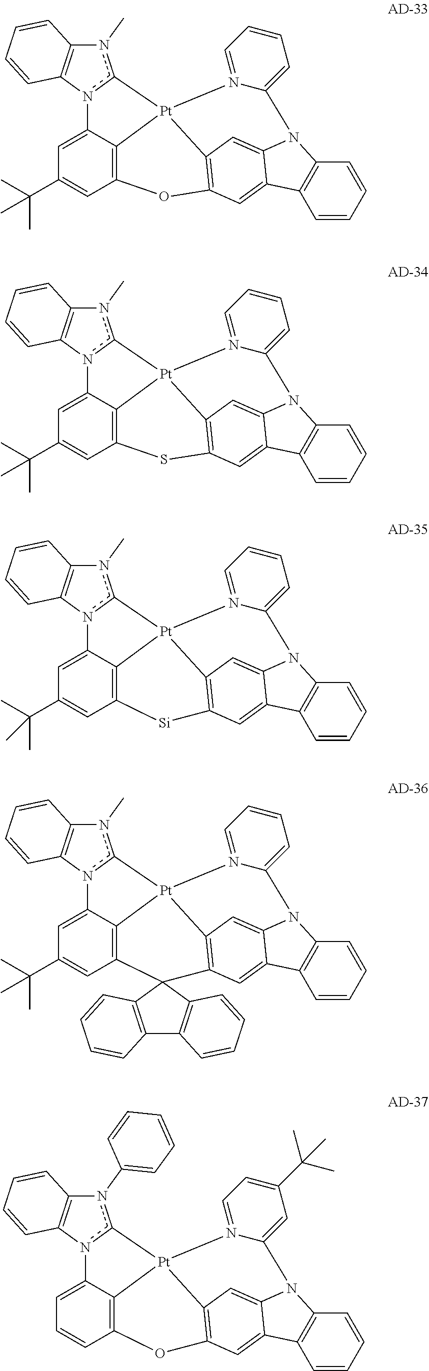

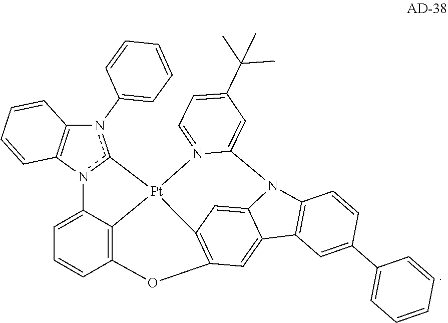

15. The light emitting device of claim 1, further comprising a hole transport region disposed between the first electrode and the emission layer, wherein
the hole transport region comprises a compound represented by Formula H-a: 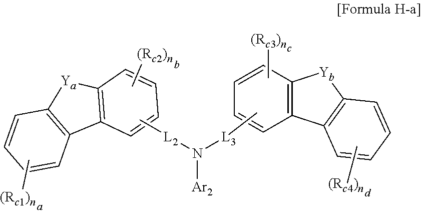
wherein in Formula H-a,
Yₐ and Y_{b} are each independently C(R_{c5})(R_{c6}), N(R_{c7}), O, or S,
Ar₂ is a substituted or unsubstituted aryl group of 6 to 30 ring-forming carbon atoms, or a substituted or unsubstituted heteroaryl group of 2 to 30 ring-forming carbon atoms,
L₂ and L₃ are each independently a direct linkage, a substituted or unsubstituted arylene group of 6 to 30 ring-forming carbon atoms, or a substituted or unsubstituted heteroarylene group of 2 to 30 ring-forming carbon atoms,
R_{c1} to R_{c7} are each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or are combined with an adjacent group to form a ring,
nₐ and n_{d} are each independently an integer from 0 to 4, and
n_{b} and n_{c} are each independently an integer from 0 to 3.

16. A light emitting device, comprising:
a first electrode;
a second electrode facing the first electrode; and
an emission layer disposed between the first electrode and the second electrode, wherein
the emission layer comprises a host and a dopant, and
the dopant comprises:
a first dopant represented by Formula 1; and
a second dopant represented by Formula D-2: 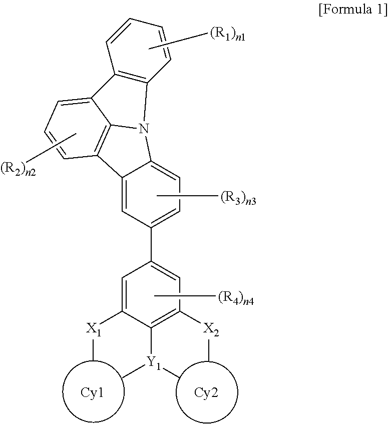
wherein in Formula 1,
X₁ and X₂ are each independently N(R₅), O, or S,
Y₁ is B,
Cy1 and Cy2 are each independently a substituted or unsubstituted aromatic hydrocarbon ring of 6 to 30 ring-forming carbon atoms, or a substituted or unsubstituted aromatic heterocycle of 2 to 30 ring-forming carbon atoms, or are combined with an adjacent group to form a ring,
R₁ to R₄ are each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or are combined with an adjacent group to form a ring,
R₅ is a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or is combined with an adjacent group to form a ring,
n₁ is an integer from 0 to 4,
n₂ and n₃ are each independently an integer from 0 to 3, and
n₄ is an integer from 0 to 2, 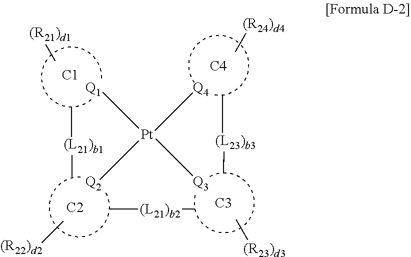
wherein in Formula D-2,
Q₁ to Q₄ are each independently C or N,
C1 to C4 are each independently a substituted or unsubstituted hydrocarbon ring of 5 to 30 ring-forming carbon atoms, or a substituted or unsubstituted heterocycle of 2 to 30 ring-forming carbon atoms,
L₂₁ to L₂₃ are each independently a direct linkage, 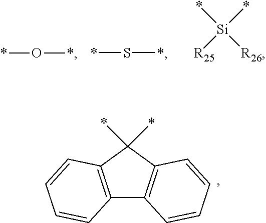
a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms,
b1 to b3 are each independently 0 or 1,
R₂₁ to R₂₆ are each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 1 to 30 ring-forming carbon atoms, or are combined with an adjacent group to form a ring, and
d1 to d4 are each independently an integer from 0 to 4.

17. The light emitting device of claim 16, wherein Cy1 and Cy2 are each independently a group represented by Formula 2: 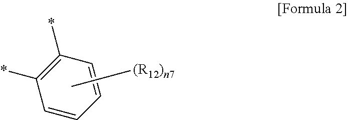
wherein in Formula 2,
R₁₂ is a hydrogen atom, a deuterium atom, a halogen atom, a cyano group, a substituted or unsubstituted amine group, a substituted or unsubstituted oxy group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or is combined with an adjacent group to form a ring,
n₇ is an integer from 0 to 4, and [symbol]
are bonding sites to X₁ and Y₁ of Formula 1, or are bonding sites to X₂ and Y₁ of Formula 1.

18. The light emitting device of claim 17, wherein
if Cy1 and Cy2 are each a group represented by Formula 2, then Cy1 and Cy2 are each independently a group represented by Formula 2-1 or Formula 2-2: 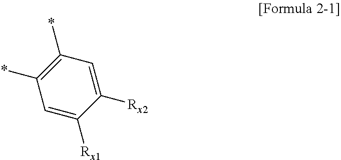
wherein in Formula 2-1,
Rₓ₁ and Rₓ₂ are each independently a hydrogen atom, a deuterium atom, a halogen atom, a substituted or unsubstituted alkyl group of 1 to 20 carbon atoms, a substituted or unsubstituted phenyl group, a substituted or unsubstituted amine group, a substituted or unsubstituted carbazole group, or a substituted or unsubstituted indolocarbazole group, or are combined with an adjacent group to form a ring, and [symbol]
are bonding sites to X₁ and Y₁ of Formula 1, or are bonding sites to X₂ and Y₁ of Formula 1, 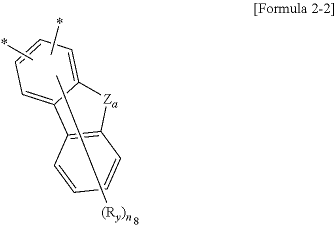
wherein in Formula 2-2,
Zₐ is N(R₁₃) or O,
R_{y} is a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or is combined with an adjacent group to form a ring,
R₁₃ is a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n₈ is an integer from 0 to 6, and [symbol]
are bonding sites to X₁ and Y₁ of Formula 1, or are bonding sites to X₂ and Y₁ of Formula 1.

19. The light emitting device of claim 16, wherein
the host comprises:
a first host represented by Formula H-1; and
a second host represented by Formula H-2: 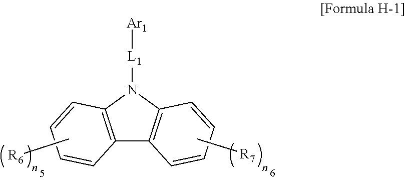
wherein in Formula H-1,
L₁ is a direct linkage, a substituted or unsubstituted arylene group of 6 to 30 ring-forming carbon atoms, or a substituted or unsubstituted heteroarylene group of 2 to 30 ring-forming carbon atoms,
Ar₁ is a substituted or unsubstituted aryl group of 6 to 30 ring-forming carbon atoms, or a substituted or unsubstituted heteroaryl group of 2 to 30 ring-forming carbon atoms,
R₆ and R₇ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and
n₅ and n₆ are each independently an integer from 0 to 4, 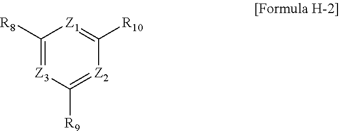
wherein in Formula H-2,
Z₁ to Z₃ are each independently C(R₁₁) or N,
at least one of Z₁ to Z₃ is N, and
R₈ to R₁₁ are each independently a hydrogen atom, a deuterium atom, a cyano group, a substituted or unsubstituted silyl group, a substituted or unsubstituted aryl group of 6 to 30 ring-forming carbon atoms, or a substituted or unsubstituted heteroaryl group of 2 to 30 ring-forming carbon atoms.

20. The light emitting device of claim 16, wherein the first dopant comprises at least one selected from Compound Group 1: 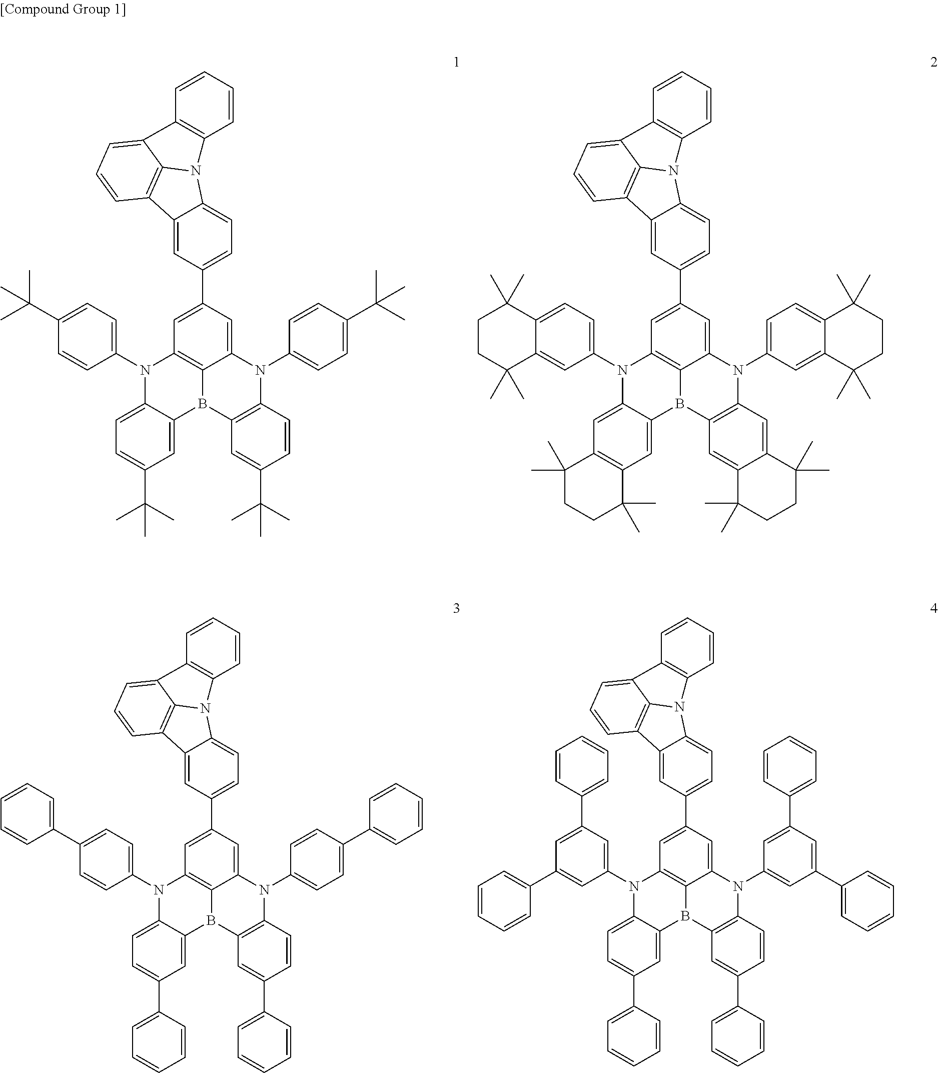 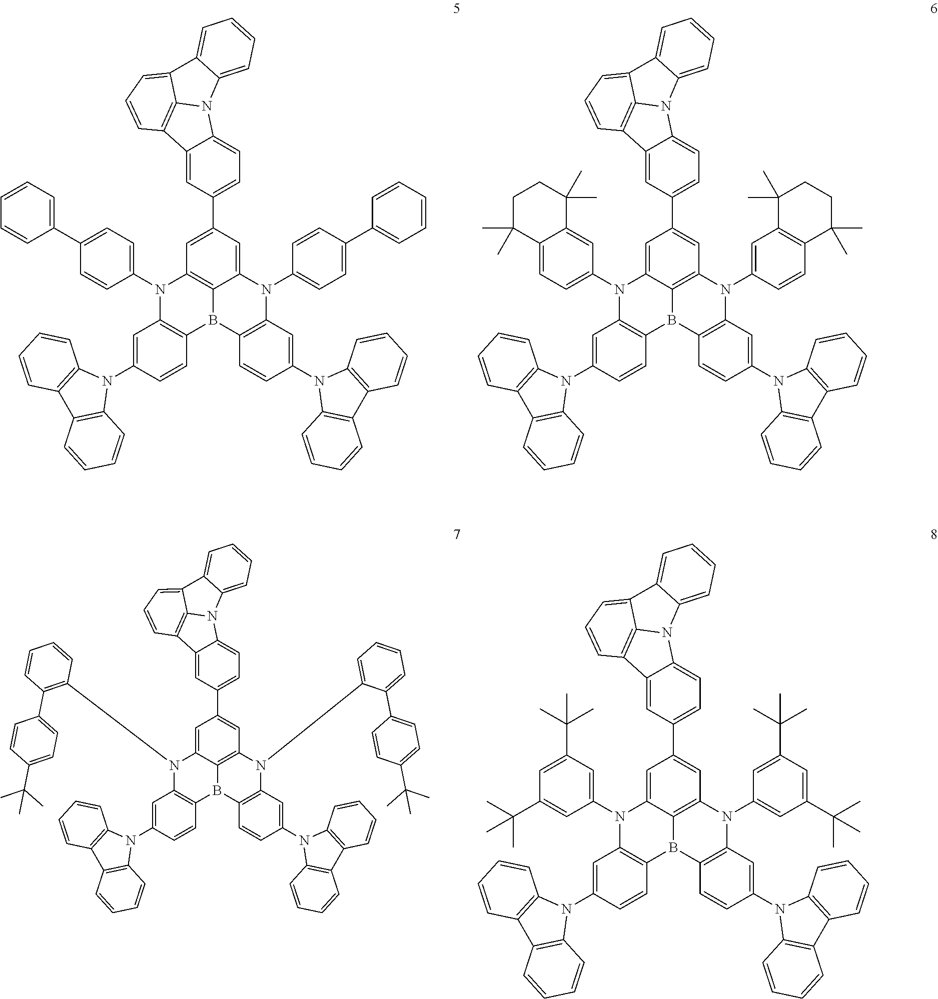 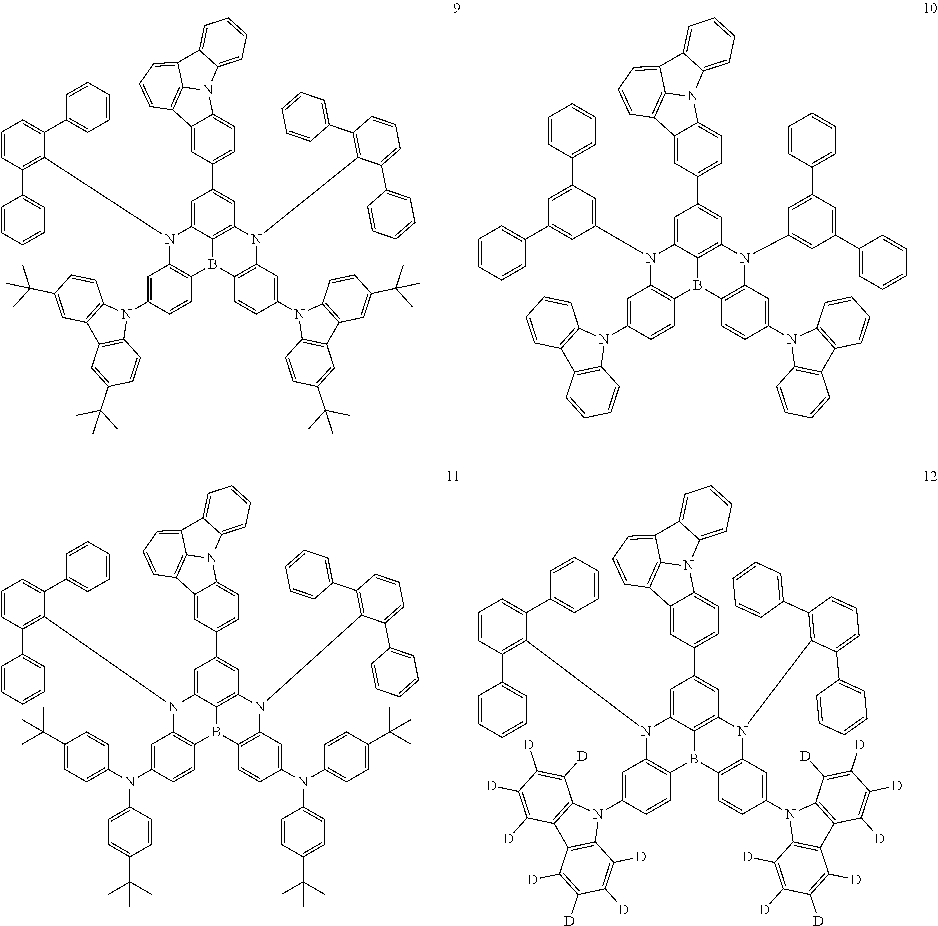 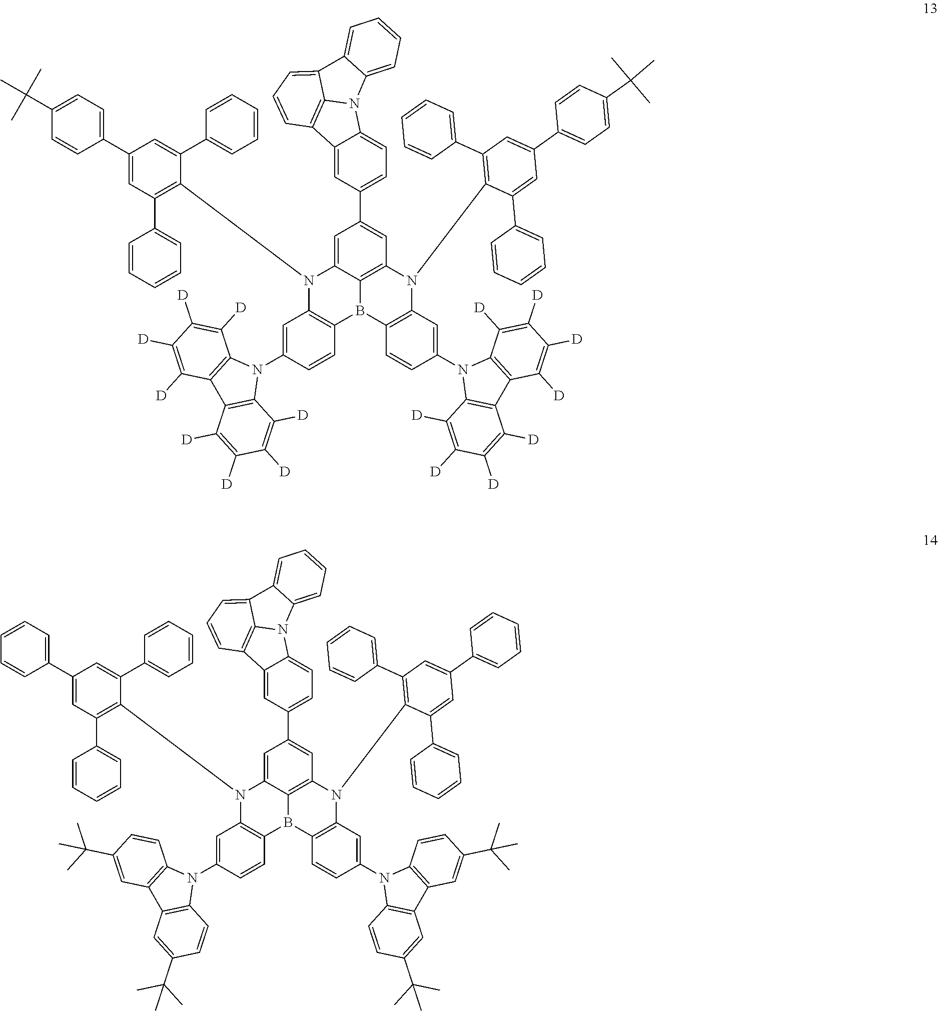 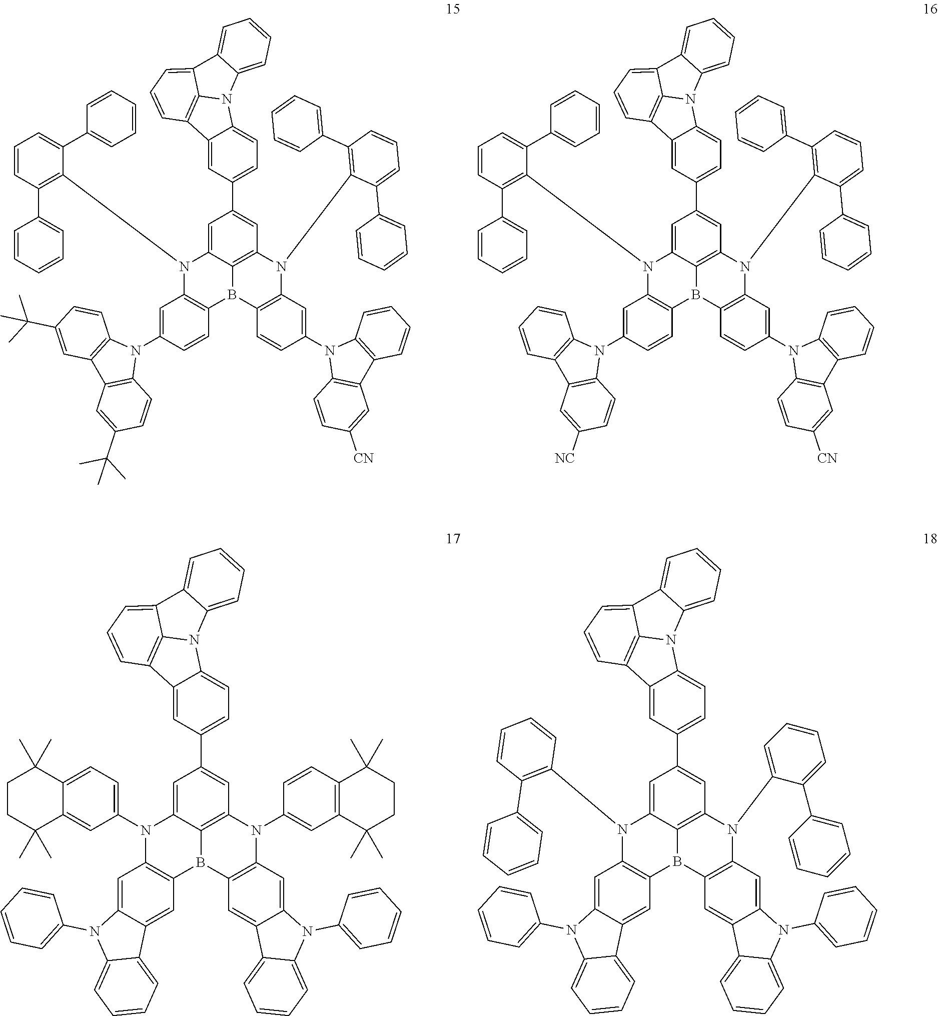 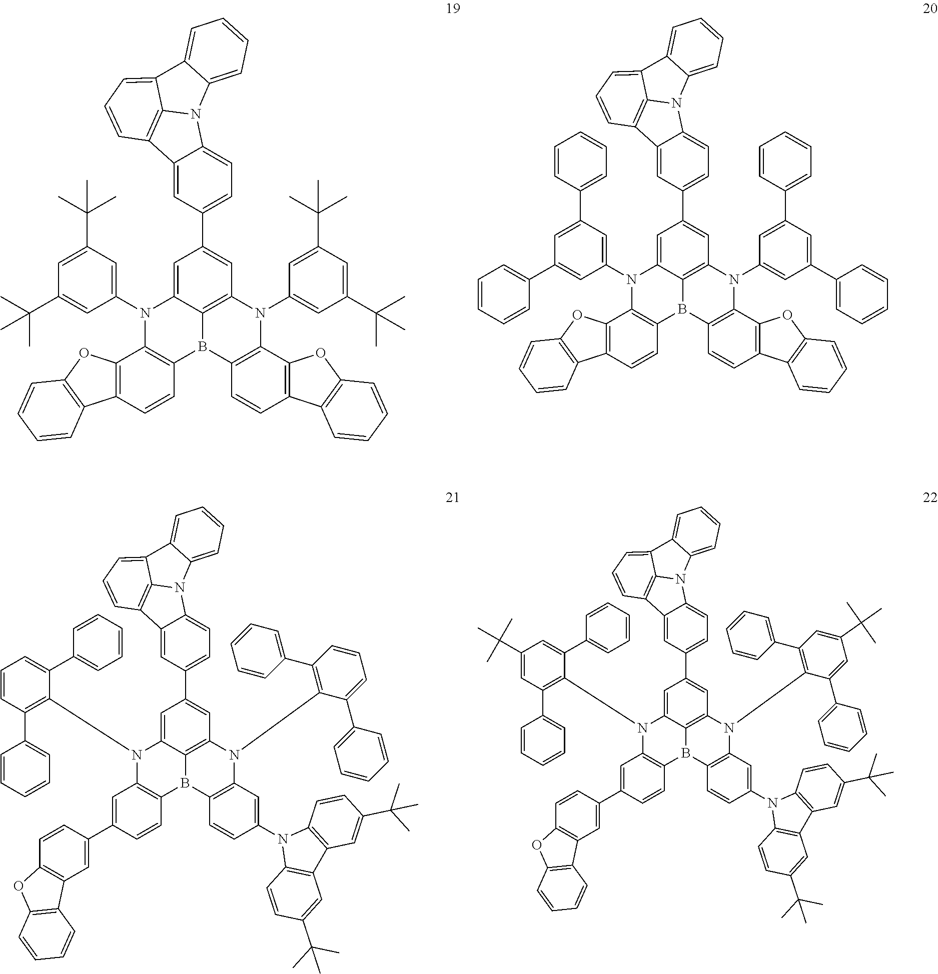 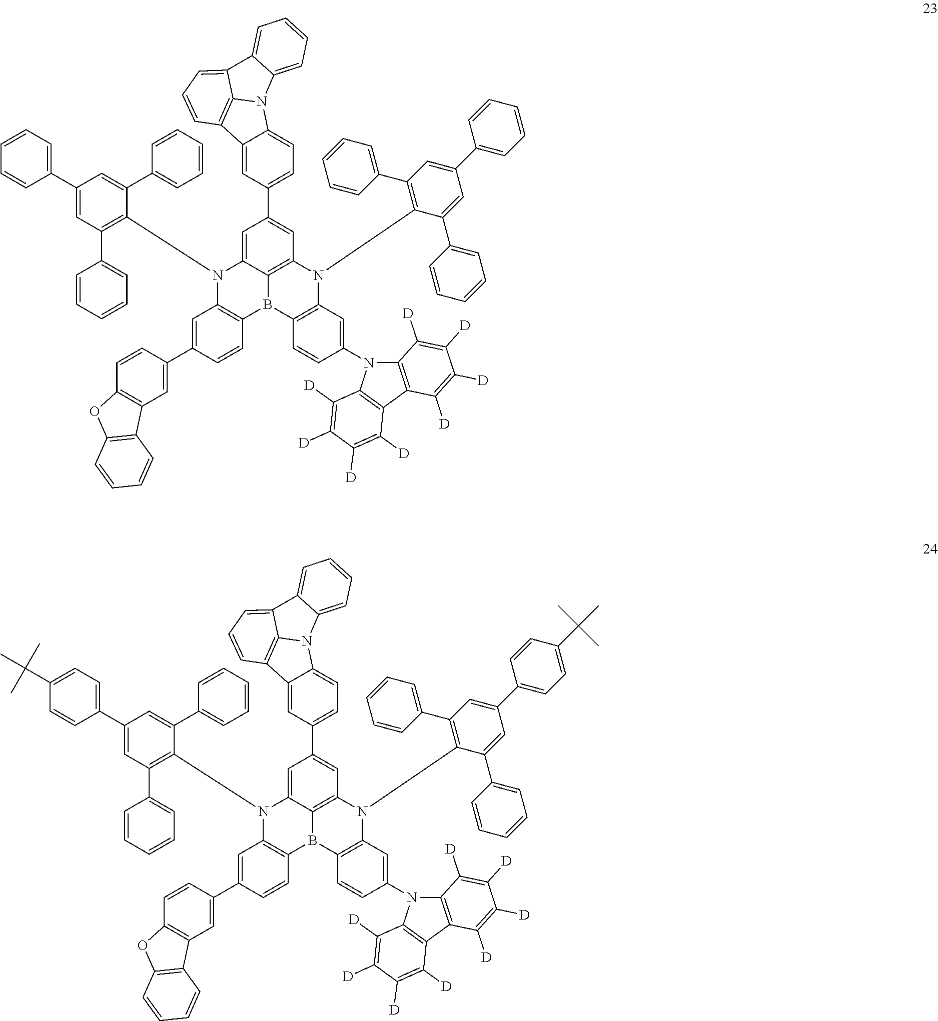 [SMILES: [2H]c1c([2H])c([2H])c2c(c1[2H])c1c([2H])c([2H])c([2H])c([2H])c1n2-c1ccc2c(c1)N(c1c(-c3ccccc3)cc(-c3ccccc3)cc1-c1ccccc1)c1cc(-c3ccc4c(c3)c3cccc5c6ccccc6n4c53)cc3c1B2c1ccc(-c2ccc4oc5ccccc5c4c2)cc1N3c1c(-c2ccccc2)cc(-c2ccccc2)cc1-c1ccccc1 ; [2H]c1c([2H])c([2H])c2c(c1[2H])c1c([2H])c([2H])c([2H])c([2H])c1n2-c1ccc2c(c1)N(c1c(-c3ccccc3)cc(-c3ccc(C(C)(C)C)cc3)cc1-c1ccccc1)c1cc(-c3ccc4c(c3)c3cccc5c6ccccc6n4c53)cc3c1B2c1ccc(-c2ccc4oc5ccccc5c4c2)cc1N3c1c(-c2ccccc2)cc(-c2ccc(C(C)(C)C)cc2)cc1-c1ccccc1] 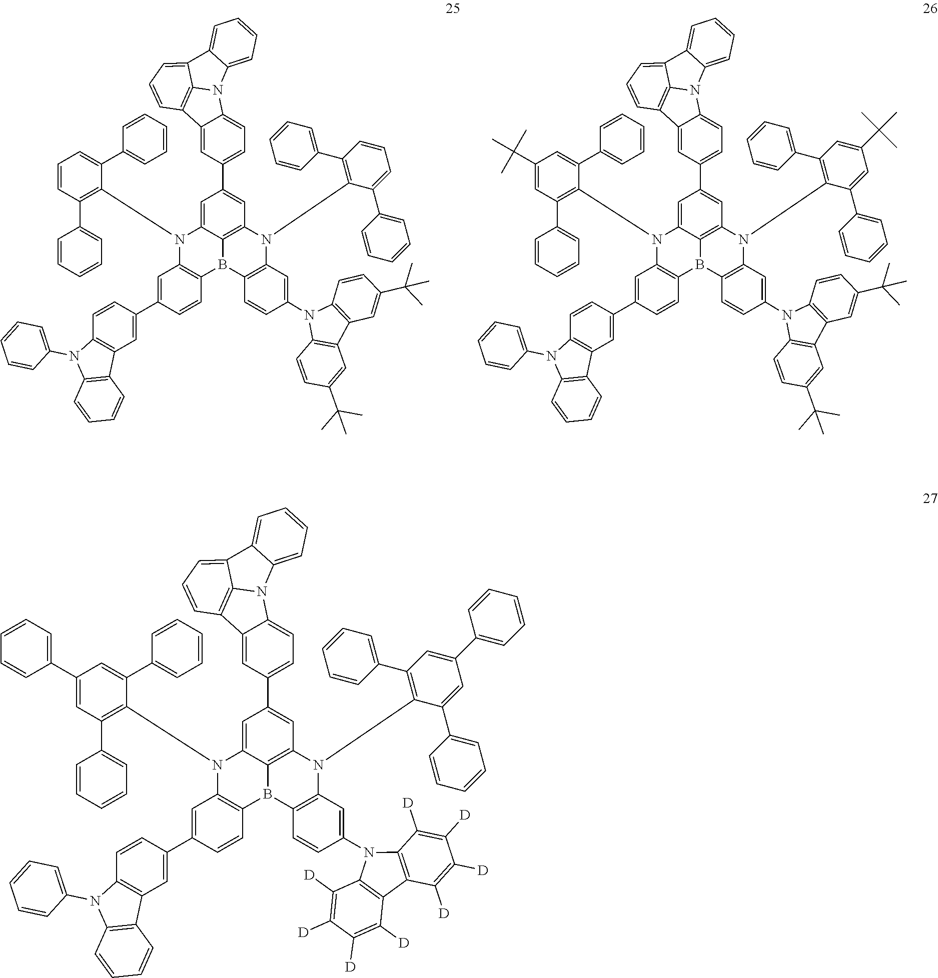 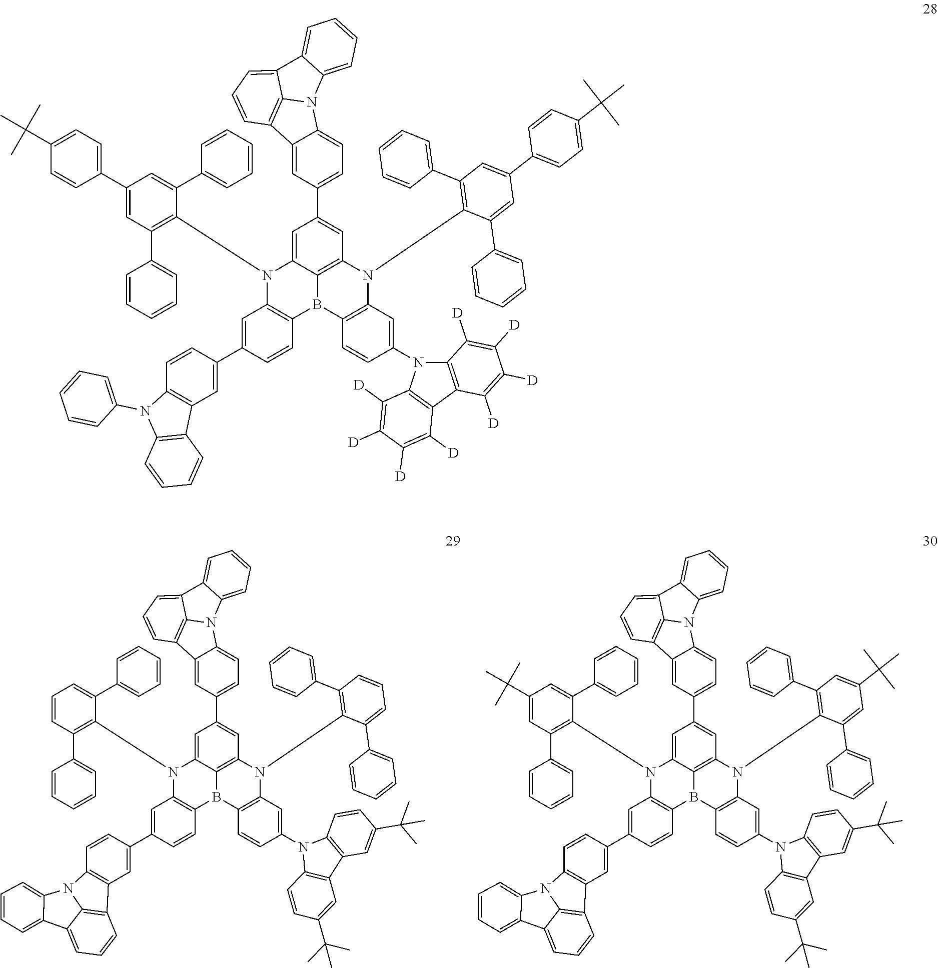 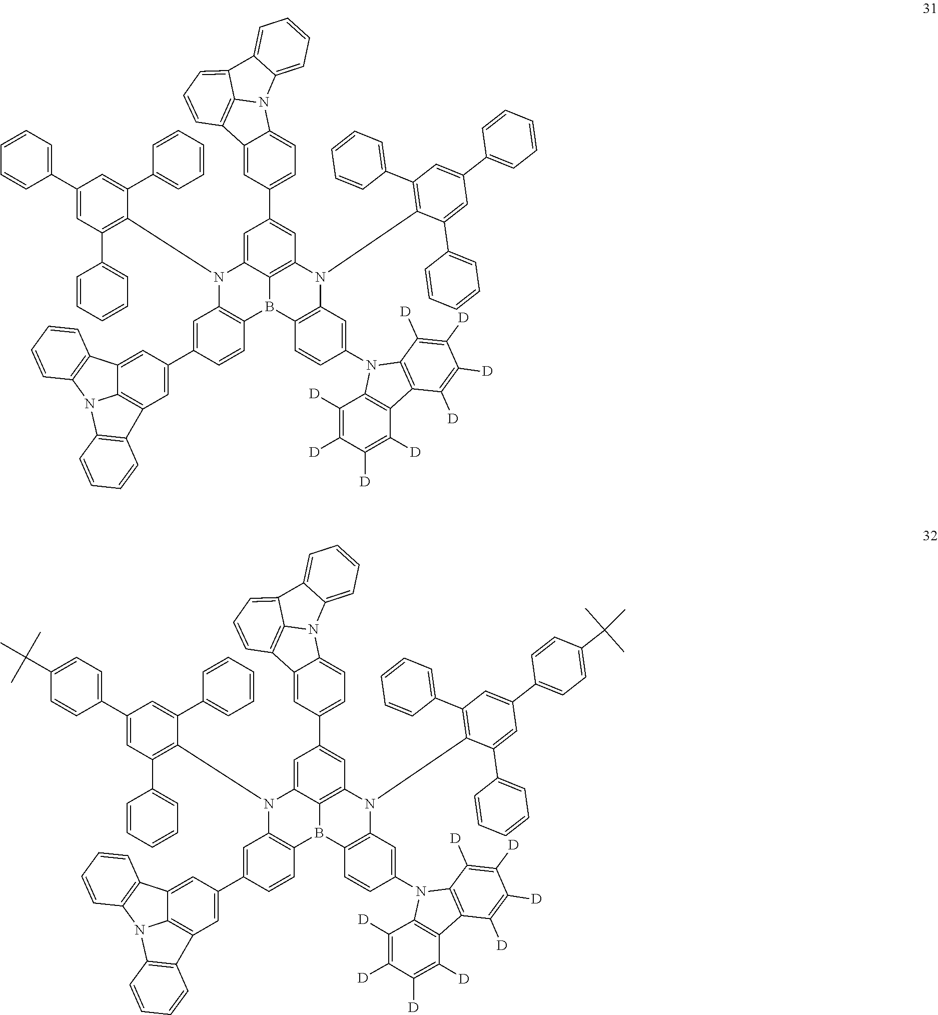 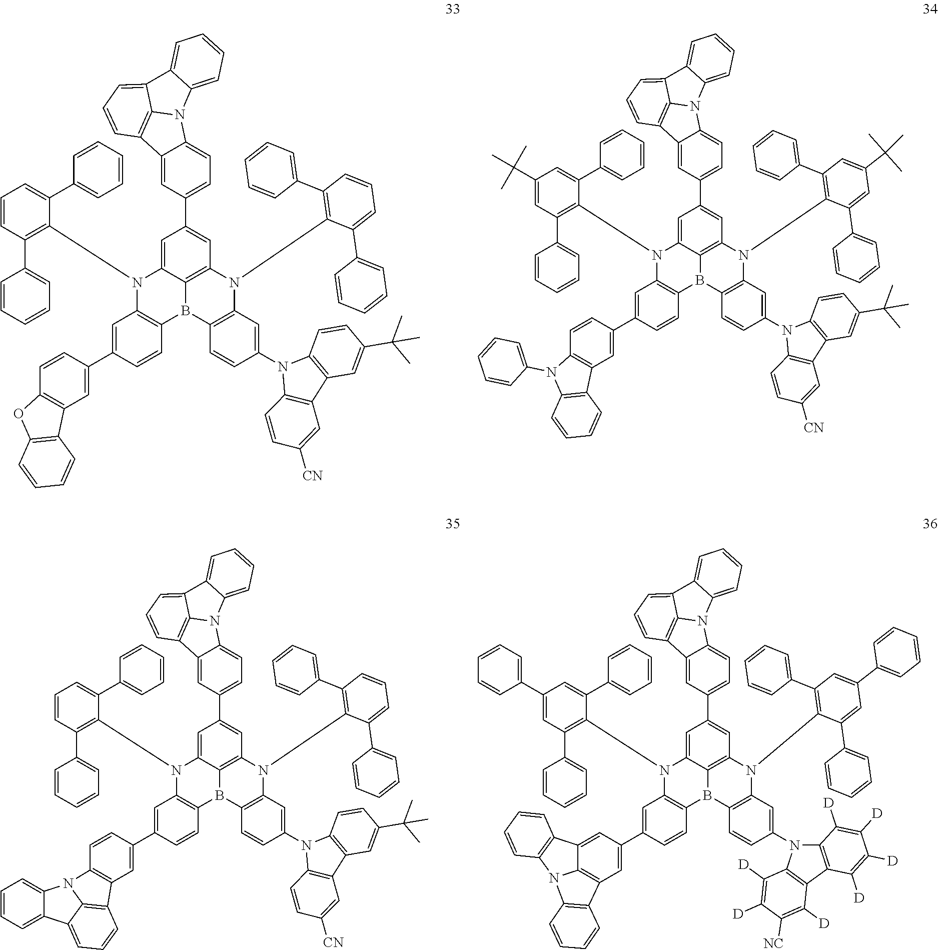 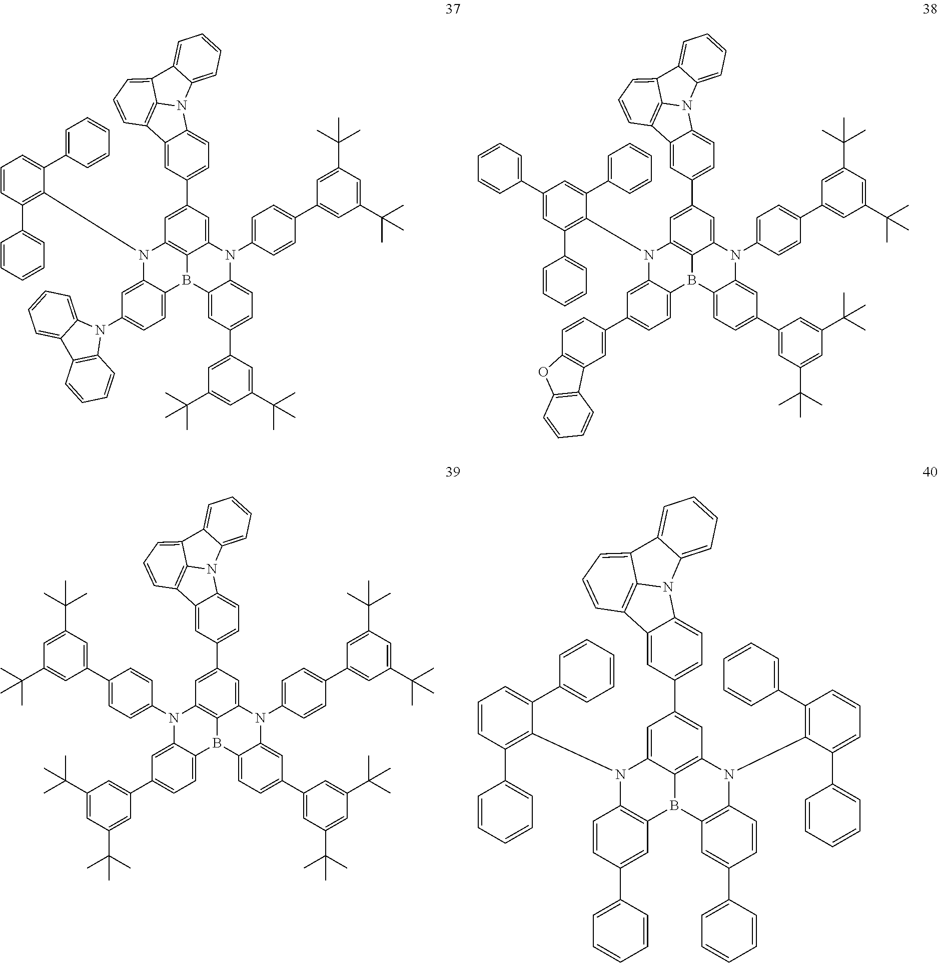 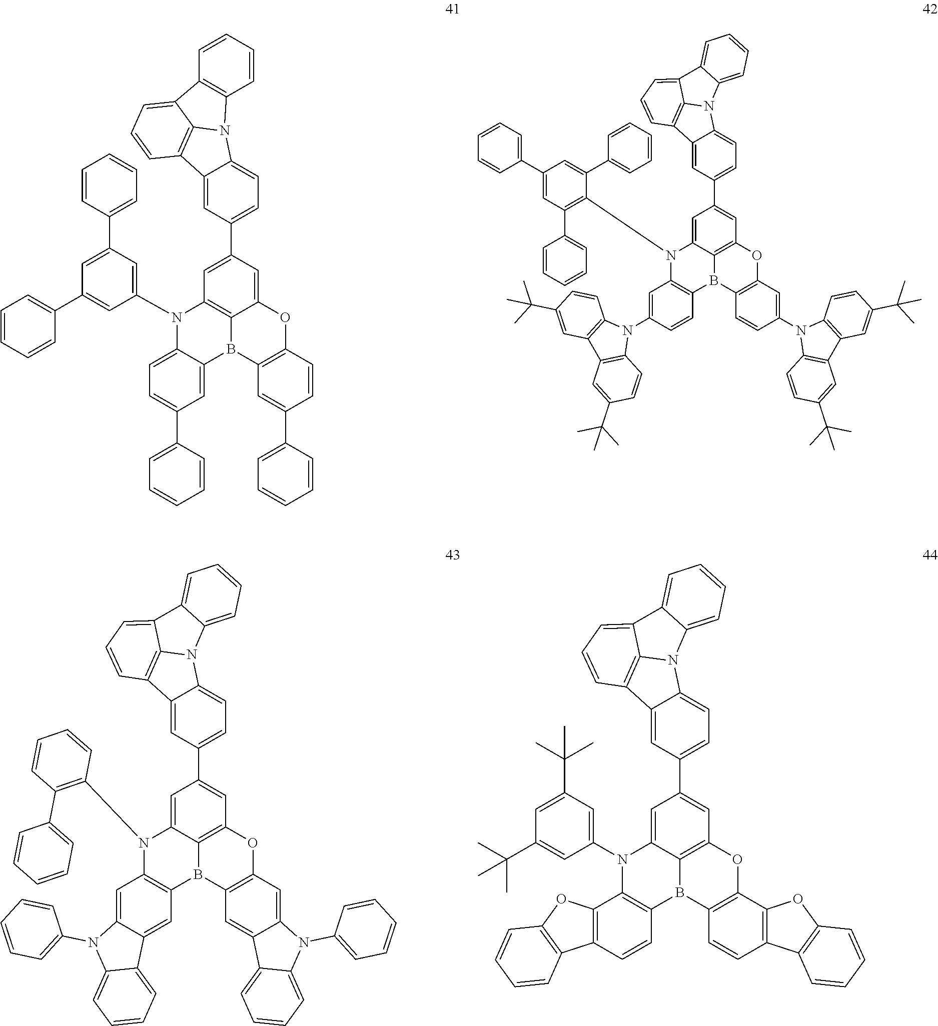 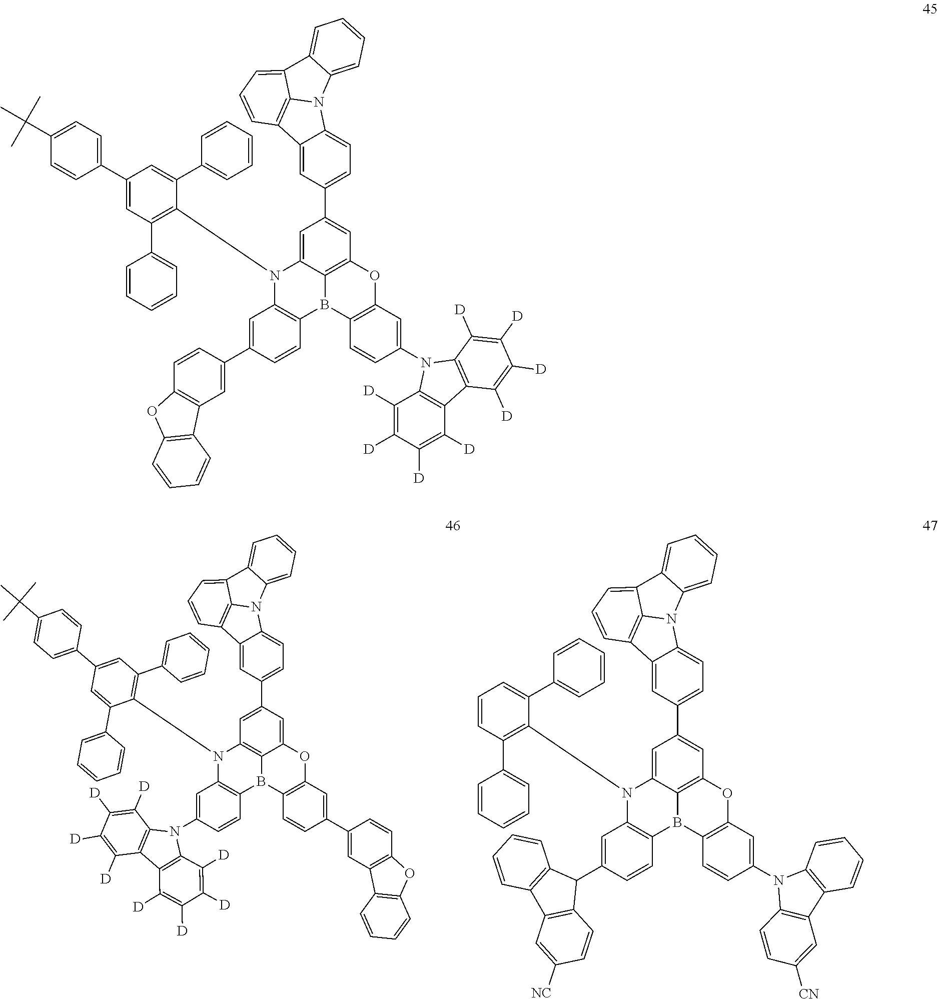 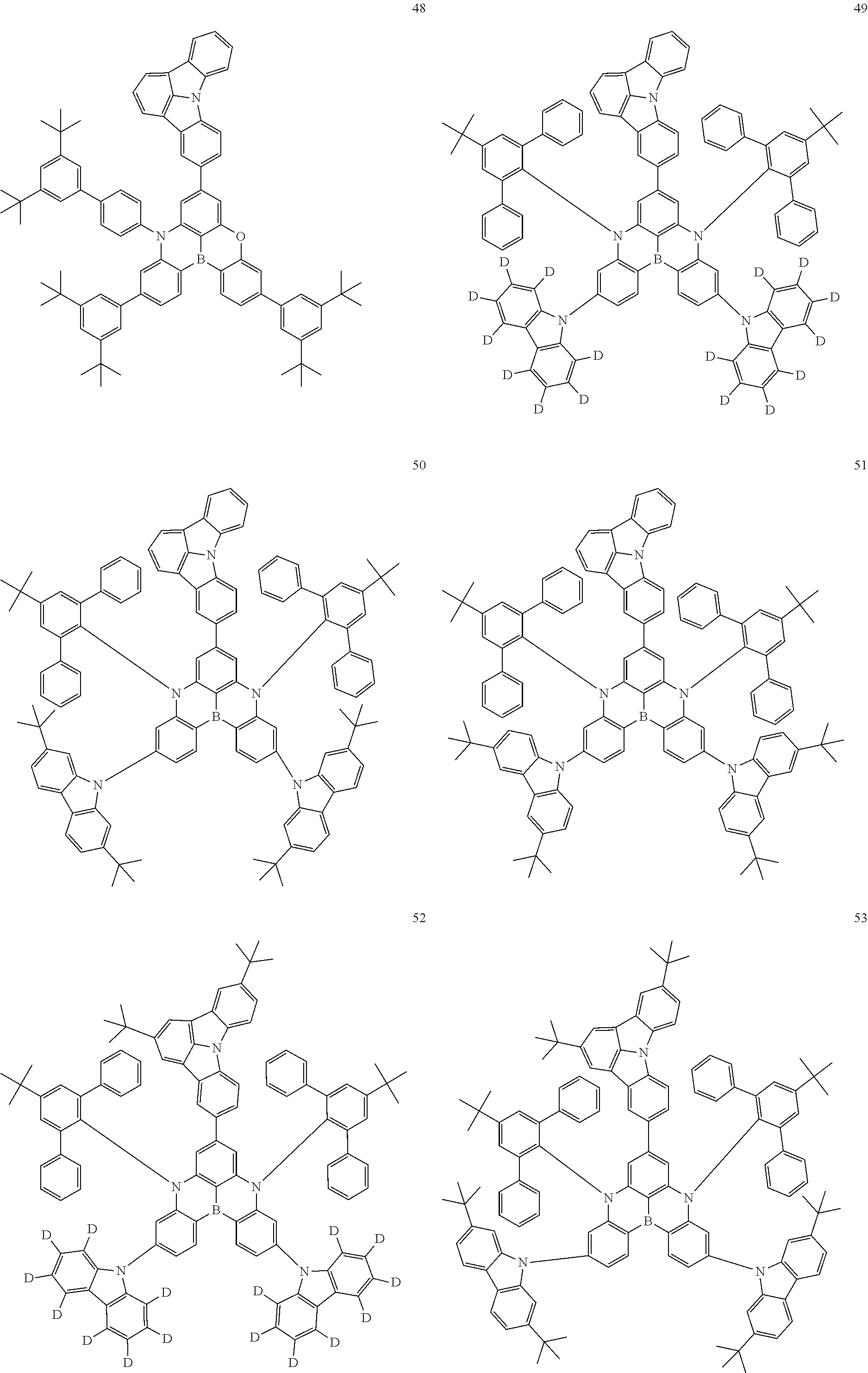 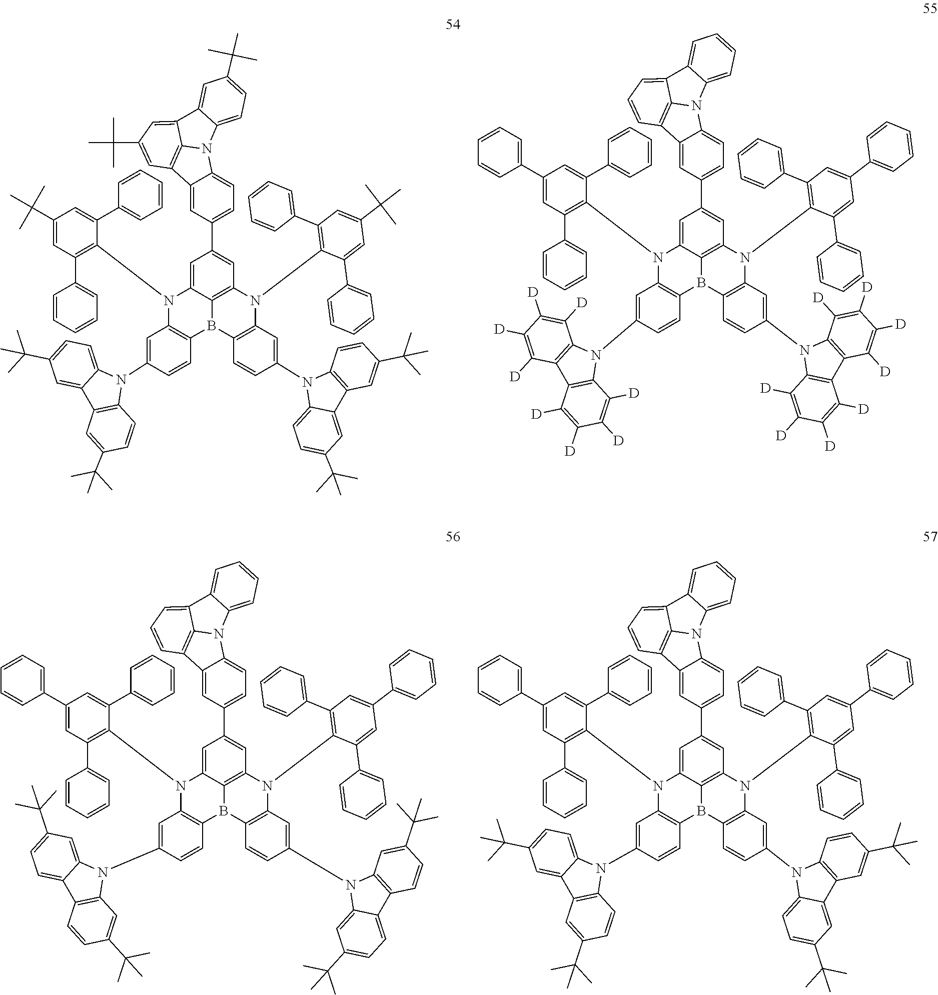 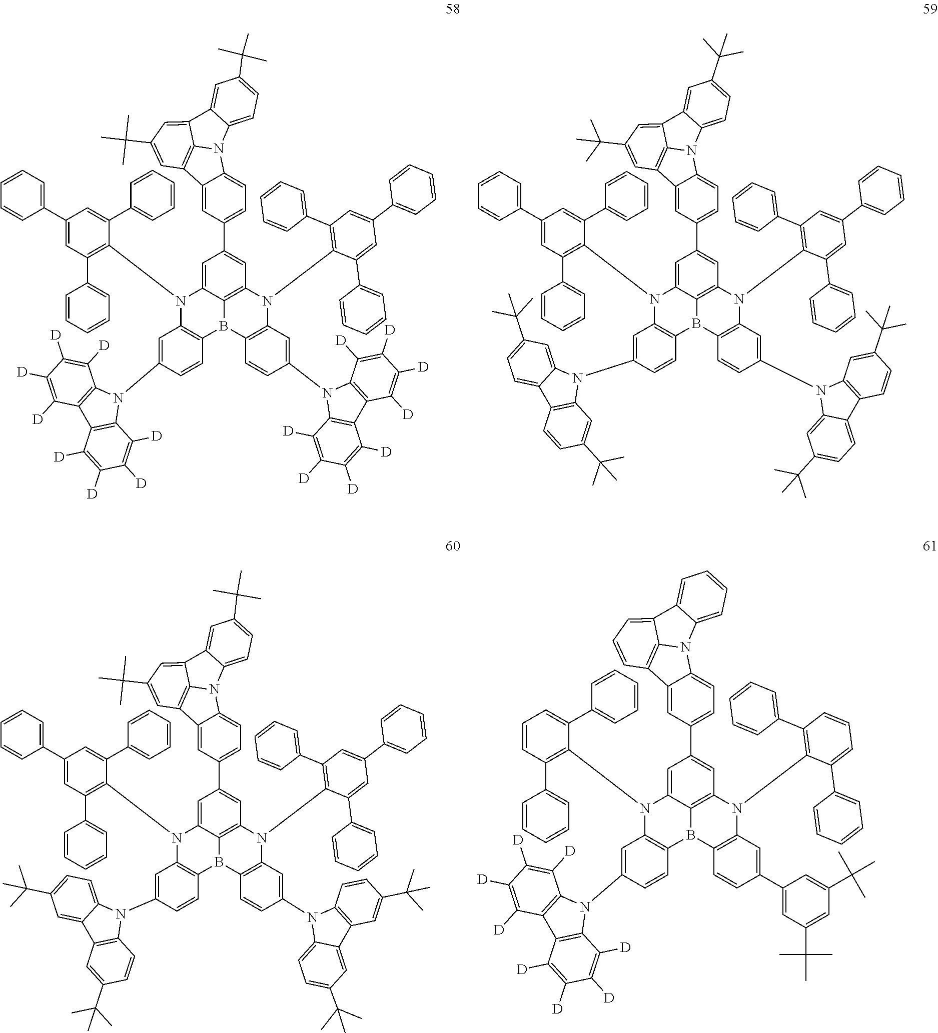 [chemical structure]
